SEQUENCE LISTING (102 sequences)

SEQ ID NO: 1 - DNA, 534 bp, Pseudomonas aeruginosa
atgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaa

SEQ ID NO: 2 - DNA, 1221 bp, Escherichia coli
gaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagc

SEQ ID NO: 3 - DNA, 2559 bp, Pseudomonas aeruginosa
taaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctcgcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggcagcgcaggccgcccagcggcaacgcatcgaggcagaagcacgccccggtgaatcgtggcaagcagccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcaggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctat

SEQ ID NO: 4 - DNA, 720 bp, Agrobacterium tumefaciens
atgcttaagagatcggggtcgctttctcttgccttgatggtctccttctgttcgtcgagccttgccacgccactctcatctgctgagtttgaccatgttgctcgcaagtgtgccccatcagttgcgacatctacgcttgcggcgatagctaaggtggagagtcgctttgatcctttagcgattcatgacaacacgaccggcgaaacgcttcactggcaagatcacagccaagcaacccaagtcgtcaggcaccgtctcgatgcacggcattcgctggatgttggcctcatgcaaataaactctcgaaatttttctatgctcggtctgacacctgacggtgcgctccaggcgtgcacatcattatctgccgctgcaaacatgctgaaaagtcgttatgcaggcggcgaaacgattgacgagaagcaatttgcgcttcgtcgggcgatctccgcttacaacaccggtaatttcatcggcggttttgcaaacggctacgtgcgaaaagttgaaacagctgctcaatcgctggtgcccgcgttaatcgagcctccaaaagacgatcacgaggcgctaaaatccgaagagacgtgggatgtttgggggtcatatcagcgccgctcgcaggaggatggcgctggcggtttaatcgctccgccaccgccacaccaggacaacggcaaatccgcagacgacaatcaagtcttattcgacttatactaa

SEQ ID NO: 5 - DNA, 365 bp, Agrobacterium tumefaciens
atgcgatgctttgagagataccgtttacatctaaatcgcctctcgctctcgaatgcgatgatgcgcgtgatatcgagctgcgccccaagcttgtgcggtgcaattgcatggagcatttcctcatccggacccgccgcagcgcaatctgcgggtggcggcactgaccccgccacaatggttaacaatatatgcacgtttatccttggtccgttcggccagtcactcgctgttctcggcattgtcgctatcgggatctcctggatgttcgggcgggcttcgcttgggctggttgccggcgtcgtcggcggcattgttatcatgtttggggcgagcttcctcggccaaacgctcactggcggtagttg

SEQ ID NO: 6 - DNA, 326 bp, Agrobacterium tumefaciens
atggctgatcgtttggaagaatcgaccctttacctcgcagccacacggcccgcattgtttcttggggtgccactgacattggcagggttattcatgatgttcgccggctttgtcatcgttatcgttcagaacccgctctacgaagtcgttctcgtgccgttatggtttgcagcccggctcatcgtggagcgagactacaatgcggcgagcgtcgtcctgctatttttgcggaccgcgggaagaagcattgatagtgcagtttgggggggcgctactgttagcccaaatccaattagggttcccccacgagggagaggaatggtgtg

SEQ ID NO: 7 - DNA, 2370 bp, Agrobacterium tumefaciens
atgctcggcgcgagtggaacgaccgaaagatccggtgagatctatctcccttatattggccacctcagcgaccatatcgtccttcttgaagacggatcgatcatgaccattgcgagaattgatggcgttgcattcgagcttgaggaaactgaaatgcgcaatgcgcgttgtcgtgcgttcaacacgctgttgcgcaatatcgctgatgatcatgtgtcaatatatgctcacctcgtacgtcatgccgacgtgccatcatcggcgccgcgacacttccgtagtgttttcgccgctagcctgaacgaagcttttgaacagcgcgtgctctccggccaactcctccgcaatgaacacttccttacgttgattgtctacccacaggcggctttagggaaggtaaagaggaggttcaccaagctaagcggaaaaagggaaaacgatctcacgggccagatcaggaacatggaagatctttggcatgttgtcgctggctctcttaaagcgtatggcctgcatcgtcttggcatccgcgagaagcagggtgtgctcttcaccgaaattggcgaagcgctacggttgatcatgactggtcggttcacaccggttccggtcgtcagcggctcactcggcgcttcgatttataccgacagagtcatttgcggcaagcgaggactcgagatcagaacgccaaaagacagttacgttggatccatctattcgtttcgcgaataccctgcaaaaacacggccgggcatgctcaacgcgctgctatccctcgattttccacttgttctcacgcagagtttttcgttcctgactcgcccgcaagcgcacgcgaaacttagcctcaaatcgagccagatgctgagttccggcgataaagccgtgactcaaatcggcaaattatccgaggctgaggacgcacttgcgagcaacgaattcgttatgggctcacatcatttgagcctttgcgtctatgcagacgatctcaatagtcttggggacaggggcgcgcgggctcggacacgaatggcggatgcaggtgccgtggttgtccaagaaggtattggtatggaagcggcctattggtcccaattgccggggaattttaagtggcgcacacgccctggcgcaatcacttcacgcaatttcgcagggtttgtctctttcgaaaactttccagagggcgccagctcaggccactggggcaacgcgattgcccgatttcgtaccaatggcggaacgcctttcgactatatcccgcatgagcacgatgttggcatgacggcaatattcgggcctatcgggaggggtaagacgacgctcatgatgtttgttctagccatgctcgaacagagcatggtcgaccgtgcaggtacggtcgtgttctttgacaaggaccggggtggcgaattgctggttcgcgccacaggaggaacatatttggcacttcacagaggcacacccagcgggttggcgccgttgcgtggcctagaaaacacagcagcctcacacgattttctgcgcgaatggatcgtggctctcatcgagagtgatggtcggggtgggatttctccggaagagaaccgccgtctggtccgtggtatccatcgtcagctctcgtttgatccacaaatgcgttcaatcgcggggttacgtgaatttttgttgcatgggcccgccgaaggcgcaggagcgcggctccaacgctggtgccggggccatgcgcttggctgggcatttgacggcgaagttgacgaagtaaagttagatccgtcgattaccggcttcgacatgacgcatcttctcgaatacgaggaagtatgcgctcccgctgcagcatatctcctgcatcggattggagccatgatcgacggccgccgttttgtgatgagctgcgatgagtttcgcgcctatttgttaaaccctaaattttcgactgtcgtcgacaaattcctcctgaccgttcgaaaaaacaacgggatgctaatactggcaacgcagcaaccagagcatgttctggaatcgccgctaggagccagcttggttgcgcaatgtatgacgaagattttctatccatcaccaaccgcagatcgatcggcttatgtcgatggactgaaatgtaccgaaaaggaatttcaggcgatccgtgaagacatgacggtcggcagccgtaagtttcttcttaaacgagaaagtggaagcgtcatctgcgaatttgatctgcgggatatgcgtgaatatgtcgccgtgctttcggggcgtgccaacacggtgcgctttgcaactcgactacgcgaggcacaagaaggcaactcatctggctggctcagcgaattcatggcccgtcaccacgaggcagaagattga

SEQ ID NO: 8 - DNA, 663 bp, Agrobacterium tumefaciens
atgaagacgacgcaacttattgcaacagttttgacctgcagctttctatatattcagcccgcgcgggcgcagtttgttgttagcgacccggcaacggaggctgagacgctcgcgactgcgctcgcgactgcggagaatctcactcagactatagcgatggttacgatgttgacgtcggcctacggcgttactggactactgacttcgctcaaccagaaaaatcagtatccttcgacgaaggacctagacaatgaaatgttttcgccgcgaatgccaatgtcgaccacggcacgtgcgatcaccagcgatacagatcgtgcagtcgtgggtagtgatgctgaagcggacctgttgcgatcgcagatcaccggttccgcaaacagcgctggcattgcggctgacaatctggaaacgatggacaaacgcttgacggcgaatgctgatacgtctgctcagctttcccgatctcgcaatatcatgcaggcaaccgtgaccaatggtttgcttctcaagcagatccatgacgcaatgattcaaaatgtacaggcgacaagcctattaacgatgactaccgcgcaggccggccttcacgaggcggaagaggcggccgctcaacgcaaggagcatcaaaagaccgctgtcatctttggtgccctcccctaa

SEQ ID NO: 9 - DNA, 888 bp, Agrobacterium tumefaciens
atgaatttcacgattccggcgccgtttacggccattcatacgatcttcgatgtagccttcacgacaggcttggactcgatgcttgagactatccaggaggcggtgagtgcgccattgatcgcctgtgtcactctttggattattgttcagggtattttagtcatacgcggcgaagtcgatacccgtagcggtatcactcgggtgatcacggtcaccatcgttgttgctctaattgttgggcaggctaactaccaagactatgtggtttccatcttcgaaaagacggtcccaaactttgttcagcagtttagtgtaaccggcttgcctctgcagactgttccggcacagttggatacaatgttcgccgtgacccaggccgtttttcagaaaatcgcatccgaaatcggtccgatgaacgaccaggacatccttgctttccaaggggcacagtgggtcctttacggcacgctctggtctgccttcggagtttacgacgccgttggaattctcacgaaagtgcttctcgcgatcgggcctctgatcctcgtcggatatatttttgatcgcacgcgggacatcgcagctaagtggatcgggcaacttatcacctacggtctcttgcttctcctcttaaacctcgtggcaacgatcgtcatcctaaccgaagcgactgcgctcacccttatgcttggtgtaatcaccttcgccggtacgaccgcggccaagatcattggtctttacgaactcgatatgttttttctgacaggggatgcgctcattgtcgctttgccggcgatcgccggcaacattggaggcagttactggagcggcgcaacccaatctgccagcagcttgtaccgtcgcttcgctcaggttgagcgaggctag

SEQ ID NO: 10 - DNA, 154 bp, Agrobacterium tumefaciens
atgaagtattgcctgctgtgcctagttgtcgctttgagcggctgccagacaaacgacacattagcgagctgcaaaggcccgatcttcccgctgaatgtggggcgatggcagcctactccgtcagatcttcagctcggcaattcgggtggacgct

SEQ ID NO: 11 - DNA, 710 bp, Agrobacterium tumefaciens
atgacggggcctgaatatgccatgctagtggcgcgcgaaagccttgccgagcactataaggaagtagaagcctttcaaaccgcgcgagcgaaatcggcgcgacgtctctccaaactcattgcagctgtcgcagctatcgcgattttgggaaatgttgctcaagcgttcgctatagccacaatggtgccgttgagcaggcttgtgcccgtatatctatggatacggccggacggcaccgttgacagcgaggtgtctgtctcgcgattgcctgcaactcaagaggaggccgtcgttaacgcctcattgtgggagtacgttcgcctgcgcgagagttatgatgccgacaccgctcagtacgcctacgacctggtatcgaacttcagtgccccaacagtgcgccaggattaccagcaattcttcaactatcccaatcccagttcgcctcaagtcattcttggcaaacgcggcagggtggaggtcgagcacatcgcttcaaatgatgtaactccaagcacgcagcaaattcgctataaaaggaccctcgtcgttgacggcaaaatgcctgtggtgagtacgtggaccgcgacagttcgctacgaaaaggtgaccagcttgcccggcagattgagactaaccaacccggcaggtctggttgtcacctcctatcagacatcggaagataccgtttcaaacgtaggccacagcgaacc

SEQ ID NO: 12 - DNA, 878 bp, Agrobacterium tumefaciens
atgatcagaaaagcacttttcattttagcatgtttatttgccgctgcgactggtgcggaggctgaagacactccaatggcgggcaagctagatccgcgcatgcgttatttggcttacaatcccgatcaagtggtgcgcctctcgacggcggttggagctactttggtcgtaacattcgccacgaacgaaacggtgacagcggttgccgtttcaaatagcaaagatctagcagccctaccgcggggaaattatctatttttcaaggcaagccaggtcctcacgcctcagccagtaatcgtgctaaccgcaagcgactccgggatgcgccgttatgttttcagtataagttccaagactctgtcccacctcgataaagagcagcccgatctctattacagcgtccaattcgcctaccccgccgacgatgcggcggctcggcgaagggaggcacaacagaaggctgttgtggacagactacacgcggaagcacaatatcaacggaaagctgagaatttattggatcagcctgtcacagcccttggtgcggcggacagtaattggcactacgtcgcccaaggcgatcgttcgctgttgccactcgaagtcttcgacaatggatttacgacggtattccactttccgggcaatgtacgcataccctccatctacaccatcaatcctgatggcaaggaagctgttgccaactattcagttaaagggagcgatgtcgagatttcttcggtttcccgaggttggcgtctgagggatggccacacagtactatgtatctggaacaccgcttacgatcccgttggccaaaggccgcaaacgggcacggtgaggcccgatgtgaaacgcgtcctgaagggggcgaaggg

SEQ ID NO: 13 - DNA, 1134 bp, Agrobacterium tumefaciens
atgaataacgatagtcagcaagcggcacatgaggttgatgcatctggatccctggtctccgacaaacatcgccggcgtctttcggggtctcagaaattgatcgtcggaggtgtcgttctcgcgttatcattaagcctcatttggctaggtgggcgccaaaagaaggtgaatgagaacgcatcgccgtcaactttgatcgcaacaaacaccaagccatttcatccagctccgattgaggtgccgccggatcctccagcggttcaagaggctgttcagcctgctgctcctctaccgccgaggggcgaaccggagcggcatgagccacggccggaagaaacaccgatttttgcatatagcagcggcgatcaaggggtcagcaaacgcgccattcagggcgacacgggccgaagacaagaaggcaagcgtgacgacaactccttgccgaatggcgaagtgtccggcgagaacgatttgtcgatacgtatgaaacccaccgagctgcagcccagcagcgccacgctcttgccgcaccccgattttatggtaacgcaagggacaataattccgtgcatcttgcaaaccgcaatcgacacaaatttggcaggctatgtaaagtgtgtcttgcctcaggatattcgtggaacaacgaacaatatcgtgcttcttgatcgtggcaccaccgttgttggcgaaatacagcgtggcttgcaacagggagatgggcgcgtttttgtgttgtgggatcgcgccgagacacctgaccatgcgatgatctcgttaacatcgccaagcgcggacgaactcggtcgctcaggattgccgggctcggtcgacagccacttctggcagcgttttagcggagctatgctcttgagtgttgttcaaggcgccttccaggcagctagcacctacgccggcagctcgggtggcgggatgagcttcaacagctttcaaaataacggtgagcagacaactgagacagcccttaaggcaaccatcaacataccgccaaccctgaagaagaatcagggtgacaccgtttccattttcgtagcacgggacctcgatttctttggtgtttaccagctccgcctgactggcggcgccacgcgggggaggaaccgccgctcttaa

SEQ ID NO: 14 - DNA, 1032 bp, Agrobacterium tumefaciens
atggaagtggatccgcaactacgctttcttctgaagccgattttggaatggctcgatgacccgaagactgaagaaattgcgatcaatcgacctggagaggcatttgtgcgccaagccggcatttttaccaagatgcctttgcccgtctcttatgatgatcttgaagatatcgctattttagcgggcgcgctgagaaagcaggatgtcggaccacgtaaccccctctgcgccactgaacttcctggtggtgaacgactacaaatctgtctgccgccgaccgttccctcgggcaccgtcagcttgaccattcgacggccaagctcgcgtgtttctggtcttaaagaagtctcctcccgttatgatgcttcgaggtggaaccagtggcagacacgaaggaaacgccaaaatcaggatgatgaagctatccttcagcattttgacaacggggatttggaagcgtttctgcacgcatgcgtcgtcagccgactgacgatgttgctatgtggccctaccggaagcggcaagacaacaatgagcaagaccttgatcagcgccatccccccccaggaaaggctaatcaccatagaagatacgctcgaactcgtcattccacacgataatcatgttagactactctactccaagaacggtgctgggctgggtgctgtgagcgccgagcacttgctccaagcaagtctgcgtatgcggccggaccggatattgcttggcgagatgcgcgacgatgcagcatgggcttatctgagtgaagtcgtctcgggacatccgggatcgatttcaacaatacacggcgcgaatccaatccaaggattcaagaaactgttttcccttgtcaaaagtagcgcccaaggtgctagcttggaagatcgcacactgattgacatgctctctacggcgatcgatgtcatcattccattccgtgcctatgaggacgtttatgaagtaggcgagatctggctcgcggcggacgcacgacgccggggcgagaccataggcgatctccttaatcaatag

SEQ ID NO: 15 - DNA, 804 bp, Agrobacterium tumefaciens
atgattgtacatccttcacgtgaaaatttctcaagcgctgtgaacaagggttcagattttagattgagaggtgagccgttgaaacacgttcttcttatcgatgacgatgtcgctatgcggcatcttattatcgaataccttacgatccacgccttcaaagtgaccgcggtagccgacagcacccagttcactagagtactctcttccgcgacggtcgatgtcgtggttgttgatctaaatttaggtcgtgaagatgggcttgagatcgttcgaaatctggcggcaaagtctgatattccaatcataattatcagtggcgaccgccttgaggagacggataaagttgttgcactcgagctaggagcaagtgattttatcgctaagccgtttagtacgagagagtttcttgcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttacgacaggagtatagatgttctcattttgcggctgcgccgcaaacttgaggcggatccgtcaagccctcaactgataaaaacagcaagaggtgccggttatttctttgacgcggacgtgcaggtttcgcacggggggacgatggcagcctga

SEQ ID NO: 16 - DNA, 696 bp, Agrobacterium tumefaciens
atgcaacttttgacgttttgttctttcaaagggggtgctggcaaaaccaccgcactcatgggcctttgcgctgctttggcaaatgacggtaaacgagtggccctctttgatgccgacgaaaaccggcctctgacgcgatggagagaaaacgccttacaaagcagtacctgggatcctcgctgtgaagtctattccgccgacgaaatgccccttcttgaagcagcctatgaaaatgccgagctcgaaggatttgattatgcgttggccgatacgcgtggcggctcgagcgagctcaacaacacaatcatcgctagctcaaacctgcttctgatccccaccatgctaacgccgctcgacatcgatgaggcactatctacctaccgctacgtcatcgagctgctgttgagtgaaaatttggcaattcctacagctgttttgcgccaacgcgtcccggtcggccgattgacaacatcgcaacgcaggatgtcagagacgctagagagccttccagttgtaccgtctcccatgcatgaaagagatgcatttgccgcgatgaaagaacgcggcatgttgcatcttacattactaaacacgggaactgatccgacgatgcgcctcatagagaggaatcttcggattgcgatggaggaagtcgtggtcatttcgaaactgatcagcaaaatcttggaggcttga

SEQ ID NO: 17 - DNA, 609 bp, Agrobacterium tumefaciens
atggcaattcgcaagcccgcattgtcggtcggcgaagcacggcggcttgctggtgctcgacccgagatccaccatcccaacccgacacttgttccccagaagctggacctccagcacttgcctgaaaaagccgacgagaaagaccagcaacgtgagcctctcgtcgccgatcacatttacagtcccgatcgacaacttaagctaactgtggatgcccttagtccacctccgtccccgaaaaagctccaggtttttctttcagcgcgaccgcccgcgcctcaagtgtcgaaaacatatgacaacctcgttcggcaatacagtccctcgaagtcgctacaaatgattttaaggcgcgcgttggacgatttcgaaagcatgctggcagatggatcatttcgcgtggccccgaaaagttatccgatcccttcaactacagaaaaatccgttctcgttcagacctcacgcatgttcccggttgcgttgctcgaggtcgctcgaagtcattttgatccgttggggttggagaccgctcgagctttcggccacaagctggctaccgccgcgctcgcgtcattctttgctggagagaagccatcgagcaattggtga

SEQ ID NO: 18 - DNA, 444 bp, Agrobacterium tumefaciens
atgtcaaaacacaccagagccacgtcgagtgagactaccatcaaccagcatcgatccctgaaagttgaagggttcaaggtcgtgagtgcccgtctgcgatcggccgagtatgaaaccttttcctatcaagcgcgcctgctgggactttcggatagtatggcaattcgcgttgcggtgcgccgcatcgggggctttctcgaaatagatgcagacacacgagaaaagatggaagccatacttcagtccatcggaatactctcaagcaatgtatccatgcttctatctgcctacgccgaagaccctcgatcggatctggaggctgtgcgagatgaacgtattgcttttggcgaggctttcgccgccctcgatggactactgcgctccattttgtccgtatcccggcgacggatcgacggtcgctcgttactgaaaggtgccttgtag

SEQ ID NO: 19 - DNA, 1275 bp, Agrobacterium tumefaciens
atgcccgatcgcgctcaagtaatcattcgcattgtgccaggaggtggaaccaagacccttcagcagataatcaatcagctggagtacctgtcccgaaagggaaagctggaactgcagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtaagcttccccgcaggtaccgaccaaaccgcagcttatgaagcaagccgggaatgggcagccgagatgtttgggtcaggatacgggggtggccgctataactatctgacagcctaccacgtcgaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaatatcgacgccttgagcggatgcaggctcaaaagattcaattcgaagatacagattttgatgagacctcgcctgaggaagatcgtcgggacctcagtcaatcgttcgatccatttcgatcggacgcatctgccggcgaaccggaccgtgcaacccgacatgacaaacaaccgcttgaaccgcacgcccgtttccaggagcccgccggctccagcatcaaagccgacgcacggatccgcgtaccattggagagcgagcggggtgcccaaccatccgcgtccaaaatccctgtaactgggcatttcgggattgagacttcgtatgtcgctgaagccagcgtgcccaaacaaagcggcaattccgatacttctcgcccggtgactgacgttgccatgcacacagtcgagcgccagcagcgatcaaaacgacgtcatgacgaggaggcaggtccgagcggagcaaaccgtaaaagattgaaggccgcgcaagttgattccgaggcaaatgtcggtgagcccgacggtcgcgatgacagcaacaaggcggctgatccggtgtctgcttccatccgtaccgagcaaccggaagcttctccaacgtgtccgcgtgaccgtcacgatggagaattgggagaacgcaaacgtgcaagaggtaatcgtcgcgacgatgggcgcggggggacctag

SEQ ID NO: 20 - DNA, 635 bp, Agrobacterium tumefaciens
atggcaaatggtcagttcacgatacgctctgctcgcccggcctccgtcggactgacaggcgaacggcgtggagccgcatccgcctctagctctgcactgtccaatgttcaaagagatgttagggataggctgattccaactagctcaccaagattaccaaatgcagccatattgcgtgattcctcgggaagagcgtcgactggtctgcggtacatggcggctactcttcattggtctgcgatcgcgccattatcgctaataaacagcaacgacctggctccggccgcttatgactttgagacgcgaaataacgcaagaaatgtgactgccaaagtcggcagggcagtccctgttcccaagcaaggcgggctcggcaaaacgctcgcacccgtaccccttagtacacgtatatcaagggtcaattccgaccgaagactgcccgctgacgcagaagaccgccctgaaacgcgcgacccccagaaaggacgtggcagtcatggtgcgacgccaaccttacatgaaaagattggaaccgcgtttgctcgaagattgcgaaagcatacgtactatattgtttgcagttgctgccagacccggagcgcgttgacgatgggtgcaaagatttcggtgaagtc

SEQ ID NO: 21 - DNA, 1959 bp, Agrobacterium tumefaciens
atgaactccagcaagacttcgccccagcgtatgaccctgagcatcgtatgttcgctggcagccggtttttgtgcggccagctgctatgtaacgttccgccggggcttcaacggcgaagcgatgatgacgttcgacgttttcgctttttggtatgagaccccgctttacttgggttatgccagcaccgtcttctggcgtggtttatctgttgtcatctttacctcgctgatcgttctttcaagtcagctcatcatatcgctgcgcaatcagaagcatcatgggacagctcgttgggcagaaattggcgaaatgcggcatgctggttatctgcagcgttacagtcgcatcaaggggccgatctttggaaagacatgtggtcccctttggttcggcagttatttgaccaatggcgaacagccacacagtcttgtcgtcgcgccaacgcgtgctggcaaaggcgtcggcatcgtcattccaacgctgttgaccttcaagggctcggtaatcgcccttgacgtcaagggagaattgtttgaactgacgtccagagcacgcaaagcgagcggcgacgcagttttcaagttctcccccctagatcctgagcggaagactcattgttacaatccggtcctggatattgccgcacttccgcccgaacgccagttcactgaaacacgccgtctagctgcgaaccttattacggctaagggaaagggagcagaaggctttattgacggcgcacgtgacctgttcgtcgcgggaatccttacctgcattgagcgtggcacaccaacgattggcgcggtatatgacctatttgcgcagcctggcgaaaagtataagctttttgcgcaactcgcggaggaaagcctaaacaaagaggctcagcgtatcttcgataatatggcgggcaacgacacgaaaattctgacatcgtacacctctgtgctgggcgacggtggactgaacctgtgggctgatccgcttatcaaagcagcgacaagccggtcagacttttccgtttacgatctccggaggaagaagacctgcatttatctttgtgtcagtcccaacgatctggaggtcttggcaccacttatgcgcctgatgtttcagcagctcgtgtcaatcttgcagagatcgctgccaggtgaagacgagtgccatgaagttttatttctcctcgacgaattcaaacacctgggcaagcttgaggccatagagaccgcgatcacaaccatcgccggttacagaggccgctttatgtttattattcaaagtctttcggccttgtcgggcacatacgatgacgcaggaaaacaaaactttctgagcaatactggcgtacaagtatttatggccacggctgatgacgaaactccaacctacatctcaaaagctatcggcgaatatacgtttaaagcgcgttcgacctcttacagtcaagccagaatgttcgaccacaacatccagatttctgatcaaggtgcagcccttttgcgccccgaacaagtgcgcctgctagacgatcagagtgaaatcgttctcatcaaagggcgacctccactcaaattacgaaaggtgcagtattattccgatcgtacgctgaaaggccttttcgaacgccagatgggctctctgcctgagcccgcacccttgatgctttccgactatagcaacgatcaagttcaataccacttggctccgatagcaaattttaatgaggatgctgcaccgcaaaacagaactgtggccgaggaccatggaagtgttaaagtcggtgctgatatccctgaacgcgtgatgggaataaatggtgacgaggaacaagccgatgcgggcgagataccgccggaatcggttgtgcctccagaattgacgctcgctctgaccgctcaacagcaattgttggaccagattattgcacttcagcaaagatcgaggtccgcaccggcatag

SEQ ID NO: 22 - DNA, 2511 bp, Agrobacterium tumefaciens
atgacaggaaagtcgaaagttcacataagaggttcggctgacgcgcttcctgacgttcctggcggaagtactaccgccccttttttaaccgaaccttctcgggatcaggttgatgcctcgtttgaggtccaaaccgactacagccagtctacttccgtgtcgtttacctatgatggtgttggacttggtcctgccgagcgtgcggcttacgagaactggtgcgaaccgggccggcccacttggaaagatcttataatcaaggcacgtgtcgatccgattgacgatgtgacctggctccgagatttagaagaggacaccccctcaaccttcagatacgaagggatgcctctgggcatcggggaacgacaggcctacgaaaattggcaagaggacgctcagccgacatgggaagaccttgttgtcagcgcacgcttgacggaacttggccgtccacacgggattaccggcgagtatacatccctcgcaggatcgaagaatacaagttcaatttcattgaagcgaaagcggagcaacttaattgatgatgagaattcatccggatcgttttcatatgacgggatgaagctcggggaagccgagcgttctgcatatggtgactgggccgaggcggagccacccacgtggaaagatttggtattgagggcacgcgtttcctcgatcaatgactctgcttggctttttgattcacaaacatcttcatcatcatttgaatacaacggtgttcccttgggcgagccggaacggcaggctctcagacaatggcaaggagacgctcagcctacctgggaagatctcgttgttaacgcgcgtatggcagaactttgccatgctggttggattgaaggtcaaaaaggttgctttgaagagcgcggggaggctctgcccgcgtcggaacgcggttcgcaacgccccattggtcaacggacagattcctccgattcttttgtgtatgatggcacaaggctcggagcacctgagcgaactgcttatgaacgctggagtaagagggaacgcccgacttgggaagatctcatcttagatgcacaccaggccaggactgaaagtgacgctgttacgacccaagcgattggtcagtcgtcctcaccggttttcttatatgaaggaaagtcgctcggagacagggaacgaaaggcttacgaaaaatggcggcagccagcccaaccgcgatggcaaaatcttgtagtgaacgctcgtctggcagaaatcgatccctcagcctggattgccgatgagcgcgatccgcttgatgatagcgacgcgcttggtcgcccgtcgtacacaagcttgacggatagatcagacgtccctttagacgatcaatcaatctatcgtcgttccgacctagtaagggagcaggtgccagaatcgtctcaaaggcaattcgcagcatgttcagaatctgaaacgaggcctgtgcaatggtttactgcttctgggtcagatgcaaacaatacggaaaatatcaccgccagcgatcccgtcgatcgcacgggtggagttaagcggctaggctccaaaagcgacagaaccgttacagcttctatccatgacgtgaattccagcacaaggcgactgttgcttaacgaatttggatcggaggctccgcgcccttcgccagaaaagactgttcgcttaagaagcgacaatattggcacctatgggagccggaaaaatgaacgagcgcggctcgcgaccgaaaccggtgcgtatgagtcggagcatattttcgggttcaaggctgtccacgatactgcgagagcgacgaaagagggccggcgtctcgaaaggcccatgcccgcctaccttgaggataaggggcttcatcgccaacatattggcaccgggagaggacggaccaaacttgtcgggcgcggatggccggatgacacaagctatcgctcggatcaaagggcaactctgtcggaccccgttgcgcgctcggaaggcgcgacggcctcaaatgggtatcaattgaaccaattgggttacgcgcaccaactcgctagcgatggcctgcaaagtgaatcgcccgatggtgttgccttgccaattcaagtggcaacaacgagctacaactatacagtgagccgcgatcctgtccttgttccgccggataaaaacgaagcccctcaattgctgcatcttggtccccgtggtcaaaccgaagctgttcttgcccgcgaaacagcattgactggaaaatggccgactctcgagcgtgagcagcaagtgtatcgcgagtttttggccttatatgacgtaaaaaaagatcttgaggccaaatcagtcggcgtaagacggaaaaaaaaagaagttatttctgcgttagaccgaactgcgcgcttgataagcacgtcgccttcgaaagctcgatccaaagcagagactgaaaaagccattgatgagctcgatgatcgacgagtttatgatccgcgtgatcgagctcaagacaaagcgtttaaacgctga

SEQ ID NO: 23 - DNA, 198 bp, Agrobacterium tumefaciens
atggccatcatcaagccgcatgtgaacaaaaataggacaacctcgccgatagagagaccggagtctctcatagaggaaatgagcggcagtcatccgccgagtggttttaccaacctggatctcgctatgatcgagctggaggactttgtccatcggtgcccgctcccagaagacaatcttgctggtcagaaggagtga

SEQ ID NO: 24 - DNA, 1650 bp, Agrobacterium tumefaciens
atggatccgtctagcaatgagaatgtctatgtgggtcgcggtcacaacatcgaaaatgatgatgacactgaccccaggcgttggaagaaggcgaatatcagttccaacaccatctccgatattcagatgacgaatggcgaagacgtacaatcagggagccctacccgaacggaagttgtaagcccacgtctggattatggatcggtcgactcctcctccagcctttattctggcagcgagcacggaaatcaagctgagattcaaaaagagctgtccgtcttgttctcgaacatgtctttgccaggcaacgatcggcgcccggacgaatacattctcgtgcatcaaacgggacaagatgcttttactggtattgccaaaggcaacctcgaccaaatgcccaccaaggcggaatttaacgcgtgctgccgtctctacagggacggagccggtaattactacccgccacctctcgcattcgacaagattagcgttccagagcaactggaggaaaaatgggggatgatggaggcgaaggaacgtaacaaactgcggtttcagtacaagttggacgtatggaatcatgcgcacgctgatatggggatcacgggcacagagatcttttatcaaacagataagaacataaagctcgaccggaattataaactaagacctgaagaccgatacgtacaaacagaaaaatacgggcgccgggaaattcaaaagcgatatcaacacgaactccaggctggttcgctgctgcccgatattatgatcaaaactccccaaaatgacatccacttcgtgtacaggtttgccggcgacaattacgccaacaaacagttcagcgagtttgaacacaccgtcaagcgcaggtatggcgacgagactgagatcaaattgaagtcaaagtcaggcattatgcatgactcgaaatatctggaatcctgggaacggggcagtgcggatattcgcttcgcggaattcgttggggaaaatagagctcacaatcggcagtttccaactgcgacagtaaatatgggacagcagccagacgggcagggcggtttgacccgcgaccgtcatgtgagcgttgacttcctaatgcaaagcgcacccaattcgccttgggcgcaagctttgaaaaagggagaactgtgggatcgcgttcagttgcttgctcgcgacggcaaccgctatctgtcgccgcccagattggaatattctgaccctgcacatttcaccgagttgatgaaccgggttggtttacccgcatcgatgggtcggcaaagccatgcggctagtatcaaattcgaaaagtttgacgcgcaggcagcggttattgtcttaaatggcccagagttacgtgacattcatgacttgtctcctgaaaaactgcaaaatttgtccaccaaagatgtcatcgtcgccgatcgcaatgagaatggtcagagaactggcacgtacaccagcgtcgcggaatatgagcgcttgcagttaaggctgccacccgatgcagcgggggtgcttggtgaagcaactgacaaatattcacgtgatttcgttcggccagagccggcgtcgcgtccaatcagtgacagccgcaggatatacgaaagtcgaccgcgtagccaaagcgtcaacagcttttga

SEQ ID NO: 25 - DNA, 2019 bp, Agrobacterium tumefaciens
atggtgaacactacaaagaaaagttttgcgaagtcgcttacggcagatatgcgccgttctgctcagcgcgttgtcgagcaaatgcgaaaagcattgattaccgaagaagaggcgctcaagcggcaagccagactggagagtcccgataggaagcgaaagtatgctgctgatatggcgatagtcgacaaactcgacgtagggtttcgaggcgaaataggctataaaattcttggaaataaccggcttcgagtagacaaccataaagaattaacgcgtgagcacggtagacttcgcaaaaccaaaacggttctgaagcgtaacccggtgacgcaggaagtctatttgggtttatatgaaaggaagtcctggttaagtgtcagcagccatttgtatgctgcggacggcacactccgcatgaagcacgtgaaatacaaagacggacgttttgaggaaaaatgggagcgcgacgaaaatggcgacctgatccgcacaaggtacgccaaccgtggcaggctctttcaacctgtatccgagaaaatgggcgcgccgtatcggagcggccctgacgaccggctctatcgcgatctaacccgtcgaaacggtttcagacgggagacattcgaacgggacgatcacggaaacctcgagcgtatcggcagcaaccatgtcggcttttccaagatttcagtgaaggcacccaatcgtcaaacctcccagacgaagattcaaaaacttggtggcgctttcaacaaatcttttaggtcccttctggacaaggagggcaatgaaatgggccgcgatattttgagccatcgacggctctataacaagcggtctgctgtctacgatgaagctaccggacaattgaagagtgccaagcataccttcggcaagatctacaggagcgaaaccgaatatctcagcgcgggcctcaagaaggtttcaaaaaagatactcggggtgacggtctaccggaaatttgcggcgctcagcgagcgagaatccgaggctgagagactgcgtagttttgaatccggtgcgcatcgccagatctggcaggagcgggcagcgattcccggttcgcccctcccggagactgatgacattcatttcgcacagcagtcgcacctagccaaagccaaccctgatcacgtcgaagctgacgtcacgcgtgtgacagatcaacatgttgatgttgctggacaaacatcatcgtctccccaacggaacttggaaggatggttagattctcaatcacgatacaagccagcaaacatgctgttgtcaaatccagaccttcaagcgaacggacctcgcccatacgaagggttagctcatctcaccctccggcgcgataatgaatctgacgggcacaaggagaatgatcagcggctgcgacatttctcccagccagagccgttggtgttaccgcatcccgggtcgccggaaataactaaggtgtttggctcgcggggagagccggcacacccgagtggaaatctgcacacggcggttggagaaacggcttgcgaaggaccggtgatgtcttcatcctcggacaatcatcagccagctccaggacagcaagaacttttaagtttccttcataatgcgccagccccagtttctgtggcaatacatgatgatcaagagcgacttgcgggggaggcgcccggcggctctttcagaggtagctcagggcgaacgagttcaatgtcggagagtatcttcgacgaagatgtacaagggcatttggtacgggattattcgatcaatactactaacgggtttattgacccgcaatcgttgttcggtgaaccggacttatcgagaggtccaaaatcggggccagaaattccatcggaagattaccatttgtcagcttcggaacaggaaaatttgctgaatcaattgcttagtgtgccactgccggttccttcaccgaagcccgaatgcgcgaggtctatgattttcgaaggttcacgttcaagagagcgttccacctccagagggttctaa

SEQ ID NO: 26 - DNA, 2490 bp, Agrobacterium tumefaciens
atgaacggaaggtattcaccgacgcggcaggattttaagacaggcgcgaagccttggtctatattagcccttatcgttgccgcaatgattttcgcgttcatggcggttgcgtcctggcaggacaatgggactacccaggcaatccttagccaaatacgatcgattaacgctgacagcgcctcactgcagcgcgatgtactccgcgctcacacgggcaccgtggcgaactaccgccccattatctccaggctgggagctctgcggaagaatctggaagatttgaagcaattatttaggcaatctcatattgtaagtgagagcaatgctgctcaactgctacgccggctagaagtgtctctaaattcggctgacgcggcggtcgccgcctttggtgcgcaaaatgtccgcctgcaagattcgctggccagtttcactcgtgctttaagcaatcttccaggaaaggcctcaggcgatcagactttagaaaaaccaacagaattggctagcatgatgctccaatttcttcggcaaccaagcccggctatttcattcgagatcagccttgaactagagaggctccaaaaacaacgcggtcttgatgaagctcccgtgcgcatactttcgcgtgaaggtcccattatcttatcgcttttgccacaggtgaacgatctggtgaacatgattcagacgtctgacaccgcagaaattgcggaaatgttgcagcgtgagtgtttggaggtctatagcttgaaaaatgtggaggagcggagcgcacgtatctttcttgggtccgcttcagtgggtctttgcctgtacatcatcaccttagtctataggctacgcaaaaaaaccgattggttagcgcggcgtttagattacgaagagctaatcaaagagatcggagtatgttttgaaggtgaggcggccactacgtcgtccgcgcaagctgcacttggtattattcagcgcttctttgatgccgatacgtgcgcgttagctctagtggaccatgaccgtaggtgggctgtcgaaacattcggtgcgaagcacccaaaacccgtgtgggacgacagggtgctacgcgaaatagtctctcgtaccaaagcgaacgaacgggcgacggtattccgcatcgtatcgacgcaaaaaatcgtacatttgcctcccgaaattccaggtctttcgatactactggctcacaaatccacagataaactaattgcggtttgttcactgggttaccaaagctatcgccctcgaccttgccaaggcgaaattcagcttcttgaactcgccaccgcctgcctctgtcactatatcgatgttcggcgtaagcagaccgaatgcgacgttttggccagacgattggagcatgcgcaacgccttgaggcagttggtacacttgccggcggaatagcacatgaatttaataacattttggggtcaatcctcgggcacgcagaattggcccaaaactcggtgtctcgaacatctgtcacccgaagatacattgactatatcatttcgtcaggcgacagagccatgctcattatcgatcagatcttgacgctgagccgaaaacaagagcgcgtgatcaagccatttagtgtctcagagcttgtgaccgaaatcgctcccttgctacgtatggctcttcccccaaccatcgagcttagtttcagatttgatcaaatgcagagcgtgatcgaaggaagcccgcttgaacttcaacaggtactaattaacctctgcaagaatgcttcccaagccatgactgcaaatggtcaaatcgacatcttcgtcggccaagcttatttaccagctaagaaaattctggcgcatggtgttatgccacctggcgactatgttctactatctgtcagcgacaatggtggaggcatttcagaggctgtgctaccctacatttttgaacccttctttacgacacgtgctcgcaacggtggaacgggtctcggcctcgcttctgtgcatggtcatatcagcgcgtttgcaggttacatcgacgttagttcaactgttgggcatgggacgcgctttgacatttatctccctccgtcttctaaggagcccgtcaatcccgacagctttttcggccgcaataaggcaccgcgtggaaacggggagattgtggcacttgttgagcccgatgacctcctgcgggaggcgtatgaagacaagatcgccgctctgggatatgagccggtcggttttcgtacctttagtgaaattcgcgattggatttcaaaaggcaatgaagccgatctggtcatggtcgaccaagcgtctcttcctgaagatcaaagtcctaattccgtggatttagtgctcaagaccgcctccatcatcattggcggaaatgatctcaaaatgcccctttcaagggagaatgcgaccagggacctttatctgccgaagccgatatcgtccagaactatggcgcatgcaatcctaaccaaaatcaagacgtag

SEQ ID NO: 27 - DNA, 744 bp, Agrobacterium tumefaciens
atggcgataaaattggtattgatactcgtattcacactctttcccgcggcagacgctgcatatgcgaatgaccgcgccaacggtttcatgtggtcaaacgggggcgaaactggagtgaggcttcctcttcgggtgttcaatgccaagccagccaagaacacggtggcgatcatttattccggagacgctggatggcaaaatatcgatgaggcgattggtacctatctgcagacggaagggattcctgtcattggcgtcagttcacttcggtatttctggtcggagcggtccccaagcgaaactgctaaggatcttggtcacataatcgatgtctacaccaagcatttcggtgtgcagaatgttttacttgtaggatattctttcggcgcggacgtcatgccggcaagcttcaataggcttacgcttgagcaaaaaaatcgggttaagcaaatctctctcttggcattgtcacatcaagtcgactatgtcgtctcatttaggggctggctccaactcgaaacggaaggtaagggcggcaatcctctggatgatctcagatccattgaccctgcaatcgtccaatgcatgtacgggcgcgaagaccgtaataatgcttgcccatctctccgacagaccggcgcagaggtgataggcttcagcggaggccatcactttgataatgatttcaaaaaactgtctacgcgcgtcgtctcaggcctcgtggcacgcctaagtcatcagtaa

SEQ ID NO: 28 - DNA, 7743 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacgctgtttaaacgctcttcaactggaagagcggttactaccggttcactagctagctgctaaggttaccagagctggtcacctttgtccaccaacttattaagtatctagttgaagacacgttcttcttcacgtaagaagacactcagtagtcttcggccagaatggcctcttgattcagcgggcctagaaggccggatcactgactagctaatttaaatcctgaggatatcgctatcaactttgtatagaaaagttgggccgaattcgagctcggtacggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccggtcgccaccatggcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgaagcggccgcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggtcacccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgcgtttacaacaagctgtaaatcactagcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggtcacccggtccgggcctagaaggccagcttcggccgccccgggcaactttattatacaaagttgatagatatctggtctaactaactagtcctaaggacccggcggaccgattaaactgattcggtccgaagcttaagccatggcccgggaatcttagcggccgcctgcagagttaacggcgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcct

SEQ ID NO: 29 - DNA, 6496 bp, Artificial sequence
  Synthetic construct
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctagcgaagttcctattccgaagttcctattctctagaaagtataggaacttcagatccaccggctagaggatccaccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggatccaccatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagaatggcccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgaagcggccgcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccgatctcccgggcacccagctttcttgtacaaagtggccgttaacggatcccggtgaagttcctattccgaagttcctattctccagaaagtataggaacttc

SEQ ID NO: 30 - DNA, 1991 bp, Artificial sequence
  Synthetic construct
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 31 - DNA, 685 bp, Unknown
  Discosoma sp.
cgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtag

SEQ ID NO: 32 - DNA, 48 bp, Saccharomyces cerevisiae
gaagttcctattccgaagttcctattctctagaaagtataggaacttc

SEQ ID NO: 33 - DNA, 48 bp, Saccharomyces cerevisiae
gaagttcctattccgaagttcctattctccagaaagtataggaacttc

SEQ ID NO: 34 - DNA, 18676 bp, Artificial sequence
  Synthetic construct
gcatggcatgccgaatccatgtgggagtttattcttgacacagatatttatgatataataactgagtaagcttaacataaggaggaaaaacatatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaataatgtctaacaattcgttcaagccgacgccgcttcgcggcgcggcttaactcaagcgttagatgcactatacgtaccaatccaaatcggatccccctgcagggctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatctatcgatggtacctatctgcagacggaagggattcctgttaattaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctcgcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggcagcgcaggccgcccagcggcaacgcatcgaggcagaagcacgccccggtgaatcgtggcaagcagccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcaggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatgcatataattgtggtttcaaaatcggctccgtcgatactatgttatacgccaactctgaaaacaactttgaaaaagctgttttctggtatttgacccgcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatgcttaagagatcggggtcgctttctcttgccttgatggtctccttctgttcgtcgagccttgccacgccactctcatctgctgagtttgaccatgttgctcgcaagtgtgccccatcagttgcgacatctacgcttgcggcgatagctaaggtggagagtcgctttgatcctttagcgattcatgacaacacgaccggcgaaacgcttcactggcaagatcacagccaagcaacccaagtcgtcaggcaccgtctcgatgcacggcattcgctggatgttggcctcatgcaaataaactctcgaaatttttctatgctcggtctgacacctgacggtgcgctccaggcgtgcacatcattatctgccgctgcaaacatgctgaaaagtcgttatgcaggcggcgaaacgattgacgagaagcaatttgcgcttcgtcgggcgatctccgcttacaacaccggtaatttcatcggcggttttgcaaacggctacgtgcgaaaagttgaaacagctgctcaatcgctggtgcccgcgttaatcgagcctccaaaagacgatcacgaggcgctaaaatccgaagagacgtgggatgtttgggggtcatatcagcgccgctcgcaggaggatggcgctggcggtttaatcgctccgccaccgccacaccaggacaacggcaaatccgcagacgacaatcaagtcttattcgacttatactaaggaggtgcgcattgatgcgatgctttgagagataccgtttacatctaaatcgcctctcgctctcgaatgcgatgatgcgcgtgatatcgagctgcgccccaagcttgtgcggtgcaattgcatggagcatttcctcatccggacccgccgcagcgcaatctgcgggtggcggcactgaccccgccacaatggttaacaatatatgcacgtttatccttggtccgttcggccagtcactcgctgttctcggcattgtcgctatcgggatctcctggatgttcgggcgggcttcgcttgggctggttgccggcgtcgtcggcggcattgttatcatgtttggggcgagcttcctcggccaaacgctcactggcggtagttgatggctgatcgtttggaagaatcgaccctttacctcgcagccacacggcccgcattgtttcttggggtgccactgacattggcagggttattcatgatgttcgccggctttgtcatcgttatcgttcagaacccgctctacgaagtcgttctcgtgccgttatggtttgcagcccggctcatcgtggagcgagactacaatgcggcgagcgtcgtcctgctatttttgcggaccgcgggaagaagcattgatagtgcagtttgggggggcgctactgttagcccaaatccaattagggttcccccacgagggagaggaatggtgtgatgctcggcgcgagtggaacgaccgaaagatccggtgagatctatctcccttatattggccacctcagcgaccatatcgtccttcttgaagacggatcgatcatgaccattgcgagaattgatggcgttgcattcgagcttgaggaaactgaaatgcgcaatgcgcgttgtcgtgcgttcaacacgctgttgcgcaatatcgctgatgatcatgtgtcaatatatgctcacctcgtacgtcatgccgacgtgccatcatcggcgccgcgacacttccgtagtgttttcgccgctagcctgaacgaagcttttgaacagcgcgtgctctccggccaactcctccgcaatgaacacttccttacgttgattgtctacccacaggcggctttagggaaggtaaagaggaggttcaccaagctaagcggaaaaagggaaaacgatctcacgggccagatcaggaacatggaagatctttggcatgttgtcgctggctctcttaaagcgtatggcctgcatcgtcttggcatccgcgagaagcagggtgtgctcttcaccgaaattggcgaagcgctacggttgatcatgactggtcggttcacaccggttccggtcgtcagcggctcactcggcgcttcgatttataccgacagagtcatttgcggcaagcgaggactcgagatcagaacgccaaaagacagttacgttggatccatctattcgtttcgcgaataccctgcaaaaacacggccgggcatgctcaacgcgctgctatccctcgattttccacttgttctcacgcagagtttttcgttcctgactcgcccgcaagcgcacgcgaaacttagcctcaaatcgagccagatgctgagttccggcgataaagccgtgactcaaatcggcaaattatccgaggctgaggacgcacttgcgagcaacgaattcgttatgggctcacatcatttgagcctttgcgtctatgcagacgatctcaatagtcttggggacaggggcgcgcgggctcggacacgaatggcggatgcaggtgccgtggttgtccaagaaggtattggtatggaagcggcctattggtcccaattgccggggaattttaagtggcgcacacgccctggcgcaatcacttcacgcaatttcgcagggtttgtctctttcgaaaactttccagagggcgccagctcaggccactggggcaacgcgattgcccgatttcgtaccaatggcggaacgcctttcgactatatcccgcatgagcacgatgttggcatgacggcaatattcgggcctatcgggaggggtaagacgacgctcatgatgtttgttctagccatgctcgaacagagcatggtcgaccgtgcaggtacggtcgtgttctttgacaaggaccggggtggcgaattgctggttcgcgccacaggaggaacatatttggcacttcacagaggcacacccagcgggttggcgccgttgcgtggcctagaaaacacagcagcctcacacgattttctgcgcgaatggatcgtggctctcatcgagagtgatggtcggggtgggatttctccggaagagaaccgccgtctggtccgtggtatccatcgtcagctctcgtttgatccacaaatgcgttcaatcgcggggttacgtgaatttttgttgcatgggcccgccgaaggcgcaggagcgcggctccaacgctggtgccggggccatgcgcttggctgggcatttgacggcgaagttgacgaagtaaagttagatccgtcgattaccggcttcgacatgacgcatcttctcgaatacgaggaagtatgcgctcccgctgcagcatatctcctgcatcggattggagccatgatcgacggccgccgttttgtgatgagctgcgatgagtttcgcgcctatttgttaaaccctaaattttcgactgtcgtcgacaaattcctcctgaccgttcgaaaaaacaacgggatgctaatactggcaacgcagcaaccagagcatgttctggaatcgccgctaggagccagcttggttgcgcaatgtatgacgaagattttctatccatcaccaaccgcagatcgatcggcttatgtcgatggactgaaatgtaccgaaaaggaatttcaggcgatccgtgaagacatgacggtcggcagccgtaagtttcttcttaaacgagaaagtggaagcgtcatctgcgaatttgatctgcgggatatgcgtgaatatgtcgccgtgctttcggggcgtgccaacacggtgcgctttgcaactcgactacgcgaggcacaagaaggcaactcatctggctggctcagcgaattcatggcccgtcaccacgaggcagaagattgataaggtaggaaacgatgaagacgacgcaacttattgcaacagttttgacctgcagctttctatatattcagcccgcgcgggcgcagtttgttgttagcgacccggcaacggaggctgagacgctcgcgactgcgctcgcgactgcggagaatctcactcagactatagcgatggttacgatgttgacgtcggcctacggcgttactggactactgacttcgctcaaccagaaaaatcagtatccttcgacgaaggacctagacaatgaaatgttttcgccgcgaatgccaatgtcgaccacggcacgtgcgatcaccagcgatacagatcgtgcagtcgtgggtagtgatgctgaagcggacctgttgcgatcgcagatcaccggttccgcaaacagcgctggcattgcggctgacaatctggaaacgatggacaaacgcttgacggcgaatgctgatacgtctgctcagctttcccgatctcgcaatatcatgcaggcaaccgtgaccaatggtttgcttctcaagcagatccatgacgcaatgattcaaaatgtacaggcgacaagcctattaacgatgactaccgcgcaggccggccttcacgaggcggaagaggcggccgctcaacgcaaggagcatcaaaagaccgctgtcatctttggtgccctcccctaaggctgggcgatttgttcatccgcccgcatcctcgccgaatgcgagctcattttatccaacattatgcgacaaaccagtcaagttcaggtccaatcgatgaatttcacgattccggcgccgtttacggccattcatacgatcttcgatgtagccttcacgacaggcttggactcgatgcttgagactatccaggaggcggtgagtgcgccattgatcgcctgtgtcactctttggattattgttcagggtattttagtcatacgcggcgaagtcgatacccgtagcggtatcactcgggtgatcacggtcaccatcgttgttgctctaattgttgggcaggctaactaccaagactatgtggtttccatcttcgaaaagacggtcccaaactttgttcagcagtttagtgtaaccggcttgcctctgcagactgttccggcacagttggatacaatgttcgccgtgacccaggccgtttttcagaaaatcgcatccgaaatcggtccgatgaacgaccaggacatccttgctttccaaggggcacagtgggtcctttacggcacgctctggtctgccttcggagtttacgacgccgttggaattctcacgaaagtgcttctcgcgatcgggcctctgatcctcgtcggatatatttttgatcgcacgcgggacatcgcagctaagtggatcgggcaacttatcacctacggtctcttgcttctcctcttaaacctcgtggcaacgatcgtcatcctaaccgaagcgactgcgctcacccttatgcttggtgtaatcaccttcgccggtacgaccgcggccaagatcattggtctttacgaactcgatatgttttttctgacaggggatgcgctcattgtcgctttgccggcgatcgccggcaacattggaggcagttactggagcggcgcaacccaatctgccagcagcttgtaccgtcgcttcgctcaggttgagcgaggctaggtcgcgcaaaaattcgcctcaatggagaattctatgaagtattgcctgctgtgcctagttgtcgctttgagcggctgccagacaaacgacacattagcgagctgcaaaggcccgatcttcccgctgaatgtggggcgatggcagcctactccgtcagatcttcagctcggcaattcgggtggacgctatgacggggcctgaatatgccatgctagtggcgcgcgaaagccttgccgagcactataaggaagtagaagcctttcaaaccgcgcgagcgaaatcggcgcgacgtctctccaaactcattgcagctgtcgcagctatcgcgattttgggaaatgttgctcaagcgttcgctatagccacaatggtgccgttgagcaggcttgtgcccgtatatctatggatacggccggacggcaccgttgacagcgaggtgtctgtctcgcgattgcctgcaactcaagaggaggccgtcgttaacgcctcattgtgggagtacgttcgcctgcgcgagagttatgatgccgacaccgctcagtacgcctacgacctggtatcgaacttcagtgccccaacagtgcgccaggattaccagcaattcttcaactatcccaatcccagttcgcctcaagtcattcttggcaaacgcggcagggtggaggtcgagcacatcgcttcaaatgatgtaactccaagcacgcagcaaattcgctataaaaggaccctcgtcgttgacggcaaaatgcctgtggtgagtacgtggaccgcgacagttcgctacgaaaaggtgaccagcttgcccggcagattgagactaaccaacccggcaggtctggttgtcacctcctatcagacatcggaagataccgtttcaaacgtaggccacagcgaaccatgatcagaaaagcacttttcattttagcatgtttatttgccgctgcgactggtgcggaggctgaagacactccaatggcgggcaagctagatccgcgcatgcgttatttggcttacaatcccgatcaagtggtgcgcctctcgacggcggttggagctactttggtcgtaacattcgccacgaacgaaacggtgacagcggttgccgtttcaaatagcaaagatctagcagccctaccgcggggaaattatctatttttcaaggcaagccaggtcctcacgcctcagccagtaatcgtgctaaccgcaagcgactccgggatgcgccgttatgttttcagtataagttccaagactctgtcccacctcgataaagagcagcccgatctctattacagcgtccaattcgcctaccccgccgacgatgcggcggctcggcgaagggaggcacaacagaaggctgttgtggacagactacacgcggaagcacaatatcaacggaaagctgagaatttattggatcagcctgtcacagcccttggtgcggcggacagtaattggcactacgtcgcccaaggcgatcgttcgctgttgccactcgaagtcttcgacaatggatttacgacggtattccactttccgggcaatgtacgcataccctccatctacaccatcaatcctgatggcaaggaagctgttgccaactattcagttaaagggagcgatgtcgagatttcttcggtttcccgaggttggcgtctgagggatggccacacagtactatgtatctggaacaccgcttacgatcccgttggccaaaggccgcaaacgggcacggtgaggcccgatgtgaaacgcgtcctgaagggggcgaagggatgaataacgatagtcagcaagcggcacatgaggttgatgcatctggatccctggtctccgacaaacatcgccggcgtctttcggggtctcagaaattgatcgtcggaggtgtcgttctcgcgttatcattaagcctcatttggctaggtgggcgccaaaagaaggtgaatgagaacgcatcgccgtcaactttgatcgcaacaaacaccaagccatttcatccagctccgattgaggtgccgccggatcctccagcggttcaagaggctgttcagcctgctgctcctctaccgccgaggggcgaaccggagcggcatgagccacggccggaagaaacaccgatttttgcatatagcagcggcgatcaaggggtcagcaaacgcgccattcagggcgacacgggccgaagacaagaaggcaagcgtgacgacaactccttgccgaatggcgaagtgtccggcgagaacgatttgtcgatacgtatgaaacccaccgagctgcagcccagcagcgccacgctcttgccgcaccccgattttatggtaacgcaagggacaataattccgtgcatcttgcaaaccgcaatcgacacaaatttggcaggctatgtaaagtgtgtcttgcctcaggatattcgtggaacaacgaacaatatcgtgcttcttgatcgtggcaccaccgttgttggcgaaatacagcgtggcttgcaacagggagatgggcgcgtttttgtgttgtgggatcgcgccgagacacctgaccatgcgatgatctcgttaacatcgccaagcgcggacgaactcggtcgctcaggattgccgggctcggtcgacagccacttctggcagcgttttagcggagctatgctcttgagtgttgttcaaggcgccttccaggcagctagcacctacgccggcagctcgggtggcgggatgagcttcaacagctttcaaaataacggtgagcagacaactgagacagcccttaaggcaaccatcaacataccgccaaccctgaagaagaatcagggtgacaccgtttccattttcgtagcacgggacctcgatttctttggtgtttaccagctccgcctgactggcggcgccacgcgggggaggaaccgccgctcttaatgaattcaaatttccgcttagagataggatacattgtaaatggaagtggatccgcaactacgctttcttctgaagccgattttggaatggctcgatgacccgaagactgaagaaattgcgatcaatcgacctggagaggcatttgtgcgccaagccggcatttttaccaagatgcctttgcccgtctcttatgatgatcttgaagatatcgctattttagcgggcgcgctgagaaagcaggatgtcggaccacgtaaccccctctgcgccactgaacttcctggtggtgaacgactacaaatctgtctgccgccgaccgttccctcgggcaccgtcagcttgaccattcgacggccaagctcgcgtgtttctggtcttaaagaagtctcctcccgttatgatgcttcgaggtggaaccagtggcagacacgaaggaaacgccaaaatcaggatgatgaagctatccttcagcattttgacaacggggatttggaagcgtttctgcacgcatgcgtcgtcagccgactgacgatgttgctatgtggccctaccggaagcggcaagacaacaatgagcaagaccttgatcagcgccatccccccccaggaaaggctaatcaccatagaagatacgctcgaactcgtcattccacacgataatcatgttagactactctactccaagaacggtgctgggctgggtgctgtgagcgccgagcacttgctccaagcaagtctgcgtatgcggccggaccggatattgcttggcgagatgcgcgacgatgcagcatgggcttatctgagtgaagtcgtctcgggacatccgggatcgatttcaacaatacacggcgcgaatccaatccaaggattcaagaaactgttttcccttgtcaaaagtagcgcccaaggtgctagcttggaagatcgcacactgattgacatgctctctacggcgatcgatgtcatcattccattccgtgcctatgaggacgtttatgaagtaggcgagatctggctcgcggcggacgcacgacgccggggcgagaccataggcgatctccttaatcaatagtagctgtaacctcgaagcgtttcacttgtaacaacgattgagaacttttgtcataaaattgaaatacttggttcgcattttcgtcatccgcggtcagccgcaattctgacgaactgcccatttagctggagatgattgtacatccttcacgtgaaaatttctcaagcgctgtgaacaagggttcagattttagattgagaggtgagccgttgaaacacgttcttcttatcgatgacgatgtcgctatgcggcatcttattatcgaataccttacgatccacgccttcaaagtgaccgcggtagccgacagcacccagttcactagagtactctcttccgcgacggtcgatgtcgtggttgttgatctaaatttaggtcgtgaagatgggcttgagatcgttcgaaatctggcggcaaagtctgatattccaatcataattatcagtggcgaccgccttgaggagacggataaagttgttgcactcgagctaggagcaagtgattttatcgctaagccgtttagtacgagagagtttcttgcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttacgacaggagtatagatgttctcattttgcggctgcgccgcaaacttgaggcggatccgtcaagccctcaactgataaaaacagcaagaggtgccggttatttctttgacgcggacgtgcaggtttcgcacggggggacgatggcagcctgagccaattgcatttgattaatttaggtgactgaggacgcggccagcggcctcaaacctacactcaatatttggtgaggggttccgataggtccctcttcaccaattgctcgatggcttctctccagcaaagaatgacgcgagcgcggcggtagccagcttgtggccgaaagctcgagcggtctccaaccccaacggatcaaaatgacttcgagcgacctcgagcaacgcaaccgggaacatgcgtgaggtctgaacgagaacggatttttctgtagttgaagggatcggataacttttcggggccacgcgaaatgatccatctgccagcatgctttcgaaatcgtccaacgcgcgccttaaaatcatttgtagcgacttcgagggactgtattgccgaacgaggttgtcatatgttttcgacacttgaggcgcgggcggtcgcgctgaaagaaaaacctggagctttttcggggacggaggtggactaagggcatccacagttagcttaagttgtcgatcgggactgtaaatgtgatcggcgacgagaggctcacgttgctggtctttctcgtcggctttttcaggcaagtgctggaggtccagcttctggggaacaagtgtcgggttgggatggtggatctcgggtcgagcaccagcaagccgccgtgcttcgccgaccgacaatgcgggcttgcgaattgccatcttcaagcctccaagattttgctgatcagtttcgaaatgaccacgacttcctccatcgcaatccgaagattcctctctatgaggcgcatcgtcggatcagttcccgtgtttagtaatgtaagatgcaacatgccgcgttctttcatcgcggcaaatgcatctctttcatgcatgggagacggtacaactggaaggctctctagcgtctctgacatcctgcgttgcgatgttgtcaatcggccgaccgggacgcgttggcgcaaaacagctgtaggaattgccaaattttcactcaacagcagctcgatgacgtagcggtaggtagatagtgcctcatcgatgtcgagcggcgttagcatggtggggatcagaagcaggtttgagctagcgatgattgtgttgttgagctcgctcgagccgccacgcgtatcggccaacgcataatcaaatccttcgagctcggcattttcataggctgcttcaagaaggggcatttcgtcggcggaatagacttcacagcgaggatcccaggtactgctttgtaaggcgttttctctccatcgcgtcagaggccggttttcgtcggcatcaaagagggccactcgtttaccgtcatttgccaaagcagcgcaaaggcccatgagtgcggtggttttgccagcaccccctttgaaagaacaaaacgtcaaaagttgcatattctgatcccgcctatcctgtgaaaccggagtgcatttgtatttttgttcgtataaatgtttttgtgattatcgatgagtaaaagcgttgttacactatttttatttcacattcgttataagacaattgcaaatgtagcaagtatattcagtattgactgtaaatgtactgttgatttcatattgagcagggctagacttccatccgtctacccgggcacatttcgtgctggagtatccagaccttccgctttctttggaggaagctatgtcaaaacacaccagagccacgtcgagtgagactaccatcaaccagcatcgatccctgaaagttgaagggttcaaggtcgtgagtgcccgtctgcgatcggccgagtatgaaaccttttcctatcaagcgcgcctgctgggactttcggatagtatggcaattcgcgttgcggtgcgccgcatcgggggctttctcgaaatagatgcagacacacgagaaaagatggaagccatacttcagtccatcggaatactctcaagcaatgtatccatgcttctatctgcctacgccgaagaccctcgatcggatctggaggctgtgcgagatgaacgtattgcttttggcgaggctttcgccgccctcgatggactactgcgctccattttgtccgtatcccggcgacggatcgacggtcgctcgttactgaaaggtgccttgtagcacttgaccacgcacctgacgggagaaaattggatgcccgatcgcgctcaagtaatcattcgcattgtgccaggaggtggaaccaagacccttcagcagataatcaatcagctggagtacctgtcccgaaagggaaagctggaactgcagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtaagcttccccgcaggtaccgaccaaaccgcagcttatgaagcaagccgggaatgggcagccgagatgtttgggtcaggatacgggggtggccgctataactatctgacagcctaccacgtcgaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaatatcgacgccttgagcggatgcaggctcaaaagattcaattcgaagatacagattttgatgagacctcgcctgaggaagatcgtcgggacctcagtcaatcgttcgatccatttcgatcggacgcatctgccggcgaaccggaccgtgcaacccgacatgacaaacaaccgcttgaaccgcacgcccgtttccaggagcccgccggctccagcatcaaagccgacgcacggatccgcgtaccattggagagcgagcggggtgcccaaccatccgcgtccaaaatccctgtaactgggcatttcgggattgagacttcgtatgtcgctgaagccagcgtgcccaaacaaagcggcaattccgatacttctcgcccggtgactgacgttgccatgcacacagtcgagcgccagcagcgatcaaaacgacgtcatgacgaggaggcaggtccgagcggagcaaaccgtaaaagattgaaggccgcgcaagttgattccgaggcaaatgtcggtgagcccgacggtcgcgatgacagcaacaaggcggctgatccggtgtctgcttccatccgtaccgagcaaccggaagcttctccaacgtgtccgcgtgaccgtcacgatggagaattgggagaacgcaaacgtgcaagaggtaatcgtcgcgacgatgggcgcggggggacctagactagtgtacctcgcgaatgcatctagatccaatccaatat

SEQ ID NO: 35 - DNA, 28390 bp, Artificial sequence
  Synthetic construct
gacccgcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatgcttaagagatcggggtcgctttctcttgccttgatggtctccttctgttcgtcgagccttgccacgccactctcatctgctgagtttgaccatgttgctcgcaagtgtgccccatcagttgcgacatctacgcttgcggcgatagctaaggtggagagtcgctttgatcctttagcgattcatgacaacacgaccggcgaaacgcttcactggcaagatcacagccaagcaacccaagtcgtcaggcaccgtctcgatgcacggcattcgctggatgttggcctcatgcaaataaactctcgaaatttttctatgctcggtctgacacctgacggtgcgctccaggcgtgcacatcattatctgccgctgcaaacatgctgaaaagtcgttatgcaggcggcgaaacgattgacgagaagcaatttgcgcttcgtcgggcgatctccgcttacaacaccggtaatttcatcggcggttttgcaaacggctacgtgcgaaaagttgaaacagctgctcaatcgctggtgcccgcgttaatcgagcctccaaaagacgatcacgaggcgctaaaatccgaagagacgtgggatgtttgggggtcatatcagcgccgctcgcaggaggatggcgctggcggtttaatcgctccgccaccgccacaccaggacaacggcaaatccgcagacgacaatcaagtcttattcgacttatactaaggaggtgcgcattgatgcgatgctttgagagataccgtttacatctaaatcgcctctcgctctcgaatgcgatgatgcgcgtgatatcgagctgcgccccaagcttgtgcggtgcaattgcatggagcatttcctcatccggacccgccgcagcgcaatctgcgggtggcggcactgaccccgccacaatggttaacaatatatgcacgtttatccttggtccgttcggccagtcactcgctgttctcggcattgtcgctatcgggatctcctggatgttcgggcgggcttcgcttgggctggttgccggcgtcgtcggcggcattgttatcatgtttggggcgagcttcctcggccaaacgctcactggcggtagttgatggctgatcgtttggaagaatcgaccctttacctcgcagccacacggcccgcattgtttcttggggtgccactgacattggcagggttattcatgatgttcgccggctttgtcatcgttatcgttcagaacccgctctacgaagtcgttctcgtgccgttatggtttgcagcccggctcatcgtggagcgagactacaatgcggcgagcgtcgtcctgctatttttgcggaccgcgggaagaagcattgatagtgcagtttgggggggcgctactgttagcccaaatccaattagggttcccccacgagggagaggaatggtgtgatgctcggcgcgagtggaacgaccgaaagatccggtgagatctatctcccttatattggccacctcagcgaccatatcgtccttcttgaagacggatcgatcatgaccattgcgagaattgatggcgttgcattcgagcttgaggaaactgaaatgcgcaatgcgcgttgtcgtgcgttcaacacgctgttgcgcaatatcgctgatgatcatgtgtcaatatatgctcacctcgtacgtcatgccgacgtgccatcatcggcgccgcgacacttccgtagtgttttcgccgctagcctgaacgaagcttttgaacagcgcgtgctctccggccaactcctccgcaatgaacacttccttacgttgattgtctacccacaggcggctttagggaaggtaaagaggaggttcaccaagctaagcggaaaaagggaaaacgatctcacgggccagatcaggaacatggaagatctttggcatgttgtcgctggctctcttaaagcgtatggcctgcatcgtcttggcatccgcgagaagcagggtgtgctcttcaccgaaattggcgaagcgctacggttgatcatgactggtcggttcacaccggttccggtcgtcagcggctcactcggcgcttcgatttataccgacagagtcatttgcggcaagcgaggactcgagatcagaacgccaaaagacagttacgttggatccatctattcgtttcgcgaataccctgcaaaaacacggccgggcatgctcaacgcgctgctatccctcgattttccacttgttctcacgcagagtttttcgttcctgactcgcccgcaagcgcacgcgaaacttagcctcaaatcgagccagatgctgagttccggcgataaagccgtgactcaaatcggcaaattatccgaggctgaggacgcacttgcgagcaacgaattcgttatgggctcacatcatttgagcctttgcgtctatgcagacgatctcaatagtcttggggacaggggcgcgcgggctcggacacgaatggcggatgcaggtgccgtggttgtccaagaaggtattggtatggaagcggcctattggtcccaattgccggggaattttaagtggcgcacacgccctggcgcaatcacttcacgcaatttcgcagggtttgtctctttcgaaaactttccagagggcgccagctcaggccactggggcaacgcgattgcccgatttcgtaccaatggcggaacgcctttcgactatatcccgcatgagcacgatgttggcatgacggcaatattcgggcctatcgggaggggtaagacgacgctcatgatgtttgttctagccatgctcgaacagagcatggtcgaccgtgcaggtacggtcgtgttctttgacaaggaccggggtggcgaattgctggttcgcgccacaggaggaacatatttggcacttcacagaggcacacccagcgggttggcgccgttgcgtggcctagaaaacacagcagcctcacacgattttctgcgcgaatggatcgtggctctcatcgagagtgatggtcggggtgggatttctccggaagagaaccgccgtctggtccgtggtatccatcgtcagctctcgtttgatccacaaatgcgttcaatcgcggggttacgtgaatttttgttgcatgggcccgccgaaggcgcaggagcgcggctccaacgctggtgccggggccatgcgcttggctgggcatttgacggcgaagttgacgaagtaaagttagatccgtcgattaccggcttcgacatgacgcatcttctcgaatacgaggaagtatgcgctcccgctgcagcatatctcctgcatcggattggagccatgatcgacggccgccgttttgtgatgagctgcgatgagtttcgcgcctatttgttaaaccctaaattttcgactgtcgtcgacaaattcctcctgaccgttcgaaaaaacaacgggatgctaatactggcaacgcagcaaccagagcatgttctggaatcgccgctaggagccagcttggttgcgcaatgtatgacgaagattttctatccatcaccaaccgcagatcgatcggcttatgtcgatggactgaaatgtaccgaaaaggaatttcaggcgatccgtgaagacatgacggtcggcagccgtaagtttcttcttaaacgagaaagtggaagcgtcatctgcgaatttgatctgcgggatatgcgtgaatatgtcgccgtgctttcggggcgtgccaacacggtgcgctttgcaactcgactacgcgaggcacaagaaggcaactcatctggctggctcagcgaattcatggcccgtcaccacgaggcagaagattgataaggtaggaaacgatgaagacgacgcaacttattgcaacagttttgacctgcagctttctatatattcagcccgcgcgggcgcagtttgttgttagcgacccggcaacggaggctgagacgctcgcgactgcgctcgcgactgcggagaatctcactcagactatagcgatggttacgatgttgacgtcggcctacggcgttactggactactgacttcgctcaaccagaaaaatcagtatccttcgacgaaggacctagacaatgaaatgttttcgccgcgaatgccaatgtcgaccacggcacgtgcgatcaccagcgatacagatcgtgcagtcgtgggtagtgatgctgaagcggacctgttgcgatcgcagatcaccggttccgcaaacagcgctggcattgcggctgacaatctggaaacgatggacaaacgcttgacggcgaatgctgatacgtctgctcagctttcccgatctcgcaatatcatgcaggcaaccgtgaccaatggtttgcttctcaagcagatccatgacgcaatgattcaaaatgtacaggcgacaagcctattaacgatgactaccgcgcaggccggccttcacgaggcggaagaggcggccgctcaacgcaaggagcatcaaaagaccgctgtcatctttggtgccctcccctaaggctgggcgatttgttcatccgcccgcatcctcgccgaatgcgagctcattttatccaacattatgcgacaaaccagtcaagttcaggtccaatcgatgaatttcacgattccggcgccgtttacggccattcatacgatcttcgatgtagccttcacgacaggcttggactcgatgcttgagactatccaggaggcggtgagtgcgccattgatcgcctgtgtcactctttggattattgttcagggtattttagtcatacgcggcgaagtcgatacccgtagcggtatcactcgggtgatcacggtcaccatcgttgttgctctaattgttgggcaggctaactaccaagactatgtggtttccatcttcgaaaagacggtcccaaactttgttcagcagtttagtgtaaccggcttgcctctgcagactgttccggcacagttggatacaatgttcgccgtgacccaggccgtttttcagaaaatcgcatccgaaatcggtccgatgaacgaccaggacatccttgctttccaaggggcacagtgggtcctttacggcacgctctggtctgccttcggagtttacgacgccgttggaattctcacgaaagtgcttctcgcgatcgggcctctgatcctcgtcggatatatttttgatcgcacgcgggacatcgcagctaagtggatcgggcaacttatcacctacggtctcttgcttctcctcttaaacctcgtggcaacgatcgtcatcctaaccgaagcgactgcgctcacccttatgcttggtgtaatcaccttcgccggtacgaccgcggccaagatcattggtctttacgaactcgatatgttttttctgacaggggatgcgctcattgtcgctttgccggcgatcgccggcaacattggaggcagttactggagcggcgcaacccaatctgccagcagcttgtaccgtcgcttcgctcaggttgagcgaggctaggtcgcgcaaaaattcgcctcaatggagaattctatgaagtattgcctgctgtgcctagttgtcgctttgagcggctgccagacaaacgacacattagcgagctgcaaaggcccgatcttcccgctgaatgtggggcgatggcagcctactccgtcagatcttcagctcggcaattcgggtggacgctatgacggggcctgaatatgccatgctagtggcgcgcgaaagccttgccgagcactataaggaagtagaagcctttcaaaccgcgcgagcgaaatcggcgcgacgtctctccaaactcattgcagctgtcgcagctatcgcgattttgggaaatgttgctcaagcgttcgctatagccacaatggtgccgttgagcaggcttgtgcccgtatatctatggatacggccggacggcaccgttgacagcgaggtgtctgtctcgcgattgcctgcaactcaagaggaggccgtcgttaacgcctcattgtgggagtacgttcgcctgcgcgagagttatgatgccgacaccgctcagtacgcctacgacctggtatcgaacttcagtgccccaacagtgcgccaggattaccagcaattcttcaactatcccaatcccagttcgcctcaagtcattcttggcaaacgcggcagggtggaggtcgagcacatcgcttcaaatgatgtaactccaagcacgcagcaaattcgctataaaaggaccctcgtcgttgacggcaaaatgcctgtggtgagtacgtggaccgcgacagttcgctacgaaaaggtgaccagcttgcccggcagattgagactaaccaacccggcaggtctggttgtcacctcctatcagacatcggaagataccgtttcaaacgtaggccacagcgaaccatgatcagaaaagcacttttcattttagcatgtttatttgccgctgcgactggtgcggaggctgaagacactccaatggcgggcaagctagatccgcgcatgcgttatttggcttacaatcccgatcaagtggtgcgcctctcgacggcggttggagctactttggtcgtaacattcgccacgaacgaaacggtgacagcggttgccgtttcaaatagcaaagatctagcagccctaccgcggggaaattatctatttttcaaggcaagccaggtcctcacgcctcagccagtaatcgtgctaaccgcaagcgactccgggatgcgccgttatgttttcagtataagttccaagactctgtcccacctcgataaagagcagcccgatctctattacagcgtccaattcgcctaccccgccgacgatgcggcggctcggcgaagggaggcacaacagaaggctgttgtggacagactacacgcggaagcacaatatcaacggaaagctgagaatttattggatcagcctgtcacagcccttggtgcggcggacagtaattggcactacgtcgcccaaggcgatcgttcgctgttgccactcgaagtcttcgacaatggatttacgacggtattccactttccgggcaatgtacgcataccctccatctacaccatcaatcctgatggcaaggaagctgttgccaactattcagttaaagggagcgatgtcgagatttcttcggtttcccgaggttggcgtctgagggatggccacacagtactatgtatctggaacaccgcttacgatcccgttggccaaaggccgcaaacgggcacggtgaggcccgatgtgaaacgcgtcctgaagggggcgaagggatgaataacgatagtcagcaagcggcacatgaggttgatgcatctggatccctggtctccgacaaacatcgccggcgtctttcggggtctcagaaattgatcgtcggaggtgtcgttctcgcgttatcattaagcctcatttggctaggtgggcgccaaaagaaggtgaatgagaacgcatcgccgtcaactttgatcgcaacaaacaccaagccatttcatccagctccgattgaggtgccgccggatcctccagcggttcaagaggctgttcagcctgctgctcctctaccgccgaggggcgaaccggagcggcatgagccacggccggaagaaacaccgatttttgcatatagcagcggcgatcaaggggtcagcaaacgcgccattcagggcgacacgggccgaagacaagaaggcaagcgtgacgacaactccttgccgaatggcgaagtgtccggcgagaacgatttgtcgatacgtatgaaacccaccgagctgcagcccagcagcgccacgctcttgccgcaccccgattttatggtaacgcaagggacaataattccgtgcatcttgcaaaccgcaatcgacacaaatttggcaggctatgtaaagtgtgtcttgcctcaggatattcgtggaacaacgaacaatatcgtgcttcttgatcgtggcaccaccgttgttggcgaaatacagcgtggcttgcaacagggagatgggcgcgtttttgtgttgtgggatcgcgccgagacacctgaccatgcgatgatctcgttaacatcgccaagcgcggacgaactcggtcgctcaggattgccgggctcggtcgacagccacttctggcagcgttttagcggagctatgctcttgagtgttgttcaaggcgccttccaggcagctagcacctacgccggcagctcgggtggcgggatgagcttcaacagctttcaaaataacggtgagcagacaactgagacagcccttaaggcaaccatcaacataccgccaaccctgaagaagaatcagggtgacaccgtttccattttcgtagcacgggacctcgatttctttggtgtttaccagctccgcctgactggcggcgccacgcgggggaggaaccgccgctcttaatgaattcaaatttccgcttagagataggatacattgtaaatggaagtggatccgcaactacgctttcttctgaagccgattttggaatggctcgatgacccgaagactgaagaaattgcgatcaatcgacctggagaggcatttgtgcgccaagccggcatttttaccaagatgcctttgcccgtctcttatgatgatcttgaagatatcgctattttagcgggcgcgctgagaaagcaggatgtcggaccacgtaaccccctctgcgccactgaacttcctggtggtgaacgactacaaatctgtctgccgccgaccgttccctcgggcaccgtcagcttgaccattcgacggccaagctcgcgtgtttctggtcttaaagaagtctcctcccgttatgatgcttcgaggtggaaccagtggcagacacgaaggaaacgccaaaatcaggatgatgaagctatccttcagcattttgacaacggggatttggaagcgtttctgcacgcatgcgtcgtcagccgactgacgatgttgctatgtggccctaccggaagcggcaagacaacaatgagcaagaccttgatcagcgccatccccccccaggaaaggctaatcaccatagaagatacgctcgaactcgtcattccacacgataatcatgttagactactctactccaagaacggtgctgggctgggtgctgtgagcgccgagcacttgctccaagcaagtctgcgtatgcggccggaccggatattgcttggcgagatgcgcgacgatgcagcatgggcttatctgagtgaagtcgtctcgggacatccgggatcgatttcaacaatacacggcgcgaatccaatccaaggattcaagaaactgttttcccttgtcaaaagtagcgcccaaggtgctagcttggaagatcgcacactgattgacatgctctctacggcgatcgatgtcatcattccattccgtgcctatgaggacgtttatgaagtaggcgagatctggctcgcggcggacgcacgacgccggggcgagaccataggcgatctccttaatcaatagtagctgtaacctcgaagcgtttcacttgtaacaacgattgagaacttttgtcataaaattgaaatacttggttcgcattttcgtcatccgcggtcagccgcaattctgacgaactgcccatttagctggagatgattgtacatccttcacgtgaaaatttctcaagcgctgtgaacaagggttcagattttagattgagaggtgagccgttgaaacacgttcttcttatcgatgacgatgtcgctatgcggcatcttattatcgaataccttacgatccacgccttcaaagtgaccgcggtagccgacagcacccagttcactagagtactctcttccgcgacggtcgatgtcgtggttgttgatctaaatttaggtcgtgaagatgggcttgagatcgttcgaaatctggcggcaaagtctgatattccaatcataattatcagtggcgaccgccttgaggagacggataaagttgttgcactcgagctaggagcaagtgattttatcgctaagccgtttagtacgagagagtttcttgcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttacgacaggagtatagatgttctcattttgcggctgcgccgcaaacttgaggcggatccgtcaagccctcaactgataaaaacagcaagaggtgccggttatttctttgacgcggacgtgcaggtttcgcacggggggacgatggcagcctgagccaattgcatttgattaatttaggtgactgaggacgcggccagcggcctcaaacctacactcaatatttggtgaggggttccgataggtccctcttcaccaattgctcgatggcttctctccagcaaagaatgacgcgagcgcggcggtagccagcttgtggccgaaagctcgagcggtctccaaccccaacggatcaaaatgacttcgagcgacctcgagcaacgcaaccgggaacatgcgtgaggtctgaacgagaacggatttttctgtagttgaagggatcggataacttttcggggccacgcgaaatgatccatctgccagcatgctttcgaaatcgtccaacgcgcgccttaaaatcatttgtagcgacttcgagggactgtattgccgaacgaggttgtcatatgttttcgacacttgaggcgcgggcggtcgcgctgaaagaaaaacctggagctttttcggggacggaggtggactaagggcatccacagttagcttaagttgtcgatcgggactgtaaatgtgatcggcgacgagaggctcacgttgctggtctttctcgtcggctttttcaggcaagtgctggaggtccagcttctggggaacaagtgtcgggttgggatggtggatctcgggtcgagcaccagcaagccgccgtgcttcgccgaccgacaatgcgggcttgcgaattgccatcttcaagcctccaagattttgctgatcagtttcgaaatgaccacgacttcctccatcgcaatccgaagattcctctctatgaggcgcatcgtcggatcagttcccgtgtttagtaatgtaagatgcaacatgccgcgttctttcatcgcggcaaatgcatctctttcatgcatgggagacggtacaactggaaggctctctagcgtctctgacatcctgcgttgcgatgttgtcaatcggccgaccgggacgcgttggcgcaaaacagctgtaggaattgccaaattttcactcaacagcagctcgatgacgtagcggtaggtagatagtgcctcatcgatgtcgagcggcgttagcatggtggggatcagaagcaggtttgagctagcgatgattgtgttgttgagctcgctcgagccgccacgcgtatcggccaacgcataatcaaatccttcgagctcggcattttcataggctgcttcaagaaggggcatttcgtcggcggaatagacttcacagcgaggatcccaggtactgctttgtaaggcgttttctctccatcgcgtcagaggccggttttcgtcggcatcaaagagggccactcgtttaccgtcatttgccaaagcagcgcaaaggcccatgagtgcggtggttttgccagcaccccctttgaaagaacaaaacgtcaaaagttgcatattctgatcccgcctatcctgtgaaaccggagtgcatttgtatttttgttcgtataaatgtttttgtgattatcgatgagtaaaagcgttgttacactatttttatttcacattcgttataagacaattgcaaatgtagcaagtatattcagtattgactgtaaatgtactgttgatttcatattgagcagggctagacttccatccgtctacccgggcacatttcgtgctggagtatccagaccttccgctttctttggaggaagctatgtcaaaacacaccagagccacgtcgagtgagactaccatcaaccagcatcgatccctgaaagttgaagggttcaaggtcgtgagtgcccgtctgcgatcggccgagtatgaaaccttttcctatcaagcgcgcctgctgggactttcggatagtatggcaattcgcgttgcggtgcgccgcatcgggggctttctcgaaatagatgcagacacacgagaaaagatggaagccatacttcagtccatcggaatactctcaagcaatgtatccatgcttctatctgcctacgccgaagaccctcgatcggatctggaggctgtgcgagatgaacgtattgcttttggcgaggctttcgccgccctcgatggactactgcgctccattttgtccgtatcccggcgacggatcgacggtcgctcgttactgaaaggtgccttgtagcacttgaccacgcacctgacgggagaaaattggatgcccgatcgcgctcaagtaatcattcgcattgtgccaggaggtggaaccaagacccttcagcagataatcaatcagctggagtacctgtcccgaaagggaaagctggaactgcagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtaagcttccccgcaggtaccgaccaaaccgcagcttatgaagcaagccgggaatgggcagccgagatgtttgggtcaggatacgggggtggccgctataactatctgacagcctaccacgtcgaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaatatcgacgccttgagcggatgcaggctcaaaagattcaattcgaagatacagattttgatgagacctcgcctgaggaagatcgtcgggacctcagtcaatcgttcgatccatttcgatcggacgcatctgccggcgaaccggaccgtgcaacccgacatgacaaacaaccgcttgaaccgcacgcccgtttccaggagcccgccggctccagcatcaaagccgacgcacggatccgcgtaccattggagagcgagcggggtgcccaaccatccgcgtccaaaatccctgtaactgggcatttcgggattgagacttcgtatgtcgctgaagccagcgtgcccaaacaaagcggcaattccgatacttctcgcccggtgactgacgttgccatgcacacagtcgagcgccagcagcgatcaaaacgacgtcatgacgaggaggcaggtccgagcggagcaaaccgtaaaagattgaaggccgcgcaagttgattccgaggcaaatgtcggtgagcccgacggtcgcgatgacagcaacaaggcggctgatccggtgtctgcttccatccgtaccgagcaaccggaagcttctccaacgtgtccgcgtgaccgtcacgatggagaattgggagaacgcaaacgtgcaagaggtaatcgtcgcgacgatgggcgcggggggacctagactagtagacaggaaggaccgaataatggcaaatggtcagttcacgatacgctctgctcgcccggcctccgtcggactgacaggcgaacggcgtggagccgcatccgcctctagctctgcactgtccaatgttcaaagagatgttagggataggctgattccaactagctcaccaagattaccaaatgcagccatattgcgtgattcctcgggaagagcgtcgactggtctgcggtacatggcggctactcttcattggtctgcgatcgcgccattatcgctaataaacagcaacgacctggctccggccgcttatgactttgagacgcgaaataacgcaagaaatgtgactgccaaagtcggcagggcagtccctgttcccaagcaaggcgggctcggcaaaacgctcgcacccgtaccccttagtacacgtatatcaagggtcaattccgaccgaagactgcccgctgacgcagaagaccgccctgaaacgcgcgacccccagaaaggacgtggcagtcatggtgcgacgccaaccttacatgaaaagattggaaccgcgtttgctcgaagattgcgaaagcatacgtactatattgtttgcagttgctgccagacccggagcgcgttgacgatgggtgcaaagatttcggtgaagtcatgaactccagcaagacttcgccccagcgtatgaccctgagcatcgtatgttcgctggcagccggtttttgtgcggccagctgctatgtaacgttccgccggggcttcaacggcgaagcgatgatgacgttcgacgttttcgctttttggtatgagaccccgctttacttgggttatgccagcaccgtcttctggcgtggtttatctgttgtcatctttacctcgctgatcgttctttcaagtcagctcatcatatcgctgcgcaatcagaagcatcatgggacagctcgttgggcagaaattggcgaaatgcggcatgctggttatctgcagcgttacagtcgcatcaaggggccgatctttggaaagacatgtggtcccctttggttcggcagttatttgaccaatggcgaacagccacacagtcttgtcgtcgcgccaacgcgtgctggcaaaggcgtcggcatcgtcattccaacgctgttgaccttcaagggctcggtaatcgcccttgacgtcaagggagaattgtttgaactgacgtccagagcacgcaaagcgagcggcgacgcagttttcaagttctcccccctagatcctgagcggaagactcattgttacaatccggtcctggatattgccgcacttccgcccgaacgccagttcactgaaacacgccgtctagctgcgaaccttattacggctaagggaaagggagcagaaggctttattgacggcgcacgtgacctgttcgtcgcgggaatccttacctgcattgagcgtggcacaccaacgattggcgcggtatatgacctatttgcgcagcctggcgaaaagtataagctttttgcgcaactcgcggaggaaagcctaaacaaagaggctcagcgtatcttcgataatatggcgggcaacgacacgaaaattctgacatcgtacacctctgtgctgggcgacggtggactgaacctgtgggctgatccgcttatcaaagcagcgacaagccggtcagacttttccgtttacgatctccggaggaagaagacctgcatttatctttgtgtcagtcccaacgatctggaggtcttggcaccacttatgcgcctgatgtttcagcagctcgtgtcaatcttgcagagatcgctgccaggtgaagacgagtgccatgaagttttatttctcctcgacgaattcaaacacctgggcaagcttgaggccatagagaccgcgatcacaaccatcgccggttacagaggccgctttatgtttattattcaaagtctttcggccttgtcgggcacatacgatgacgcaggaaaacaaaactttctgagcaatactggcgtacaagtatttatggccacggctgatgacgaaactccaacctacatctcaaaagctatcggcgaatatacgtttaaagcgcgttcgacctcttacagtcaagccagaatgttcgaccacaacatccagatttctgatcaaggtgcagcccttttgcgccccgaacaagtgcgcctgctagacgatcagagtgaaatcgttctcatcaaagggcgacctccactcaaattacgaaaggtgcagtattattccgatcgtacgctgaaaggccttttcgaacgccagatgggctctctgcctgagcccgcacccttgatgctttccgactatagcaacgatcaagttcaataccacttggctccgatagcaaattttaatgaggatgctgcaccgcaaaacagaactgtggccgaggaccatggaagtgttaaagtcggtgctgatatccctgaacgcgtgatgggaataaatggtgacgaggaacaagccgatgcgggcgagataccgccggaatcggttgtgcctccagaattgacgctcgctctgaccgctcaacagcaattgttggaccagattattgcacttcagcaaagatcgaggtccgcaccggcatagcctgcgaaatgatcttaatggtgcaattcgtttcaggcggcatgttgcggttcaacaaagtgtacgccagatcccaattggccctttacgaggtgtcggtatgacaggaaagtcgaaagttcacataagaggttcggctgacgcgcttcctgacgttcctggcggaagtactaccgccccttttttaaccgaaccttctcgggatcaggttgatgcctcgtttgaggtccaaaccgactacagccagtctacttccgtgtcgtttacctatgatggtgttggacttggtcctgccgagcgtgcggcttacgagaactggtgcgaaccgggccggcccacttggaaagatcttataatcaaggcacgtgtcgatccgattgacgatgtgacctggctccgagatttagaagaggacaccccctcaaccttcagatacgaagggatgcctctgggcatcggggaacgacaggcctacgaaaattggcaagaggacgctcagccgacatgggaagaccttgttgtcagcgcacgcttgacggaacttggccgtccacacgggattaccggcgagtatacatccctcgcaggatcgaagaatacaagttcaatttcattgaagcgaaagcggagcaacttaattgatgatgagaattcatccggatcgttttcatatgacgggatgaagctcggggaagccgagcgttctgcatatggtgactgggccgaggcggagccacccacgtggaaagatttggtattgagggcacgcgtttcctcgatcaatgactctgcttggctttttgattcacaaacatcttcatcatcatttgaatacaacggtgttcccttgggcgagccggaacggcaggctctcagacaatggcaaggagacgctcagcctacctgggaagatctcgttgttaacgcgcgtatggcagaactttgccatgctggttggattgaaggtcaaaaaggttgctttgaagagcgcggggaggctctgcccgcgtcggaacgcggttcgcaacgccccattggtcaacggacagattcctccgattcttttgtgtatgatggcacaaggctcggagcacctgagcgaactgcttatgaacgctggagtaagagggaacgcccgacttgggaagatctcatcttagatgcacaccaggccaggactgaaagtgacgctgttacgacccaagcgattggtcagtcgtcctcaccggttttcttatatgaaggaaagtcgctcggagacagggaacgaaaggcttacgaaaaatggcggcagccagcccaaccgcgatggcaaaatcttgtagtgaacgctcgtctggcagaaatcgatccctcagcctggattgccgatgagcgcgatccgcttgatgatagcgacgcgcttggtcgcccgtcgtacacaagcttgacggatagatcagacgtccctttagacgatcaatcaatctatcgtcgttccgacctagtaagggagcaggtgccagaatcgtctcaaaggcaattcgcagcatgttcagaatctgaaacgaggcctgtgcaatggtttactgcttctgggtcagatgcaaacaatacggaaaatatcaccgccagcgatcccgtcgatcgcacgggtggagttaagcggctaggctccaaaagcgacagaaccgttacagcttctatccatgacgtgaattccagcacaaggcgactgttgcttaacgaatttggatcggaggctccgcgcccttcgccagaaaagactgttcgcttaagaagcgacaatattggcacctatgggagccggaaaaatgaacgagcgcggctcgcgaccgaaaccggtgcgtatgagtcggagcatattttcgggttcaaggctgtccacgatactgcgagagcgacgaaagagggccggcgtctcgaaaggcccatgcccgcctaccttgaggataaggggcttcatcgccaacatattggcaccgggagaggacggaccaaacttgtcgggcgcggatggccggatgacacaagctatcgctcggatcaaagggcaactctgtcggaccccgttgcgcgctcggaaggcgcgacggcctcaaatgggtatcaattgaaccaattgggttacgcgcaccaactcgctagcgatggcctgcaaagtgaatcgcccgatggtgttgccttgccaattcaagtggcaacaacgagctacaactatacagtgagccgcgatcctgtccttgttccgccggataaaaacgaagcccctcaattgctgcatcttggtccccgtggtcaaaccgaagctgttcttgcccgcgaaacagcattgactggaaaatggccgactctcgagcgtgagcagcaagtgtatcgcgagtttttggccttatatgacgtaaaaaaagatcttgaggccaaatcagtcggcgtaagacggaaaaaaaaagaagttatttctgcgttagaccgaactgcgcgcttgataagcacgtcgccttcgaaagctcgatccaaagcagagactgaaaaagccattgatgagctcgatgatcgacgagtttatgatccgcgtgatcgagctcaagacaaagcgtttaaacgctgataagtcgccaatatagtgatcatttgcagtattcgcatcgatcgctggttgatattctgccgctggtcgaccggctgctcgtcgccaaaaatgctcacagggatacgatggcctcggtcaggccgcgtgcgtcctgtctttccagttcctccctttcagctcgattgtggcatcatttattgcctgctcattgcagttgaaacgcgatatccgtttcaagacccgggtatggatggtactttggagatatgagcacttgccagacttcccggtcggagacttaaaaattccgataagaggttacggacgacggaccttataagtggatcgtctagtggtggcgccgatcaaaacagttccgcccccggctttgctcaaagtagcaaagcagctttatctcgggttgcggaggattttctagaaaaccgcaattttgcaggagagaacatatggccatcatcaagccgcatgtgaacaaaaataggacaacctcgccgatagagagaccggagtctctcatagaggaaatgagcggcagtcatccgccgagtggttttaccaacctggatctcgctatgatcgagctggaggactttgtccatcggtgcccgctcccagaagacaatcttgctggtcagaaggagtgagacgatggatccgtctagcaatgagaatgtctatgtgggtcgcggtcacaacatcgaaaatgatgatgacactgaccccaggcgttggaagaaggcgaatatcagttccaacaccatctccgatattcagatgacgaatggcgaagacgtacaatcagggagccctacccgaacggaagttgtaagcccacgtctggattatggatcggtcgactcctcctccagcctttattctggcagcgagcacggaaatcaagctgagattcaaaaagagctgtccgtcttgttctcgaacatgtctttgccaggcaacgatcggcgcccggacgaatacattctcgtgcatcaaacgggacaagatgcttttactggtattgccaaaggcaacctcgaccaaatgcccaccaaggcggaatttaacgcgtgctgccgtctctacagggacggagccggtaattactacccgccacctctcgcattcgacaagattagcgttccagagcaactggaggaaaaatgggggatgatggaggcgaaggaacgtaacaaactgcggtttcagtacaagttggacgtatggaatcatgcgcacgctgatatggggatcacgggcacagagatcttttatcaaacagataagaacataaagctcgaccggaattataaactaagacctgaagaccgatacgtacaaacagaaaaatacgggcgccgggaaattcaaaagcgatatcaacacgaactccaggctggttcgctgctgcccgatattatgatcaaaactccccaaaatgacatccacttcgtgtacaggtttgccggcgacaattacgccaacaaacagttcagcgagtttgaacacaccgtcaagcgcaggtatggcgacgagactgagatcaaattgaagtcaaagtcaggcattatgcatgactcgaaatatctggaatcctgggaacggggcagtgcggatattcgcttcgcggaattcgttggggaaaatagagctcacaatcggcagtttccaactgcgacagtaaatatgggacagcagccagacgggcagggcggtttgacccgcgaccgtcatgtgagcgttgacttcctaatgcaaagcgcacccaattcgccttgggcgcaagctttgaaaaagggagaactgtgggatcgcgttcagttgcttgctcgcgacggcaaccgctatctgtcgccgcccagattggaatattctgaccctgcacatttcaccgagttgatgaaccgggttggtttacccgcatcgatgggtcggcaaagccatgcggctagtatcaaattcgaaaagtttgacgcgcaggcagcggttattgtcttaaatggcccagagttacgtgacattcatgacttgtctcctgaaaaactgcaaaatttgtccaccaaagatgtcatcgtcgccgatcgcaatgagaatggtcagagaactggcacgtacaccagcgtcgcggaatatgagcgcttgcagttaaggctgccacccgatgcagcgggggtgcttggtgaagcaactgacaaatattcacgtgatttcgttcggccagagccggcgtcgcgtccaatcagtgacagccgcaggatatacgaaagtcgaccgcgtagccaaagcgtcaacagcttttgacgttcctgctgccgcgtcaacgaggaagctcgtttgacccgggtttgccaatgaaagggctcaatcatggtgaacactacaaagaaaagttttgcgaagtcgcttacggcagatatgcgccgttctgctcagcgcgttgtcgagcaaatgcgaaaagcattgattaccgaagaagaggcgctcaagcggcaagccagactggagagtcccgataggaagcgaaagtatgctgctgatatggcgatagtcgacaaactcgacgtagggtttcgaggcgaaataggctataaaattcttggaaataaccggcttcgagtagacaaccataaagaattaacgcgtgagcacggtagacttcgcaaaaccaaaacggttctgaagcgtaacccggtgacgcaggaagtctatttgggtttatatgaaaggaagtcctggttaagtgtcagcagccatttgtatgctgcggacggcacactccgcatgaagcacgtgaaatacaaagacggacgttttgaggaaaaatgggagcgcgacgaaaatggcgacctgatccgcacaaggtacgccaaccgtggcaggctctttcaacctgtatccgagaaaatgggcgcgccgtatcggagcggccctgacgaccggctctatcgcgatctaacccgtcgaaacggtttcagacgggagacattcgaacgggacgatcacggaaacctcgagcgtatcggcagcaaccatgtcggcttttccaagatttcagtgaaggcacccaatcgtcaaacctcccagacgaagattcaaaaacttggtggcgctttcaacaaatcttttaggtcccttctggacaaggagggcaatgaaatgggccgcgatattttgagccatcgacggctctataacaagcggtctgctgtctacgatgaagctaccggacaattgaagagtgccaagcataccttcggcaagatctacaggagcgaaaccgaatatctcagcgcgggcctcaagaaggtttcaaaaaagatactcggggtgacggtctaccggaaatttgcggcgctcagcgagcgagaatccgaggctgagagactgcgtagttttgaatccggtgcgcatcgccagatctggcaggagcgggcagcgattcccggttcgcccctcccggagactgatgacattcatttcgcacagcagtcgcacctagccaaagccaaccctgatcacgtcgaagctgacgtcacgcgtgtgacagatcaacatgttgatgttgctggacaaacatcatcgtctccccaacggaacttggaaggatggttagattctcaatcacgatacaagccagcaaacatgctgttgtcaaatccagaccttcaagcgaacggacctcgcccatacgaagggttagctcatctcaccctccggcgcgataatgaatctgacgggcacaaggagaatgatcagcggctgcgacatttctcccagccagagccgttggtgttaccgcatcccgggtcgccggaaataactaaggtgtttggctcgcggggagagccggcacacccgagtggaaatctgcacacggcggttggagaaacggcttgcgaaggaccggtgatgtcttcatcctcggacaatcatcagccagctccaggacagcaagaacttttaagtttccttcataatgcgccagccccagtttctgtggcaatacatgatgatcaagagcgacttgcgggggaggcgcccggcggctctttcagaggtagctcagggcgaacgagttcaatgtcggagagtatcttcgacgaagatgtacaagggcatttggtacgggattattcgatcaatactactaacgggtttattgacccgcaatcgttgttcggtgaaccggacttatcgagaggtccaaaatcggggccagaaattccatcggaagattaccatttgtcagcttcggaacaggaaaatttgctgaatcaattgcttagtgtgccactgccggttccttcaccgaagcccgaatgcgcgaggtctatgattttcgaaggttcacgttcaagagagcgttccacctccagagggttctaaggtggaacgcgtctaggcttgcctatcatcaccacctcatgatggtcgcggagaccttcgaagagatatccgacatcattggagaagctgggccggaaactagtgtacctcgcgaatgcatctagatccaatccaatatgcatggcatgccgaatccatgtgggagtttattcttgacacagatatttatgatataataactgagtaagcttaacataaggaggaaaaacatatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaataatgtctaacaattcgttcaagccgacgccgcttcgcggcgcggcttaactcaagcgttagatgcactatacgtaccaatccaaatcggatccccctgcagggctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatctatcgatggtacctatctgcagacggaagggattcctgttaattaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctcgcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggcagcgcaggccgcccagcggcaacgcatcgaggcagaagcacgccccggtgaatcgtggcaagcagccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcaggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatgcatataattgtggtttcaaaatcggctccgtcgatactatgttatacgccaactctgaaaacaactttgaaaaagctgttttctggtattt

SEQ ID NO: 36 - DNA, 31939 bp, Artificial sequence
  Synthetic construct
cgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatctatcgatggtacctatctgcagacggaagggattcctgttaattaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctcgcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggcagcgcaggccgcccagcggcaacgcatcgaggcagaagcacgccccggtgaatcgtggcaagcagccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcaggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatgcatataattgtggtttcaaaatcggctccgtcgatactatgttatacgccaactctgaaaacaactttgaaaaagctgttttctggtatttaaatcctaggaaatatcagccgcttgtgtgttcaattcttctctgtttcacttgaaacaaattgaatagatattcccgctttcaaagccatttacaaatcctctcgtgcagcctaacgccattggcgtggaacaagcgttggcacgaggaagtaagtgcgatgaacggaaggtattcaccgacgcggcaggattttaagacaggcgcgaagccttggtctatattagcccttatcgttgccgcaatgattttcgcgttcatggcggttgcgtcctggcaggacaatgggactacccaggcaatccttagccaaatacgatcgattaacgctgacagcgcctcactgcagcgcgatgtactccgcgctcacacgggcaccgtggcgaactaccgccccattatctccaggctgggagctctgcggaagaatctggaagatttgaagcaattatttaggcaatctcatattgtaagtgagagcaatgctgctcaactgctacgccggctagaagtgtctctaaattcggctgacgcggcggtcgccgcctttggtgcgcaaaatgtccgcctgcaagattcgctggccagtttcactcgtgctttaagcaatcttccaggaaaggcctcaggcgatcagactttagaaaaaccaacagaattggctagcatgatgctccaatttcttcggcaaccaagcccggctatttcattcgagatcagccttgaactagagaggctccaaaaacaacgcggtcttgatgaagctcccgtgcgcatactttcgcgtgaaggtcccattatcttatcgcttttgccacaggtgaacgatctggtgaacatgattcagacgtctgacaccgcagaaattgcggaaatgttgcagcgtgagtgtttggaggtctatagcttgaaaaatgtggaggagcggagcgcacgtatctttcttgggtccgcttcagtgggtctttgcctgtacatcatcaccttagtctataggctacgcaaaaaaaccgattggttagcgcggcgtttagattacgaagagctaatcaaagagatcggagtatgttttgaaggtgaggcggccactacgtcgtccgcgcaagctgcacttggtattattcagcgcttctttgatgccgatacgtgcgcgttagctctagtggaccatgaccgtaggtgggctgtcgaaacattcggtgcgaagcacccaaaacccgtgtgggacgacagggtgctacgcgaaatagtctctcgtaccaaagcgaacgaacgggcgacggtattccgcatcgtatcgacgcaaaaaatcgtacatttgcctcccgaaattccaggtctttcgatactactggctcacaaatccacagataaactaattgcggtttgttcactgggttaccaaagctatcgccctcgaccttgccaaggcgaaattcagcttcttgaactcgccaccgcctgcctctgtcactatatcgatgttcggcgtaagcagaccgaatgcgacgttttggccagacgattggagcatgcgcaacgccttgaggcagttggtacacttgccggcggaatagcacatgaatttaataacattttggggtcaatcctcgggcacgcagaattggcccaaaactcggtgtctcgaacatctgtcacccgaagatacattgactatatcatttcgtcaggcgacagagccatgctcattatcgatcagatcttgacgctgagccgaaaacaagagcgcgtgatcaagccatttagtgtctcagagcttgtgaccgaaatcgctcccttgctacgtatggctcttcccccaaccatcgagcttagtttcagatttgatcaaatgcagagcgtgatcgaaggaagcccgcttgaacttcaacaggtactaattaacctctgcaagaatgcttcccaagccatgactgcaaatggtcaaatcgacatcttcgtcggccaagcttatttaccagctaagaaaattctggcgcatggtgttatgccacctggcgactatgttctactatctgtcagcgacaatggtggaggcatttcagaggctgtgctaccctacatttttgaacccttctttacgacacgtgctcgcaacggtggaacgggtctcggcctcgcttctgtgcatggtcatatcagcgcgtttgcaggttacatcgacgttagttcaactgttgggcatgggacgcgctttgacatttatctccctccgtcttctaaggagcccgtcaatcccgacagctttttcggccgcaataaggcaccgcgtggaaacggggagattgtggcacttgttgagcccgatgacctcctgcgggaggcgtatgaagacaagatcgccgctctgggatatgagccggtcggttttcgtacctttagtgaaattcgcgattggatttcaaaaggcaatgaagccgatctggtcatggtcgaccaagcgtctcttcctgaagatcaaagtcctaattccgtggatttagtgctcaagaccgcctccatcatcattggcggaaatgatctcaaaatgcccctttcaagggagaatgcgaccagggacctttatctgccgaagccgatatcgtccagaactatggcgcatgcaatcctaaccaaaatcaagacgtagagttgcgacgtatcaggactgggcaatcgcggccgccgggcttacgtgtcttttgtaacaaattgcgacgcagttgatatccacttgaaacattagtccgaaattatcgagatctccgctgataaggtatcgaaatggcgataaaattggtattgatactcgtattcacactctttcccgcggcagacgctgcatatgcgaatgaccgcgccaacggtttcatgtggtcaaacgggggcgaaactggagtgaggcttcctcttcgggtgttcaatgccaagccagccaagaacacggtggcgatcatttattccggagacgctggatggcaaaatatcgatgaggcgattggtacctatctgcagacggaagggattcctgtcattggcgtcagttcacttcggtatttctggtcggagcggtccccaagcgaaactgctaaggatcttggtcacataatcgatgtctacaccaagcatttcggtgtgcagaatgttttacttgtaggatattctttcggcgcggacgtcatgccggcaagcttcaataggcttacgcttgagcaaaaaaatcgggttaagcaaatctctctcttggcattgtcacatcaagtcgactatgtcgtctcatttaggggctggctccaactcgaaacggaaggtaagggcggcaatcctctggatgatctcagatccattgaccctgcaatcgtccaatgcatgtacgggcgcgaagaccgtaataatgcttgcccatctctccgacagaccggcgcagaggtgataggcttcagcggaggccatcactttgataatgatttcaaaaaactgtctacgcgcgtcgtctcaggcctcgtggcacgcctaagtcatcagtaatctttagttcctgcaccgcgggacccgcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatgcttaagagatcggggtcgctttctcttgccttgatggtctccttctgttcgtcgagccttgccacgccactctcatctgctgagtttgaccatgttgctcgcaagtgtgccccatcagttgcgacatctacgcttgcggcgatagctaaggtggagagtcgctttgatcctttagcgattcatgacaacacgaccggcgaaacgcttcactggcaagatcacagccaagcaacccaagtcgtcaggcaccgtctcgatgcacggcattcgctggatgttggcctcatgcaaataaactctcgaaatttttctatgctcggtctgacacctgacggtgcgctccaggcgtgcacatcattatctgccgctgcaaacatgctgaaaagtcgttatgcaggcggcgaaacgattgacgagaagcaatttgcgcttcgtcgggcgatctccgcttacaacaccggtaatttcatcggcggttttgcaaacggctacgtgcgaaaagttgaaacagctgctcaatcgctggtgcccgcgttaatcgagcctccaaaagacgatcacgaggcgctaaaatccgaagagacgtgggatgtttgggggtcatatcagcgccgctcgcaggaggatggcgctggcggtttaatcgctccgccaccgccacaccaggacaacggcaaatccgcagacgacaatcaagtcttattcgacttatactaaggaggtgcgcattgatgcgatgctttgagagataccgtttacatctaaatcgcctctcgctctcgaatgcgatgatgcgcgtgatatcgagctgcgccccaagcttgtgcggtgcaattgcatggagcatttcctcatccggacccgccgcagcgcaatctgcgggtggcggcactgaccccgccacaatggttaacaatatatgcacgtttatccttggtccgttcggccagtcactcgctgttctcggcattgtcgctatcgggatctcctggatgttcgggcgggcttcgcttgggctggttgccggcgtcgtcggcggcattgttatcatgtttggggcgagcttcctcggccaaacgctcactggcggtagttgatggctgatcgtttggaagaatcgaccctttacctcgcagccacacggcccgcattgtttcttggggtgccactgacattggcagggttattcatgatgttcgccggctttgtcatcgttatcgttcagaacccgctctacgaagtcgttctcgtgccgttatggtttgcagcccggctcatcgtggagcgagactacaatgcggcgagcgtcgtcctgctatttttgcggaccgcgggaagaagcattgatagtgcagtttgggggggcgctactgttagcccaaatccaattagggttcccccacgagggagaggaatggtgtgatgctcggcgcgagtggaacgaccgaaagatccggtgagatctatctcccttatattggccacctcagcgaccatatcgtccttcttgaagacggatcgatcatgaccattgcgagaattgatggcgttgcattcgagcttgaggaaactgaaatgcgcaatgcgcgttgtcgtgcgttcaacacgctgttgcgcaatatcgctgatgatcatgtgtcaatatatgctcacctcgtacgtcatgccgacgtgccatcatcggcgccgcgacacttccgtagtgttttcgccgctagcctgaacgaagcttttgaacagcgcgtgctctccggccaactcctccgcaatgaacacttccttacgttgattgtctacccacaggcggctttagggaaggtaaagaggaggttcaccaagctaagcggaaaaagggaaaacgatctcacgggccagatcaggaacatggaagatctttggcatgttgtcgctggctctcttaaagcgtatggcctgcatcgtcttggcatccgcgagaagcagggtgtgctcttcaccgaaattggcgaagcgctacggttgatcatgactggtcggttcacaccggttccggtcgtcagcggctcactcggcgcttcgatttataccgacagagtcatttgcggcaagcgaggactcgagatcagaacgccaaaagacagttacgttggatccatctattcgtttcgcgaataccctgcaaaaacacggccgggcatgctcaacgcgctgctatccctcgattttccacttgttctcacgcagagtttttcgttcctgactcgcccgcaagcgcacgcgaaacttagcctcaaatcgagccagatgctgagttccggcgataaagccgtgactcaaatcggcaaattatccgaggctgaggacgcacttgcgagcaacgaattcgttatgggctcacatcatttgagcctttgcgtctatgcagacgatctcaatagtcttggggacaggggcgcgcgggctcggacacgaatggcggatgcaggtgccgtggttgtccaagaaggtattggtatggaagcggcctattggtcccaattgccggggaattttaagtggcgcacacgccctggcgcaatcacttcacgcaatttcgcagggtttgtctctttcgaaaactttccagagggcgccagctcaggccactggggcaacgcgattgcccgatttcgtaccaatggcggaacgcctttcgactatatcccgcatgagcacgatgttggcatgacggcaatattcgggcctatcgggaggggtaagacgacgctcatgatgtttgttctagccatgctcgaacagagcatggtcgaccgtgcaggtacggtcgtgttctttgacaaggaccggggtggcgaattgctggttcgcgccacaggaggaacatatttggcacttcacagaggcacacccagcgggttggcgccgttgcgtggcctagaaaacacagcagcctcacacgattttctgcgcgaatggatcgtggctctcatcgagagtgatggtcggggtgggatttctccggaagagaaccgccgtctggtccgtggtatccatcgtcagctctcgtttgatccacaaatgcgttcaatcgcggggttacgtgaatttttgttgcatgggcccgccgaaggcgcaggagcgcggctccaacgctggtgccggggccatgcgcttggctgggcatttgacggcgaagttgacgaagtaaagttagatccgtcgattaccggcttcgacatgacgcatcttctcgaatacgaggaagtatgcgctcccgctgcagcatatctcctgcatcggattggagccatgatcgacggccgccgttttgtgatgagctgcgatgagtttcgcgcctatttgttaaaccctaaattttcgactgtcgtcgacaaattcctcctgaccgttcgaaaaaacaacgggatgctaatactggcaacgcagcaaccagagcatgttctggaatcgccgctaggagccagcttggttgcgcaatgtatgacgaagattttctatccatcaccaaccgcagatcgatcggcttatgtcgatggactgaaatgtaccgaaaaggaatttcaggcgatccgtgaagacatgacggtcggcagccgtaagtttcttcttaaacgagaaagtggaagcgtcatctgcgaatttgatctgcgggatatgcgtgaatatgtcgccgtgctttcggggcgtgccaacacggtgcgctttgcaactcgactacgcgaggcacaagaaggcaactcatctggctggctcagcgaattcatggcccgtcaccacgaggcagaagattgataaggtaggaaacgatgaagacgacgcaacttattgcaacagttttgacctgcagctttctatatattcagcccgcgcgggcgcagtttgttgttagcgacccggcaacggaggctgagacgctcgcgactgcgctcgcgactgcggagaatctcactcagactatagcgatggttacgatgttgacgtcggcctacggcgttactggactactgacttcgctcaaccagaaaaatcagtatccttcgacgaaggacctagacaatgaaatgttttcgccgcgaatgccaatgtcgaccacggcacgtgcgatcaccagcgatacagatcgtgcagtcgtgggtagtgatgctgaagcggacctgttgcgatcgcagatcaccggttccgcaaacagcgctggcattgcggctgacaatctggaaacgatggacaaacgcttgacggcgaatgctgatacgtctgctcagctttcccgatctcgcaatatcatgcaggcaaccgtgaccaatggtttgcttctcaagcagatccatgacgcaatgattcaaaatgtacaggcgacaagcctattaacgatgactaccgcgcaggccggccttcacgaggcggaagaggcggccgctcaacgcaaggagcatcaaaagaccgctgtcatctttggtgccctcccctaaggctgggcgatttgttcatccgcccgcatcctcgccgaatgcgagctcattttatccaacattatgcgacaaaccagtcaagttcaggtccaatcgatgaatttcacgattccggcgccgtttacggccattcatacgatcttcgatgtagccttcacgacaggcttggactcgatgcttgagactatccaggaggcggtgagtgcgccattgatcgcctgtgtcactctttggattattgttcagggtattttagtcatacgcggcgaagtcgatacccgtagcggtatcactcgggtgatcacggtcaccatcgttgttgctctaattgttgggcaggctaactaccaagactatgtggtttccatcttcgaaaagacggtcccaaactttgttcagcagtttagtgtaaccggcttgcctctgcagactgttccggcacagttggatacaatgttcgccgtgacccaggccgtttttcagaaaatcgcatccgaaatcggtccgatgaacgaccaggacatccttgctttccaaggggcacagtgggtcctttacggcacgctctggtctgccttcggagtttacgacgccgttggaattctcacgaaagtgcttctcgcgatcgggcctctgatcctcgtcggatatatttttgatcgcacgcgggacatcgcagctaagtggatcgggcaacttatcacctacggtctcttgcttctcctcttaaacctcgtggcaacgatcgtcatcctaaccgaagcgactgcgctcacccttatgcttggtgtaatcaccttcgccggtacgaccgcggccaagatcattggtctttacgaactcgatatgttttttctgacaggggatgcgctcattgtcgctttgccggcgatcgccggcaacattggaggcagttactggagcggcgcaacccaatctgccagcagcttgtaccgtcgcttcgctcaggttgagcgaggctaggtcgcgcaaaaattcgcctcaatggagaattctatgaagtattgcctgctgtgcctagttgtcgctttgagcggctgccagacaaacgacacattagcgagctgcaaaggcccgatcttcccgctgaatgtggggcgatggcagcctactccgtcagatcttcagctcggcaattcgggtggacgctatgacggggcctgaatatgccatgctagtggcgcgcgaaagccttgccgagcactataaggaagtagaagcctttcaaaccgcgcgagcgaaatcggcgcgacgtctctccaaactcattgcagctgtcgcagctatcgcgattttgggaaatgttgctcaagcgttcgctatagccacaatggtgccgttgagcaggcttgtgcccgtatatctatggatacggccggacggcaccgttgacagcgaggtgtctgtctcgcgattgcctgcaactcaagaggaggccgtcgttaacgcctcattgtgggagtacgttcgcctgcgcgagagttatgatgccgacaccgctcagtacgcctacgacctggtatcgaacttcagtgccccaacagtgcgccaggattaccagcaattcttcaactatcccaatcccagttcgcctcaagtcattcttggcaaacgcggcagggtggaggtcgagcacatcgcttcaaatgatgtaactccaagcacgcagcaaattcgctataaaaggaccctcgtcgttgacggcaaaatgcctgtggtgagtacgtggaccgcgacagttcgctacgaaaaggtgaccagcttgcccggcagattgagactaaccaacccggcaggtctggttgtcacctcctatcagacatcggaagataccgtttcaaacgtaggccacagcgaaccatgatcagaaaagcacttttcattttagcatgtttatttgccgctgcgactggtgcggaggctgaagacactccaatggcgggcaagctagatccgcgcatgcgttatttggcttacaatcccgatcaagtggtgcgcctctcgacggcggttggagctactttggtcgtaacattcgccacgaacgaaacggtgacagcggttgccgtttcaaatagcaaagatctagcagccctaccgcggggaaattatctatttttcaaggcaagccaggtcctcacgcctcagccagtaatcgtgctaaccgcaagcgactccgggatgcgccgttatgttttcagtataagttccaagactctgtcccacctcgataaagagcagcccgatctctattacagcgtccaattcgcctaccccgccgacgatgcggcggctcggcgaagggaggcacaacagaaggctgttgtggacagactacacgcggaagcacaatatcaacggaaagctgagaatttattggatcagcctgtcacagcccttggtgcggcggacagtaattggcactacgtcgcccaaggcgatcgttcgctgttgccactcgaagtcttcgacaatggatttacgacggtattccactttccgggcaatgtacgcataccctccatctacaccatcaatcctgatggcaaggaagctgttgccaactattcagttaaagggagcgatgtcgagatttcttcggtttcccgaggttggcgtctgagggatggccacacagtactatgtatctggaacaccgcttacgatcccgttggccaaaggccgcaaacgggcacggtgaggcccgatgtgaaacgcgtcctgaagggggcgaagggatgaataacgatagtcagcaagcggcacatgaggttgatgcatctggatccctggtctccgacaaacatcgccggcgtctttcggggtctcagaaattgatcgtcggaggtgtcgttctcgcgttatcattaagcctcatttggctaggtgggcgccaaaagaaggtgaatgagaacgcatcgccgtcaactttgatcgcaacaaacaccaagccatttcatccagctccgattgaggtgccgccggatcctccagcggttcaagaggctgttcagcctgctgctcctctaccgccgaggggcgaaccggagcggcatgagccacggccggaagaaacaccgatttttgcatatagcagcggcgatcaaggggtcagcaaacgcgccattcagggcgacacgggccgaagacaagaaggcaagcgtgacgacaactccttgccgaatggcgaagtgtccggcgagaacgatttgtcgatacgtatgaaacccaccgagctgcagcccagcagcgccacgctcttgccgcaccccgattttatggtaacgcaagggacaataattccgtgcatcttgcaaaccgcaatcgacacaaatttggcaggctatgtaaagtgtgtcttgcctcaggatattcgtggaacaacgaacaatatcgtgcttcttgatcgtggcaccaccgttgttggcgaaatacagcgtggcttgcaacagggagatgggcgcgtttttgtgttgtgggatcgcgccgagacacctgaccatgcgatgatctcgttaacatcgccaagcgcggacgaactcggtcgctcaggattgccgggctcggtcgacagccacttctggcagcgttttagcggagctatgctcttgagtgttgttcaaggcgccttccaggcagctagcacctacgccggcagctcgggtggcgggatgagcttcaacagctttcaaaataacggtgagcagacaactgagacagcccttaaggcaaccatcaacataccgccaaccctgaagaagaatcagggtgacaccgtttccattttcgtagcacgggacctcgatttctttggtgtttaccagctccgcctgactggcggcgccacgcgggggaggaaccgccgctcttaatgaattcaaatttccgcttagagataggatacattgtaaatggaagtggatccgcaactacgctttcttctgaagccgattttggaatggctcgatgacccgaagactgaagaaattgcgatcaatcgacctggagaggcatttgtgcgccaagccggcatttttaccaagatgcctttgcccgtctcttatgatgatcttgaagatatcgctattttagcgggcgcgctgagaaagcaggatgtcggaccacgtaaccccctctgcgccactgaacttcctggtggtgaacgactacaaatctgtctgccgccgaccgttccctcgggcaccgtcagcttgaccattcgacggccaagctcgcgtgtttctggtcttaaagaagtctcctcccgttatgatgcttcgaggtggaaccagtggcagacacgaaggaaacgccaaaatcaggatgatgaagctatccttcagcattttgacaacggggatttggaagcgtttctgcacgcatgcgtcgtcagccgactgacgatgttgctatgtggccctaccggaagcggcaagacaacaatgagcaagaccttgatcagcgccatccccccccaggaaaggctaatcaccatagaagatacgctcgaactcgtcattccacacgataatcatgttagactactctactccaagaacggtgctgggctgggtgctgtgagcgccgagcacttgctccaagcaagtctgcgtatgcggccggaccggatattgcttggcgagatgcgcgacgatgcagcatgggcttatctgagtgaagtcgtctcgggacatccgggatcgatttcaacaatacacggcgcgaatccaatccaaggattcaagaaactgttttcccttgtcaaaagtagcgcccaaggtgctagcttggaagatcgcacactgattgacatgctctctacggcgatcgatgtcatcattccattccgtgcctatgaggacgtttatgaagtaggcgagatctggctcgcggcggacgcacgacgccggggcgagaccataggcgatctccttaatcaatagtagctgtaacctcgaagcgtttcacttgtaacaacgattgagaacttttgtcataaaattgaaatacttggttcgcattttcgtcatccgcggtcagccgcaattctgacgaactgcccatttagctggagatgattgtacatccttcacgtgaaaatttctcaagcgctgtgaacaagggttcagattttagattgagaggtgagccgttgaaacacgttcttcttatcgatgacgatgtcgctatgcggcatcttattatcgaataccttacgatccacgccttcaaagtgaccgcggtagccgacagcacccagttcactagagtactctcttccgcgacggtcgatgtcgtggttgttgatctaaatttaggtcgtgaagatgggcttgagatcgttcgaaatctggcggcaaagtctgatattccaatcataattatcagtggcgaccgccttgaggagacggataaagttgttgcactcgagctaggagcaagtgattttatcgctaagccgtttagtacgagagagtttcttgcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttacgacaggagtatagatgttctcattttgcggctgcgccgcaaacttgaggcggatccgtcaagccctcaactgataaaaacagcaagaggtgccggttatttctttgacgcggacgtgcaggtttcgcacggggggacgatggcagcctgagccaattgcatttgattaatttaggtgactgaggacgcggccagcggcctcaaacctacactcaatatttggtgaggggttccgataggtccctcttcaccaattgctcgatggcttctctccagcaaagaatgacgcgagcgcggcggtagccagcttgtggccgaaagctcgagcggtctccaaccccaacggatcaaaatgacttcgagcgacctcgagcaacgcaaccgggaacatgcgtgaggtctgaacgagaacggatttttctgtagttgaagggatcggataacttttcggggccacgcgaaatgatccatctgccagcatgctttcgaaatcgtccaacgcgcgccttaaaatcatttgtagcgacttcgagggactgtattgccgaacgaggttgtcatatgttttcgacacttgaggcgcgggcggtcgcgctgaaagaaaaacctggagctttttcggggacggaggtggactaagggcatccacagttagcttaagttgtcgatcgggactgtaaatgtgatcggcgacgagaggctcacgttgctggtctttctcgtcggctttttcaggcaagtgctggaggtccagcttctggggaacaagtgtcgggttgggatggtggatctcgggtcgagcaccagcaagccgccgtgcttcgccgaccgacaatgcgggcttgcgaattgccatcttcaagcctccaagattttgctgatcagtttcgaaatgaccacgacttcctccatcgcaatccgaagattcctctctatgaggcgcatcgtcggatcagttcccgtgtttagtaatgtaagatgcaacatgccgcgttctttcatcgcggcaaatgcatctctttcatgcatgggagacggtacaactggaaggctctctagcgtctctgacatcctgcgttgcgatgttgtcaatcggccgaccgggacgcgttggcgcaaaacagctgtaggaattgccaaattttcactcaacagcagctcgatgacgtagcggtaggtagatagtgcctcatcgatgtcgagcggcgttagcatggtggggatcagaagcaggtttgagctagcgatgattgtgttgttgagctcgctcgagccgccacgcgtatcggccaacgcataatcaaatccttcgagctcggcattttcataggctgcttcaagaaggggcatttcgtcggcggaatagacttcacagcgaggatcccaggtactgctttgtaaggcgttttctctccatcgcgtcagaggccggttttcgtcggcatcaaagagggccactcgtttaccgtcatttgccaaagcagcgcaaaggcccatgagtgcggtggttttgccagcaccccctttgaaagaacaaaacgtcaaaagttgcatattctgatcccgcctatcctgtgaaaccggagtgcatttgtatttttgttcgtataaatgtttttgtgattatcgatgagtaaaagcgttgttacactatttttatttcacattcgttataagacaattgcaaatgtagcaagtatattcagtattgactgtaaatgtactgttgatttcatattgagcagggctagacttccatccgtctacccgggcacatttcgtgctggagtatccagaccttccgctttctttggaggaagctatgtcaaaacacaccagagccacgtcgagtgagactaccatcaaccagcatcgatccctgaaagttgaagggttcaaggtcgtgagtgcccgtctgcgatcggccgagtatgaaaccttttcctatcaagcgcgcctgctgggactttcggatagtatggcaattcgcgttgcggtgcgccgcatcgggggctttctcgaaatagatgcagacacacgagaaaagatggaagccatacttcagtccatcggaatactctcaagcaatgtatccatgcttctatctgcctacgccgaagaccctcgatcggatctggaggctgtgcgagatgaacgtattgcttttggcgaggctttcgccgccctcgatggactactgcgctccattttgtccgtatcccggcgacggatcgacggtcgctcgttactgaaaggtgccttgtagcacttgaccacgcacctgacgggagaaaattggatgcccgatcgcgctcaagtaatcattcgcattgtgccaggaggtggaaccaagacccttcagcagataatcaatcagctggagtacctgtcccgaaagggaaagctggaactgcagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtaagcttccccgcaggtaccgaccaaaccgcagcttatgaagcaagccgggaatgggcagccgagatgtttgggtcaggatacgggggtggccgctataactatctgacagcctaccacgtcgaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaatatcgacgccttgagcggatgcaggctcaaaagattcaattcgaagatacagattttgatgagacctcgcctgaggaagatcgtcgggacctcagtcaatcgttcgatccatttcgatcggacgcatctgccggcgaaccggaccgtgcaacccgacatgacaaacaaccgcttgaaccgcacgcccgtttccaggagcccgccggctccagcatcaaagccgacgcacggatccgcgtaccattggagagcgagcggggtgcccaaccatccgcgtccaaaatccctgtaactgggcatttcgggattgagacttcgtatgtcgctgaagccagcgtgcccaaacaaagcggcaattccgatacttctcgcccggtgactgacgttgccatgcacacagtcgagcgccagcagcgatcaaaacgacgtcatgacgaggaggcaggtccgagcggagcaaaccgtaaaagattgaaggccgcgcaagttgattccgaggcaaatgtcggtgagcccgacggtcgcgatgacagcaacaaggcggctgatccggtgtctgcttccatccgtaccgagcaaccggaagcttctccaacgtgtccgcgtgaccgtcacgatggagaattgggagaacgcaaacgtgcaagaggtaatcgtcgcgacgatgggcgcggggggacctagactagtagacaggaaggaccgaataatggcaaatggtcagttcacgatacgctctgctcgcccggcctccgtcggactgacaggcgaacggcgtggagccgcatccgcctctagctctgcactgtccaatgttcaaagagatgttagggataggctgattccaactagctcaccaagattaccaaatgcagccatattgcgtgattcctcgggaagagcgtcgactggtctgcggtacatggcggctactcttcattggtctgcgatcgcgccattatcgctaataaacagcaacgacctggctccggccgcttatgactttgagacgcgaaataacgcaagaaatgtgactgccaaagtcggcagggcagtccctgttcccaagcaaggcgggctcggcaaaacgctcgcacccgtaccccttagtacacgtatatcaagggtcaattccgaccgaagactgcccgctgacgcagaagaccgccctgaaacgcgcgacccccagaaaggacgtggcagtcatggtgcgacgccaaccttacatgaaaagattggaaccgcgtttgctcgaagattgcgaaagcatacgtactatattgtttgcagttgctgccagacccggagcgcgttgacgatgggtgcaaagatttcggtgaagtcatgaactccagcaagacttcgccccagcgtatgaccctgagcatcgtatgttcgctggcagccggtttttgtgcggccagctgctatgtaacgttccgccggggcttcaacggcgaagcgatgatgacgttcgacgttttcgctttttggtatgagaccccgctttacttgggttatgccagcaccgtcttctggcgtggtttatctgttgtcatctttacctcgctgatcgttctttcaagtcagctcatcatatcgctgcgcaatcagaagcatcatgggacagctcgttgggcagaaattggcgaaatgcggcatgctggttatctgcagcgttacagtcgcatcaaggggccgatctttggaaagacatgtggtcccctttggttcggcagttatttgaccaatggcgaacagccacacagtcttgtcgtcgcgccaacgcgtgctggcaaaggcgtcggcatcgtcattccaacgctgttgaccttcaagggctcggtaatcgcccttgacgtcaagggagaattgtttgaactgacgtccagagcacgcaaagcgagcggcgacgcagttttcaagttctcccccctagatcctgagcggaagactcattgttacaatccggtcctggatattgccgcacttccgcccgaacgccagttcactgaaacacgccgtctagctgcgaaccttattacggctaagggaaagggagcagaaggctttattgacggcgcacgtgacctgttcgtcgcgggaatccttacctgcattgagcgtggcacaccaacgattggcgcggtatatgacctatttgcgcagcctggcgaaaagtataagctttttgcgcaactcgcggaggaaagcctaaacaaagaggctcagcgtatcttcgataatatggcgggcaacgacacgaaaattctgacatcgtacacctctgtgctgggcgacggtggactgaacctgtgggctgatccgcttatcaaagcagcgacaagccggtcagacttttccgtttacgatctccggaggaagaagacctgcatttatctttgtgtcagtcccaacgatctggaggtcttggcaccacttatgcgcctgatgtttcagcagctcgtgtcaatcttgcagagatcgctgccaggtgaagacgagtgccatgaagttttatttctcctcgacgaattcaaacacctgggcaagcttgaggccatagagaccgcgatcacaaccatcgccggttacagaggccgctttatgtttattattcaaagtctttcggccttgtcgggcacatacgatgacgcaggaaaacaaaactttctgagcaatactggcgtacaagtatttatggccacggctgatgacgaaactccaacctacatctcaaaagctatcggcgaatatacgtttaaagcgcgttcgacctcttacagtcaagccagaatgttcgaccacaacatccagatttctgatcaaggtgcagcccttttgcgccccgaacaagtgcgcctgctagacgatcagagtgaaatcgttctcatcaaagggcgacctccactcaaattacgaaaggtgcagtattattccgatcgtacgctgaaaggccttttcgaacgccagatgggctctctgcctgagcccgcacccttgatgctttccgactatagcaacgatcaagttcaataccacttggctccgatagcaaattttaatgaggatgctgcaccgcaaaacagaactgtggccgaggaccatggaagtgttaaagtcggtgctgatatccctgaacgcgtgatgggaataaatggtgacgaggaacaagccgatgcgggcgagataccgccggaatcggttgtgcctccagaattgacgctcgctctgaccgctcaacagcaattgttggaccagattattgcacttcagcaaagatcgaggtccgcaccggcatagcctgcgaaatgatcttaatggtgcaattcgtttcaggcggcatgttgcggttcaacaaagtgtacgccagatcccaattggccctttacgaggtgtcggtatgacaggaaagtcgaaagttcacataagaggttcggctgacgcgcttcctgacgttcctggcggaagtactaccgccccttttttaaccgaaccttctcgggatcaggttgatgcctcgtttgaggtccaaaccgactacagccagtctacttccgtgtcgtttacctatgatggtgttggacttggtcctgccgagcgtgcggcttacgagaactggtgcgaaccgggccggcccacttggaaagatcttataatcaaggcacgtgtcgatccgattgacgatgtgacctggctccgagatttagaagaggacaccccctcaaccttcagatacgaagggatgcctctgggcatcggggaacgacaggcctacgaaaattggcaagaggacgctcagccgacatgggaagaccttgttgtcagcgcacgcttgacggaacttggccgtccacacgggattaccggcgagtatacatccctcgcaggatcgaagaatacaagttcaatttcattgaagcgaaagcggagcaacttaattgatgatgagaattcatccggatcgttttcatatgacgggatgaagctcggggaagccgagcgttctgcatatggtgactgggccgaggcggagccacccacgtggaaagatttggtattgagggcacgcgtttcctcgatcaatgactctgcttggctttttgattcacaaacatcttcatcatcatttgaatacaacggtgttcccttgggcgagccggaacggcaggctctcagacaatggcaaggagacgctcagcctacctgggaagatctcgttgttaacgcgcgtatggcagaactttgccatgctggttggattgaaggtcaaaaaggttgctttgaagagcgcggggaggctctgcccgcgtcggaacgcggttcgcaacgccccattggtcaacggacagattcctccgattcttttgtgtatgatggcacaaggctcggagcacctgagcgaactgcttatgaacgctggagtaagagggaacgcccgacttgggaagatctcatcttagatgcacaccaggccaggactgaaagtgacgctgttacgacccaagcgattggtcagtcgtcctcaccggttttcttatatgaaggaaagtcgctcggagacagggaacgaaaggcttacgaaaaatggcggcagccagcccaaccgcgatggcaaaatcttgtagtgaacgctcgtctggcagaaatcgatccctcagcctggattgccgatgagcgcgatccgcttgatgatagcgacgcgcttggtcgcccgtcgtacacaagcttgacggatagatcagacgtccctttagacgatcaatcaatctatcgtcgttccgacctagtaagggagcaggtgccagaatcgtctcaaaggcaattcgcagcatgttcagaatctgaaacgaggcctgtgcaatggtttactgcttctgggtcagatgcaaacaatacggaaaatatcaccgccagcgatcccgtcgatcgcacgggtggagttaagcggctaggctccaaaagcgacagaaccgttacagcttctatccatgacgtgaattccagcacaaggcgactgttgcttaacgaatttggatcggaggctccgcgcccttcgccagaaaagactgttcgcttaagaagcgacaatattggcacctatgggagccggaaaaatgaacgagcgcggctcgcgaccgaaaccggtgcgtatgagtcggagcatattttcgggttcaaggctgtccacgatactgcgagagcgacgaaagagggccggcgtctcgaaaggcccatgcccgcctaccttgaggataaggggcttcatcgccaacatattggcaccgggagaggacggaccaaacttgtcgggcgcggatggccggatgacacaagctatcgctcggatcaaagggcaactctgtcggaccccgttgcgcgctcggaaggcgcgacggcctcaaatgggtatcaattgaaccaattgggttacgcgcaccaactcgctagcgatggcctgcaaagtgaatcgcccgatggtgttgccttgccaattcaagtggcaacaacgagctacaactatacagtgagccgcgatcctgtccttgttccgccggataaaaacgaagcccctcaattgctgcatcttggtccccgtggtcaaaccgaagctgttcttgcccgcgaaacagcattgactggaaaatggccgactctcgagcgtgagcagcaagtgtatcgcgagtttttggccttatatgacgtaaaaaaagatcttgaggccaaatcagtcggcgtaagacggaaaaaaaaagaagttatttctgcgttagaccgaactgcgcgcttgataagcacgtcgccttcgaaagctcgatccaaagcagagactgaaaaagccattgatgagctcgatgatcgacgagtttatgatccgcgtgatcgagctcaagacaaagcgtttaaacgctgataagtcgccaatatagtgatcatttgcagtattcgcatcgatcgctggttgatattctgccgctggtcgaccggctgctcgtcgccaaaaatgctcacagggatacgatggcctcggtcaggccgcgtgcgtcctgtctttccagttcctccctttcagctcgattgtggcatcatttattgcctgctcattgcagttgaaacgcgatatccgtttcaagacccgggtatggatggtactttggagatatgagcacttgccagacttcccggtcggagacttaaaaattccgataagaggttacggacgacggaccttataagtggatcgtctagtggtggcgccgatcaaaacagttccgcccccggctttgctcaaagtagcaaagcagctttatctcgggttgcggaggattttctagaaaaccgcaattttgcaggagagaacatatggccatcatcaagccgcatgtgaacaaaaataggacaacctcgccgatagagagaccggagtctctcatagaggaaatgagcggcagtcatccgccgagtggttttaccaacctggatctcgctatgatcgagctggaggactttgtccatcggtgcccgctcccagaagacaatcttgctggtcagaaggagtgagacgatggatccgtctagcaatgagaatgtctatgtgggtcgcggtcacaacatcgaaaatgatgatgacactgaccccaggcgttggaagaaggcgaatatcagttccaacaccatctccgatattcagatgacgaatggcgaagacgtacaatcagggagccctacccgaacggaagttgtaagcccacgtctggattatggatcggtcgactcctcctccagcctttattctggcagcgagcacggaaatcaagctgagattcaaaaagagctgtccgtcttgttctcgaacatgtctttgccaggcaacgatcggcgcccggacgaatacattctcgtgcatcaaacgggacaagatgcttttactggtattgccaaaggcaacctcgaccaaatgcccaccaaggcggaatttaacgcgtgctgccgtctctacagggacggagccggtaattactacccgccacctctcgcattcgacaagattagcgttccagagcaactggaggaaaaatgggggatgatggaggcgaaggaacgtaacaaactgcggtttcagtacaagttggacgtatggaatcatgcgcacgctgatatggggatcacgggcacagagatcttttatcaaacagataagaacataaagctcgaccggaattataaactaagacctgaagaccgatacgtacaaacagaaaaatacgggcgccgggaaattcaaaagcgatatcaacacgaactccaggctggttcgctgctgcccgatattatgatcaaaactccccaaaatgacatccacttcgtgtacaggtttgccggcgacaattacgccaacaaacagttcagcgagtttgaacacaccgtcaagcgcaggtatggcgacgagactgagatcaaattgaagtcaaagtcaggcattatgcatgactcgaaatatctggaatcctgggaacggggcagtgcggatattcgcttcgcggaattcgttggggaaaatagagctcacaatcggcagtttccaactgcgacagtaaatatgggacagcagccagacgggcagggcggtttgacccgcgaccgtcatgtgagcgttgacttcctaatgcaaagcgcacccaattcgccttgggcgcaagctttgaaaaagggagaactgtgggatcgcgttcagttgcttgctcgcgacggcaaccgctatctgtcgccgcccagattggaatattctgaccctgcacatttcaccgagttgatgaaccgggttggtttacccgcatcgatgggtcggcaaagccatgcggctagtatcaaattcgaaaagtttgacgcgcaggcagcggttattgtcttaaatggcccagagttacgtgacattcatgacttgtctcctgaaaaactgcaaaatttgtccaccaaagatgtcatcgtcgccgatcgcaatgagaatggtcagagaactggcacgtacaccagcgtcgcggaatatgagcgcttgcagttaaggctgccacccgatgcagcgggggtgcttggtgaagcaactgacaaatattcacgtgatttcgttcggccagagccggcgtcgcgtccaatcagtgacagccgcaggatatacgaaagtcgaccgcgtagccaaagcgtcaacagcttttgacgttcctgctgccgcgtcaacgaggaagctcgtttgacccgggtttgccaatgaaagggctcaatcatggtgaacactacaaagaaaagttttgcgaagtcgcttacggcagatatgcgccgttctgctcagcgcgttgtcgagcaaatgcgaaaagcattgattaccgaagaagaggcgctcaagcggcaagccagactggagagtcccgataggaagcgaaagtatgctgctgatatggcgatagtcgacaaactcgacgtagggtttcgaggcgaaataggctataaaattcttggaaataaccggcttcgagtagacaaccataaagaattaacgcgtgagcacggtagacttcgcaaaaccaaaacggttctgaagcgtaacccggtgacgcaggaagtctatttgggtttatatgaaaggaagtcctggttaagtgtcagcagccatttgtatgctgcggacggcacactccgcatgaagcacgtgaaatacaaagacggacgttttgaggaaaaatgggagcgcgacgaaaatggcgacctgatccgcacaaggtacgccaaccgtggcaggctctttcaacctgtatccgagaaaatgggcgcgccgtatcggagcggccctgacgaccggctctatcgcgatctaacccgtcgaaacggtttcagacgggagacattcgaacgggacgatcacggaaacctcgagcgtatcggcagcaaccatgtcggcttttccaagatttcagtgaaggcacccaatcgtcaaacctcccagacgaagattcaaaaacttggtggcgctttcaacaaatcttttaggtcccttctggacaaggagggcaatgaaatgggccgcgatattttgagccatcgacggctctataacaagcggtctgctgtctacgatgaagctaccggacaattgaagagtgccaagcataccttcggcaagatctacaggagcgaaaccgaatatctcagcgcgggcctcaagaaggtttcaaaaaagatactcggggtgacggtctaccggaaatttgcggcgctcagcgagcgagaatccgaggctgagagactgcgtagttttgaatccggtgcgcatcgccagatctggcaggagcgggcagcgattcccggttcgcccctcccggagactgatgacattcatttcgcacagcagtcgcacctagccaaagccaaccctgatcacgtcgaagctgacgtcacgcgtgtgacagatcaacatgttgatgttgctggacaaacatcatcgtctccccaacggaacttggaaggatggttagattctcaatcacgatacaagccagcaaacatgctgttgtcaaatccagaccttcaagcgaacggacctcgcccatacgaagggttagctcatctcaccctccggcgcgataatgaatctgacgggcacaaggagaatgatcagcggctgcgacatttctcccagccagagccgttggtgttaccgcatcccgggtcgccggaaataactaaggtgtttggctcgcggggagagccggcacacccgagtggaaatctgcacacggcggttggagaaacggcttgcgaaggaccggtgatgtcttcatcctcggacaatcatcagccagctccaggacagcaagaacttttaagtttccttcataatgcgccagccccagtttctgtggcaatacatgatgatcaagagcgacttgcgggggaggcgcccggcggctctttcagaggtagctcagggcgaacgagttcaatgtcggagagtatcttcgacgaagatgtacaagggcatttggtacgggattattcgatcaatactactaacgggtttattgacccgcaatcgttgttcggtgaaccggacttatcgagaggtccaaaatcggggccagaaattccatcggaagattaccatttgtcagcttcggaacaggaaaatttgctgaatcaattgcttagtgtgccactgccggttccttcaccgaagcccgaatgcgcgaggtctatgattttcgaaggttcacgttcaagagagcgttccacctccagagggttctaaggtggaacgcgtctaggcttgcctatcatcaccacctcatgatggtcgcggagaccttcgaagagatatccgacatcattggagaagctgggccggaaactagtgtacctcgcgaatgcatctagatccaatccaatatgcatggcatgccgaatccatgtgggagtttattcttgacacagatatttatgatataataactgagtaagcttaacataaggaggaaaaacatatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaataatgtctaacaattcgttcaagccgacgccgcttcgcggcgcggcttaactcaagcgttagatgcactatacgtaccaatccaaatcggatccccctgcagggctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacc

SEQ ID NO: 37 - DNA, 3671 bp, Escherichia coli
tcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccaccatggcatcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgattcccacacattatacgagccggaagcataaagtgtagctagatccgaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttag

SEQ ID NO: 38 - DNA, 15028 bp, Escherichia coli
ctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccactcttcattaaccgctatatcgaaaattgcttgcggcttgttagaattgccatgacgtacctcggtgtcacgggtaagattaccgataaactggaactgattatggctcatatcgaaagtctccttgagaaaggagactctagtttagctaaacattggttccgctgtcaagaactttagcggctaaaattttgcgggccgcgaccaaaggtgcgaggggcggcttccgctgtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacgccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggctttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcattgggtgactcctgacggttgcctctggtgttaaacgtgtcctggtcgcttgccggctaaaaaaaagccgacctcggcagttcgaggccggctttccctagagccgggcgcgtcaaggttgttccatctattttagtgaactgcgttcgatttatcagttactttcctcccgctttgtgtttcctcccactcgtttccgcgtctagccgacccctcaacatagcggcctcttcttgggctgcctttgcctcttgccgcgcttcgtcacgctcggcttgcaccgtcgtaaagcgctcggcctgcctggccgcctcttgcgccgccaacttcctttgctcctggtgggcctcggcgtcggcctgcgccttcgctttcaccgctgccaactccgtgcgcaaactctccgcttcgcgcctggtggcgtcgcgctcgccgcgaagcgcctgcatttcctggttggccgcgtccagggtcttgcggctctcttctttgaatgcgcgggcgtcctggtgagcgtagtccagctcggcgcgcagctcctgcgctcgacgctccacctcgtcggcccgctgcgtcgccagcgcggcccgctgctcggctcctgccagggcggtgcgtgcttcggccagggcttgccgctggcgtgcggccagctcggccgcctcggcggcctgctgctctagcaatgtaacgcgcgcctgggcttcttccagctcgcgggcctgcgcctcgaaggcgtcggccagctccccgcgcacggcttccaactcgttgcgctcacgatcccagccggcttgcgctgcctgcaacgattcattggcaagggcctgggcggcttgccagagggcggccacggcctggttgccggcctgctgcaccgcgtccggcacctggactgccagcggggcggcctgcgccgtgcgctggcgtcgccattcgcgcatgccggcgctggcgtcgttcatgttgacgcgggcggccttacgcactgcatccacggtcgggaagttctcccggtcgccttgctcgaacagctcgtccgcagccgcaaaaatgcggtcgcgcgtctctttgttcagttccatgttggctccggtaattggtaagaataataatactcttacctaccttatcagcgcaagagtttagctgaacagttctcgacttaacggcaggttttttagcggctgaagggcaggcaaaaaaagccccgcacggtcggcgggggcaaagggtcagcgggaaggggattagcgggcgtcgggcttcttcatgcgtcggggccgcgcttcttgggatggagcacgacgaagcgcgcacgcgcatcgtcctcggccctatcggcccgcgtcgcggtcaggaacttgtcgcgcgctaggtcctccctggtgggcaccaggggcatgaactcggcctgctcgatgtaggtccactccatgaccgcatcgcagtcgaggccgcgttccttcaccgtctcttgcaggtcgcggtacgcccgctcgttgagcggctggtaacgggccaattggtcgtaaatggctgtcggccatgagcggcctttcctgttgagccagcagccgacgacgaagccggcaatgcaggcccctggcacaaccaggccgacgccgggggcaggggatggcagcagctcgccaaccaggaaccccgccgcgatgatgccgatgccggtcaaccagcccttgaaactatccggccccgaaacacccctgcgcattgcctggatgctgcgccggatagcttgcaacatcaggagccgtttcttttgttcgtcagtcatggtccgccctcaccagttgttcgtatcggtgtcggacgaactgaaatcgcaagagctgccggtatcggtccagccgctgtccgtgtcgctgctgccgaagcacggcgaggggtccgcgaacgccgcagacggcgtatccggccgcagcgcatcgcccagcatggccccggtcagcgagccgccggccaggtagcccagcatggtgctgttggtcgccccggccaccagggccgacgtgacgaaatcgccgtcattccctctggattgttcgctgctcggcggggcagtgcgccgcgccggcggcgtcgtggatggctcgggttggctggcctgcgacggccggcgaaaggtgcgcagcagctcgttatcgaccggctgcggcgtcggggccgccgccttgcgctgcggtcggtgttccttcttcggctcgcgcagcttgaacagcatgatcgcggaaaccagcagcaacgccgcgcctacgcctcccgcgatgtagaacagcatcggattcattcttcggtcctccttgtagcggaaccgttgtctgtgcggcgcgggtggcccgcgccgctgtctttggggatcagccctcgatgagcgcgaccagtttcacgtcggcaaggttcgcctcgaactcctggccgtcgtcctcgtacttcaaccaggcatagccttccgccggcggccgacggttgaggataaggcgggcagggcgctcgtcgtgctcgacctggacgatggcctttttcagcttgtccgggtccggctccttcgcgcccttttccttggcgtccttaccgtcctggtcgccgtcctcgccgtcctggccgtcgccggcctccgcgtcacgctcggcatcagtctggccgttgaaggcatcgacggtgttgggatcgcggcccttctcgtccaggaactcgcgcagcagcttgaccgtgccgcgcgtgatttcctgggtgtcgtcgtcaagccacgcctcgacttcctccgggcgcttcttgaaggccgtcaccagctcgttcaccacggtcacgtcgcgcacgcggccggtgttgaacgcatcggcgatcttctccggcaggtccagcagcgtgacgtgctgggtgatgaacgccggcgacttgccgatttccttggcgatatcgcctttcttcttgcccttcgccagctcgcggccaatgaagtcggcaatttcgcgcggggtcagctcgttgcgttgcaggttctcgataacctggtcggcttcgttgtagtcgttgtcgatgaacgccgggatggacttcttgccggcccacttcgagccacggtagcggcgggcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgctgaattgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtca

SEQ ID NO: 39 - DNA, 1011 bp, Escherichia coli
atgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgacggtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataa

SEQ ID NO: 40 - DNA, 816 bp, Escherichia coli
atgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 41 - DNA, 795 bp, Escherichia coli
atggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 42 - DNA, 492 bp, Agrobacterium tumefaciens
atgcactcaaccagattgcgaacaaacgaagtcgcggtcacgctacccgaagaaggcgacgcatcgcttttctttgttggaaagatccggaccccttggaaaagtcgagctgacactcctcggcagggcagtgaatccgggccaccatgcacacttgaaatttcagaaccgtgggccgtggcgctaaagggggtcgaggcgtattcgcaacttgaggttctatattggttgcacgagtcaccacgcgatatcgtcctactaagtcccgctgacgatggagagattcacggtgctttctccctacgagcacctgtgaggcccaatccgatcggaacatcgattgtgaaagtggagagggtatctgggaactcgattgttgtacgtggtcttgattgcctggatggaacaccgctgttggatataaaaccggaccgctcgctatcaaagccgctagccccggtgaggaaaaggttctcgattcctgccgtctga

SEQ ID NO: 43 - DNA, 1269 bp, Agrobacterium tumefaciens
atgattgcgaacagctcttccgatgtctctatggccgaccagaagtttctaaatgtcgcgaagtcaaatgaaatcgatcccgacgccgttcctataagcagacttgattctgaaggtcacagtatttttgcagaatggcgaccgaagcgcccgtttcttcggagagaagatggcgtctttctcgttctccgcgcagaccatatctttctgctgggcaccgatccacgcacccggcaaatagaaactgagctcatgctgaatcgaggcgtcaaagctggtgccgtttttgactttataggtcacagcatgttgttttcgaacggtgaaacgcatgggaagcggcgctcaggtctttcaaaggcgttctcattccgcatggttgaagcgttacgcccggagattgcgaagataacggagcgtttgtgggacgaactacaaaaagttgacgatttcaattttactgaaatgtacgcgtcgcaattgcctgcgctgacgatcgcaagtgtccttggcttgccgtctgaggacacgccgtttttcacacgacttgtttataaggtttcccgctgcttgagcccgtcgtggcgagatgaggaattcgaggagattgaagcttccgctatcgagcttcaggattacgttcggggcgtgatcgcggatagtggccgtcggatgagggatgattttctctcgcgctacttgaaggcggtacgggaagccggaacgctttcgcccattgaggagatcatgcaactcatgcttatcatactcgctgggagcgatacaacgcgcactgcaatggttatggtgacggctcttgtgctccaaaatcccgcgctctggtcttctttaactggcaatcaatcctatgtcgcagccgccgtggaggaagggctccgattcgagccgccagttggctcttttccgcggttggccctcgaggatatcgatctggatggatacgtgttgccaaagggaagcctcctcgcgctcagtgtcatgtctggcctgcgagacgaaaaacactacgagaaccctcatctttttgatgttgggcgtcaacaaatgcgttggcacctcgggtttggcgcgggagttcatcgttgcctcggcgagacgttggctcggattgaattgcaagaaggacttggaacacttttgcgccgcgcgccgaatcttacggtggtaggtgactggccacggatgatgggtcacggaggcatccggcgcgccaccggcatgacggtcaaattgagcgtcgaccggtga

SEQ ID NO: 44 - DNA, 1224 bp, Agrobacterium tumefaciens
gtggaagagaggcgcgtatcaatatcaagcatcacttggagatttccaatgccatgtgcgccagtggacgacgtgacgaccatcgatgacctaacactcgatccctatccgatttatcgcagaatgcgcgtgcaaaacccggtggtacacgtcgcctcggtcagacggacgtttctaacgaaggcctgcgatactaagatggtgaaggatgatccttcacgctttagttctgatgatcccaacacgccgatgaagccggcgcttcaggctcatactttgatgcgtaaggatggagttgaacacgctcgggaacgaatggccatgactagagcattcgcacccaaagcgatcgcggagcattgggtgccgatctatcgtgacatcgtgaacgagtatctagatcgccttccgcgcggagacaccgtggatctttttgcagagatctgtggtccggttgctgctcgcatactggcacatattctaggaatttgtgaggcatcagatgtcgagatgatccgctggtcgcaacggcttattgacggagccggcaacttcggatggcgacccgagctctttgagcgatcggacgaggccaatacggagatgaactgcctcttcaacgacttggtcgaaaaacaccgatccgcgccgaacccctcggcttttgcgatcatgttgaatgcacctgatcctatacccctaagccagatctatgccaacatcaagatcgccatcggcggaggggtcaacgagcctcgtgacgctctcggcacgatcctctatggattgctgaccaatccggagcaactcgaggaggtcaagagacagcaatgctgggggcaggccttcgaggaagggctgcgctgggttgctccgattcaagcgagttcgcgtctcgtgcgggaggaaacggaaattcgtggctttatcgttccgaagggcgacattgtgatgactattcaggcctccgccaatcgtgatgaagatgtctttgaggatggagaaagcttcaatgtctttcgcccgaagaatgcgcatcagtcctttggctccggcccgcatcactgcccaggcgcgcaaatctcgcggcaaaccgtcggcgcgatcatgctgcccatactgttcgatcgatttccggatatgatattgccccaccctgaattagtgcagtggcgcggcttcggcttccgcgggcccattaatctacctgtgacactaagatga

SEQ ID NO: 45 - DNA, 438 bp, Agrobacterium tumefaciens
atgaaacgtatcagtacaatccttgtcggcgtatttttggctgcgccggtttatgcagccgacaatattcatacattagggacgttgtctgaaattgaactagcgctcactgccggaaagccggtgaacgtcacggttgatttaagtttgtgtgctcccggggtggccgatactccggcgacgaaaacgcaaggaggcatgcgcatagatgcgtatcgaataacaactgacggcacccttgcgtttgcagatcagcacttcaccattgaccgtgacggtaaacccataactcaatttattcgttatcagattcggtcaaacggtgacgccgatttcacgatggttacattcaacatgccaacatatgagcgaaaaggtaccagcttggcctacaagtgcgcgatcgaccacgggttgagttttcgtactccacagtga

SEQ ID NO: 46 - DNA, 1134 bp, Agrobacterium tumefaciens
atggatcatcgaaatcgcccgctggacgagagacagcagcactgggttaacagcgtttgggagaggctctgtcgaagcgtcgcattcgcccaacatccagattccgcccgcctgccaatcgcttcatatcaaagtgacatcgcaacaaggctgagagaacacagtgatctcagttcgttcgaaacacgtcccgaatatatgcgagagttttcgaacaagttttcatggaatttaaagcgcgacatttcttttgtggttaatcgcaagagtaagaacggtgaattccgagaagatgcgtttgaagagctgttttgcgaaaggataacgtctgtccatcattctcttcaggaggcgatcacggcacggtttgaatccgccgtcggaaaatcgaatgacgaaggcgatgtacgttctcgcgatgtttcgacattttacgctcgcactgctggggagagttctacaaccaagcgattgaacatcagcacaaacccacgaggcgtggtgccgagtgctgacgtcgtcaatatctctgaacagcaggcacagtctgtcaagtatcggcgtcctaagtttctcgttgaggtctcaaagcacgtctatagaccaatcgaacagtataatcggatggtcttcgatcgagtcagatttccaaacatcactcgtgaccggcgtaagttgaatgtattgatccgcacggaagacggcactggctacgaaacacctagctcgttaaaaacgcgcctgggatcggagcagcataatctcttcaccaagacaatgaacgagggtcagccaccccatagtctcgcgtccacgaagactggtaaactgcctatagttttggttgacgcgggaaatggtgcaacctttatcgccggcgagaaattccctttgttggacaacggacgccggataccaacgactatttctgccgatagaattttagtcagaaatgaaaatgggactttcagcgagctttgtcagcgtttggctggaaacaccgaactattgcttccaccagaagcgatagttcaactgggactgcagcgtcaacaagctgtcaatcgagctcgtgccagagaagcatcggagttggagcaacgaactcgcgaacattcctctggaagagtttga

SEQ ID NO: 47 - DNA, 588 bp, Agrobacterium tumefaciens
atgagagatgatccaggagttgctcgagcgctttccctctcgttacaagataagcacgacattttcgagacccagatcattccggcaatcggtgcgcgggggccgtctgttgcaaagccaactttcaccgttgtatgcggccagcagggtgccggaaagagcaccttagttcgccaaatcaagagtagaacgggcggtgaaagcacccagcgaataatagctgatgatcttaatgcatacattcctggtaacaatgcagcccttctgcagggctcgcatgcgttagagcgagcgaattcgacggcagtgacagaatggtatcatcaattatttgatcgcagtatcactaatagatataatattatactggagtcttgctatccgccaaatcattacgcgtcgctcttagataaggcgcgctccaatggatacagaacagaactaaatattatcgcgacggacaggataacgagctttacagctattcacgatcgctttgaaagggcactggcgaactcgtttattgcctcaaccgtattgccagatgtcgaaactcacgatcattattattcatttggccacgtgtag

SEQ ID NO: 48 - DNA, 633 bp, Agrobacterium tumefaciens
atggctaaaaatgatgcttataagctaattatatttgattttgatggtacattggccgacagcggcgcctggatgatacgcgctctgagggaaatgtccgaacgtcaccgattcgtatcgcccagcgacactcagattgaatatctgcgtggcctttcagtttgccaggttctgaagtggatgagagtaccggtgtggcgcattcctctgatcgtgagagacttgcgtaaactggctcgcgaagccacgttcgatatgttccctaggacagaaaaggtcttgtttctcctccaagatcaaggggtcgagcttgcaatcttaagttcgaacagcgttgaaaatattcagcgcgtgctcgggccattggagagatactttgcttatatagagggcagctcgccaatatttgggaagggcaaaaggatcaataagatacttcgtcggtttgaaaggtcccggaatgaagtgcttcttgtcggagatgaggtacgtgatattgaggcagcgatgagtcaaaacgttgcctccgcgggagttacgtggggttatgctaagagagaggctttggcgtgcagcaatcccacgcacatcctagaagatattgaggaactaatagacttgaaacctctctaa

SEQ ID NO: 49 - DNA, 213 bp, Agrobacterium tumefaciens
atgaaaacgtcgaccaaaacaatgcttgtctacgccgcgataattttcactccaggatttatatattttccactatataaaattttgctcactctttatagtgaagattcaggagatacatatttttgtcagttctatacggagttctcatttttttggataatgggattactggtttttgccttgtcaacgatgcttaaaaagaagccttag

SEQ ID NO: 50 - DNA, 2101 bp, Salmonella tymphimurium
gacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcat

SEQ ID NO: 51 - DNA, 1554 bp, Escherichia coli
atggaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgtt

SEQ ID NO: 52 - DNA, 1576 bp, Escherichia coli
gatcgctagtttgttttgactccatccattagggcttctaaaacgccttctaaggccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatctagcttaaaacaggtggctttttaatcatctttgccaagcatggcgcgggtttggggtaatatagcgactcataaaagcgttaaacatgagtggatagtacgttgctaaaacatgagataaaaattgactctcatgttattggcgttaagatatacagaatgatgaggtttttttatgagactcaaggtcatgatggacgtgaacaaaaaaacgaaaattcgccaccgaaacgagctaaatcacaccctggctcaacttcctttgcccgcaaagcgagtgatgtatatggcgcttgctcccattgatagcaaagaacctcttgaacgagggcgagttttcaaaattagggctgaagaccttgcagcgctcgccaaaatcaccccatcgcttgcttatcgacaattaaaagagggtggtaaattacttggtgccagcaaaatttcgctaagaggggatgatatcattgctttagctaaagagcttaacctgccctttactgctaaaaactcccctgaagagttagatcttaacattattgagtggatagcttattcaaatgatgaaggatacttgtctttaaaattcaccagaaccatagaaccatatatctctagccttattgggaaaaaaaataaattcacaacgcaattgttaacggcaagcttacgcttaagtagccagtattcatcttctctttatcaacttatcaggaagcattactctaattttaagaagaaaaattattttattatttccgttgatgagttaaaggaagagttaacagcttatacttttgataaagatggaaatattgagtacaaataccctgactttcctatttttaaaagggatgtgttaaataaagccattgctgaaattaaaaagaaaacagaaatatcgtttgttggcttcactgttcatgaaaaagaaggaagaaaaattagtaagctgaagttcgaatttgtcgttgatgaagatgaattttctggcgataaagatgatgaagctttttttatgaatttatctgaagctgatgcagcttttctcaaggtatttgatgaaaccgtacctcccaaaaaagctaaggggtgatatatggctaaaatttacgatttccctcaaggagcc

SEQ ID NO: 53 - DNA, 2400 bp, Escherichia coli
cacctaaacgaaggccgggccactcacccggccttttttgtacgctcatagggcagaacaaaccaacgttttatctataccgctacagggtatttaattcctatttaatctgcgctagaatgaggcatgtttaaccgaatctgacgttttccctgcaaatgccaaaatactatgcctatctccgggtttcgcgtgacggccaagacccggaaaaccaaaaatacggtttgctcgaatacgcgaacgccaaaggcttcgcgccgctacagatcgaggaagaaattgccagcagagcaaaggactggcgcaagcgcaagctcggagcaatcatcgaaaaggccgagcgtggcgacgtgctactgacgccggagattacgcgcattgccggttccgccctcgccgccttggaaattctcaaagcggcgagcgagcgcggcctaatcgtccatgtgaccaaacagaagatcatcatggacggcagcctacaaagcgacatcatggcaaccgtgcttggcttggctgcacagatcgagcggcatttcattcaggcacgtaccaccgaggcgctacaagtcgccagagagcgcggcaagacgctcgggcgacccaagggcagcaaatcgagcgccttgaagctggacagccgtattgatgaagtacaggcatacgtgaaccttggcttgccgcaaagtcgcgcagccgagttgttaggcgtcagccctcacaccttgcgcctgttcatcaaacgccggaacatcaaacccacaaacactagaccaaccatcaccatgccggggagggaacaacatgcctaagaacaacaaagcccccggccatcgtatcaacgagatcatcaagacgagcctcgcgctcgaaatggaggatgcccgcgaagctggcttagtcggctacatggcccgttgccttgtgcaagcgaccatgccccacaccgaccccaagaccagctactttgagcgcaccaatggcatcgtcaccttgtcgatcatgggcaagccgagcatcggcctgccctacggttctatgccgcgcaccttgcttgcttggatatgcaccgaggccgtgcgaacgaaagaccccgtgttgaaccttggccggtcgcaatcggaatttctacaaaggctcggaatgcacaccgatggccgttacacggccacccttcgcaatcaggcgcaacgcctgttttcatccatgatttcgcttgccggcgagcaaggcaatgacttcggcattgagaacgtcgtcattgccaagcgcgcttttctattctggaatcccaagcggccagaagatcgggcgctatgggatagcaccctcaccctcacaggcgatttcttcgaggaagtcacccgctcaccggttcctatccgaatcgactacctgcatgccttgcggcagtctccgcttgcgatggacatttacacgtggctgacctatcgcgtgttcctgttgcgggccaagggccgccccttcgtgcaaatcccttgggtcgccctgcaagcgcaattcggctcatcctatggcagccgcgcacgcaactcgcccgaactggacgataaggcccgagagcgggcagagcgggcagcactcgccagcttcaaatacaacttcaaaaagcgcctacgcgaagtgttgattgtctatcccgaggcaagcgactgcatcgaagatgacggcgaatgcctgcgcatcaaatccacacgcctgcatgtcacccgcgcacccggcaagggcgctcgcatcggcccccctccgacttgaccaggccaacgctacgcttggcttggtcaagccttcccatccaacagcccgccgtcgagcgggcttttttatccccggaagcctgtggatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccgcaacagatcgttttataaatttttttttttgaaaaagaaaaagcccgaaaggcggcaacctctcgggcttctggatttccgatcaacgcaggagtcgttcggaaagtagctgttccagaa

SEQ ID NO: 54 - DNA, 599 bp, Artificial sequence
  Synthetic construct
cggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgc

SEQ ID NO: 55 - DNA, 801 bp, Escherichia coli
tgcgcgtcccccttggtcaaattgggtatacccatttgggcctagtctagccggcatggcgcattacagcaatacgcaatttaaatgcgcctagcgcattttcccgaccttaatgcgcctcgcgctgtagcctcacgcccacatatgtgctaatgtggttacgtgtattttatggaggttatccaatgagccgcctgacaatcgacatgacggaccagcagcaccagagcctgaaagccctggccgccttgcagggcaagaccattaagcaatacgccctcgaacgtctgttccccggtgacgctgatgccgatcaggcatggcaggaactgaaaaccatgctggggaaccgcatcaacgatgggcttgccggcaaggtgtccaccaagagcgtcggcgaaattcttgatgaagaactcagcggggatcgcgcttgacggcctacatcctcacggctgaggccgaagccgatctacgcggcatcatccgctacacgcgccgggagtggggcgcggcgcaggtgcgccgctatatcgctaagctggaacagggcatagccaggcttgccgccggcgaaggcccgtttaaggacatgagcgaactctttcccgcgctgcggatggcccgctgcgaacaccactacgttttttgcctgccgcgtgcgggcgaacccgcgttggtcgtggcgatcctgcatgagcgcatggacctcatgacgcgacttgccgacaggctcaagggctgatttcagccgctaaaaatcgcgccactcacaacgtcctgatggcgtactaagctggg

SEQ ID NO: 56 - DNA, 3208 bp, Artificial sequence
  Synthetic construct
cccagcttagtacgccatcaggacgttgtgagtggcgcgatttttagcggctgaaatcagcccttgagcctgtcggcaagtcgcgtcatgaggtccatgcgctcatgcaggatcgccacgaccaacgcgggttcgcccgcacgcggcaggcaaaaaacgtagtggtgttcgcagcgggccatccgcagcgcgggaaagagttcgctcatgtccttaaacgggccttcgccggcggcaagcctggctatgccctgttccagcttagcgatatagcggcgcacctgcgccgcgccccactcccggcgcgtgtagcggatgatgccgcgtagatcggcttcggcctcagccgtgaggatgtaggccgtcaagcgcgatccccgctgagttcttcatcaagaatttcgccgacgctcttggtggacaccttgccggcaagcccatcgttgatgcggttccccagcatggttttcagttcctgccatgcctgatcggcatcagcgtcaccggggaacagacgttcgagggcgtattgcttaatggtcttgccctgcaaggcggccagggctttcaggctctggtgctgctggtccgtcatgtcgattgtcaggcggctcattggataacctccataaaatacacgtaaccacattagcacatatgtgggcgtgaggctacagcgcgaggcgcattaaggtcgggaaaatgcgctaggcgcatttaaattgcgtattgctgtaatgcgccatgccggctagactaggcccaaatgggtatacccaatttgaccaagggggacgcgcaatctaggttctggaacagctactttccgaacgactcctgcgttgatcggaaatccagaagcccgagaggttgccgcctttcgggctttttctttttcaaaaaaaaaaatttataaaacgatctgttgcggccgccgggttgtgggcaaaggcgctggcgctcgacggtgggcaaccgcttgcggttgtccacgggcggagccggtgcgcgtagcgcattgtccacaagccaagggcgaccaataattgatatatatattcataattgaaaagctaattgaacatactacttgctgtaactacttgccggagcgaggggtgtttgcaagctgttgatctgaaagggctattagcgttctcacgtgcctttttgattagcgatttcacgtgaccttattagcgatttcacgtactccgattagcgatttcacgtaccctgattagcgatttcacgtggatagtttttggagcgggccggaaagccccgtgaatcaaggctttgcggggcattagcggtttcacgtggataactaccctctatccacaggcttccggggataaaaaagcccgctcgacggcgggctgttggatgggaaggcttgaccaagccaagcgtagcgttggcctggtcaagtcggaggggggccgatgcgagcgcccttgccgggtgcgcgggtgacatgcaggcgtgtggatttgatgcgcaggcattcgccgtcatcttcgatgcagtcgcttgcctcgggatagacaatcaacacttcgcgtaggcgctttttgaagttgtatttgaagctggcgagtgctgcccgctctgcccgctctcgggccttatcgtccagttcgggcgagttgcgtgcgcggctgccataggatgagccgaattgcgcttgcagggcgacccaagggatttgcacgaaggggcggcccttggcccgcaacaggaacacgcgataggtcagccacgtgtaaatgtccatcgcaagcggagactgccgcaaggcatgcaggtagtcgattcggataggaaccggtgagcgggtgacttcctcgaagaaatcgcctgtgagggtgagggtgctatcccatagcgcccgatcttctggccgcttgggattccagaatagaaaagcgcgcttggcaatgacgacgttctcaatgccgaagtcattgccttgctcgccggcaagcgaaatcatggatgaaaacaggcgttgcgcctgattgcgaagggtggccgtgtaacggccatcggtgtgcattccgagcctttgtagaaattccgattgcgaccggccaaggttcaacacggggtctttcgttcgcacggcctcggtgcatatccaagcaagcaaggtgcgcggcatagaaccgtagggcaggccgatgctcggcttgcccatgatcgacaaggtgacgatgccattggtgcgctcaaagtagctggtcttggggtcggtgtggggcatggtcgcttgcacaaggcaacgggccatgtagccgactaagccagcttcgcgggcatcctccatttcgagcgcgaggctcgtcttgatgatctcgttgatacgatggccgggggctttgttgttcttaggcatgttgttccctccccggcatggtgatggttggtctagtgtttgtgggtttgatgttccggcgtttgatgaacaggcgcaaggtgtgagggctgacgcctaacaactcggctgcgcgactttgcggcaagccaaggttcacgtatgcctgtacttcatcaatacggctgtccagcttcaaggcgctcgatttgctgcccttgggtcgcccgagcgtcttgccgcgctctctggcgacttgtagcgcctcggtggtacgtgcctgaatgaaatgccgctcgatctgtgcagccaagccaagcacggttgccatgatgtcgctttgtaggctgccgtccatgatgatcttctgtttggtcacatggacgattaggccgcgctcgctcgccgctttgagaatttccaaggcggcgagggcggaaccggcaatgcgcgtaatctccggcgtcagtagcacgtcgccacgctcggccttttcgatgattgctccgagcttgcgcttgcgccagtcctttgctctgctggcaatttcttcctcgatctgtagcggcgcgaagcctttggcgttcgcgtattcgagcaaaccgtatttttggttttccgggtcttggccgtcacgcgaaacccggagataggcatagtattttggcatttgcagggaaaacgtcagattcggttaaacatgcctcattctagcgcagattaaataggaattaaataccctgtagcggtatagataaaacgttggtttgttctgccctatgagcgtacaaaaaaggccgggtgagtggcccggccttcgtttaggtg

SEQ ID NO: 57 - DNA, 3754 bp, Agrobacterium rhizogenes
atgcgttatcttgagacggcaataacgcgtctgacgctaataaaccgtctgaaacctgaggcccaagtgaacgtgatcgaccggcacattaatcgagcatcaacttccgcacacatcacccagcgcgcggaagcgttgtctgcgagactcagggcggttggtgagcgcgcgtttccccctaccgcacaaaagtcgcttcgatctttcacttccggcgaggtcgccgaaattgtcggcgtgtcagacggctatcttcgccagctttcgctcgatggactcggcccttcgccagatttgggaagcgccggtcgccgctcttacactttggagcaaatcaatcaactccgtgagtatcttgccggagctcgacctaaggaagcaagtagattttggccacgtcggcgcagtggcgaaaaacttcaagtgatcacggtcgcaaatttcaaaggcggctccgcgaagacaacaactgctgtttatctcgcacaaggacttgcgttgcagggctaccgtgtcttggcggttgatcttgatcctcaggcttccctctccgcaatgttcggttatcagccagagttcgacgttgccgaaaacacgaccctttatggtgcgatccgctacgacgaccagagggtgacgatgaaggacgttatccggcgcacctactttaccgggataagcattgtcccgggtaatctcgaactgatggagtttgaacatcagacccctcgattcatgctgcagaatcgagggcgtccggaggatctatttttcagacgtgtcgcaggcgcaatcaatcaggtcgaacaggacttcgacgtcgttgtcgtcgattgccccccacaactgggctttttgaccatgggcgcccttaacgcagcgagtggcatgattgtgactgtccatccccaaatggtcgatgtcgcatccatgagccagttcctcttgatgacgtccgatctcgtgtcggtaattgaagaagccggcggacggctcgactacgactttctaaggtttcttattacgaggcatgatccgcgcgacgtccccgagcaggaaatcgtggcgctgcttcgtgacgtcttcggaaccgacgtcatggcggcatccgcatggaagtcgacggcaatcgccaatgccggtcttaccaagcagtcgctctatgaactctcgcgcggcgcggtaggacggacgacgtatgatcgagcaatggaatccatcaatgccgtgaatctggaaattatcgaccagataaacaaggtctggggtcgatgatggcggctcaatcgctgataaatcaaaaccttagtgtgttgtcagctgactacacggtcttactagaactgttgtttggatagggtcgaaacgatgagcaaacgcacacaatccattcgctcaatgtttgccgggggcaacgatcagaccgcttctaatgacgcacagcagcctgtgcagcgagttgcctcgggtgcggtccggtcgttgaaagacacgttctcagaggtggaaagacattacgaagagctcaagcagcaagttgctgacggagctgttccagtcgaaatagacccagaattggtcgacccctcgcccttcgcggaccggtttgcggaccaagacatggctgtcttggaggtccttaagacttcgatcaaagatcatggacaagaaattccgatccttgttcgaccgcatccagctgagatcggacgatatcaggttgcctacggccaccgcagactacgtgcgactgccgaactcggcttgaaagtcaaagcctatgtccgcgaactggatgacgatcgtcttgtcgtcgcgcagggcatcgagaattccgctcgtgaagacctgactttcatcgaacgggcggcgtttgcactcaaactggacgagggtggttttcaacggtcgctaatacaaatggctttgtctgttgaccgacacgaagctcaaaagctggtaacagttgctcgggcagttccgcagtggttgattgattccattggccgtgcgccgaagatcggtcgaccgaggtggctggagctcgcagaacttttgaaagtcccgggcgcagagaagaaggcccgcaaagccacccaggacaaatccttctcacataaggattctgacaatcgtttcctggtcgtcctaagagcgtccaagatagatgatgtgacggcgtcgtctgtagcccccacccgcttggtcgcaaaatctgctgacgggcttcaaatcgcgacgctcgccatcggcggcaagcagtgcaaaatcgaaatgaaccgagatagagacgagacttttgcaaagtttgttatggagcagcttcctgcgctctacgcgaacttcagaaaggaaaatccgtcatctgaaggataaataaggataggaacagcaaaagaaaggcccccaaacgtcgccgtcgtggaagcctctctcaatggtctaagcagcctgagaatcgcatttccatgaatcgcagtcaagagtctttggcaccgtttttggtgagcagatttcttttgcctattgaagggtgaaagacatgcagagtggaaatgtgacgacgccgtttgggcggcggccgatgacgcttgccttagtggcggcacagttcaaatccgccgacatcaggcagggtaaatcggccgacaagtggaagatctatcgcgatgcttgtgacgcgcgatcattgttgggtttgcgcgatcgggcacttgcagtgctcaacgcgcttctatcattctaccctgaggctgaactcaaggaggaagcgaatcttatcgtgtttccgtccaatgcccaactcgcggcacgggcaaacggtatcgccggcacgacgctacgcgagaacttggccctcttggtcgatgcaggtctcgtccaacggaatgatagcccgaacgggaaacgctatgcgcgcaagagctcagatgggtccatcgagacagcctttggcttcagtctagcgccgcttctggcccgatcagaagaacttgcggttatggcccagcaggtcgcagagggtaatcgcaagctcaaaatcgtcaaagagcgcatcacaattgcccgacgggatgtccggaaattgatctctgcggcaatggaagatcacgttcccggggattgggccaaggtcgaggagctttatgttgaagccatggcacggctgcggcgggcaaaaggggcggacgaattgatcggaattctcgacgaagtcgagatgctgaaggaagaagtagtcaatctattggaacggcacgtcaatcttgaaaaatccgacgccaatgatgacggattccgtcagcacatacagaattcaaataccgaatccagcaatgaacttgaaccttgctctgaaaacgagcagggcgcgaagcagagtaaaaacgttgaactaagagccgaatcgataaaatcattcccgcttggtatggtccttcgggcatgcccggagatttccatgtatggtccgggcggagcaatcggcagctggcgggaccttatgatggccgccgtggtcgtgcgatcaatgctgggggtaagcccgtcggcctaccaggacgcctgcgaagtaatagggcctgaaaacgcggccgcaatgatggctgcaatcctggagcgggcggggcacatcacatcggccggtggatatcttcgcgatttgacgtcgagggcaaaacgcggcgagttttctctcggaccaatgttgatggcgttgctgcgggccaatggcggcgaacaaaggcgggcttaa

SEQ ID NO: 58 - DNA, 3992 bp, Agrobacterium tumefaciens
gtgagcaaagccgctgccatatcccgaaatgatcgcccgtcggtagatgttaccattggtgagcatgctgagcagctcagctctcagcttcaagcgatgagcgaggctttgtttcctccgacgtcgcacaagagcttgcgcaaattcacctcgggtgaagccgcacgcttgatgaaaatatctgactcaactcttcgaaagatgacactggctggcgaagggccgcaacctgaactcgccagcaacggacggcgcttttacaccctcggtcagataaacgaaatccggcagatgcttgccggctcgactcgaggacgtgaaagcattgattttgtgcctcatcgccgaggttctgagcatttgcaagtcgttgctgtaaccaacttcaaaggtggctctgggaagacgacgacgtccgctcatcttgcacagtatctggcgttgcaaggttacagggttctcgcagtcgatctcgatccgcaggctagtctttcagcactcctcggcgttctgccagaaactgatgtcggtgcaaacgaaacgctctatgcggctattcggtacgacgacacacgtcgtccgttgcgagatgtgatccgaccgacgtattttgatggtcttcaccttgttcctggaaatctcgagcttatggagttcgagcataccaccccgaaagcattgactgacaaaggtacgcgcgacggattgttcttcactcgcgtggcccaagcctttgatgaggtcgccgacgattacgatgtcgtggtcatcgactgccctcctcagcttgggtttttgactctcagcgggttgtgtgctgcaacatcaatggtaatcaccgtacatcctcagatgctggatatcgcttccatgagccagtttctcctcatgacacgcgaccttctgggtgtcgtgaaagaggcggggggcaatctccagtacgatttcatacgctatctcttgacgcgctatgagccccaggacgcgccgcagacgaaagtgacggcactgctgcgcaacatgttcgaggatcacgtccttacaaatcctatggtcaagtcggcagcggtatctgatgccggtttaaccaagcagacgctctatgagatagggcgagagaaccttacgcgatcgacatacgaccgggcgatggaatctttagatgcggtgaattcggagatcgaggctttgatcaagatggcgtgggggcgggtctaatgaaaggctttgcgttcctcacagatctgttgggagctcccaacagacaggtgttgattcgccccctggacatggggcactggagaagccggggtaatttgagacgacgacgcacgcccatcgctaattggccagggtgcagttgtcttgtcttgttgggagctcccaaccaagcgcatttgcaatcaaaaatgcgacgccacgacgccaaacccaagaggctgatatcatgagccgcaaagacgcaatcgatactttgttcctcaagaagcaacctgcgaccgatagagcagcagtcgacaagtcgaccgctcgtgttcgtaccggagcgatttcggccatgggttcgtctttgcaagagatggctgagggcgcaaaggctgcagctcggctgcaggatcaactggctacaggcgaagccgtcgtgtccctggatccatccatgatcgacgggtcgccgatcgcggatcggctgccctcagacgtggatccgaaattcgagcagcttgaggcgagcatttcgcaggaggggcagcaggtgccggttcttgtcagaccgcaccctgaggctgccggtcgatatcagatcgtatatggaaggcggcggctgcgcgcggcagtaaatctgcggagagaggtttctgccattgttcgaaatctcacggactgtgaactggtcgtggcccagggccgcgaaaatcttaaccgtgctgacctctcgttcattgagaaggctctcttcgccctgcgcctcgaagatgcgggttttgatagagccaccatcattgccgcgctatccactgacaaggccgacctcagccgctacataactgtagcaaggggcataccgctgaacctcgccacacaaatcggcccagcgtcgaaagcgggtcgatcgcgttgggtcgcacttgccgaggggcttgggaagcctaaggcaacggacgcaatcgaagcgatgcttgggtcagagcagttcaagcaatctgatagcgatacccgctttaacctcattttcaacgccgtttcaaggccacctgcgaagactccaaaaaaggtaagggcctggagcacgccaaaggggaaaaaggcagcgacgatccgacaagaaactggacgaacggcgctggttttcgacgagagactggtgccaacttttggcgaatatgtcgctgaccagttggacagtctgtacgcccagttcattgaaaccaacggaggaggcaagctcgaccaatagtcagggtttcatccaatttaaagctccgctcgactgagatggactggctctcaccgcaaaagaaaaaggcccccgaaacggcgttccggaagaccttctctgtagtctcgcagctaagagaatcgcatttccaggaatcgtagtcaagggtcccgtaagggaaagcgtcatttcgacgggcggatttcaattgcctaacaaaaggtaaaaggaaatgcagacgcatatctcaacgacgtcctttgggcggcggccgatgacactcggccatattgcaagccagatggcagcaaaagcggtcgcatcagacactgtcgcccacaaatggcaggtcttccagcacatccgtgaatcccggggactgatcggagccacggaccgctcactctcgatcctgaacgcgctgttgacgttttacccggagaccgccttgactggtggtgccgaactggtcgtatggccttctaacgaacagctgatggctcgcgccaacggcatgcccgccacgacactgcgccggcatcttgccatactggttgattgcgggctcatcattcgccgcgacagccccaatggcaagcggttcgcccgcaagggaaggggaggggagattgagcaggcctatgggttcgatctgtcgccgatcgtcgcgcgggccgaggagttccgagatctggcccagacagtgcaagctgaaaaaaaggccttccgggtggccaaggagcgcttgactcttcttcgtcgtgacattgtcaaaatgatcgaaactggcgtcgaagagagcgttcctggaaactggggaagagttacccagacctatcaggggatcatcggccgcctgccacgctcggcacctcggcagcttgtcgagagtattgggcaagagcttcaggaactttgcatcgagatccgtgacgtattggaatctttcacaaaaacgatgaatctggacgccaatgagtcccatatcggtcgccacaaacagaattcaaatccagactctaaatttgaatctgaatacggctctggaaaaaaagatgaagcgggcggcagcgttgcggaaaccgacaatgtacggagcttgccgaaacgcgagctgcctttgggaatcgtgctggatgcctgccccgaaatgcgggaattggcccagggaggtccaattcgacattggcgcgacttgctggcggcggctgagcttgcccggccgatgctggggattagtccgagcgcctggcgggaggcccgcgaaaccatgggcgagcaacacgcggcgatcacgctggcttcgatctatcagcgggccggtcagatcaataacgctgggggctatctgcgcagcctgaccgaccgggccaaggatgggaagttttcgacctggccgatggtcatggcgttgctccgggcgaagctggacgagcagaagaatgcagtcggcgctggaaagccgcgaactgctgaggaggtcgaggatgacagccgcctccacgtatcggaatcgctgctcaaaaacctgcgaaagccgagatcttggtga

SEQ ID NO: 59 - DNA, 865 bp, artificial sequence
  PR1b plasmid, Ruegeria sp.
gcgccggctggtaatcgatgatgatgcaatccagcgtttcgttcagcgcctgctcgagacggacaggatcgacctttccatctgcaacaagttcgttgggagccgttgccggcggatacgcctctttccagcgatcaatcgcatcccgcagatagcgatagaaactcttccccgcaggcgcttcgcgaaccaagcgggcgatctgaatttcgccctcagaagtctctccgtgcgccggcaccaatcgaagccccggccaagaagtcttcaaaaagaagctatccagcgtctccgcagagtgatccgtgtacggcgcctcctcagactggaagaggcctgcgaagtccaccatcgatggcgtctcttcagtaggcatctgagacaaaccctcaccgcctacaaagtaaagtgtgatcgtgctttgcggatccgcatccatcacaccaacgcgcattccgtagtgcaagctcaggtactgagcgaaatgtgcagcgctaaggctcttggctgtgccgcccttttggctggcaaatgaaatgaccggaagcggatcgccgggcttgcgccacgctagatagtcaaaagggcgctttgcagacgccgccatcaaagcgcgcatatgcatcagttcgtgaagattgaagacacgctcgcgtccgacgtactcaccctttggaaagtcgtcgttgccattcgcaaacttagtcaagtattggatgctgacgttcaaaaagcgagcgcactgtcgcatcgaccatgtacgcttgatacgatggcgcaacgtcaactctttgttgacggcgaccattgtgcgctccagacctcgtgacaggtcggtcaaaaactgttctagacttccgctcat

SEQ ID NO: 60 - DNA, 3120 bp, Sinorhizobium fredii
atgaacgctaattcgccgcccatcgctcccgcacagccgatgcatttcgaggatctcatcctcgaacagggcgatctgatttcaaagaaattgcatcttttaagcatgcagcagttcccaccgaatgccaagaaactgcttcgccagttctcgctctctgaagtggcgcagttcctcggcgtctctcaaagtacgctgaagaaacttcatcttgagggaaaaggccccctcccccagacatcgtcgtcgggtcgccgttcttatagtgctgagcagatggcggaactgcgtcagtatctcgatcagcatggacgatcggaagccaggaactacgtgccgcaccgtcggtcgggcgaaaagctccaggtcattgccgttgtcaatttcaaaggcggttcgggaaagacgacgacggccgcccatctcgcgcagtacatggcgttgaccgggcaccgtgtcttggcggtggacctcgacccgcaggcgtcactttcgtcgcttcacggttttcagcccgaactcgacatgtcgccgtcgctctatgaggcgctcagatatgacgatcaacgccgatcgatcagtgagatcatccagcctaccaatttcccgggcctcgatatcgtgcctgcgaacctcgaacttcaggagtacgagtacgacaccccgcttgcgatgtcgaacaagagctcgaacgatggcaagaccttcttcacgcggatttcgcgcgcgctgtcggaagtcaacgaccgctatgacgttgtcgtcatcgactgccctccccagctcggctatctcacgatcaccgcgctgactgcggctacgagcgtcctgatcacgatccatccacagatgctcgacgtcatgtcgatgggccagttccttttgatgctgggcgggatcctgaagccgatccgggatgtgggtgctgcggtcaatctcgaatggtatcgctatctgatcacccggtacgagccaacggatgggccgcaggcgcagatggtgggcttcatgcagaccttgttccaccagtttgtgctgaagaaccagatgctgaaatcgacagcggtttcagacgccggaatcaccaagcagaccctttacgaagtcgacaagagtcagatgacgcggtccacctatgagcgcgcgatggactccttgaacgcagtcaacgccgagatcgtcgaactcgtccatgcctcttgggggaggaaggttgtcagttgagaagtgtaaacttccccgggacggcccggtagatctcaggcaaacggagcaggcacatggcgagaaagaacctactggcgggactggtagacacagcggaaatcccgcacgcagacgttgcgcccgcctacccgatgcgcggggcttcgaagagcatggtgcgctcgctcgacgagttgtcgcgccaggccgagaaatttctcgaaggagaaacggtcgtcgaactcgaccctgagacactcgatggttcgttcgtctctgatcgcatgggcgatagttccgagcagttcgaggaattgaagcaggcgattgccgagcgcgggcaggatacgcccatccttgtgcgcccccacccgtcggcagccgatcgttaccagatcgtctttggacaccgccgagcccgcgttgcccgcgagctcggccgcaaggttaaggcggtcgtcaaggcgctggatgaccgcacgcacgttatcgcccagggacaggaaaactcggcgcgcgccaacctctccttcattgaacgggcgaacttcgcctcgcacctcgagaagcttggctatgaccggacaatcataggatcggcgcttgctgccaatgcggccgctatctcgaaaatgatcgccgtaatagatcgtattcctgaagagacaatcgcaagaatcgggccctgcccggcggtcggccgagaacgctgggtcgagttgtcattgcttgtgggtaaaaccgccaacgaagcgaaggtgaaggcaatcgtctctgatccttccttcaatgaactgagcactgacgatcgcttcaattccctgttttctggtctgaacagcgcggcgaagcctgtccggaagacgactccaaagatcctggagaactggcaaccggcggacaagaccgtctccgctaagtattcgaactccgccaaggcgtttgccttgtctatgaaatcaaggaatgccggcccctttgggcggtatattgcagacaacctggaccggctctatgccgagttcctggagcagggtaatcggaaggaagactgacgcaaaagaaaaaggcccccagacggtaaccatcgtggaagcctctctcatcgtttagcagcctgagaatcgcatttccacgaatcgcagtcaagagtctttggcaccggaacgggtgagcggacgtcttttgcctgaggataggtgaaagaagatgcaagatggaagtgtgacgacgcccttcgggcggcggtcgatgacgcttggcatgttggcaagccaatatatgtcgcgtgaactcgaacctgaaacatcggctgacaagtggaagctgtttcgagcgctctgcgaagctaagccgaagctcggcatcagcgaacgtgcgctctctgttatgaacgcacttttgagcttttatcccgagacgaccttgtctgaggaaaacggcctcatcgtgttcccctctaacatgcagttgtcgcttcgtgcgcatggcatggctgaagcgacgttgcgtcgccacatcgcagcgctcgtcgacgctggccttctggcacgccgggatagcccaaatggcaaacgctacgcccgcaaggacggagacggttcgatcgatgaggcctatgggttttcgttggctccgttgctgtcacgcgcacgcgagatcgagcaaatagctgcctatgtcaaaattgagcgactgcaattgcgtagacttcgcgagcgcctgacgatctgccggcgcgatatcggtaagctgattgaagtcgccattgaggagggagtggacggtaattgggacggaattcaccagcactaccgcagcctagtagcgaccattcctcgagtggcaacggccgcgaccgtcgcccccatcctcgaagagatggagatgttgcgcgaggaaatctccaacctt

SEQ ID NO: 61 - DNA, 32 bp, Artificial sequence
  Synthetic construct
cgttcctcgaggcctcgaggcctcgaggaacg

SEQ ID NO: 62 - DNA, 146 bp, Artificial sequence
  Synthetic construct
gagtccacgctagatgagagctttgttgtaggtggaccagttggtgattttgaacttttgctttgccacggaacggtctgcgttgtcgggaagatgcgtgatctgatccttcaactcagcaaaagttcgatttattcaacaaagcc

SEQ ID NO: 63 - DNA, 6968 bp, Artificial sequence
  Synthetic construct
gtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacgccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggctttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcat

SEQ ID NO: 64 - DNA, 1660 bp, Escherichia coli
gtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgaga

SEQ ID NO: 65 - DNA, 868 bp, Escherichia coli
agaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgc

SEQ ID NO: 66 - DNA, 3129 bp, Artificial sequence
  Synthetic construct
cgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagccagcgctttac

SEQ ID NO: 67 - DNA, 1026 bp, Artificial sequence
  Synthetic construct
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 68 - DNA, 4148 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttaccagagctggtcacctttgtccaccaagatggaactgcggcctcgagatccactagagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctccggtggaggcggcagcggtggcggaggctccggaggcggtggctccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagtcaggatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 69 - DNA, 18762 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacgctgtttaaacgctcttcaactggaagagcggttactaccggttcactagctagctgctaaggttaccagagctggtcacctttgtccaccaacttattaagtatctagttgaagacacgttcttcttcacgtaagaagacactcagtagtcttcggccagaatggcctcttgattcagcgggcctagaaggccggatcactgactagctaatttaaatcctgaggatctggtcttcctaaggacccgggatatcggaccgattaaactttaattcggtccgataacttcgtatagcatacattatacgaagttatatgcatacgcgtcttaagtcgatcgctatcaactttgtatagaaaagttgggccgagctcggtacggccagaatggcccggaccgggttaccgaattcaggcgacccatcgctgctttgtctacatcatgttcttcatcatcctccccaggcgacgcgtgctgctgttcttattcagactaccgttcgagtgactgcatggcgtacatctttctgcatcgactttgtacggctacatcgaacatatacacgagatgtctcgtgtgaatagagtcactaatgccttaagcatcggttactccgtagggtacattctgttcttcttatttgtgcatatttttattgttgtttactgattatacgagtagttatacatacatgcacatacatatcatcacatatatcacaatatttttctaaattaaattaaaactaaaaatgactaaatttctaacaccaacgacattgtaatgttttctccaacaactttacctattctacattgttctatttcgaatttcactctataaacaacatagtctacaatggaaaacagtgctttgtacgactatatacgcgatgtgtggctacaacataagacaatatagtcgtttgaagattgaacctatatatcggtacggttaatccgtctatgtacgtgggcatgacgaacacccgtgataacgaaggattaacgtgcacaatcataaatccaaagtaggagcggtgcatgatgagaatcgctctcagtactcgacataatgaaccttacgaggtacaacaggcaggcaggcagggaccaggggccgcctttatttcaggctcgctggccccacgggcgtgctgcgtgcacgaagggcactaccccaacctctcaccgaaaaccgcgctggatcggcaaatcaaacgaggtggtgccccgtgcccactctccacgtccacggcaccatccctctgcagccgctcaccagccatgccgtgtcgcggaacggcacaaccacccccaacccactcacgaaaccccgtcccggccgtgcccgtgtcggtccgcgctcggcaacgaggcggcccgcgctgctgagtcccctggacacccgacaccctgtcggccctttgtttattcatcccgaaatctcatctgcccccacggccgactgcgctgcgccgcccggatatatatacccatcgttatcgatcgactggggactctatcagtgatagagtctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactggtaccgttaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgacccaccttgctagttggatattacctcttctcttcaaagtatccttgaacgctcaccggttatcaaatctctacactatagctctgtagtcttgctagatagttagttctttagctctcggtgaccaagcttggcgcgatcaagcttatcgataccgtcgacctcgaagcttggtcacccggtccgggcctagaaggccagcttcaagtttgtacaaaaaagcaggctccggccagaatggcccggaccgggttaccgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcaccgggatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctagatcgatgggccctggccgaagcttggtcacccggtccgggcctagaaggccagcttcaagtttgtacaaaaaagcaggctccggccagaatggcccggaccgggttaccgaattcgagctcggtaccctgggatccgattgactatctcattcctcaaaccaaacacctcaaatatatctgctatcgggattggcattcctgtatccctacgcccgtgtaccccctgtttagagaacctccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctcaagcatcagcggcagcggcagcggcaggagctctgggcagcgtgcgcacgtggggtacctagctcgctctgctagcctaccttaagatatcggatccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgatccgatatcgatgggccctggccgaagcttggtcacccggtccgggcctagaaggccgatctcccgggcacccagctttcttgtacaaagtggccgttaacggatcggccagaatggcccggaccgggttaccgaattcctgcccttaaggccaattgttcaagattcattcaacaattgaaacatctcccatgattaaatcagtataaggttgctatggtcttgcttgacaaagtttttttttgagggaatttcaactaaatttttgagtgaaactatcaaatactgattttaaaaaatttttataaaaggaagcgcagagataaaaggccatctatgctacaaaagtacccaaaaatgtaatcctaaagtatgaattgcattttttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggatatgtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcaggtcgactttaacttagcctaggatccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagggtaccaatggccagttaacagatccagctgctgctgttctagggttcacaagtctgcctatttgtcttccccaatggagctatggttgtctggtctggtccttggtcgtgtcccgtttcattgtgtactatttacctgtaatgtgtatccttaagtctggtttgatggtgtctgaaacgttttgctgtggtagagcagcatggaagaactataatgaataagtgatccctaatcattgtgtccaaattttgcttctgctatacccttttgtgctgtttcttatgttttgcttaaaaatttgatctgacaaacaaatttgtctaaattatgctcttgttctgactgtgtactgtacacatttgtattgctaccggtttggcacatagttttctctttgattgcagatcaaacccttctgattgcagattgcttggctatggctgatgtgctcactgttcttctttagcttgtatattgctgatggtggcttggctacggctgaaaaagttgctgctgcgccgacttggcaactgttgctccagtaatgctttgtcctctcttcctagccgccgacttggcacttggcagctgttgctcctgtaatgcgttgttatctcttatctcttgagatgtactatgattttgaggctgtttgacttgtgccagattgccagtgctgatgctatattatctcaggtttttatgctagtatatgttttactgtgtgctgcctgtgtcgagttacgtaatctattgaaccgctatgcgtgttgatgccctgttgttgcttgccagattgatgcccctgttgcttgcagcttgcaagctgtagtgaaatcctaaaattcgaaaccaaatctattctgaacttttgaagatctgtgatgtccggaagaaaagattcaaacagataaacgggactctccagaatctataaaataaatgatcgacaaacgctttgctttggtaggcttcctagttagttagcggaccgaagcttcggtccgggcctagaaggccagcttcggccgccccgggcaactttattatacaaagttgatagatcgaataacttcgtatagcatacattatacgaagttatacctggtggcgccgctaggggctgcaggaattcgatatcaagcttatcgataccgtcgacctcgagggggggcccggtaccctgggatctgcccttactttaacgcctctaaccaacacccctttatctttataaggaacaataaacagaatttgccccactgttctaaatcacctaataatatccccagctaaaaacaataaaggtttcctagaattaagacaagcatgactgttcctccaggagggtttggaacattgttgcagtcttgcagatacgggcgaagggtgagaaacagagcggagggctggaggtgacctcggtagtcgacgccggagttgagcttgacaacgacggggcggcccctgatggacttgaggaagtccgatggcgtcttcaccgtcccgccggcgcccgaggcgggcctgtcgctgccgccgccgccgctgctcatcttgcgcgctgtgcccccggcggtgtccctgtgttgcggatcgcgggtgggccaggtggatgcgagggcgacccgtttggactccggccggagccgccggatccctggtcggtgtcagtgccgtttactctgggccccacgtgtcagtaccgtctgtagatgacaacaacccgtcgtccacagtcatgtccaaaatatcctttcttcttttttttcgattcggatatctatcttccttttttttttccaaaaatcttcttgacgcaccagcgcgcacgtttgtggtaaacgccgacacgtcggtcccacgtcgatagaccccacccaccagtgagtagcgtgtacgtattcgggggtgacggacgtgtcgccgtcgtcttgctagtcccattcccatctgagccacacatctctgaacaaaaaaaaggagggaggcctccacgcacatccccctccgtgccacccgccccaaaccctcgcgccgcctccgagacagccgccgcaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgagccccgcaagggtgctgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtggcgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgcctgcagagttaacggcgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 70 - DNA, 18716 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacgctgtttaaacgctcttcaactggaagagcggttactaccggttcactagctagctgctaaggttaccagagctggtcacctttgtccaccaacttattaagtatctagttgaagacacgttcttcttcacgtaagaagacactcagtagtcttcggccagaatggcctcttgattcagcgggcctagaaggccggatcactgactagctaatttaaatcctgaggatctggtcttcctaaggacccgggatatcggaccgattaaactttaattcggtccgataacttcgtatagcatacattatacgaagttatatgcatacgcgtcttaagtcgatcgctatcaactttgtatagaaaagttgggccgagctcggtacggccagaatggcccggaccgggttaccgaattcaggcgacccatcgctgctttgtctacatcatgttcttcatcatcctccccaggcgacgcgtgctgctgttcttattcagactaccgttcgagtgactgcatggcgtacatctttctgcatcgactttgtacggctacatcgaacatatacacgagatgtctcgtgtgaatagagtcactaatgccttaagcatcggttactccgtagggtacattctgttcttcttatttgtgcatatttttattgttgtttactgattatacgagtagttatacatacatgcacatacatatcatcacatatatcacaatatttttctaaattaaattaaaactaaaaatgactaaatttctaacaccaacgacattgtaatgttttctccaacaactttacctattctacattgttctatttcgaatttcactctataaacaacatagtctacaatggaaaacagtgctttgtacgactatatacgcgatgtgtggctacaacataagacaatatagtcgtttgaagattgaacctatatatcggtacggttaatccgtctatgtacgtgggcatgacgaacacccgtgataacgaaggattaacgtgcacaatcataaatccaaagtaggagcggtgcatgatgagaatcgctctcagtactcgacataatgaaccttacgaggtacaacaggcaggcaggcagggaccaggggccgcctttatttcaggctcgctggccccacgggcgtgctgcgtgcacgaagggcactaccccaacctctcaccgaaaaccgcgctggatcggcaaatcaaacgaggtggtgccccgtgcccactctccacgtccacggcaccatccctctgcagccgctcaccagccatgccgtgtcgcggaacggcacaaccacccccaacccactcacgaaaccccgtcccggccgtgcccgtgtcggtccgcgctcggcaacgaggcggcccgcgctgctgagtcccctggacacccgacaccctgtcggccctttgtttattcatcccgaaatctcatctgcccccacggccgactgcgctgcgccgcccggatatatatacccatcgttatcgatcgactggggactctatcagtgatagagtctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactggtaccgttaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgacccaccttgctagttggatattacctcttctcttcaaagtatccttgaacgctcaccggttatcaaatctctacactatagctctgtagtcttgctagatagttagttctttagctctcggtgaccaagcttggcgcgatcaagcttatcgataccgtcgacctcgaagcttggtcacccggtccgggcctagaaggccagcttcaagtttgtacaaaaaagcaggctccggccagaatggcccggaccgggttaccgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcaccgggatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctagatcgatgggccctggccgaagcttggtcacccggtccgggcctagaaggccagcttcaagtttgtacaaaaaagcaggctccggccagaatggcccggaccgaagctggccgctctagaactagtggatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcccccgggctgcaggtcgactctagaggatccgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaagcttcggccgccccagcttggtcacccggtccgggcctagaaggccgatctcccgggcacccagctttcttgtacaaagtggccgttaacggatcggccagaatggcccggaccgggttaccgaattcctgcccttaaggccaattgttcaagattcattcaacaattgaaacatctcccatgattaaatcagtataaggttgctatggtcttgcttgacaaagtttttttttgagggaatttcaactaaatttttgagtgaaactatcaaatactgattttaaaaaatttttataaaaggaagcgcagagataaaaggccatctatgctacaaaagtacccaaaaatgtaatcctaaagtatgaattgcattttttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggatatgtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcaggtcgactttaacttagcctaggatccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagggtaccaatggccagttaacagatccagctgctgctgttctagggttcacaagtctgcctatttgtcttccccaatggagctatggttgtctggtctggtccttggtcgtgtcccgtttcattgtgtactatttacctgtaatgtgtatccttaagtctggtttgatggtgtctgaaacgttttgctgtggtagagcagcatggaagaactataatgaataagtgatccctaatcattgtgtccaaattttgcttctgctatacccttttgtgctgtttcttatgttttgcttaaaaatttgatctgacaaacaaatttgtctaaattatgctcttgttctgactgtgtactgtacacatttgtattgctaccggtttggcacatagttttctctttgattgcagatcaaacccttctgattgcagattgcttggctatggctgatgtgctcactgttcttctttagcttgtatattgctgatggtggcttggctacggctgaaaaagttgctgctgcgccgacttggcaactgttgctccagtaatgctttgtcctctcttcctagccgccgacttggcacttggcagctgttgctcctgtaatgcgttgttatctcttatctcttgagatgtactatgattttgaggctgtttgacttgtgccagattgccagtgctgatgctatattatctcaggtttttatgctagtatatgttttactgtgtgctgcctgtgtcgagttacgtaatctattgaaccgctatgcgtgttgatgccctgttgttgcttgccagattgatgcccctgttgcttgcagcttgcaagctgtagtgaaatcctaaaattcgaaaccaaatctattctgaacttttgaagatctgtgatgtccggaagaaaagattcaaacagataaacgggactctccagaatctataaaataaatgatcgacaaacgctttgctttggtaggcttcctagttagttagcggaccgaagcttcggtccgggcctagaaggccagcttcggccgccccgggcaactttattatacaaagttgatagatcgaataacttcgtatagcatacattatacgaagttatacctggtggcgccgctaggggctgcaggaattcgatatcaagcttatcgataccgtcgacctcgagggggggcccggtaccctgggatctgcccttactttaacgcctctaaccaacacccctttatctttataaggaacaataaacagaatttgccccactgttctaaatcacctaataatatccccagctaaaaacaataaaggtttcctagaattaagacaagcatgactgttcctccaggagggtttggaacattgttgcagtcttgcagatacgggcgaagggtgagaaacagagcggagggctggaggtgacctcggtagtcgacgccggagttgagcttgacaacgacggggcggcccctgatggacttgaggaagtccgatggcgtcttcaccgtcccgccggcgcccgaggcgggcctgtcgctgccgccgccgccgctgctcatcttgcgcgctgtgcccccggcggtgtccctgtgttgcggatcgcgggtgggccaggtggatgcgagggcgacccgtttggactccggccggagccgccggatccctggtcggtgtcagtgccgtttactctgggccccacgtgtcagtaccgtctgtagatgacaacaacccgtcgtccacagtcatgtccaaaatatcctttcttcttttttttcgattcggatatctatcttccttttttttttccaaaaatcttcttgacgcaccagcgcgcacgtttgtggtaaacgccgacacgtcggtcccacgtcgatagaccccacccaccagtgagtagcgtgtacgtattcgggggtgacggacgtgtcgccgtcgtcttgctagtcccattcccatctgagccacacatctctgaacaaaaaaaaggagggaggcctccacgcacatccccctccgtgccacccgccccaaaccctcgcgccgcctccgagacagccgccgcaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgagccccgcaagggtgctgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtggcgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgcctgcagagttaacggcgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 71 - DNA, 13825 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacgctgtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagcgatttaaatcctgaggatatcgctatcaactttgtatagaaaagttgggccgagctcggtacggccagaatggcccggaccgggttaccgaattcaggcgacccatcgctgctttgtctacatcatgttcttcatcatcctccccaggcgacgcgtgctgctgttcttattcagactaccgttcgagtgactgcatggcgtacatctttctgcatcgactttgtacggctacatcgaacatatacacgagatgtctcgtgtgaatagagtcactaatgccttaagcatcggttactccgtagggtacattctgttcttcttatttgtgcatatttttattgttgtttactgattatacgagtagttatacatacatgcacatacatatcatcacatatatcacaatatttttctaaattaaattaaaactaaaaatgactaaatttctaacaccaacgacattgtaatgttttctccaacaactttacctattctacattgttctatttcgaatttcactctataaacaacatagtctacaatggaaaacagtgctttgtacgactatatacgcgatgtgtggctacaacataagacaatatagtcgtttgaagattgaacctatatatcggtacggttaatccgtctatgtacgtgggcatgacgaacacccgtgataacgaaggattaacgtgcacaatcataaatccaaagtaggagcggtgcatgatgagaatcgctctcagtactcgacataatgaaccttacgaggtacaacaggcaggcaggcagggaccaggggccgcctttatttcaggctcgctggccccacgggcgtgctgcgtgcacgaagggcactaccccaacctctcaccgaaaaccgcgctggatcggcaaatcaaacgaggtggtgccccgtgcccactctccacgtccacggcaccatccctctgcagccgctcaccagccatgccgtgtcgcggaacggcacaaccacccccaacccactcacgaaaccccgtcccggccgtgcccgtgtcggtccgcgctcggcaacgaggcggcccgcgctgctgagtcccctggacacccgacaccctgtcggccctttgtttattcatcccgaaatctcatctgcccccacggccgactgcgctgcgccgcccggatatatatacccatcgttatcgatcgactggggactctatcagtgatagagtctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactggtaccgttaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgacccaccttgctagttggatattacctcttctcttcaaagtatccttgaacgctcaccggttatcaaatctctacactatagctctgtagtcttgctagatagttagttctttagctctcggtgaccaagcttggcgcgatcaagcttatcgataccgtcgacctcgaagcttggtcacccggtccgggcctagaaggccagcttcaagtttgtacaaaaaagcaggctccggccagaatggcccggaccgggttaccgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcaccggtaccgagctcgaattccggtccgggcctagaaggccgatctcccgggcacccagctttcttgtacaaagtggccgttaacggatcggccagaatggcccggaccgggttaccgaattcgagctcggtaccctgggatctgcccttactttaacgcctctaaccaacacccctttatctttataaggaacaataaacagaatttgccccactgttctaaatcacctaataatatccccagctaaaaacaataaaggtttcctagaattaagacaagcatgactgttcctccaggagggtttggaacattgttgcagtcttgcagatacgggcgaagggtgagaaacagagcggagggctggaggtgacctcggtagtcgacgccggagttgagcttgacaacgacggggcggcccctgatggacttgaggaagtccgatggcgtcttcaccgtcccgccggcgcccgaggcgggcctgtcgctgccgccgccgccgctgctcatcttgcgcgctgtgcccccggcggtgtccctgtgttgcggatcgcgggtgggccaggtggatgcgagggcgacccgtttggactccggccggagccgccggatccctggtcggtgtcagtgccgtttactctgggccccacgtgtcagtaccgtctgtagatgacaacaacccgtcgtccacagtcatgtccaaaatatcctttcttcttttttttcgattcggatatctatcttccttttttttttccaaaaatcttcttgacgcaccagcgcgcacgtttgtggtaaacgccgacacgtcggtcccacgtcgatagaccccacccaccagtgagtagcgtgtacgtattcgggggtgacggacgtgtcgccgtcgtcttgctagtcccattcccatctgagccacacatctctgaacaaaaaaaaggagggaggcctccacgcacatccccctccgtgccacccgccccaaaccctcgcgccgcctccgagacagccgccgcaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgagccccgcaagggtgctgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtggcgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgactagctagtcagttaacagatctgccagatcctcggtgtacaaataacccgtcttatcctatgagacgggccggcgtcagtgtgttctggaggaatttttatgtcagagcctttttctttgtgtgcttgatgtagatgccaagggaagcttattggctgttgaagcttgatgcaaaataaattatggaactctgttttttgtttatctaataataactagcaaatatgcttccattgcattgaaactaacagccttttgtgtttccaagttttattttgtgacaatgtcatctatttcaattagttgtggaatcggaaacttgcaggactaacttggaaactccaatccctcagcatcctggactttttcctggtgtaatccatgtagatattattttaatcatcattttagttctggaggtttttccatctccggttttgctcccctttcttcaaaaaaaaaaaaaaaatgccgtaggcgccgcaacgcccacctgttgttcaaactcatgggcacgagtggctcgaagattttatacaacaattgctgtagtttcaccgttgctggtgaagaagcatttttttaaaaaaatatagtggtattcattttaattagtttagttgtgcagcgagcaaaatttggacatgctctgctcggaatctgatcgacctagacacagattagcagcagtagctttgtcatctgttccaagagttgcgatctgatagaagaaaaaaaaaccttcccttcaatgtaaaaccgaaactaacaagaaagaagcacagtgccgtttaggcaagtatggagtacgtattaagcatgtagaaggccatgcatgaaccactaacaagaaagaagcgcagtgccattcaggcaagcataagcatgtagattggcatgcatgaacaactaacagtagatcgtctctggtctgattagaagttttttgggaagccaagaaatcatgtacaactggttccatctcaaattccgtggcaaaaagaggcctaagcaacataaagcttcggtccgggcctagaaggccattgggtcatcggatcccgggcaactttattatacaaagttgatagatatctggtctaactaactagtcctaaggacccggcggaccgaagctggccgctctagaactagtggatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcccccgggctgcaggtcgactctagaggatccaccggtcgccaccatggcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgaagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagcttaagccatggcccgggaatcttagcggccgcctgcagagttaacggcgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 72 - DNA, 20570 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacgctgtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggcctccggaatttatcgctatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccggtcgccaccatggcaccgaagaagaagcgcaaggtgatggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagacaagagaccacgggaccgccacgatggcgagctgggaggccgcaagcgggcaaggtaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgctgatccgatatcgatgggccctggccgaagcttggtcacccggtccgggcctagaaggccagcttcaagtttgtacaaaaaagcaggctccggccagaatggcccggaccgggttaccgaattcgagctcggtaccctgggatccgatatcgatgggccctggccgaagctttgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgcatcatatctatccgtagccgttttagagctagaaatagcaagttaaaataaggctagtccgttatcaacttgaaaaagtggcaccgagtcggtgctttttttttgcggccgcgaattcctgcagggccctcttgtcggaccagttgcccaccacgttggtgaggggcacccagctttcttgtacaaagtggccgttaacggatcggccagaatggcccggaccgggttaccgaattcgagctcggtaccctgggatccgatatcgatgggccctggccgaagctttgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgttacttctctaagcacggcgttttagagctagaaatagcaagttaaaataaggctagtccgttatcaacttgaaaaagtggcaccgagtcggtgctttttttttgcggccgcgaattcctgcagggccctcttgtcggaccagttgcccaccacgttggtgaggggcaactttattatacaaagttgatagataatacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggcctaaggggtcaccggcctagaaggccatttaaatcagatatctggttcctaaggacccggcggaccgattaaactgattcggtccgacgcgttacctaataacttcgtatagcatacattatacgaagttattagctaactaactagtaagcttaagccatgggtcgacggatccgttaactcacttaagatgtactcgacaatggtgccctcataccggcatgtgtttcctagaaataatcaatatattgattgagatttatctcgatatatttctgaactatgttcatcatataaataactgaaaacatcaaatcgtaattttaaactcatgcttggtcaatacatagataatacaatattacttcatcatcccaatgatgtcctagcacaacctattgaatgttaatgtttggttgtgtgggggtgtgtttataacatagatgtgattatttgtgctttttgttgagtatatacatatatggtatgttgatttgatatagtgatggacacatgctttggccttggatattcaaatcacttgtacttgcacgaagcaaaacataatataagtttagaagtaaacttgtaactgtgtccaaacatgctcacacaaagtcatatcgcattatatttttttggtaaatattcaacacatgtattttttacaagaacccaaattttacagacaaatgcagcattgtagacatgtagaattctttgaagcatgtgaacttaacaacactaatgtcattaaatcaactggaccctatgagtaacaatttcgatattgcaaacaccaaattatggaacttatttgctgaaaaaattatgatcaatgtgaagtttaaattattataccataaatatatcaaagattttttttgaggaaggtaaaaattgcatggaatgggctgcccaacgtgatagctcacttttatgctaggtagcattaccaaagatgggaatgttctgatgaacaccaaacccactcaaataatatttatatttgggttgtttagttgtaaaagtgaagacccaagattaaagtaccaattgtcgatgccatacactagaagtgaagaatgttgttagcgttaggagtagatgtgtcaatggggaccgatactcgttatcgatgggccgcacgtaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggatccaccatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctagggcgatccgatatcgatgggccctggccgaagcttggtcacccggtccgggcctagaaggccagcttcggccgccccaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactaagctgaccgaagctggccgctctagaactagtggatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcccccgggctgcaggtcgactctagaggatccaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgaagcggcccatggatattcgaacgcgtcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctcgagccagggtgcttcctgagctgattccgatgacttcgtaggttcctagctcaagccgctcgtgtccaagcgtcacttacgattagctaatgattacggcatctaggaccgactagctaactaactagtacgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 73 - DNA, 20572 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacgctgtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggcctccggaatttatcgctatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccggtcgccaccatggcaccgaagaagaagcgcaaggtgatggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagacaagagaccacgggaccgccacgatggcgagctgggaggccgcaagcgggcaaggtaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgctgatccgatatcgatgggccctggccgaagcttggtcacccggtccgggcctagaaggccagcttcaagtttgtacaaaaaagcaggctccggccagaatggcccggaccgggttaccgaattcgagctcggtaccctgggatccgatatcgatgggccctggccgaagctttgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgctccagctgctctaatcgatggttttagagctagaaatagcaagttaaaataaggctagtccgttatcaacttgaaaaagtggcaccgagtcggtgctttttttttgcggccgcgaattcctgcagggccctcttgtcggaccagttgcccaccacgttggtgaggggcacccagctttcttgtacaaagtggccgttaacggatcggccagaatggcccggaccgggttaccgaattcgagctcggtaccctgggatccgatatcgatgggccctggccgaagctttgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgcgtggtccttggctgccttggttttagagctagaaatagcaagttaaaataaggctagtccgttatcaacttgaaaaagtggcaccgagtcggtgctttttttttgcggccgcgaattcctgcagggccctcttgtcggaccagttgcccaccacgttggtgaggggcaactttattatacaaagttgatagataatacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggcctaaggggtcaccggcctagaaggccatttaaatcagatatctggttcctaaggacccggcggaccgattaaactgattcggtccgacgcgttacctaataacttcgtatagcatacattatacgaagttattagctaactaactagtaagcttaagccatgggtcgacggatccgttaactcacttaagatgtactcgacaatggtgccctcataccggcatgtgtttcctagaaataatcaatatattgattgagatttatctcgatatatttctgaactatgttcatcatataaataactgaaaacatcaaatcgtaattttaaactcatgcttggtcaatacatagataatacaatattacttcatcatcccaatgatgtcctagcacaacctattgaatgttaatgtttggttgtgtgggggtgtgtttataacatagatgtgattatttgtgctttttgttgagtatatacatatatggtatgttgatttgatatagtgatggacacatgctttggccttggatattcaaatcacttgtacttgcacgaagcaaaacataatataagtttagaagtaaacttgtaactgtgtccaaacatgctcacacaaagtcatatcgcattatatttttttggtaaatattcaacacatgtattttttacaagaacccaaattttacagacaaatgcagcattgtagacatgtagaattctttgaagcatgtgaacttaacaacactaatgtcattaaatcaactggaccctatgagtaacaatttcgatattgcaaacaccaaattatggaacttatttgctgaaaaaattatgatcaatgtgaagtttaaattattataccataaatatatcaaagattttttttgaggaaggtaaaaattgcatggaatgggctgcccaacgtgatagctcacttttatgctaggtagcattaccaaagatgggaatgttctgatgaacaccaaacccactcaaataatatttatatttgggttgtttagttgtaaaagtgaagacccaagattaaagtaccaattgtcgatgccatacactagaagtgaagaatgttgttagcgttaggagtagatgtgtcaatggggaccgatactcgttatcgatgggccgcacgtaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggatccaccatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctagggcgatccgatatcgatgggccctggccgaagcttggtcacccggtccgggcctagaaggccagcttcggccgccccaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactaagctgaccgaagctggccgctctagaactagtggatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcccccgggctgcaggtcgactctagaggatccaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgaagcggcccatggatattcgaacgcgtcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctcgagccagggtgcttcctgagctgattccgatgacttcgtaggttcctagctcaagccgctcgtgtccaagcgtcacttacgattagctaatgattacggcatctaggaccgactagctaactaactagtacgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 74 - DNA, 26187 bp, Artificial sequence
  Synthetic construct
gtttacccgccaatatatcctgtcaaacactgatagtttaaactgaaggcgggaaacgacaatctgatcatgagcctagttagttaggggcagctaacgtcccctagctaactagttgctattaacctttagcctagtttagtgcggtcgcgctagtgaactagtctaatgggtgcctagcttagattagatccccgttcggcttagactaggttagtcggatagggctagtctagtggcttagcttggaactgttaagctaggctgttagtgcgggacttctaggctaacctattgacccctaacgcctagaacctaagaaacgctaaggacacctagctaactttggcgacgtccagaggtccgattccggctaactaactagcttaagcttgatatcgaattcctgcagcccatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgaaaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtcacggtctcgatctttggccttggtagtttgggtgggcgagaggcggcttcgtgcgcgcccagatcggtgcgcgggaggggcgggatctcgcggctggggctctcgccggcgtggatcaggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaaggatccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggcgatcgcggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaaggcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggcctccggattcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcacgcgtaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggtcacaccggttaaaaccaaaatccagtggcgagctctcgagtcgatcgctatcaactttgtatagaaaagttgggccgaattcgagctcggtacggccagaatggcccggaccgggttaccgaattcgagctcggtaccctgggatcagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctaggcttaggctactgctagtccgcggtgtatccgtgatcgagttggcgccagacggaatctgttctccatcgctgacatcctcgagtagatcacattcaagcttgatatcgaattcctgcagcccatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtcacggtctcgatctttggccttggtagtttgggtgggcgagaggcggcttcgtgcgcgcccagatcggtgcgcgggaggggcgggatctcgcggctggggctctcgccggcgtggatcaggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctgcgcgccgccggctacaagcacggcggctggcacgacgtcggcttctggcagcgcgacttcgagctgccggccccgccgcgcccggtgcgcccggtgacgcagatctgagtcgacctgcaggcatgccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctaatgcggcccgaataacttcgtatagcatacattatacgaagttatacctggtggcgccgctaggggctgcaggaattcctgcagcccgggggatccactagttctagagcggccgacctcgacagatctaagcttactagtgccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattcactagtgattcagatctgatatcgatgggcccactaactatctatactgtaataatgttgtatagccgccggatagctagctagtttagtcattcagcggcgatgggtaataataaagtgtcatccatccatcaccatgggtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgggtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttggtcacccggtccgggcctagaaggccagcttcaagtttgtacaaaaaagcaggctccggccagaatggcccggaccgggtgaccggcgcgccaagcttggtgacggcgacgcgatcgaacaggtggtgatcgatgctgcaacgtgtgtaaatatacagcgccggctgggtcaagagatggctcgggtgacgcgcgcgcggcgtgtcctggcgttggcgccggggcattctttagtttttcatcttttcatcatctcagatggtagatacaaaacagtgtatgtatgtagctctgtttctctctatagaaccccaacaaattttgttgttgatgttgtttatcttcatatgctttgatcttgaaatcgtctaccttactactgccgatcgttgctcaaaagtgtggaagtttgaagcatcttccacgggcgttgctctactcccatctcgtttcaccacgaaatccccttcgcatagaggcatctcagccgtacatctccaaacccacgttctggattcagcaaacggacggcatatgccagcggactgactaggatttgtggttcagaataaaaatatggtttgcagtgtcaattttccaggagagtgactatgtcatactaacttcatactccaataatgaagctaagctatctccatgtacatatcaaatacgaaatcatatccaagggaactaacacagtcacaaacaacaggtacacagacaattacagcacaagcgcaggagggaaataattttaactgaactaggaagaaaggaaacacaactcattttttattgatatatgttggatgaatccaataaaaccgatacaagtcacgaaaaatcagactagatgaatccttcgagatcacatgagcaaaacctttcgacgaaagctgtcctatagtcgtggaagcaataacacttgataaagataggaattcagacacgagaggttgcagactataagatgtcaggtacctacgcgttcgaatcaatgagatgatgcagtagaggtagagcgaagagggaaggctgagatcgacgcatcactgccatgtccctgatgatgatgaggtagcaatggagatctacagagcgggcaattgaagttgagatgttgaagccagccttccaaacactgcttatggaagacgtgtctgcaatctagcttcctcacttcttctccggtcttgagtttagacagacacacgatgcagtcagaggctgcgttgtcggagtagcggtacgagaagaggcgatttaggttgagctggtcggcgaggacgctgaggtttgaagtaacaacaacaacgggggcagaagaagggaagaggagaagagaccggacgtgtctgaagaaagttgcgaggagagccagtagcatcagtgggatcgaatctgatgacacgtcggagagctgaccttgtagtcccatggatcctaggctaagttaaagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatgcatggaccggaacgccgatctagagaaggtagagaggggggggggggaggacgagcggcgtaccttgaagcggaggtgccgacgggtggatttgggggagatcctcgctttcctggaggaggcgtggagcgcacgcggttggaggcgaaggcggatgaaaaacgagaggggctgacgggctgtcttctgtctggcttgcgcccctccgacgggttggttggctttataggcgtgtggtgcatataattaggtgcgcgaaccgccgttccgatggaaaaaactggctatggaggctctcgattcgtgccggcctatcgagacggagctttttttattatttattttcatctcgcatgtatgttgtttgttcgctgatggaatttctgtttgttcgcgctgaaaacattggacagggttcgtttatgctgtgcccagtgaggaaccataaaaggaacgctgtgcatatggagtcacgtacttagtaaaaaatccttcgctctggattaaactaggttgattatactgttgcacgtagtatctatagagacaaaaatattataaaaatagtagaaaccgatacaaattaaagttaagtaaacatgatgtttatctgatactctaagtcaagcaagtcatttctagtgttttcttctgtgctactagcagagcaagagattgggacccgaggcgggggccatgcagacatagggggatgctccttaggagagagataaaaataaataatagaatatatgattattggtatagagtacagatttaaggtaaatattgcttcaaaagattgtggaatagagtagagaatctcgttgacaaaaatgaaatattacttttagaataaaaatttagagttgcataagtatagatatcattagtttggattgagggctagagctattccagctatttaggctctgtttataatcctacagccaataattaccagaagcttcggtccgggcctagaaggccgatctcccgggcacccagctttcttgtacaaagtggccgttaacggatcggccagaatggcccggaccgggttaccgaattgctaactaactaggagctccctttaatctggcgcttgatctgcatccgcggcttgcaaagataaatggcacatttagtgtgttattttgcaatacctttcatagtagatatccttaaatgcagttttaggcatgtttgggtaattaaataacatttttaggaggagttttagatttacctttctttcgtgatgactgatgacagacgtggggaattcaaatgcaactctagcgaaagttcatatatttttcataaatagctgaggctggggtaattattttttgtagaaaaatagaataggtggaatggtttggggaaggcgtaggcgctcgtggacgacgccccataaaagacaagaggcggaattgccatgaattcgaggtagctaagtaaggcgcatatatatgccaaaaattctactgtcactttccaatttcaatgcgctgccaaacaagccgccgacccttggatactgacttgaattcagcccaattctgtagatccaaacagggccggcgtcagtgcctcaggtgagagagcagcagacgatgcaaagagccaaaagtggaagcagacgcagccgaagccgaagcccaagcccaaaactgttttgtctttgcccagaaccgcgacgagcctaaactgccgcttcctcctatctacaagtccctggcacatcacgcatagtccaaccatcgcgcgcaggcgataaggcgcgccacggggacgcgacatgtggtggcggacgcgatcaggatagggccaggctggccgggcgcggccacgggagaacggtggccactcgtcccacatccgcttcgtcctgtcctgtactgcgtcctgcccccaacgagagccggagccggccatcccgtcgcacactctccccctctatatatgccgtcggtgtgggggagcctactacaggacgacccaagcaagcaagcaagcagcgagtacatacatactaggcagccaggcagccatgatgctgcactgcacatttgctatatctgaggctcctgcgcgcgccttggcccttggccaggtgtctgtcatgcgggcgatgccgcaggaagaagaagccgcggtggctacgacgaccatggccgggggcaaggtggcggcgctgctggccacggcggccgcgctgctgctgctgctcccgctggcgctgccgccgctgccgccgccgcccacgcagctgttgttcgtccccgtggtcttgctgctcctcgtggcgtccctcgcgttctgccccgccgcgacctcctcgccgtcgccgatgcatgccgccgaccacgggtcgttcgggaccactggatcaccgcacctatgttgaggtacccggggatcctctagtgccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattcaatcactagagtcgacctgcaggcatgcccgcggtggagctcgaattccggtccgggcctagaaggccagcttcggccgccccgggcaactttattatacaaagttgatagatcgagcgactaattaactagctgtcccacggcctaactagcacttaatcccctagcctaacctaagagcgctaatctaggctagtggtcacttagggctttaaggctagcgtatacgaagttcctattccgaagttcctattctccagaaagtataggaacttctgtacacctgagcctaactaactaggacgtcccgaggtccgattccgggctaattaacacctagctcgccactcgactaattagggactaagcctagcgcttagccgttaaaacctagcacaccctaagcacccttagttaggttcccctcttaattaagccctagtgagcccctaagttaaggggacgctaagagccccctaacctagtattcggctagaggcgaactaggctaaacacctaagcgcacctctttaagctagatcgctagggggctaggctagagcttcgctagattagtctaagggcagctaactaactaggtttaaactacattaaaaacgtccgcaatgtgttattaagttgtctaagcgtcaatttgtttacaccacaatatatcctgcca

SEQ ID NO: 75 - DNA, 9982 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggttcactagctagctgctaaggttaccagagctggtcacctttgtccaccaacttattaagtatctagttgaagacacgttcttcttcacgtaagaagacactcagtagtcttcggccagaatggcctcttgattcagcgggcctagaaggccggatcactgactagctaatttaaatcctgaggatatcgctatcaactttgtatagaaaagttgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagatccatgcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagagttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccgaagctaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggtcacccggtccgggcctagaaggccgatctcccgggcacccagctttcttgtacaaagtggccgttaacggatcccggtgaagttcctattccgaagttcctattctccagaaagtataggaacttcactagagcttgcggccgccccggagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccccgggcaactttattatacaaagttgatagatatctggtctaactaactagtcctaaggacccggcggaccgattaaactgattcggtccgaagcttaagccatggcccgggaatcttagcggccgcctgcagagttaacggcgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 76 - DNA, 10955 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttaccagagctggtcacctttgtccaccaagatggaactgcggccgtaagtgactagggtcacgtgaccctagtcacttatcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcgcctcattaattaagtcagcggccgctctagttgaagacacgttcatgtcttcatcgtaagaagacactcagtagtcttcggccagaatggccatctggattcagcaggcctagaaggccatttaaatcctgaggatctggtcttcctaaggacccgggatatcggaccgattaaactttaattcggtccgacctggtggcgccgctagcgtatacgaagttcctattccgaagttcctattctccagaaagtataggaacttctgtacaataacttcgtatagcatacattatacgaagttatatgcatacgcgtcttaagtcgatcgctatcaactttgtatagaaaagttgggccgaattcgagctcggtacggccagaatggcccggaccgggttaccgaattcgagctcggtaccctgggatccgatatcgatgggccctggccgaagcttggtcacccggtccgggcctagaaggccagcttcaagtttgtacaaaaaagcaggctccggccagaatggcccggaccggaattcgagctcggatccactagtaacggccgccagtgtgctggaattcgcccttgacggcccgggctggtatttcaaaactatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaagcttggtcacccggtccgggcctagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaagcttgcggccgccccagcttggtcacccggtccgggcctagaaggccgatctcccgggcacccagctttcttgtacaaagtggccgttaacggatcggccagaatggcccggaccgggttaccgaattcgagctcggtaccctgggatcgaccgaagctgaccgaagcttgcggccgcacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccgggggatctcgactctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgcgactctagatcataatcagccataccacattcgaatgtgagttgatccccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgagtcgacctcgagggggggcccggtaccaagatatcaaccgcggaaagatctaagcatgcaagggcccaagtcgacctgcagaagcttcggtccgggcctagaaggccgatctcccgggcacccagctttcttgtacaaagtggccgttaacggatcggccagaatggcccggaccgggttaccgaattcgagctcggtaccctgggatcgaccgaagctgaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaagcgtacgtgccggcctggctctccgaaagggcgaattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacaaacgtgcaagcgctactagacaattcagtacattaaaaacgtccgcaatgtgttattaagttgtctaagcgtcaatttgtttacaccacaatatatcct

SEQ ID NO: 77 - DNA, 965 bp, Streptomyces spectabilis
atgcagaatcttcctgaaaagttcgatgaaacacgcctatgggaagctttgaggggatttggtgtgtccccgtcgcgtgtcacctacgcccccgtcggattcggggactaccactggacggtgaccgacgaggacggccggccgtggttcgccaccgtctccgacctggagcacaaggagcactgcgggcagggggcacaggccgcgctgaaggggctccgacaggccatggacacggcgctggccctgcgcgaccgtgacggactgcggttcgtcgtggcaccggtggccgccaccgacggcggcggtccagtactgcccctggacgcccggtacgcgctcacggtgttcccccatgtcccgggccgcacaggggagttcgggcagcgcctgacggaggccgagcgggaccggctgctcgccctgctcgcggagctgcacggccggacgcccccggagaccacaccgcccgccgacatggagcccccgggccttcccggcgtgcgcgcggccctggccgagtcggaagggccctggtcgggcggcccgttcgccgagcccgcgcggctgctgctgcgcgagcacgaggcgacgctccacgcgcgcctcgccgagttcgaggccctcgtcgcgcgcgtgaagggccggggcgcgccgctcgtcgtcacgcacggcgagccgcatccgggcaacctcatcctgggtgaggagggctatctcctggtggactgggacacggtgggcctcgccccggccgagcgcgacctctccctgatctcggacgacccggcggacctcgcccgctacgccgagctgaccggccgcaccccggacccggacgccctcgcgctctaccggtggagtggttccgcgccgagcaccagcgcacgcaggacaccgagtccgcgtggaagggcttcacggacacgctggggcaactggcggcgggcggatccgcagcctag

SEQ ID NO: 78 - DNA, 993 bp, Legionella pneumophila
atgctaaaacaaccaattcaagctcaacaacttatcgaacttttgaaagtgcattatggaattgatattcatacagcacaattcatccagggtggtgctgatacgaatgcatttgcatatcaagcagattcagaatccaagtcttatttcataaagctaaaatacggctatcatgatgaaattaatttatcgataatccgtcttttacatgattctggaataaaagaaattatttttcctatccatacacttgaagcaaaattattccagcaactaaagcattttaaaataattgcgtatccatttattcatgcgcccaatggtttcacccaaaatttaacaggaaaacagtggaaacagcttggaaaagtattaagacaaattcatgaaacatcagttcccatctcgattcaacaacaattaagaaaagaaatatactcccctaaatggcgtgaaatagtcagatccttttataatcaaattgaatttgataattcagatgataagctcacggctgcctttaaatctttttttaaccaaaatagtgctgcaattcatcgattagttgatacttcagaaaaactatctaaaaaaattcaacctgatttagataaatacgtactatgtcattctgatatacatgcgggcaatgtgttagtcggtaatgaagagtcgatttacattattgattgggatgagcctatgttagctccaaaagaacgtgatttgatgttcataggtggtggcgttggtaatgtatggaataaaccccatgaaatccaatatttttatgaaggttatggtgaaataaatgtcgataaaacaattttgtcttattacaggcatgaacgaattgtcgaagatatcgcagtatacgggcaagacttgctttcacgtaatcaaaacaatcagtccagacttgaaagttttaaatattttaaagaaatgtttgatccaaacaacgttgttgaaatagcttttgctacagagcag

SEQ ID NO: 79 - DNA, 2556 bp, Agrobacterium rhizogenes
atgatttgggatcatttatcaccttcccgaagggttctgcttgatatcaaatcgtggtctgtattggtactggtaattgcctcgatatcttttgcggttctggccataggatcttggcaagacaacgaaagcaacgaagcgatcctgacagagctgcaatcgatcgacgtggattgcgcgatgctgcagcggaatgtgttgcgtgctcacgccggccttcttcggaattaccatcctcttatcgtccctctggggcgactgcgcacgagcgtcgcagatttacagcagctttttaggcgggcgggcctcgacggggccggtgaattttctgaattgctggcgcgggtgaagagctcagtagatgcgaccgacgcagccgttgcagcgttcggtacgcaaaatgtaatgttccaggattcgttggcgacctttaaccagtcgatagcttcgcttccgaggtctttggatacgagggatttgaactcgctagaagtcgctgagttgggctatctgatgctccagttttcgtctcagccaaatttcgagtttgcacgggaaatcaaccaacgtctcgaccggcttcaggtttcggcgaacggggataaggcaggcgtcaaggagatcgttcgcagtgggaggattatcctgactctactgccgcgattgaaagacaccgttggagtgatccaaacttctgaaaccatcgacaacacgaagaagttgcagcgagcataccttgaagcttatagcttggccagcgcgggtgaacagcgggtacgggtcttcctcggcgcagtttcggttttcttttgcttttgcataatttccctagtccataggttacatctgcgaaccaaggtgttgacgagacgactggacttcgaagaagtgatcaaaaagattgggctttgcttcgaagatgcttcagaagcaaaaccgtcgttggaatcctcaacgaatgccgcgttgggaattatccaattattctttgaagctcatcaatgcgcgctgggactggtcaacatcaatgagaatgacgttggcgtcaccttttttggaagcactcctgcgccagagtggaacgaacaacgggtgcgcgaaattgtttccacggtaggggccgaggacggcgggcggattttccgagcctatcctcaacgaaaagcgagctgcttcggtgaggactctccatttctgtgggtgctgcttgcgttcaaggtatccgatcgaatagtcgctgtctttggcctgggatttgatcgagagcatctacagacgccgaccgcacgtgaaatgcagctgatggaactagcggctgcatgtgttagccactatgcggtcattcggcgcaagcagtcccagcgagatattctggaacgtcgtttgaaacatgcggaacggcttgaagcagttggtacgcttgccggcggcattgcgcatgaatttaacaacattttgggtgcagttctgggttacgcggaaatggcacacaacgttctgcgccgtcgtactcatgctcgagactacatcgatcacatcatttctgaaggcaatcgagcgcggttgattgtcaaccagatccttgcccttagtcgaaagcgggatcgaacaacaaaaccattcgatctttcggagttagtgaccgtgatcgcgccgtcgttacgtgttgcgttgcctccgggcgtcgagctcgatttcaaatatcagagcgctccaatggttatcgaaggaaactcacttgaggtcgagcaaatactgatgaatttgtgcaaaaactctgctgaagcatgccgagagttagggcgtgtggaggtcagcgttaggaggtctgtcgtacggaaacacaaggttcttacaaacggtactattcccaccggcgagtatatccttctttcagttgaggataacggcgcaggcataacagaggcggtattatcgcacattttcgagcctttttttactacccgcgcccgcagcggtggcacgggcctgggattatccacggttcacggtcacgttagcgcgatggctggttacatcgacgtggtatcaactgtcggcagaggtacgcgcttcgacgtctacttgccaccttcgtcgaagcagccggtaaattcagagagtttttttgaccctggaagaatcccgcttgggaacggcgagattgtcgcggtggtcgaacccgacttagccacgctggaaatgtacgaggaaaagatcgcggcgctgggctatgagccagtcggctttaacacgttggatggtttaatcgactgggtgttggaaggaaaagaacctgatcttgttctaattgatcaaccgtctttgctggatggagacggtgcagggtccttaatctcaaagctggcaaaagcgcccatcatcattattggcgaaaaccagaaaaatgttcccctttctgcggatcgcgagggatctgcccgttttttggaaaagccgatttcagcaaagactctcgcctatgtcgttcgtgcgaatatcagaactgaacgaatggtcggctcgaatagcagcggaatcgtgattgcgtcataa

SEQ ID NO: 80 - DNA, 720 bp, Agrobacterium rhizogenes
atgcggaagacggcaggatctttcgggatcatttttctgatggtccagccaagcaaagctgcgccgctctcgttcgctgaatttaaccaactcgctcgcgaatgcgcaccttcagtcgctccatcgacgcttggagcgattgcgaaggttgaaagtcagtttgatccgctcgtgctgcatgacaataccaccggcgaaactttacatggcaagaatccggcggatgctacgcaaagcgtaaacaatcgcgtcgcagcagggcattcagtcgatgtaggcctaatgcaagtgaactcgaaaaatttcgcgaacttgggcttgactgccggcaatgcaatcaacccttgcgtgtccctgtcggcggcggcggacctgctcgtacggcattatagtggcggcgacacggtagagtcagaacaacttgccatccggcgagcaatttcggcttacaataccggcaacccgacacgcggcttcgcgaacggctatgtgcggagggtcgaagtggcggctcagctactcgtccccccgctggctcagtcggggaaaaaaggcgaccgtgatgagcggagtccagaagaaccctggaacgtctggggttcatacgaccgctcccactcagcagtcggttcgtccgcgccgccagaggcgcagcagccgaatgagcgcaaatcccccgaacaagatcaagttttcgaaccgaatgatggagatgcacca

SEQ ID NO: 81 - DNA, 360 bp, Agrobacterium rhizogenes
atggcatggcttgaaggataccgcgcaccgtcgaaattccgtggcctctggcacagagctgtgcgcctgctggctccgcacgtgccgagcgtcaccggcgcgatcggctggagcttgtttttctgcgagccagctgccgcgcaggccgctgggggcactgaccccgccaccatggtcaacaatatctgcacgtttattctcggtccgtttggtcaatcgctcgccgttcttggtatcgtagccatcggggtatcgtggatgttcggccgagcttcccttggtctggtagcaggtgttgttggcgggatcgttatcatgtttggagccagctttctcggccaaacactgacagggggcggg

SEQ ID NO: 82 - DNA, 324 bp, Agrobacterium rhizogenes
atgaatgccgatcttgaagaagccacactctacttggcggcaacgagacctgcattgttctttggggtaccgctgacgcttgctggagttttcatgatgttggcggggtttctcatcgttgttgtgcagaaccccctctatgaaattgttctcttgccgctgtggcttggagcgcggctcatcgtagagcgggactataacgcggcaagcgtggtcttgctgtttctccagacggcagggagaagcgttgacggccatgcgtggggcggcgcgagcgtcagtcccaaccccatcagagtggcgtcgcgaggaagaggaatgacg

SEQ ID NO: 83 - DNA, 2370 bp, Agrobacterium rhizogenes
atgttcggagcaagcggacggaccgagagatctggggaaatctacctgccttacgtagggcacctcagcgaccatgtcgttcttctagaggacggttccatcttgaccatggcgcatatcagtggccttcccttcgaactggaggaagtagaggtccgaaatgcgcgctgccgtgcctttaacacgctctttcgcaatattgccgatgataacgtctcagtatatgcccatctcgtccgtcacaacgatgtgccagtaccgccggtacgccattttcgcagcaccttcgcgtcaagcctaagcgaaacgttcgagcggcgtgttctgtctggtaaactgtttcgaaacgaccacttcctcacgctcattgtctctcctcgcaccgctctcggtaaagtgggaagcagattcaccaggcgctatgggaagaacccaggcgatcttgcgcatcagatccgacatctggaggatctttggaacgttgtcgctggcgcgcttgatgggtatggacttcgtcgacttggcatccgagaaaaaaatcaggtgcttttcacggaaattggtgaagcccttcgcctgataatgacttgtcgttttttaccggtgccggtcgttagcggatccctcggcgcctcagtttataccgaccgggtcatctgcggcaagcgtgccctcgagattagagcgccgaaagatcgttacgttggatctatcttttcattccgcgagtacccggcaaagacgcggcccggaatgcttaacacactcctgtcatcaagttttccgctcgttttgagccagagtttctctttcctcacacgtgcacaggctcacgccaagctcagcctcaaatcgaaccagatgactagctccggtgacaaggcggtcacccagatcggtgaactcgcccaagcagaagattcattggccagtagcgaattcgtcatgggttcgcatcatctcagcctttgtgtctatggcgatgatctcgatacccttgccgactatggtgcgcgagcccgcacgagcttgtcggacgcgggcgcggttatcgtgcaggaaagcattggaatggaggctgcatactggtctcagcttccaggaaaccacagatggcgtacccgtcccggagcgatcacttcacgcaactttgccggcttggtttcattcgagaacttccctatcggaaggaagtcgggccattggggaggtgcggttgctcgtttccgtacaaacggcgggacgcccttcgattatattccgcacgaaaacgacgtgggcatgaccgcgatcttcgggccgattggcaggggaaaaacgacacttatgaccttcatcctggcgatgcttgagcagagcgtggttgaccgcagcgggacgatcgtttttttcgataaggatcgtggcggtgaactgcttgtgcgcgccaccgggggcacgtacctcacgttacgacgaggtgtcccaagcggccttgcacctttgcgcggcctagaggatacggcagcggcgcgggactttctccgcgagtggatcgtggcgctgattgaaagcgacggcagggggggaatttcgccggaggaaaatcgccgcttagagcgcggtatccaacggcaactttcgtttgagcctaacatgagatcccttgcgggcttaagggagttcttattgcacgggccctccgaaggtgcgggagctagattgcagcggtggtgccgaggcaacgctcttggctgggcgtttgacggcgaatcggacgaagtgaagttagatccttcgattacgggcttcgatatgacccatctcctcgaatatgaggaggtatgcgcgccggccgcagcataccttttgcatcgaataggagcgatggtagatggccgacgcttcgtcatgagctgcgatgagtttcgttcctacctgcttaatcagaaattcgcagcagtcgtcgacaagttcctgctgaccgtccggaagaacaacggaatgttagttcttgcgacgcagcaacccgagcacgtacttgattcaccattgggcgcaagcctcgtagcacaatgtatgaccaaaatcttctacccatcacccactgcagaccgctctgcttacatagacgggttgaagtgcaccgagcgagaatttcaggctattcgcgaggagatggcgattggcagccgcaaatttctgctgaagcgcgagagcggaagtgttgtgtgcgaattcgatctgcgcgacatgccggaatacgtcgccgtactatccgggcgtgccaatacggtgcgttttgcccagcagttgcgcgagacgcatggagaggaaccctctgcctggctggaaaaattcatgacgcgctaccatgaggcacaggattga

SEQ ID NO: 84 - DNA, 669 bp, Agrobacterium rhizogenes
atgaatattacgaagctcgtaataaacgcacttttcatatgccttgttctgtcggggactgccaaggcgcagtttgttgtcagcgaccctgcaacggaggccgaaacgctgacgacagcgatcaatactgcggcgaacctcgagcaattgatcacgatggtgacaatgttgacctcgccctttggcgtcaccggcatgttatcagcgatcgaccagaaaaaccaatatccctctgccggccaactcgacaaggaaatgttttcgccgcagatgcctgcttcgacaactgcgcgtgcaattaccttagatgccgatcgcgcagtcgtgggcgacgacgctgaaggaaatcttttgcgacagcagattgcaggcgccgcaaatgctgccggtgtcgcggctgacaatctggatgcaatggacaagcgccttgcggcaaattctgagacatcgggccagctctcccgctcacgcaatatcatgcaggccaccgtcaccaacggtttgcttctcaaacagatccatgacgcaattattcaaaacattcaggcgaccagcctgttgacgatgaccaccgcacaggcggggctgcacgaggcggaggaggcggcgacccaacgaaaggaacatcaggcaactgcgcttatatttggcgccgctcaactacactga

SEQ ID NO: 85 - DNA, 888 bp, Agrobacterium rhizogenes
atgaattttagcattcctgcgccttttacggcgatacatacgatattcgacctcgccttcaccgtcgggctggacacgttgcttggggacattcaacgggcagtcagcgctcctctcgtggcctgcgtgacgctatggatcattgtccagggcatcctcgtaatgcgtggggaaatggacgcacggggcggaatcacccgggtaatcatggtatcggtagttgttgctcttatcgttgagcaggcggaatatcatgattacgttgtctcggttttcgaggatacgattccgaatttcattcaacagttcggtatcagtgggcttcctttgcagacgattcctgctcagctcgacacgatgttttcactcacgcaagtagcctttcagaagattgcttctgaaattgggccgatgaacgatcaagacatcctcgcctttcaaggcgcacaatggattttctatggaacgctttggactgcgtttggaatctacgatgccgtcggaatcctcaccaaggtgctattggcgatcggtccattgatgctcgttggctatctcttcgatcgcactagagacatggccgcgaaatggatcggccagctcgtcacttatggcatacttctgcttcttctaaacatagtggcaactatcgtggttctgacggaagcgacggcactcgtgctaatgctcggggttatcacttctgctggcacaacggcggccaagataataggcctctatgaactcgacatgttctttctgaccggcgacgccttgatcgtcgctcttccggcaatcgccgggaatattggagggagctattggagcggtgcgacgcaaacggccggtagcttaaatcgccatttcgcccggacaatccgccgttag

SEQ ID NO: 86 - DNA, 153 bp, Agrobacterium rhizogenes
atgaagtactttctattgtttttgatcatcggcttggcgtcttgtcagacaagcgatcagttggcgacttgtaaggggcctatttttccgctgaacgtcgggcgatggcagcctgctcagtcggaccttcagcctaccaacgcaggagaggct

SEQ ID NO: 87 - DNA, 708 bp, Agrobacterium rhizogenes
atgaacggctctgaatacgcgctgttagtagagcgggaagcattggcggaccactataaggaagtagaagcatttcagtctgcacgtgctagatcagctcggcgaatctctagagccttggctgctttggctgtcattgcagtcgcaggaaacgtggcgcaggccttcgctatcgccgtcatgcttccgctgaacaaacttgttccagtatatctgtgggtgcgcccagacggtacagttgacagtgaggtatctatttcgcggttgccggcgacgcaggagcaagcggtcgtgaatgcgtctctatgggagtatgttcgtctgcgggagagctactcggcggatacagcccaatacgcctatgatctggtctcgagcttcagtgccccaacggtgcgtcaagattatcagcagttcttcaattaccccagtcctagctccccccaaaccatcattggtaagcgcggaaagctggaagccgagcacatcggctcaaacgaacttatgactggcgtccagcagatccgctacaaacgcactctcatcatggaagggcaagctccaatagtaaccacctggaccgcaacggtacattatgaaacggtaactaacttgcccggccgattgaggctgacaaatccaggcggcttaatcgttacctcctatcaaacttcggaagataccgtttcgaacacgacgcggagccag

SEQ ID NO: 88 - DNA, 876 bp, Agrobacterium rhizogenes
atgatcaagaatttgtttctgggcctggtttgtatcctctttgtaaccagcggcgcgaacgcggaagacacgccagcggcaggcaaactggatccacgtatgcgctatctcgcctacaatcccgatgaggttgtgcacctttcaaccgctgttggagccacattggtcgtaacatttgggtccaatgaagcggtgacagccgtcgccgtttccaacagcaaagatctcgctgcgctcccacgcggcaattatctgtttttcaaggccagcaaagtgctgcagccgcagcctgtgatcgtccttaccgcaagcgacgcaggaatgcggcgctatgttttcagcctcgcgaccagaacgatgtctcggctcgataaagagcagcctgacctctactacagcgtacagttcacttatcctgccgacgttgccgctgctcgccgaaaagaagcggagcagagagatcttgcggatcggatgcgggcgcaagcacaatatcagcgtcgagccgaggacttgctcgagcgtccgccagcaggtggcagcacagatgcgaaaaattggagctatgtcgcgcaaggagaccgctcgctattaccgctcgaggttttcgacaacgggtattccacgacatttcgctttcccggaaacgtgcgtgtaccttcaatctacgtgattaacccagatggcaaggaagccacggccaactattcagttaagggagattacgttgaagtagcatcagtttccagggaatggcgtctgcgggacggccatacagtactttgcatctggaataaggcatatgacgccgtcggacggaagccgggcactggcacagtcagacccgatgtagtgcgcgtgctgaaggagacgagg

SEQ ID NO: 89 - DNA, 1131 bp, Agrobacterium rhizogenes
atggaggacgtgaatgcccaatccagagaaggtatcgacgcgcccggatccctcgtcaccgatcctcatggccggcgcctctcggggtcgcaaaagctccttgttgcgggtttagtcctggtactgtcgttgagcctcatttggctcggggcgcgttcaaagaaaaggaccgagccgtctccaccaaacacaatgatcgatgccaacacgaagccctttcggccggccccgattgatattcccgccaaaccggcaatgacaccgccagctgcggaagcaacggttttgccgtcggatcaacatcagcgagaacacaacgaactgaggcccgaagaaacaccgatctttgcctacagcggaggtgatcagaatggtgtcaaatccgccccacacgctgatattcagaacggtggccaagacaatagaaacgccaactccctagccgcgtcggaggattctgctgagaacgatctctctgtgcgcttgaaaccgacagtgttgcagcctagcctcgctatacttttgccgcacccagatttcactgttacgcaaggaacgatcattccctgcatactgcaaaccgccattgatacaaatctggcagggtatgtgaaatgtgtacttcctcaagacatacgcggagcaaccggaaatgttgtgcttctggatcgcggaacgacagttgtcggcgaaatccagcgtggactgcagcagggagacgcccgcgttttcgttctctggaatcgcgcggagacgccctcacacgccgtcgtctctctttcctccccgggggccgacgaactcggccgttccggactgccgggtacggtcgacaatcatttctggaaacgctttagcggggccatgctcctgagtgtcgtccagggcgcagtccaggcggcaagtagctacgcagggaattcgaccggcgggaccagcttcaatagtttccaaaacaatggcgaacaagcggccgatacggccctaagggcggccataaacattcccccaaccttgaaaaaaaatcagggtgacacggtctcgatcttcgtcgcgcgggatttggatttctcaggaatctaccagcttcatatgactggtggatcggtgaaacgtcggcatctgcgctaa

SEQ ID NO: 90 - DNA, 1035 bp, Agrobacterium rhizogenes
atggaagtaaatccgcaattgcgcgcccttcttaacccagtcttgcaatggctcgatgacccgaggactgaagaagttgccataaacggaccgggagaagcctttgtacgccaaagtggcgtcttcacgaggttcgccgcaccgttctcttatgatgatctcgaagacatcgccattctagcaggggcattgcgaaaacaagatgtcggcccccgcaaccctctttgcgctaccgagctcccaggtggcgagcggatgcaaatctgcttgccgccaacggtaccctctggcaccgtgagcttgacaattcgacgaccgagcaatcgtgtctctgaattgggggaagtctcggctcgctacgatgttcgtcgatggaatcagtggcaaatccggacgcagcgacgagatcaactagacgaagcgattctgcgcgactacgacaatggcgatctggaatcatttttacgtgcatgtgttatcggtaagcggacgatgttgctttgtgggccgaccggaagcggcaagaccacgatgagcaagaccttgatcagtgctattccgcgggaagaaaggctgataaccattgaagatacgctcgaactcgtcattccacatgagaaccacgtcagactgctctactccaagagtggggcagggctaggtgcagtgactgcggaacagctgctccaggcaagccttcggatgcggcctgaccgaatattgctcggtgagatgcgcgacgacgcggcgtgggcgtacctgagtgaggtggtctcaggccaccccggatcgatttcaaccatacatggggcgaatcccgtccaaggcttcaaaaagctgttctcacttgtgaagagcagtgtgcagggggcatgcttggaagatcgcacactgattgacatgttggcaacggcaatcgatgtcattgttcccttccgtgcttacggcgatgtttacgaagtgggcgaagtatggctcgccgccgatgcccgccgacgcggtgagacgataggggatcttctaaaccagcaatag

SEQ ID NO: 91 - DNA, 690 bp, Agrobacterium rhizogenes
atgcgccatcttattaccgagtatttaacgattcatgccttaagggtcaccgcggtatcagatagccagcaattcaaccgggtacttgcatcagagacggtcgatgtcgtcgtcgtggatctcaatttagggcgtgaagacggacttgagatcgttcgcagtctcgccacaaaatccgatgttccaattataatcatcagcggtgatcgccttgaggaggcggataaagtggtggcgctcgagctgggagcaacggattttatcgcgaaacctttcggaacgcgcgagttcctagcacgcatccgtgtcgcattacgcgtgcgaccgaatgtcatgcgaaccaaagaccggcgttcattttgtttcgccggctggacacttagtctcagacagcggcgattggtatccgcacaaaatggcgaggtgaagcttacggcaggggaattcaatctgctcgtcgctttcctggaaaagccacgcgacgtgctttcccgagaacagctcctcatcgcgagccgagtgcgtgaggaggaagtatatgacagaagcattgatgttctcattttgaggctccgccgaaagcttgagcaggatgcagcgagcccaaacttgatcaaaactgccaggggtgccggctatttcttcgacgctgatgtggatgtttcctatggaggtctgatggcggcctga

SEQ ID NO: 92 - DNA, 696 bp, Agrobacterium rhizogenes
atgaaacttctgacattttgctcatttaaaggaggcgccggcaaaaccacggcgctcatgggcctttgcgctgcgtttgcaagagacggcaaaagagtggctctcttcgatgcggacgagaaccggccgttaacgcgatggaaagaaaacgcgatgcgtagcaacacctgggatacatcctgcgaggtgtacgctgcagaagaaatgtcgcttcttgaagcggcttatgaggacgccgagcttcaggaatttgattatgcgctggccgatacgcatggtggttcgagcgagctcaataacacaatcatcgctagctcaaacctgcttctgatcccgacaatgttaacgccgctggatatcgatgaggcactttcgacatatcgttatgttgtcgagctgctgctgagcgagaacttagtgattccgaccgccgtgttacgccagcgagtcccggttggccgattaaccacgtcacaacgcgcgatgtcagacatgctggtgaatctgcctgtcgtacaatctccaatgcatgaacgagacgcgttcgctgcaatgaaagagcgtggcatgttgcatctcacattgctgaacacgacaaatgatccgacgatgcgcctccttgagcggaacctcagaattgcgatggaggaactcgtaacaatttcagaattgattggcgataccttggggaattga

SEQ ID NO: 93 - DNA, 606 bp, Agrobacterium rhizogenes
atggggatccgcaaacctgctttgtctgtcggtgaagcaagacggcttgctgccgcgcgaccgatcgttcacccaatcccagctctttcctcgcaaaacttggcgccttcgcaattacctgaaagagccggaaaggaaaaacgaccagcgcctcctatcgccgccaaacgtcgtgacaacttcgatcggcaatcaatgctaacggcggacgccttgagttcgactgcgccgccggaaaaagtccaggtctttctttcggcacgcccccccgctcctgaggtatcgaagatatatgacaacttgattctgcagtacagcgctaccaaggcgctgcagatgatcttgcgccgtgcgctcgccgattttgagaacatgttggcggacggatcatttagcacggggcctcaaagctacccgatctcaaaagtagtcgaaaaacctatcgttgtcctgacttctcgcatgttcccggtatcgctgttagaagtcgcacgcaatcactttgatccattgagattggagaccgccagggcttttggccacaaactggctacggccgcacttgcatctttctttgccgaagagaagataagaaaagaacgctaa

SEQ ID NO: 94 - DNA, 447 bp, Agrobacterium rhizogenes
atgtcgcaaagcgaaaaatccacttcaagtgacattgtcgaccggcgtgaaggcccgaagattgaaggtttcaaggtcgttagcactcgtttgcgatcgtcggaatatgagagcttctctcgccaagcccgtctgctggggctctccgacagcatggccataagagttgcggttcgccgcattgccggctttctcgaaatcgatgcagagactcgtcagatgatggaagccattcttcattcaataggagcactctcaaacaacattgccgcgctgctgtgtgcctatgctgaaaatccgacaacggatttgggcgctctgcaagctgaacgaaacgctttcggtaaatcgttcgctgatctcgacggtttgctccgttccattttgtccgtatcacggcgacggatcgacggttgctccatgctggcggacgccttgcagcattaa

SEQ ID NO: 95 - DNA, 1176 bp, Agrobacterium rhizogenes
atgcccgatcggtctcaagtcatcatccgcattgtgccgggaggtgggacgaagaccctccaacagattatcaaccagttggagtatctatctcggaagggcaagttggagctgcagcgttcggcccgacatctcgatactttcgtaccaccggatgaaatccgcgaactcgccctaagctgggttcaagaaaccgggacttatcacgaaagtcggccagacgaggaaaggcaacaggagttgacgacccacattattgtaagcttccctgccggtacaagtcaggtagcagcttatgcagcgagccgggagtgggcagccgagatgtttgggtcaggcgcagggggaggccgatacaactatcttacagcctttcacatcgatcgcgatcacccacatctgcatgtggtcgttaatcgtcgcgaacttttggggcacggctggctgaaaatatcccggcgccacccccaactgaattatgacgccttgcgcattacaatggccgagatttcacttcgtcatggcgtcgtcctcgaggcgacgagccgagctgagcgcggcatctcggagcggccgatgacttttgcccagtatcgacgcctggagcggcagcaggccaatcaaattcgtttcgaggatattgatttcgaagaattctcgcctgggggaaacgacggagaaccggatcaatcttttaattcttcgtacggttcgctgcctcaaaacgtgtcagaaaacttgcgacgaaacggcggtctgcaatcggtgtctgaagtacgttcccgggcactgatcggatcgaacggcaacggtgtcacgcccgatcgtgtatcatctggaaacgattcccgttctcaatccggcagcgataaagcctttgtggacgatggcaacttgaggaacggccctctcaccatcgccggagacggccaggatcttgaaggccgttctggcatgcatcgtttggcaaccgaacccgtcacgcacacaacaagcgaagatgatgttcggcaacggcctcatcgaaaacggcctcgcggtgatgaggaagagcagagcggcgcaaaactgaccagggtagacggaattcggacgggagtgacaatcagcgccgtaccggttgcccaggacgatccgattacatcgccgatccagccccctggatcaaatccgttg

SEQ ID NO: 96 - DNA, 3024 bp, Agrobacterium rhizogenes
atgacccggggcagaccaacatcgctacggacgcattgcctccgacagccgatcgccggcagcagggagagccaaattctaagcgtcctcgtgatgatgacgccgagccgagcattcgtaagcggtcaagggacgggcgcagccaaggggatgagggaaacagaaggtagaaaaggatcgatcatggcagatgaagaatttcgtcgagacttcagccgctccgctacgttgaactcgaataacgaaggcgctacaggcgcaccaatcccggatacatcggtgtcgactgccttccgacaaaaccgcagagaacacttaacgaccagttccttacggccattaattactggtggggccggcgaccttccaggcacttcccccgaaaatcaacggccgacaacccatgccccgaatacagcagaaatactcccggtgagatcagccgaagctccggctggccaatcagcggtctttccgaggagcaaggggtattttcgtacggccatgcaatacctgcgggagattgaaatgcagtccactgccagggcggaccacgaggcgacgtcgtcgtcacgttttggcaagggggaaagaaagcagctgcgcgaacacgagcaaaccggtgatcaaagtgaatcaatggcgaagcggagtaccagctcgggaatgcaggtgggaactattgccgcgtctttggctatgaaagatagctcgaccgcgacgccggcttcgcaagggccgcaccggtctgggatggagctggataatatcgaagggactggagagagccgtacgcaatctcagcacgaaattgcgacacaacctattgccacaagcgcttttggttcaggtcatgatgtgatcattcctggagagggccagcctgaagaaacattcgacggcgcaccatattggatggtagaaagcggggatagcgagaccaatagccttccgtcgatccaacaaggcttatcttggccagggttagatgaccttgatttgctcgccgacagcagttcaccgctcagtactcaggttgttcctcctgacgtagccgctacgaatttaagcactgaggcagggtccgccagcgtcgaaagcacccaaatcctcgccgacagtgcctattccgacgacgcgaccgaaaccggtggccaaacagcttcaggatcgatccatgaaggttcagttcgttcgcttggctcaccggaggaacgcttcgacgcctttcccgagttgaccgacgaggacttggcgcagattgacgcattctcgcgagcccgctctccttcgaggcgagatgctgcgcacgctattcatgcggtcagcttggatgatgccttctcacgcaaggatcttgccgcgggccacgacggccggcttacgaatcctgcgttccgtcaggaggcttctctgcaggaatcgagcccatcagaaacgaagtccgacgacttcccggaattaaccgacgaggacctggcacggatcgacgcactctccgaagcccgctcaacttcgaggctggatgttgcacgcactattcatgcggtcaggttggaggcctctcggcaggacccgagcccatcagaaatgaagtccgacgacttccccgaattgaccaaagaggacttagcccggatcgacgcactctctggaccccgcccgactacggggcgggaaggtgcgaacactattcagccagtcaggttggacgatgctttctcagccgaagattataccgtgggccgcggcggccggcatacgaatcctgcgttccatgaggaagcttctcggcaggacccgagcccatcagagatgaagttcgatggcttgccggagttgaccgacgaggatctggtaaggattgacgcaatctctgaaccgcgctcgaagagatctcgctccgagcaggttgtcgggggaaaatctcataggctcaaactgaacgaggctatcgaggaggcgggctcgcccactaccgtttcgggaacaagcgccgccagggcggcacctatcacgctcgaaagcggagtacctaggttgccgcaggctacccatacggaacttcctggctcggacgttgcgggtccactgcggatcgggaaggcgtcgtgtgccgatatacggcgagatgtttcggataaagggaaacgaaaagtctcgtcagattatcagattcagcaaggtcctggcgcgtcctatgcttaccagccgcgacgcatgccgacggaagcgattttcgatgctgaacggcttggtgacgaagtgctcatcgggaatgagccagttccgcggtggccttacccctgggagcacgttgttcgttttgaaacaggcaagcagctcttcgaaagattgcagaagtggccgactgcggaggagttggaaaacagtgtgatcctcgtcaaagcggatagcgcgcaacgatcctatatgccccgaaatcagctgctcttgcatctcggatccgaaaagtattacgaacgaatgaaagcgctcggtctatccgataataccttcagcggattgaagtccgatagcaaatatttttatgatacacctgctggaccacgggtaagtgaacatgattatgagttcccgcggcaggcaggcgtgcataatccagaggtgatggttcagcatcatagcggaaccttcttcgcgttttggccaaaacttctcaggctggaaaagcttgcgacagtaacgaatacgtatggcgcggaaaatgtttggctcaaatcgccagccaatgcttacatgacgccggaaacctataattccaaggttgcgaacggcgccgaaaaccttttcatcgagttcctacgtcccgctcctgtccacgaccacctgcgggaacctggaacttcaaacgcaaaaactgttgacggacgaaacgaagatcgcgcgcaacaggccgaccggttagcagatcagtcgatgggccggaacggggacggccgtgacgcgcagggtggtaagagaaccttggaaccgcgcgtgcgcgaaaggtatgggctgtag

SEQ ID NO: 97 - DNA, 1983 bp, Agrobacterium rhizogenes
atgaattcaggcaagtacacgccaataggcctggctgcgagcatagcatgttcattggccgtggggttttgtgcggccagtctctatgtcacatttcgccatggctttacgggcgaaacgatgatgactttcaacgtttttgcgttcctgtacgagacaccgccttatttgggatacgcaagtcctacgttctatcgcggactagccatcatcgttgcgacgtcaacgctcgtgttgctatgccaactactgttatcgatgcgcgagcgcgaacatcacggcactgctcgctgggccggctccggcgagatgcggcacgccaggtacctccggcgctacagtcacgtcacgggtcccatcttcggcaagacatgtggaccacgttggtttggcagctacctaagtaacggagaacaacctcacagccttgttgtcgcgccaactcgcgctggtaagggtgtcggcgtcgttatcccgacgttgctaaccttcaagggctcagtaattgccttggatgtgaaaggcgagctatttgaactcacatcgagggcgcgtaaatcaagtggcgacgcggttttcaagttctcgcccttagatccagagcggcgaacgcattgttacaatcccgtgctggatattgccgcgttgccgcccgaacggcgctttacagaaacgcgccgcctcgctgcgaatctcatcacagccaaaggcaagggagccgaaggtttcatcgacggggcacgggatcttttcgtagcaggcatattgagttgcattgagcgcggcacaccaacgatcggtgccgtttacgatctgtttgctcagccgggtgaaaagtacaaactatttgcgcaactcgcggaggaaacacaaaacaaagaggctcagcgtatcttcgacaatatggcggggaatgacaccaaaatcctgacatcctacacatcggtgctcggcgacggcggactcaatctttgggccgatccactcgttaaagcggctacaagcacctcggatttttccgtctacgatctgcggcggaagaggacttgtatttatctttgtgtcagcccgaacgatctcgaagtcatagcgcccttgatgcgtcttctcttccagcaggttgtatcaatcctgcagcgatcgctgccacttggagatgaacggcacgaagttctgtttctcctcgatgagttcaagcacttgggcaagctggaagctgtggagacggcaattacgacgatcgccggctacaagggccgcttcatgtttattattcagagtctttcggctttgacaggaacttatgatgaagctggtaagcaaaattttctcagcaatacgggtgtgcaggtattcatggcgactgctgatgacgagacgcccaactacatttccaaagctatcggcgactacacattccaggctcgctcaacgtcgtacagccaagcgcgcatgtttgatcataacatccaaatctccgaccaaggagcaccgcttttgcgcgccgaacaagtccgcctactcgacgacgattatgaaatagtccttatcaaaggccaacctcccctcaaactgagaaaagtgcgatattattcggatttcatattgaagcgaattttcgaaagccaacacggttcccttcccgagcccgcatctttgatgttgccgggagacacaaacctcgttgaaggcaagctcgaccagggaacagccgatacaacttcgcaaaaaccggtgcagattgaagagaacgaccatggggaagtcgtttccccccaaaacagaactgtggctgatggagtaatgccaatagaagttcgcccccgttcaaacgaagttgatgacgagcgggaataccaaggcgcggacgccgcaacctctgagaagcttccggaaattgctccggctcttttggcgcagcgcgaactcctcgaccagatcatttcgctccagctacgaggtagaacagcctccggcacttga

SEQ ID NO: 98 - DNA, 2508 bp, Agrobacterium rhizogenes
atgaaaccgtcgggaagctcaaaaactggatacgccgggtcggctagatcctcgccccaggttcgcgcaggggttacgcctgtccttcatcccaaagagccttggaaccgttttgcttgctccccatctgacggccaaatggatcaccgggaaaatttatctgcgcaattcgcttacgatggaatgagactgggcgcagccgagcggtccgcgtacgagacatgggatagagcggaccggccgagttggaaagacctgatactgagcgctcgcctgaacgcgattgacagtttcgcttggaatgtcgatgtcggagaaagtacctcttcaacttttgtttatgatggtgttcctctggcggaaggggaacggcatgcctacgaggaatgggtggagccgggacagcccagttggcaacaactcgttgtgaacgcgcgtattgaagagctaaacacctctgctgcaattccgaatgagtgtagtccccttcaagaggaattccgatcggaggcgcctaagcgtaagcggacaagctccattggtcaaaagaacggtttccctgaaccattcgaatttgatgggatgagactcggctcgcctgagcgcgaagcatatgagaattggagtaaaccgcaaccgccatcctggaaagacctgatagttgacgcccgtcttgacgcaatcgacacctccacctggctcaacgaatcaaatgacacctcggtcttcgagtacgaaggcgtcccgcttggggagggggaacgcctagcatatgaaaaatggctcgagcccgcgcaaccaggatgggaagacctggttgtggacgcacgcgttgcggaattgcaccagtctgctccgacgtggtgcgaacaccaaccaggcaacgacgtttcttccaacgagtccgggcgcatctcgggcgtcccaattaacgccgagcaggtgtcgcgtgcgttcttcgtgtacgatggagtggcgctcggagcagccgagcgcgctgcgcatgatcgttggagcaggccggaccggcccacctgggaagatcttatcatagtcgcgcgccaagctgctatcgaaggcggtgccgtttcgaatgggatgatcgggaagacatcttcctcagtctttttatacgagggaatgttgcttggggatgcggagcgtcaggcgtacggacggtggaggcagctagcccagccgcggtggcaaaatctggtggtgaacgcgcgcctggcagagctcgacccggcggcctggattcccgatgaacatgatccgtctgaggatggcggggcgactggtcttctgtcgcaagcaagcacgaccaataagtcccgcctcagtttaggtgatcaaccggaagcgcgctcgcctagtctcgcacgtgagccagcacaacagccgactcacgtgcaaaacctgacgtgcgcacaattggaagcaagacgtgctctatatttcgggccctctgggagggatgcagaccaaaccgccagcatcgccgacagtaatcgcctcgacgaggtaagcaaagttaaacggctgggtgccaagagccgtcgaggcgctaaagcaacggcctatgacgtaatttcaagtgcggaaagactgtcgtctcacgagggttgtttgacggttcagtccactcagccagaaaaagccgcttgctcgagaagcgacaatatcggcacttatggaagtcgaaaaaacgaacgagctcggcttgcgaccgagaccgggaaatacgagtcggagcatattttcgggttcaaggttgtccacgataccctgcgctcgaccaaagagggccggcggctggaaaggcctatgcccgcctatctcgaatgcaaggaacttcatcgacaacatgttggtacgggaagggggcggacccgactggtggggcgcggctggccagatgacgcaagttaccgctcggatcaaagggcaactctatcggaccctgttgcgtccacggaaggtgcaacggcgtcaaacgggtatcaattaaaccagctaggctacgcacaccagctcgctaacgacgggctgcagagcgaaacgccagatggggttatcatgccgcttcaggtcgcgactactagctacaactatacggtaagccgcgatccggtactttctccgcccagtaaagagcaagctccggaattgttgcaccttgggccacgtggccagacagaagctgtactcgctcgcgaaacagcattgacaggaagatggccaacacgcgagcgtgagcagcaagtctatcgagagtttttagccctttatgacgtcaagaaggacctggaggccaagacacttgggttgcggaagaaaaaagctgcgcttgtttccgcgttgaaccggactgccgcctcaataggcacttcacccttgaaagcccaatcgtcgagcgcagaagttgaaaaagcaactcacgagtttgatgaacgacgggtttatgatccgcgcgatcgcggtcgggacaaagcattacaacggtga

SEQ ID NO: 99 - DNA, 930 bp, Agrobacterium rhizogenes
atgcaggaacgcgagctttcggtcctcggtggtgataagaccgaaagtgcagccaccttaccgaatgacattttggtggaagttgccaaacatctgccgaccgacgatccagtcgaaacggcagccaacctcacaagctttaaacttgcgagcccgtcggttcgggcatcggtcgatcagagcgatgtcggaacgttccaccgaagcgtaaatcgactgggtgcctcaagcaaggcattgtatgacctggctgtaccccggaacggctttgccgagtatccgaactatccggaaccggaccctgctggcgagtttgcgctggcgagccagcgcatcaggacaattggtccaactttgaagttccaatcgcccgccaggaaaacagcgatcgtcaatcacattctcaatatgtcggaaggcggtgagcaggcggaggcgattatgtcgatgacgtcccatttgagtgacctcgagagggccgacaagagacgccttatcgacagagcaattgaatacttcaaggtagacgggcctatcaactacgacagtcgccattatgcagcctacgcgattgcagcggcgcacaaccagttggaactcgaacacaagtcacaaatcttcgatgcaatggcgaagagaccccagcttgcgagactctacgccgacgaacgcatccatgcgcaggagcatttggggcttgcgtcgacatccgaaggtccgcacgagcggagcaataaacaactggccacggacattcttcagatcgaacaaaaaatccgcactgagctccttcccgaaactctcagtgctcacgaccagatggtaaaagcggaaggaatcgctggatcgattgagaaggcttaccgcgatgctcgcgcgaacttaacccgggcaccgcgagggaggtcaaattctgatctcagccggtag

SEQ ID NO: 100 - DNA, 1356 bp, Agrobacterium rhizogenes
atggtcagtataaataaaaaagccccgcagaaatcttctacagcgaccacgcgcaattctgatgaacgcctggttgatcagttagggcaggcgtaccttgtggaacgcgaggcgcaggaaaggcgcgcgcgacttgaagcgccgccggcgttgaaaaaatatgcctcgaaactgcaactcctcaataagctcgatgcagatttccgtggtgtgatcgcccacaagccgtttcgcagcgagcaactgcgggtcgatagcaatggagaattgacccatagtcggggtctcataaaaaagaagaagaaggttttcattcgagatcaaacaactggctcccttcggcttattcactacgagaggtctgactggaatagtgtccgtcgctatgactacgaccagaacggcgtgttgagcgagaaacatatcaaaagcaaagccggtgccttcgaagaaaaatgggagcgggacgaaaacggtgaactgatgcgcacccgatacatcgatcggcgcagacttactggacgggcgttgcatcccatttccgaggaaatcggacgtccttatgagagtggcctggagaagaggctttatcgcgttttaacgcgtcgggagggctcccaacaaaagactttcgaacgcgatgacaagggtaatctggagctcattgcaagcaaacggatgggctattcgatgtcttcacaaaaggcaccggatcgacaaacatctagaacaagtatccgcaagcttggcggagctttcagtgaatcatatagatcccggctggataaggacggccacgagttaggtcgggatgtctccgcacatcgaagcctcctcaacaagcgttcagccgtctacgatgacgctacaggggagctaaagagcagcaagcacacgtttgggaagatatacaaagcggaagcgacatacctgaacgccgaaatcaaagaggtctccaaaaaaatcctcggagttacggtcggtaaagaattgaagacattaagtggacgcgaactcgaggctcaagtattacgtgccgcggaacgtgccctccataaacaggcctggcagcacccttcagctaacccctcgcggtcgcagcgggaaaataccaacagtcatcttggaaacgaacttgatgggccgctcgaatcggatatgaccgtcgtccatcaagccggatccgggttcgtcggcgacgatcgccacgttcatgatggcggtggatccaggttgaatgaattgcacgaatcgcgatcgtcgaatgatagggaaaagcttagttcgccgagagccgaaatttgcgaccgctcgatgaatcgggatcgcaccggctcaggtactttgtcgcgatga

SEQ ID NO: 101 - DNA, 5310 bp, Agrobacterium rhizogenes
atgccaaccgacgacattgtaatgtccgatcccggaatggctgctgttgacacgtctgtccctacgcgcttccagacagatcttcgccagttcagtaacctgctggacgagagcaatattgtggaatgggtcgtaaaccacgccgcaaatcgcaatgcgtgcttcggacaagaccaacttaagatttcggcggctcgggcactcacgcactacaaacggccggtacaagaaatcgacctgctattccaacaggtcgtcgcaagcgacaaactgacccggcttgattctccagatggccaacctctctacacctctacagagctgagagctgcggaacagaatactgtacgtcatgtcaccaatctttccgcagaagaaagcttcggcgttccgggaaccattgttgatatggtcgccgatagaggcattttcacaccggagctcagaggagcattacattatctgtctaggcgaaatcgagcttccactatggtgggcgtagcgggttccgcaaaaacaagtgtacttggagccctgaacgaagcggtggacaggttcaacgaggaagcgctcccgaccgagaaaataaagttgattgccttcgtacccaccaaccgggcggcccaggagcttcgtgagaaaggtcttcgcgacgtcgcgacgactttcaaggcgaaagaccgcaccatcggaaaaaataccatcgtcgtgatcgacgaaatgtcgatggccaagacgcaggagatcgcgcaactaatcgagacagttgcaaatgccacttcctatcacccaaccgaaaggccgaaactgatatgtgtgggagacgatcggcaactgccccctgttggacctggtgatctgcttccgattattatggagcaagccggctgctatgaactcgtccagccgcttcgacagctcgacgcgcggtcgcgaatagaaacagaaaagctcggacgtgacatacgacgcgatgccaagagcgccgtcgggcactatctcacagcgctcgaacaaatgggcctcgtccatttcgtatatgctcctcctagcggctcaaacgtcagggcgtcagaccagatcactgaccgcatgctggaggagatgaatagagtttgcgaccgatatccggtgtatgatcgtcttgtgatggcacactccaacaggagcgttaatcgcgccaatatcgctcttcatcagaggtttgttgacaaatccggattgcaagaccagcaaatgagtgtgtggtcaagcgtaatagatagtggaagttcggaatctggaatagatgagggttcgtcgagacgcgttcagcaaaggattaatctcgtcttgggagacaggattatattcagcgaaaaccgtctgcaagcggacatccgcaatggtacttttgcaactgtagtcggacttaatagcacagaagcttcccgagctgagaatggcacatctgctggggttcgcattactgcaaggttagacggatctggacgaaccgtcacttggaccgatagcgagtttaagggttttacctacggctatgctgctacaatttataagagtcagggggccacagttgatcacgccattctcctctgtgatggtgctctttcagataagctgacctatgtcggcctgacgaggcatcgcagtcgtctcgacgtcattgcttctcccctcgtggcaaacaatgttgaggctctcagtgcaaagctcactcagcaaagcgcgccaaataattccattcaattcccagcgctcgaacctgctgccttccaagagcgctcgtcggtttcgaacgcgcacttgctaaggccgctcgaccagatggaaatctctccaacgacgccgctttccttccaaggcagacaagaaagctccttcggggagaaccagctgcacgaaccatccacatcggatgtggaaatgccggaaagcgcggacctttacccgagcgccattgacttcgaagttttccagtcttctgtcatcacgactgaagagcaaatgcaaccggaattctgcgggtcggaatcgacggctaacacctcagcccacgaaagccaccaagtcacgatcgcaacggtaacaaacctggggatagcgatcgagaaacggaaacgagcggcggcaaaggaagaaatcgattctcgcaaaaaaatggcccgtcactcagttagttctgccgaggaagttgaagatatcacttccctgttcgaacgattgaaactggcaacctcggtcaccaacacagttcaaccgacgtatgcggacgtcgatcatatcatcggtactgatggccgagtgggtccaataccgcaccgggaagatgatccgggatcaaattgggaattgtcgtcaacgaacgccccctcaaacgtctcagagaaacgatcgaccaaagactcaggagaaaaaaatatggcttcgaatgaccagaaaatgcaaggcgctgactctgcaggcagcgagcgtcatttttcgcagacttccattgtggaagccctgcaggctggcgatcacaagaagctcgtggaaacgctcgccgcaaccggctcccttgtcgagaaggcggacgccgggtcggcgctgaaagcaatggcggatgcttacgaaagggatatcaaggcagatcccgaaaaggtccgcatgctctatgccaatcggcgcgttgatgtagatgcgctggccaaggagatcgaaaaacagggccttgcatccggtcgcctgagtggcgaccctgtcagcttcaaggcgttcaacaacaaggatatccgtttcagggtcggcgacaggctcagcgtcaccagcagctacaggaatctcattgccggtgaagccggccggatcgaaaagatcgaaggcaaatccgttaccttcaaacccgatggcagcagtcagagcaagaccttcgaggccagcaccggcgagccgggtgacaggggcgtcaagggctacaaaatcgaaggcccgatgacggtcatcggctccgcctatgaacttacgcgacatgatgacggcaaacgtgttgaccaaatctatctcttgaacgacgctgcccatggcatcaacgccaatagcgctttgctgagcaaggccgacgagcggacgcagcttttcacttcgctcaaggacaccgccgacagggacatcttggccaggcaactgggaaagccgggcagcgagctctaccaggacaaattcccagccttgccggactacacagccgccatcgatcggattgcaaaacgccaggaagttgtcgcggcacgtggcgaggagaaaacttccgccccgatagacctggcgccccaaacgccgcccgacgctgccgatgcggcagtcaataaccagaaaagaatcgtcgaggccttgcaggcaggcgaccacaagaagcttgtggaaacgctagccgcaaccggctcccttgttgagaacacaaatctcgtatcaacgttgaaagcgatagcgcagacttatgaaagagacattagtgcggaccccgagaaagtccgcgctctctacgccaacaaacgtgatcatgtagatttgcttgccgaagagatcgaaaagcagggccttgcatccggtcgcctgagtggcgaccctgtcagcttcaaggcgtttaacaacaaggatatccgtttcagggtcggtgacaggctcagcgtcactagcagctataggaatctcattgccggtgaagccggccggatcgaaaagatcgaaggcaaatccgttaccttcaaacccgatggcagcagtcagagcaagaccttcgaggccagcaccggcgagccgggtgacaggggtgtcaagggctataagattgaggggcccacgacaatcatcggctccgcctacgaaatcagaaatcacgacgatggcaggcgcgtcgatcaggtttatgtcttgcacagtgccattttcggctcaaatgccgcgagcgccctgttgagcaaggccgacgaacgaacccaggttttcaccgtgcaaagcgacacggctgacagacacatcttggccagccaactcagcaagcctggcagcgagctctatcaggacaaattcccagtctcgccggactacgcagccgcgatcgaccggatcgcgaaacgcgaggaacttagggccggacgtggcgaggaaagtcctactgttcaaatggagccggtgaaccaaacgccctacgaggcgggacattggacgcctgacagtcaaaaatctgtggtggaagccctgcaggctggcgatcacaagaagctcgtggaaacgctcgccgcaaccggctcccttgtcgagaaagcggacgccgggtcggcgctgaaagcaatggcggatgcttacgaaagggatatcaaggcagatcccgaaaaggtccgcatgctctatgccaatcggcgcgttgatgtagatgcgctggccaaggagatcgaaaaacagggccttgcatccggtcgcctgagtggcgaccctgtcagcttcaaggcgttcaacaacaaggatatccgtttcagggtcggcgacaggctcagcgtcaccagcagctacaggaatctcattgccggtgaagccggccggatcgaaaagatcgaaggcaaatccgttaccttcaaacccgatggcagcagtcagagcaagaccttcgaggccagcaccggcgagccgggtgacaggggcgtcaagggctacaaaatcgaaggcccgatgacggtcatcggctccgcctatgaacttacgcgacatgatgacggcaaacgtgttgaccaaatctatctcttgaacgacgctgcccatggcatcaacgccaatagcgctttgctgagcaaggccgacgagcggacgcagcttttcacttcgctcaaggacaccgccgacagggacatcttggccaggcaactgggaaagccgggcagcgagctctaccaggacaaattcccagccttgccggactacacagccgccatcgatcggattgcaaaacgccaggaagttgtcgcggcacgtggcgaggtcaagccgattgagcgcaccgaagcgcccgaaataccccaggttcaagctactatcgttggcgagacctcgatcattcggttgcccgatcggataaaaaccaatcgcgagaagccccaagaacttgatgtagtctcgaaagaagggcacgaaactacaggtagtctgaggcagagtgtccaagagagtaatgcagcaccaaaacagacttcgccgaaggcggcaaatgacgtggatcggctgacacgggattttgacgagcgcatccgcgtccgcggggatggacgtggactctaa

SEQ ID NO: 102 - DNA, 1522 bp, Bordetella bronchiseptica
ctaccggcgcggcagcgttacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtaggggg

(19) United States
(12) Patent Application Publication
(10) Pub. No.: US 2019/0078106 A1
(43) Pub. Date: Mar. 14, 2019

(54) METHODS AND COMPOSITIONS OF IMPROVED PLANT TRANSFORMATION

(71) Applicant: PIONEER HI-BRED INTERNATIONAL, INC., Johnston, IA (US)

(72) Inventors: AJITH ANAND, WEST DES MOINES, IA (US); STEVEN HENRY BASS, HILLSBOROUGH, CA (US); HYEON-JE CHO, FREMONT, CA (US); THEODORE MITCHELL KLEIN, WILMINGTON, DE (US); MICHAEL LASSNER, PORTLAND, OR (US); KEVIN E. MCBRIDE, DAVIS, CA (US)

(73) Assignee: PIONEER HI-BRED INTERNATIONAL, INC., JOHNSTON, IA (US)

(21) Appl. No.: 15/765,521

(22) PCT Filed: Aug. 26, 2016
(86) PCT No.: PCT/US2016/049132

§ 371 Date: Apr. 3, 2018

Related U.S. Application Data
(60) Provisional application No. 62/252,229, filed on Nov. 6, 2015.

(51) Int. Cl. C12N 15/82; C07K 14/245

(52) U.S. Cl. CPC C12N 15/8205; C07K 14/245

(57) ABSTRACT

The present disclosure provides methods and compositions for the vectors comprising vir genes. The present disclosure further provides a vector comprising: (a) an origin of replication for propagation and stable maintenance in Escherichia coli; (b) an origin of replication for propagation and stable maintenance in Agrobacterium spp.; (c) a selectable marker gene; and (d) Rhizobiaceae virulence genes virB1-B11 or r-virB1-B11, virC1-C2 or r-virC1-C2, virD1-D2 or r-virD1-D2, and virG or r-virG, or variants and derivatives thereof, wherein the vector comprising the virulence genes r-virB1-B11, r-virC1-C2, r-virD1-D2, and r-virG further comprises a r-galls virulence gene, or variants and derivatives thereof. This abstract is intended as a scanning tool for purposes of searching in the particular art and is not intended to be limiting of the present disclosure.

### CROSS-REFERENCE TO RELATED APPLICATIONS

This application claims priority to U.S. Provisional Application No. 62/252229, filed Nov. 6, 2015, which is hereby incorporated herein in its entirety by reference.

### FIELD OF THE DISCLOSURE

The present disclosure relates generally to the field of plant molecular biology, including genetic manipulation of plants. More specifically, the present disclosure pertains to methods and compositions for plant transformation comprising vectors that can be used to generate co-integrate vectors or as a ternary helper vector.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 20160826_6469WOPCT_SeqList.txt,”, created on Aug. 23, 2016, and having a size of 485 KB and is filed concurrently with the specification. The sequence listing contained in this ASCII formatted document is part of the specification and is herein incorporated by reference in its entirety.

### BACKGROUND

Agrobacterium, a natural plant pathogen, has been widely used for the transformation of dicotyledonous plants and more recently for transformation of monocotyledonous plants. The advantage of the Agrobacterium-mediated gene transfer system is that it offers the potential to regenerate transgenic cells at relatively high frequencies without a significant reduction in plant regeneration rates. Moreover, the process of DNA transfer to the plant genome is well characterized relative to other DNA delivery methods. DNA transferred via Agrobacterium is less likely to undergo any major rearrangements than is DNA transferred via direct delivery, and it integrates into the plant genome often in single or low copy numbers.

The most commonly used Agrobacterium-mediated gene transfer system is a binary transformation vector system where the Agrobacterium has been engineered to include a disarmed, or nononcogenic, Ti helper plasmid, which encodes the vir functions necessary for DNA transfer, and a much smaller separate plasmid called the binary vector plasmid, which carries the transferred DNA, or the T-DNA region. The T-DNA is defined by sequences at each end, called T-DNA borders, which play an important role in the production of T-DNA and in the transfer process.

Binary vectors are vectors in which the virulence genes are placed on a different plasmid than the one carrying the T-DNA region (Bevan, 1984, Nucl. Acids. Res. 12: 8711-8721). The development of T-DNA binary vectors has made the transformation of plant cells easier as they do not require recombination. The finding that some of the virulence genes exhibited gene dosage effects (Jin et al., J. Bacteriol. (1987) 169:4417-4425) led to the development of a superbinary vector, which carried additional virulence genes (Komari, T., et al., Plant Cell Rep. (1990), 9:303-306). These early superbinary vectors carried a large “vir” fragment (˜14.8 kbp) from the hypervirulenece Ti plasmid, pTiBo542, which had been introduced into a standard binary vector (ibid). The superbinary vectors resulted in vastly improved plant transformation. For example, Hiei, Y., et al. (Plant J. (1994) 6:271-282) described efficient transformation of rice by Agrobacterium, and subsequently there were reports of using this system for maize, barley and wheat (Ishida, Y., et al., Nat. Biotech. (1996) 14:745-750; Tingay, S., et al., Plant J. (1997) 11:1369-1376; and Cheng, M., et al., Plant Physiol. (1997) 115:971-980; see also U.S. Pat. No. 5,591,616 to Hiei et al). Examples of prior superbinary vectors include pTOK162 (Japanese Patent Appl. (Kokai) No. 4-222527, EP-A-504,869, EP-A-604,662, and U.S. Pat. No. 5,591,616) and pTOK233 (see Komari, T., ibid; and Ishida, Y., et al., ibid).

However, the design of prior superbinary vectors has several drawbacks. For example, the large vir fragment and vector backbone carry significant non-essential DNA. In addition, the use of the tetA tetracycline resistance gene both retards bacterial growth and is also a poor selectable marker for Agrbacterium strain C58 since this strain already has partial resistance to the antibiotic. In addition, a large origin of replication/partitioning region (derived from RK2, with a size of about ˜15 kbp) resulted in a fairly large vector which was not easily amenable to further manipulation and is associated with varying degrees of instability.

The limitation with regard to further manipulation was further exacerbated by the fact that reconstitution of the super binary T-DNA vector required homologous recombination between the “super-binary” vector and the T-DNA vector in a recipient Agrobacterium strain such as LBA4404 or C58, which is receptive to homologous recombination. The homologous recombination process is relatively inefficient and the resulting cointegrated vector often contained unexpected deletions. Moreover, screening of many candidate clones was required to identify suitable Agrobacterium isolates for plant transformation. In strains such as C58, EHA101, and the like, spontaneous mutants resistant to tetracycline are known to occur at high frequency (Luo, Z. Q. and Farrand, S. K., (1999) J. Bacteriol. 181:618-626), which further hindered the identification of true recombinant clones.

Despite advances in plant molecular biology, particularly plant transformation and vectors useful in same, there remains a need in the art of transformation to produce transgenic plants efficiently. In particular, there is a need for improved superbinary vectors that have vir genes of optimal size with minimal non-essential sequences, an optimal mix of vir genes for improved virulence, a smaller origin of replication, and improved selectable markers for Agrobacterium selection. Ideally, such improved vectors could be easily used to generate co-integrate vectors or be used as a ternary helper vector for plant transformation. These needs and others are addressed by the present disclosure.

### BRIEF SUMMARY

The present disclosure comprises methods and compositions for vectors comprising vir genes. In various aspects, the present disclosure provides a vector comprising: (a) an origin of replication for propagation and stable maintenance in Escherichia coli; (b) an origin of replication for propagation and stable maintenance in Agrobacterium spp.; (c) a selectable marker gene; and (d) Agrobacterium spp. virulence genes virB1-B11; virC1-C2; virD1-D2; and virG genes. In an aspect, the vector further comprises Agrobacterium spp. virulence genes virA, virD3, virD4, virD5, virE1, virE2, virE3, virH, virH1, virH2, virK, virL, virM, virP, or virQ, or combinations thereof. In an aspect, the vector comprises Agrobacterium sp. virulence genes virB1-B11 (SEQ ID NOS: 4-14, respectively), virC1-C2 (SEQ ID NOS: 16-17, respectively); virD]-D2 (SEQ ID NOS: 18-19, respectively), and virG (SEQ ID NO: 15) genes. In another aspect, the vector comprises Agrobacterium sp. virulence genes virA (SEQ ID NO: 26), virB1-B11 (SEQ ID NOS: 4-14, respectively), virC1-C2 (SEQ ID NOS: 16-17, respectively); virD1-D5 (SEQ ID NOS: 18-22, respectively), virE1-E3 (SEQ ID NOS: 23-25), virG (SEQ ID NO: 15), and virJ (SEQ ID NO: 27) genes.

In an aspect, the present disclosure further provides methods for transformation of a plant comprising the steps of: (a) contacting a tissue from the plant with an Agrobacterium strain comprising a first vector comprising: (i) an origin of replication for propagation and stable maintenance in Escherichia coli; (ii) an origin of replication for propagation and stable maintenance in Agrobacterium spp.; (iii) a selectable marker gene; and (iv) Agrobacterium spp. virulence genes virB1-B11; virC1-C2; virD1-D2; and virG genes, and a second vector comprising T-DNA borders and a polynucleotide sequence of interest for transfer to the plant; (b) co-cultivatiing the tissue with the Agrobacterium; and (c) regenerating a transformed plant from the tissue that expresses the polynucleotide sequence of interest.

In an aspect, the present disclosure further provides kits comprising: (a) a vector comprising: (i) an origin of replication for propagation and stable maintenance in Escherichia coli; (ii) an origin of replication for propagation and stable maintenance in Agrobacterium spp.; (iii) a selectable marker gene; and (iv) Agrobacterium spp. virulence genes virB1-B11; virC1-C2; virD1-D2; and virG genes; and (b) instructions for use in transformation of a plant using Agrobacterium.

The present disclosure comprises methods and compositions for vectors comprising vir genes. In various aspects, the present disclosure provides a vector comprising: (a) an origin of replication for propagation and stable maintenance in Escherichia coli; (b) an origin of replication for propagation and stable maintenance in Agrobacterium spp.; (c) a selectable marker gene; and (d) Rhizobiaceae virulence genes virB1-B11 or r-virB1-B11, virC1-C2 or r-virC1-C2, virD1-D2 or r-virD1-D2, and virG or r-virG, or variants and derivatives thereof, wherein the vector comprising the virulence genes r-virB1-B11, r-virC1-C2, r-virD1-D2, and r-virG further comprises a r-galls virulence gene, or variants and derivatives thereof. In an aspect, the Rhizobiaceae virulence genes are Agrobacterium spp., Rhizobium spp., Sinorhizobium spp., Mesorhizobium spp., Phyllobacterium spp., Ochrobactrum spp., or Bradyrhizobium spp. virulence genes. In an aspect, the Rhizobiaceae virulence genes are Agrobacterium spp. virulence genes. In an aspect, the Agrobacterium spp. virulence genes are Agrobacterium albertimagni, Agrobacterium larrymoorei, Agrobacterium radiobacter, Agrobacterium rhizogenes, Agrobacterium rubi, Agrobacterium tumefaciens, or Agrobacterium vitis virulence genes. In an aspect, the Agrobacterium spp. virulence genes are Agrobacterium rhizogenes or Agrobacterium tumefaciens virulence genes. In an aspect, the Agrobacterium spp. virulence genes are Agrobacterium rhizogenes virulence genes. In an aspect, the Agrobacterium spp. virulence genes are Agrobacterium tumefaciens virulence genes. In an aspect, the Rhizobiaceae virulence genes are virB1-virB11 virulence genes having SEQ ID NOS: 4-14, respectively, or r-virB1-B11 virulence genes having SEQ ID NOS: 80-90, respectively, or variants and derivatives thereof. In an aspect, the Rhizobiaceae virulence genes are virC1-C2 virulence genes having SEQ ID NOS: 16-17, respectively, or r-virC1-C2 virulence genes having SEQ ID NOS: 92-93, respectively, or variants and derivatives thereof. In an aspect, the Rhizobiaceae virulence genes are virD1-D2 virulence genes having SEQ ID NOS: 18-19, respectively, or r-virD1-D2 virulence genes having SEQ ID NOS: 94-95, respectively, or variants and derivatives thereof. In an aspect, the Rhizobiaceae virulence gene is a virG virulence gene having SEQ ID NO: 15, or a r-virG virulence gene having SEQ ID NO: 91, or variants and derivatives thereof. In an aspect, the Rhizobiaceae virulence gene is a r-galls virulence gene having SEQ ID NO: 101, or variants and derivatives thereof. In an aspect, the vector further comprises one or more of Rhizobiaceae virulence genes virA, virD3, virD4, virD5, virE1, virE2, virE3, virH, virH1, virH2, virK, virL, virM, virP, virQ, r-virA, r-virD3, r-virD4, r-virD5, r-virE3, or r-virF or variants and derivatives thereof. In an aspect, the Rhizobiaceae virulence gene is a virA virulence gene having SEQ ID NO: 26 or a r-virA virulence gene having SEQ ID NO: 79, or variants and derivatives thereof. In an aspect, the Rhizobiaceae virulence genes are virD3-D5 virulence genes having SEQ ID NOS: 20-22, respectively, or r-virD3-D5 virulence genes having SEQ ID NO: 96-98, respectively, or variants and derivatives thereof. In an aspect, the Rhizobiaceae virulence genes are virE1-E3 virulence genes having SEQ ID NOS: 23-25, respectively, or a r-virE3 virulence gene having SEQ ID NO: 100, or variants and derivatives thereof. In an aspect, the Rhizobiaceae virulence genes are virH-H1 virulence genes having, SEQ ID NOS: 42-43, respectively, or variants and derivatives thereof. In an aspect, the Rhizobiaceae virulence gene is a virK virulence gene having SEQ ID NO: 45, or variants and derivatives thereof. In an aspect, the Rhizobiaceae virulence gene is a virL virulence gene having SEQ ID NO: 46, or variants and derivatives thereof. In an aspect, the Rhizobiaceae virulence gene is a virM virulence gene having SEQ ID NO: 47, or variants and derivatives thereof. In an aspect, the Rhizobiaceae virulence gene is a virP virulence gene having SEQ ID NO: 48, or variants and derivatives thereof. In an aspect, the Rhizobiaceae virulence gene is a virQ virulence gene having SEQ ID NO: 49, or variants and derivatives thereof. In an aspect, the vector further comprises the Rhizobiaceae virulence genes virD3-D5 and virE1-E3 or r-virD3-D5 and r-vir E3, or variants and derivatives thereof. In an aspect, the vector further comprises the Rhizobiaceae virulence genes virA, virD3-D5, and virE1-E3, or r-virA, r-virD3-D5, and r-virE3, or variants and derivatives thereof. In an aspect, the origin of replication for propagation and stable maintenance in Escherichia coli is derived from a Col E1, a pSC101, a p15A, or a R6K origin of replication, or functional variants and derivatives thereof. In an aspect, the origin of replication for propagation and stable maintenance in Escherichia coli is derived from a Col E1 origin of replication. In an aspect, the origin of replication derived from the ColE1 origin of replication has SEQ ID NO: 2, or variants and fragments thereof. In an aspect, the origin of replication for propagation and stable maintenance in Escherichia coli is derived from a pSC101 origin of replication. In an aspect, the origin of replication derived from the pSC101 origin of replication has SEQ ID NO: 50, or variants and fragments thereof. In an aspect, the origin of replication for propagation and stable maintenance in Escherichia coli is derived from a p15A origin of replication. In an aspect, the origin of replication derived from the p15A origin of replication has SEQ ID NO: 51, or variants and fragments thereof. In an aspect, the origin of replication for propagation and stable maintenance in Escherichia coli is derived from a R6K origin of replication. In an aspect, the origin of replication derived from the R6K origin of replication has SEQ ID NO: 52, or variants and fragments thereof. In an aspect, the origin of replication for propagation and stable maintenance in Agrobacterium spp. is a high copy number origin of replication. In an aspect, the origin of replication for propagation and stable maintenance in Agrobacterium spp. is an intermediate copy number origin of replication. In an aspect, the origin of replication for propagation and stable maintenance in Agrobacterium spp. is a low copy number origin of replication. In an aspect, the origin of replication for propagation and stable maintenance in Agrobacterium spp. is derived from a pRi, a pVS1, a pRSF1010, a pRK2, a pSa, or a pBBR1 origin of replication. In an aspect, the origin of replication for propagation and stable maintenance in Agrobacterium spp. is a variant of the pRK2 origin of replication. In an aspect, the origin of replication for propagation and stable maintenance in Agrobacterium spp. is derived from the pRSF1010 origin of replication. In an aspect, the origin of replication for propagation and stable maintenance in Agrobacterium spp. is derived from the pVS1 origin of replication. In an aspect, the origin of replication for propagation and stable maintenance in Agrobacterium spp. is derived from the pSa origin of replication. In an aspect, the origin of replication for propagation and stable maintenance in Agrobacterium spp. is derived from the pBBR1 origin of replication. In an aspect, the origin of replication for propagation and stable maintenance in Agrobacterium spp. is an origin of replication having any one of SEQ ID NOS: 3, 37, 38, 53, 57, 58, 59, 60 or 102, or variants and fragments thereof. In an aspect, the origin of replication for propagation and stable maintenance in Agrobacterium spp. is a repABC compatible origin of replication. In an aspect, the repABC compatible origin of replication has any one of SEQ ID NOS: 57, 58, 59, or 60, or variants and fragments thereof. In an aspect, the origin of replication for propagation and stable maintenance in Escherichia coli and the origin of replication for propagation and stable maintenance in Agrobacterium spp. are the same origin of replication. In an aspect, the origin of replication is derived from a pRK2 origin of replication, from a pSa origin of replication, or a pRSF1010 origin of replication. In an aspect, the origin of replication is derived from the pRK2 origin of replication. In an aspect, the pRK2 origin of replication has SEQ ID NO: 38, or variants and fragments thereof. In an aspect, the pRK2 origin of replication is a mini or micro pRK2 origin of replication. In an aspect, the pRK2 origin of replication is a micro pRK2 origin of replication. In an aspect, the micro pRK2 origin of replication has SEQ ID NO: 54, or variants and fragments thereof. In an aspect, the pRK2 origin of replication is a mini pRK2 origin of replication. In an aspect, the mini pRK2 has SEQ ID NO: 66, or variants and fragments thereof. In an aspect, the pRK2 origin of replication comprises the trfA and OriV sequences. In an aspect, the pRK2 origin of replication comprises SEQ ID NOS: 64 and 65, or variants and fragments thereof. In an aspect, the origin of replication is derived from the pSa origin of replication. In an aspect, the pSa origin of replication has SEQ ID NO: 53, or variants and fragments thereof. In an aspect, the origin of replication is derived from the pRSF1010 origin of replication. In an aspect, the pRSF1010 origin of replication has SEQ ID NO: 37, or variants and fragments thereof. In an aspect, the vector further comprises a sequence derived from the par DE operon. In an aspect, the par DE operon has SEQ ID NO: 55, or variants and fragments thereof. In an aspect, the selectable marker gene provides resistance to gentamicin, neomycin/kanamycin, hygromycin, or spectinomycin. In an aspect, the selectable marker gene is an aacC1 gene, a npt1gene, a npt2 gene, a hpt gene, an aadA gene, a SpcN gene, or an aph gene. In an aspect, the selectable marker gene is aacC1. In an aspect, the aacC1 selectable marker gene has SEQ ID NO: 1, or variants and fragments thereof. In an aspect, the selectable marker gene is aadA. In an aspect, the aadA selectable marker gene has SEQ ID NO: 39, or variants and fragments thereof. In an aspect, the selectable marker gene is npt1. In an aspect, the nptl selectable marker gene has SEQ ID NO: 40, or variants and fragments thereof. In an aspect, the selectable marker gene is npt2. In an aspect, the npt2 selectable marker gene has SEQ ID NO: 41, or variants and fragments thereof. In an aspect, the selectable marker gene is hpt. In an aspect, the hpt selectable marker gene has SEQ ID NO: 67, or variants and fragments thereof. In an aspect, the selectable marker gene is SpcN. In an aspect, the SpcN selectable marker gene has SEQ ID NO: 77, or variants and fragments thereof. In an aspect, the selectable marker gene is aph. In an aspect, the aph selectable marker gene has SEQ ID NO: 78, or variant and fragments thereof. In an aspect, the selectable marker gene does not provide resistance to tetracycline. In an aspect, the selectable marker gene is not a tetAR gene. In an aspect, the selectable marker gene is a counter-selectable marker gene. In an aspect, the counter-selectable marker gene is a sacB gene, a rpsL (strA) gene, a pheS gene, adhfr (folA) gene, a lacY gene, a Gata-1 gene, a ccdB gene, or a thyA− gene. In an aspect, the vector does not comprise SEQ ID NO: 61, or variants or fragments thereof. In an aspect, the vector does not comprise SEQ ID NO: 62, or variants or fragments thereof. In an aspect, the vector does not comprise a tra operon sequence or a trb operon sequence, or variants or fragments thereof. In an aspect, the vector does not comprise SEQ ID NO: 63, or variants or fragments thereof. In an aspect, the vector has SEQ ID NO: 34, or variants and fragments thereof. In an aspect, the vector has SEQ ID NO: 35, or variants and fragments thereof. In an aspect, the vector has SEQ ID NO: 36, or variants and fragments thereof.

In another aspect, the disclosure further provides a vector comprising: (a) an origin of replication for propagation in Escherichia coli having SEQ ID NO: 2, or variants and fragments thereof; (b) an origin of replication for propagation in Agrobacterium spp. having SEQ ID NO: 3, or variants and fragments thereof; (c) a selectable marker gene having SEQ ID NO: 1, or variants and fragments thereof; and (d) virulence genes comprising Agrobacterium spp. virulence genes virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D2 virulence genes having SEQ ID NOS: 18-19, respectively or r-virD1-D2 virulence genes having SEQ ID NOS: 94-95, respectively, and a virG virulence gene having SEQ ID NO: 15 or a r-virG virulence gene having SEQ ID NO: 91, or variants and derivatives thereof, wherein the vector comprising the virulence genes r-virB1-B11, r-virC1-C2, r-virD1-D2, and r-virG further comprises a r-galls virulence gene having SEQ ID NO: 101, or variants and derivatives thereof.

In another aspect, the disclosure further provides a vector comprising: (a) an origin of replication for propagation in Escherichia coli having SEQ ID NO: 2, or variants and fragments thereof; (b) an origin of replication for propagation in Agrobacterium spp. having SEQ ID NO: 3, or variants and fragments thereof; (c) a selectable marker gene having SEQ ID NO: 1, or variants and fragments thereof; and (d) virulence genes comprising Agrobacterium spp. virulence genes virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D5 virulence genes having SEQ ID NOS: 18-22, respectively or r-virD1-D5 virulence genes having SEQ ID NOS: 94-98, respectively, virE1-E3 virulence genes having SEQ ID NOS: 23-25, respectively or a r-virE3 virulence gene having SEQ ID NO: 100, and a virG virulence gene having SEQ ID NO: 15 or a r-virG virulence gene having SEQ ID NO: 91, or variants and derivatives thereof, wherein the vector comprising the virulence genes r-virB1-B11, r-virC1-C2, r-virD1-D5, r-virE3, and r-virG further comprises a r-galls virulence gene having SEQ ID NO: 101, or variants and derivatives thereof.

In another aspect, the disclosure further provides a vector comprising: (a) an origin of replication for propagation in Escherichia coli having SEQ ID NO: 2, or variants and fragments thereof; (b) an origin of replication for propagation in Agrobacterium spp. having SEQ ID NO: 3, or variants and fragments thereof; (c) a selectable marker gene having SEQ ID NO: 1; and (d) virulence genes comprising Agrobacterium spp. virulence genes a virA virulence gene having SEQ ID NO: 26 or a r-virA virulence gene having SEQ ID NO: 79, virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D5 virulence genes having SEQ ID NOS: 18-22, respectively or r-virD1-D5 virulence genes having SEQ ID NOS: 94-98, respectively, virE1-E3 virulence genes having SEQ ID NOS: 23-25, respectively or a r-virE3 virulence gene having SEQ ID NOS: 100, and a virG virulence gene having SEQ ID NO: 15 or a r-virG virulence gene having SEQ ID NO: 91, or variants and derivatives thereof, wherein the vector comprising the virulence genes r-virA, r-virB1-B11, r-virC1-C2, r-virD1-D5, r-virE3, and r-virG further comprises a r-galls virulence gene having SEQ ID NO: 101, or variants and derivatives thereof.

In an aspect, the present disclosure further provides a method for transformation of a plant comprising the steps of: (a) contacting a tissue from the plant with an Agrobacterium strain or an Ochrobactrum strain comprising a first vector comprising: (i) an origin of replication for propagation and stable maintenance in Escherichia coli; (ii) an origin of replication for propagation and stable maintenance in Agrobacterium spp.; (iii) a selectable marker gene; and (iv) Rhizobiaceae virulence genes virB1-B11 or r-virB1-B11, virC1-C2 or r-virC1-C2, virD1-D2 or r-virD1-D2, and virG or r-virG, or variants and derivatives thereof, wherein the vector comprising the virulence genes r-virB1-B11, r-virC1-C2, r-virD1-D2, and r-virG further comprises a r-galls virulence gene, or variants and derivatives thereof, and a second vector comprising T-DNA borders and a polynucleotide sequence of interest for transfer to the plant; (b) co-cultivating the tissue with the Agrobacterium strain or the Ochrobactrum strain; and (c) regenerating a transformed plant from the tissue that expresses the polynucleotide sequence of interest. In an aspect, the Rhizobiaceae virulence genes are Agrobacterium spp., Rhizobium spp., Sinorhizobium spp., Mesorhizobium spp., Phyllobacterium spp., Ochrobactrum spp., or Bradyrhizobium spp. virulence genes. In an aspect, the Rhizobiaceae virulence genes are Agrobacterium spp. virulence genes. In an aspect, the Agrobacterium spp. virulence genes are Agrobacterium albertimagni, Agrobacterium larrymoorei, Agrobacterium radiobacter, Agrobacterium rhizogenes, Agrobacterium rubi, Agrobacterium tumefaciens, or Agrobacterium vitis virulence genes. In an aspect, the Agrobacterium spp. virulence genes are Agrobacterium rhizogenes or Agrobacterium tumefaciens virulence genes. In an aspect, the Agrobacterium spp. virulence genes are Agrobacterium rhizogenes virulence genes. In an aspect, the Agrobacterium spp. virulence genes are Agrobacterium tumefaciens virulence genes. In an aspect, the Rhizobiaceae virulence genes are virB1-virB11 virulence genes having SEQ ID NOS: 4-14, respectively, or r-virB1-B11 virulence genes having SEQ ID NOS: 80-90, respectively, or variants and derivatives thereof. In an aspect, the Rhizobiaceae virulence genes are virC1-C2 virulence genes having SEQ ID NOS: 16-17, respectively, or r-virC1-C2 virulence genes having SEQ ID NOS: 92-93, respectively, or variants and derivatives thereof. In an aspect, the Rhizobiaceae virulence genes are virD1-D2 virulence genes having SEQ ID NOS: 18-19, respectively, or r-virD1-D2 virulence genes having SEQ ID NOS: 94-95, respectively, or variants and derivatives thereof. In an aspect, the Rhizobiaceae virulence gene is a virG virulence gene having SEQ ID NO: 15, or a r-virG virulence gene having SEQ ID NO: 91, or variants and derivatives thereof. In an aspect, the Rhizobiaceae virulence gene is a r-galls virulence gene having SEQ ID NO: 101, or variants and derivatives thereof. In an aspect, the vector further comprises one or more of Rhizobiaceae virulence genes virA, virD3, virD4, virD5, virE1, virE2, virE3, virH, virH1, virH2, virK, virL, virM, virP, virQ, r-virA , r-virD3, r-virD4, r-virD5, r-virE3, or r-virF or variants and derivatives thereof. In an aspect, the Rhizobiaceae virulence gene is a virA virulence gene having SEQ ID NO: 26 or a r-virA virulence gene having SEQ ID NO: 79, or variants and derivatives thereof. In an aspect, the Rhizobiaceae virulence genes are virD3-D5 virulence genes having SEQ ID NOS: 20-22, respectively, or r-virD3-D5 virulence genes having SEQ ID NO: 96-98, respectively, or variants and derivatives thereof. In an aspect, the Rhizobiaceae virulence genes are virE1-E3 virulence genes having SEQ ID NOS: 23-25, respectively, or a r-virE3 virulence gene having SEQ ID NO: 100, or variants and derivatives thereof. In an aspect, the Rhizobiaceae virulence genes are virH-H1 virulence genes having, SEQ ID NOS: 42-43, respectively, or variants and derivatives thereof. In an aspect, the Rhizobiaceae virulence gene is a virK virulence gene having SEQ ID NO: 45, or variants and derivatives thereof. In an aspect, the Rhizobiaceae virulence gene is a virL virulence gene having SEQ ID NO: 46, or variants and derivatives thereof. In an aspect, the Rhizobiaceae virulence gene is a virM virulence gene having SEQ ID NO: 47, or variants and derivatives thereof. In an aspect, the Rhizobiaceae virulence gene is a virP virulence gene having SEQ ID NO: 48, or variants and derivatives thereof. In an aspect, the Rhizobiaceae virulence gene is a virQ virulence gene having SEQ ID NO: 49, or variants and derivatives thereof. In an aspect, the vector further comprises the Rhizobiaceae virulence genes virD3-D5 and virE1-E3 or r-virD3-D5 and r-vir E3, or variants and derivatives thereof. In an aspect, the vector further comprises the Rhizobiaceae virulence genes virA, virD3-D5, and virE1-E3, or r-virA, r-virD3-D5, and r-virE3, or variants and derivatives thereof. In an aspect, the origin of replication for propagation and stable maintenance in Escherichia coli is derived from a Col E1, a pSC101, a pl5A, or a R6K origin of replication, or functional variants and derivatives thereof. In an aspect, the origin of replication for propagation and stable maintenance in Escherichia coli is derived from a Col E1 origin of replication. In an aspect, the origin of replication derived from the ColE1 origin of replication has SEQ ID NO: 2, or variants and fragments thereof. In an aspect, the origin of replication for propagation and stable maintenance in Escherichia coli is derived from a pSC101 origin of replication. In an aspect, the origin of replication derived from the pSC101 origin of replication has SEQ ID NO: 50, or variants and fragments thereof. In an aspect, the origin of replication for propagation and stable maintenance in Escherichia coli is derived from a p 15A origin of replication. In an aspect, the origin of replication derived from the p15A origin of replication has SEQ ID NO: 51, or variants and fragments thereof. In an aspect, the origin of replication for propagation and stable maintenance in Escherichia coli is derived from a R6K origin of replication. In an aspect, the origin of replication derived from the R6K origin of replication has SEQ ID NO: 52, or variants and fragments thereof. In an aspect, the origin of replication for propagation and stable maintenance in Agrobacterium spp. is a high copy number origin of replication. In an aspect, the origin of replication for propagation and stable maintenance in Agrobacterium spp. is an intermediate copy number origin of replication. In an aspect, the origin of replication for propagation and stable maintenance in Agrobacterium spp. is a low copy number origin of replication. In an aspect, the origin of replication for propagation and stable maintenance in Agrobacterium spp. is derived from a pRi, a pVS1, a pRSF1010, a pRK2, a pSa, or a pBBR1 origin of replication. In an aspect, the origin of replication for propagation and stable maintenance in Agrobacterium spp. is a variant of the pRK2 origin of replication. In an aspect, the origin of replication for propagation and stable maintenance in Agrobacterium spp. is derived from the pRSF1010 origin of replication. In an aspect, the origin of replication for propagation and stable maintenance in Agrobacterium spp. is derived from the pVS1 origin of replication. In an aspect, the origin of replication for propagation and stable maintenance in Agrobacterium spp. is derived from the pSa origin of replication. In an aspect, the origin of replication for propagation and stable maintenance in Agrobacterium spp. is an origin of replication having any one of SEQ ID NOS: 3, 37, 38, 53, 57, 58, 59, or 60, or variants and fragments thereof. In an aspect, the origin of replication for propagation and stable maintenance in Agrobacterium spp. is a repABC compatible origin of replication. In an aspect, the repABC compatible origin of replication has any one of SEQ ID NOS: 57, 58, 59, or 60, or variants and fragments thereof. In an aspect, the origin of replication for propagation and stable maintenance in Escherichia coli and the origin of replication for propagation and stable maintenance in Agrobacterium spp. are the same origin of replication. In an aspect, the origin of replication is derived from a pRK2 origin of replication, from a pSa origin of replication, or a pRSF1010 origin of replication. In an aspect, the origin of replication is derived from the pRK2 origin of replication. In an aspect, the pRK2 origin of replication has SEQ ID NO: 38, or variants and fragments thereof. In an aspect, the pRK2 origin of replication is a mini or micro pRK2 origin of replication. In an aspect, the pRK2 origin of replication is a micro pRK2 origin of replication. In an aspect, the micro pRK2 origin of replication has SEQ ID NO: 54, or variants and fragments thereof. In an aspect, the pRK2 origin of replication is a mini pRK2 origin of replication. In an aspect, the mini pRK2 has SEQ ID NO: 66, or variants and fragments thereof. In an aspect, the pRK2 origin of replication comprises the trfA and OriV sequences. In an aspect, the pRK2 origin of replication comprises SEQ ID NOS: 64 and 65, or variants and fragments thereof. In an aspect, the origin of replication is derived from the pSa origin of replication. In an aspect, the pSa origin of replication has SEQ ID NO: 53, or variants and fragments thereof. In an aspect, the origin of replication is derived from the pRSF1010 origin of replication. In an aspect, the pRSF1010 origin of replication has SEQ ID NO: 37, or variants and fragments thereof. In an aspect, the vector further comprises a sequence derived from the par DE operon. In an aspect, the par DE operon has SEQ ID NO: 55, or variants and fragments thereof. In an aspect, the selectable marker gene provides resistance to gentamicin, neomycin/kanamycin, hygromycin, or spectinomycin. In an aspect, the selectable marker gene is an aacC1 gene, a npt1gene, a npt2 gene, a hpt gene, an aadA gene, a SpcN gene, or an aph gene. In an aspect, the selectable marker gene is aacC1. In an aspect, the aacC1 selectable marker gene has SEQ ID NO: 1, or variants and fragments thereof. In an aspect, the selectable marker gene is aadA. In an aspect, the aadA selectable marker gene has SEQ ID NO: 39, or variants and fragments thereof. In an aspect, the selectable marker gene is npt1. In an aspect, the nptl selectable marker gene has SEQ ID NO: 40, or variants and fragments thereof. In an aspect, the selectable marker gene is npt2. In an aspect, the npt2 selectable marker gene has SEQ ID NO: 41, or variants and fragments thereof. In an aspect, the selectable marker gene is hpt. In an aspect, the hpt selectable marker gene has SEQ ID NO: 67, or variants and fragments thereof. In an aspect, the selectable marker gene is SpcN. In an aspect, the SpcN selectable marker gene has SEQ ID NO: 77, or variants and fragments thereof. In an aspect, the selectable marker gene is aph. In an aspect, the aph selectable marker gene has SEQ ID NO: 78, or variants and fragments thereof. In an aspect, the selectable marker gene does not provide resistance to tetracycline. In an aspect, the selectable marker gene is not a tetAR gene. In an aspect, the selectable marker gene is a counter-selectable marker gene. In an aspect, the counter-selectable marker gene is a sacB gene, a rpsL (strA) gene, a pheS gene, adhfr (folA) gene, a lacY gene, a Gata-1 gene, a ccdB gene, or a thyA− gene. In an aspect, the vector does not comprise SEQ ID NO: 61, or variants or fragments thereof. In an aspect, the vector does not comprise SEQ ID NO: 62, or variants or fragments thereof. In an aspect, the vector does not comprise a tra operon sequence or a trb operon sequence, or variants or fragments thereof. In an aspect, the vector does not comprise SEQ ID NO: 63, or variants or fragments thereof. In an aspect, the vector has SEQ ID NO: 34, or variants and fragments thereof. In an aspect, the vector has SEQ ID NO: 35, or variants and fragments thereof. In an aspect, the vector has SEQ ID NO: 36, or variants and fragments thereof.

In an aspect, the present disclosure further provides a method for transformation of a plant comprising the steps of: (a) contacting a tissue from the plant with an Agrobacterium strain or an Ochrobactrum strain comprising a first vector comprising: (i) an origin of replication for propagation in Escherichia coli having SEQ ID NO: 2, or variants and fragments thereof; (ii) an origin of replication for propagation in Agrobacterium spp. having SEQ ID NO: 3, or variants and fragments thereof; (iii) a selectable marker gene having SEQ ID NO: 1, or variants and fragments thereof; and (iv) virulence genes comprising Agrobacterium spp. virulence genes virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D2 virulence genes having SEQ ID NOS: 18-19, respectively or r-virD1-D2 virulence genes having SEQ ID NOS: 94-95, respectively, and a virG virulence gene having SEQ ID NO: 15 or a r-virG virulence gene having SEQ ID NO: 91, or variants and derivatives thereof, wherein the vector comprising the virulence genes r-virB1-B11, r-virC1-C2, r-virD1-D2, and r-virG further comprises a r-galls virulence gene having SEQ ID NO: 101, or variants and derivatives thereof, and a second vector comprising T-DNA borders and a polynucleotide sequence of interest for transfer to the plant; (b) co-cultivating the tissue with the Agrobacterium strain or the Ochrobactrum strain; and (c) regenerating a transformed plant from the tissue that expresses the polynucleotide sequence of interest. In an aspect, the Rhizobiaceae virulence genes are Agrobacterium spp., Rhizobium spp., Sinorhizobium spp., Mesorhizobium spp., Phyllobacterium spp., Ochrobactrum spp., or Bradyrhizobium spp. virulence genes. In an aspect, the Rhizobiaceae virulence genes are Agrobacterium spp. virulence genes. In an aspect, the Agrobacterium spp. virulence genes are Agrobacterium albertimagni, Agrobacterium larrymoorei, Agrobacterium radiobacter, Agrobacterium rhizogenes, Agrobacterium rubi, Agrobacterium tumefaciens, or Agrobacterium vitis virulence genes. In an aspect, the Agrobacterium spp. virulence genes are Agrobacterium rhizogenes or Agrobacterium tumefaciens virulence genes. In an aspect, the Agrobacterium spp. virulence genes are Agrobacterium rhizogenes virulence genes. In an aspect, the Agrobacterium spp. virulence genes are Agrobacterium tumefaciens virulence genes. In an aspect, the Rhizobiaceae virulence genes are virB1-virB11 virulence genes having SEQ ID NOS: 4-14, respectively, or r-virB1-B11 virulence genes having SEQ ID NOS: 80-90, respectively, or variants and derivatives thereof. In an aspect, the Rhizobiaceae virulence genes are virC1-C2 virulence genes having SEQ ID NOS: 16-17, respectively, or r-virC1-C2 virulence genes having SEQ ID NOS: 92-93, respectively, or variants and derivatives thereof. In an aspect, the Rhizobiaceae virulence genes are virD1-D2 virulence genes having SEQ ID NOS: 18-19, respectively, or r-virD1-D2 virulence genes having SEQ ID NOS: 94-95, respectively, or variants and derivatives thereof. In an aspect, the Rhizobiaceae virulence gene is a virG virulence gene having SEQ ID NO: 15, or a r-virG virulence gene having SEQ ID NO: 91, or variants and derivatives thereof. In an aspect, the Rhizobiaceae virulence gene is a r-galls virulence gene having SEQ ID NO: 101, or variants and derivatives thereof. In an aspect, the vector further comprises one or more of Rhizobiaceae virulence genes virA, virD3, virD4, virD5, virE1, virE2, virE3, virH, virH1, virH2, virK, virL, virM, virP, virQ, r-virA , r-virD3, r-virD4, r-virD5, r-virE3, or r-virF or variants and derivatives thereof. In an aspect, the Rhizobiaceae virulence gene is a virA virulence gene having SEQ ID NO: 26 or a r-virA virulence gene having SEQ ID NO: 79, or variants and derivatives thereof. In an aspect, the Rhizobiaceae virulence genes are virD3-D5 virulence genes having SEQ ID NOS: 20-22, respectively, or r-virD3-D5 virulence genes having SEQ ID NO: 96-98, respectively, or variants and derivatives thereof. In an aspect, the Rhizobiaceae virulence genes are virE1-E3 virulence genes having SEQ ID NOS: 23-25, respectively, or a r-virE3 virulence gene having SEQ ID NO: 100, or variants and derivatives thereof. In an aspect, the Rhizobiaceae virulence genes are virH-H1 virulence genes having, SEQ ID NOS: 42-43, respectively, or variants and derivatives thereof. In an aspect, the Rhizobiaceae virulence gene is a virK virulence gene having SEQ ID NO: 45, or variants and derivatives thereof. In an aspect, the Rhizobiaceae virulence gene is a virL virulence gene having SEQ ID NO: 46, or variants and derivatives thereof. In an aspect, the Rhizobiaceae virulence gene is a virM virulence gene having SEQ ID NO: 47, or variants and derivatives thereof. In an aspect, the Rhizobiaceae virulence gene is a virP virulence gene having SEQ ID NO: 48, or variants and derivatives thereof. In an aspect, the

Rhizobiaceae virulence gene is a virQ virulence gene having SEQ ID NO: 49, or variants and derivatives thereof. In an aspect, the vector further comprises the Rhizobiaceae virulence genes virD3-D5 and virE1-E3 or r-virD3-D5 and r-vir E3, or variants and derivatives thereof. In an aspect, the vector further comprises the Rhizobiaceae virulence genes virA, virD3-D5, and virE1-E3, or r-virA, r-virD3-D5, and r-virE3, or variants and derivatives thereof. In an aspect, the origin of replication for propagation and stable maintenance in Escherichia coli is derived from a Col E1, a pSC101, a p15A, or a R6K origin of replication, or functional variants and derivatives thereof. In an aspect, the origin of replication for propagation and stable maintenance in Escherichia coli is derived from a Col E1 origin of replication. In an aspect, the origin of replication derived from the ColE1 origin of replication has SEQ ID NO: 2, or variants and fragments thereof. In an aspect, the origin of replication for propagation and stable maintenance in Escherichia coli is derived from a pSC101 origin of replication. In an aspect, the origin of replication derived from the pSC101 origin of replication has SEQ ID NO: 50, or variants and fragments thereof. In an aspect, the origin of replication for propagation and stable maintenance in Escherichia coli is derived from a p15A origin of replication. In an aspect, the origin of replication derived from the p15A origin of replication has SEQ ID NO: 51, or variants and fragments thereof. In an aspect, the origin of replication for propagation and stable maintenance in Escherichia coli is derived from a R6K origin of replication. In an aspect, the origin of replication derived from the R6K origin of replication has SEQ ID NO: 52, or variants and fragments thereof. In an aspect, the origin of replication for propagation and stable maintenance in Agrobacterium spp. is a high copy number origin of replication. In an aspect, the origin of replication for propagation and stable maintenance in Agrobacterium spp. is an intermediate copy number origin of replication. In an aspect, the origin of replication for propagation and stable maintenance in Agrobacterium spp. is a low copy number origin of replication. In an aspect, the origin of replication for propagation and stable maintenance in Agrobacterium spp. is derived from a pRi, a pVS1, a pRSF1010, a pRK2, a pSa, or a pBBR1 origin of replication. In an aspect, the origin of replication for propagation and stable maintenance in Agrobacterium spp. is a variant of the pRK2 origin of replication. In an aspect, the origin of replication for propagation and stable maintenance in Agrobacterium spp. is derived from the pRSF1010 origin of replication. In an aspect, the origin of replication for propagation and stable maintenance in Agrobacterium spp. is derived from the pVS1 origin of replication. In an aspect, the origin of replication for propagation and stable maintenance in Agrobacterium spp. is derived from the pSa origin of replication. In an aspect, the origin of replication for propagation and stable maintenance in Agrobacterium spp. is an origin of replication having any one of SEQ ID NOS: 3, 37, 38, 53, 57, 58, 59, or 60, or variants and fragments thereof. In an aspect, the origin of replication for propagation and stable maintenance in Agrobacterium spp. is a repABC compatible origin of replication. In an aspect, the repABC compatible origin of replication has any one of SEQ ID NOS: 57, 58, 59, or 60, or variants and fragments thereof. In an aspect, the origin of replication for propagation and stable maintenance in Escherichia coli and the origin of replication for propagation and stable maintenance in Agrobacterium spp. are the same origin of replication. In an aspect, the origin of replication is derived from a pRK2 origin of replication, from a pSa origin of replication, or a pRSF1010 origin of replication. In an aspect, the origin of replication is derived from the pRK2 origin of replication. In an aspect, the pRK2 origin of replication has SEQ ID NO: 38, or variants and fragments thereof. In an aspect, the pRK2 origin of replication is a mini or micro pRK2 origin of replication. In an aspect, the pRK2 origin of replication is a micro pRK2 origin of replication. In an aspect, the micro pRK2 origin of replication has SEQ ID NO: 54, or variants and fragments thereof. In an aspect, the pRK2 origin of replication is a mini pRK2 origin of replication. In an aspect, the mini pRK2 has SEQ ID NO: 66, or variants and fragments thereof. In an aspect, the pRK2 origin of replication comprises the trfA and OriV sequences. In an aspect, the pRK2 origin of replication comprises SEQ ID NOS: 64 and 65, or variants and fragments thereof. In an aspect, the origin of replication is derived from the pSa origin of replication. In an aspect, the pSa origin of replication has SEQ ID NO: 53, or variants and fragments thereof. In an aspect, the origin of replication is derived from the pRSF1010 origin of replication. In an aspect, the pRSF1010 origin of replication has SEQ ID NO: 37, or variants and fragments thereof. In an aspect, the vector further comprises a sequence derived from the par DE operon. In an aspect, the par DE operon has SEQ ID NO: 55, or variants and fragments thereof. In an aspect, the selectable marker gene provides resistance to gentamicin, neomycin/kanamycin, hygromycin, or spectinomycin. In an aspect, the selectable marker gene is an aacC1 gene, a npt1gene, a npt2 gene, a hpt gene, an aadA gene, a SpcN gene, or an aph gene. In an aspect, the selectable marker gene is aacC1. In an aspect, the aacC1 selectable marker gene has SEQ ID NO: 1, or variants and fragments thereof. In an aspect, the selectable marker gene is aadA. In an aspect, the aadA selectable marker gene has SEQ ID NO: 39, or variants and fragments thereof. In an aspect, the selectable marker gene is npt1. In an aspect, the nptl selectable marker gene has SEQ ID NO: 40, or variants and fragments thereof. In an aspect, the selectable marker gene is npt2. In an aspect, the npt2 selectable marker gene has SEQ ID NO: 41, or variants and fragments thereof. In an aspect, the selectable marker gene is hpt. In an aspect, the hpt selectable marker gene has SEQ ID NO: 67, or variants and fragments thereof. In an aspect, the selectable marker gene is SpcN. In an aspect, the SpcN selectable marker gene has SEQ ID NO: 77, or variants and fragments thereof. In an aspect, the selectable marker gene is aph. In an aspect, the aph selectable marker gene has SEQ ID NO: 78, or variants and fragments thereof. In an aspect, the selectable marker gene does not provide resistance to tetracycline. In an aspect, the selectable marker gene is not a tetAR gene. In an aspect, the selectable marker gene is a counter-selectable marker gene. In an aspect, the counter-selectable marker gene is a sacB gene, a rpsL (strA) gene, a pheS gene, adhfr (folA) gene, a lacY gene, a Gata-1 gene, a ccdB gene, or a thyA− gene. In an aspect, the vector does not comprise SEQ ID NO: 61, or variants or fragments thereof. In an aspect, the vector does not comprise SEQ ID NO: 62, or variants or fragments thereof. In an aspect, the vector does not comprise a tra operon sequence or a trb operon sequence, or variants or fragments thereof. In an aspect, the vector does not comprise SEQ ID NO: 63, or variants or fragments thereof. In an aspect, the vector has SEQ ID NO: 34, or variants and fragments thereof. In an aspect, the vector has SEQ ID NO: 35, or variants and fragments thereof. In an aspect, the vector has SEQ ID NO: 36, or variants and fragments thereof.

In an aspect, the present disclosure further provides a method for transformation of a plant comprising the steps of: (a) contacting a tissue from the plant with an Agrobacterium strain or an Ochrobactrum strain comprising a first vector comprising: (i) n origin of replication for propagation in Escherichia coli having SEQ ID NO: 2, or variants and fragments thereof; (ii) an origin of replication for propagation in Agrobacterium spp. having SEQ ID NO: 3, or variants and fragments thereof; (iii) a selectable marker gene having SEQ ID NO: 1, or variants and fragments thereof; and (iv) virulence genes comprising Agrobacterium spp. virulence genes virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D5 virulence genes having SEQ ID NOS: 18-22, respectively or r-virD1-D5 virulence genes having SEQ ID NOS: 94-98, respectively, virE1-E3 virulence genes having SEQ ID NOS: 23-25, respectively or a r-virE3 virulence gene having SEQ ID NO: 100, and a virG virulence gene having SEQ ID NO: 15 or a r-virG virulence gene having SEQ ID NO: 91, or variants and derivatives thereof, wherein the vector comprising the virulence genes r-virB1-B11, r-virC1-C2, r-virD1-D5, r-virE3, and r-virG further comprises a r-galls virulence gene having SEQ ID NO: 101, or variants and derivatives thereof, and a second vector comprising T-DNA borders and a polynucleotide sequence of interest for transfer to the plant; (b) co-cultivating the tissue with the Agrobacterium strain or the Ochrobactrum strain; and (c) regenerating a transformed plant from the tissue that expresses the polynucleotide sequence of interest. In an aspect, the Rhizobiaceae virulence genes are Agrobacterium spp., Rhizobium spp., Sinorhizobium spp., Mesorhizobium spp., Phyllobacterium spp., Ochrobactrum spp., or Bradyrhizobium spp. virulence genes. In an aspect, the Rhizobiaceae virulence genes are Agrobacterium spp. virulence genes. In an aspect, the Agrobacterium spp. virulence genes are Agrobacterium albertimagni, Agrobacterium larrymoorei, Agrobacterium radiobacter, Agrobacterium rhizogenes, Agrobacterium rubi, Agrobacterium tumefaciens, or Agrobacterium vitis virulence genes. In an aspect, the Agrobacterium spp. virulence genes are Agrobacterium rhizogenes or Agrobacterium tumefaciens virulence genes. In an aspect, the Agrobacterium spp. virulence genes are Agrobacterium rhizogenes virulence genes. In an aspect, the Agrobacterium spp. virulence genes are Agrobacterium tumefaciens virulence genes. In an aspect, the Rhizobiaceae virulence genes are virB1-virB11 virulence genes having SEQ ID NOS: 4-14, respectively, or r-virB1-B11 virulence genes having SEQ ID NOS: 80-90, respectively, or variants and derivatives thereof. In an aspect, the Rhizobiaceae virulence genes are virC1-C2 virulence genes having SEQ ID NOS: 16-17, respectively, or r-virC1-C2 virulence genes having SEQ ID NOS: 92-93, respectively, or variants and derivatives thereof. In an aspect, the Rhizobiaceae virulence genes are virD1-D2 virulence genes having SEQ ID NOS: 18-19, respectively, or r-virD1-D2 virulence genes having SEQ ID NOS: 94-95, respectively, or variants and derivatives thereof. In an aspect, the Rhizobiaceae virulence gene is a virG virulence gene having SEQ ID NO: 15, or a r-virG virulence gene having SEQ ID NO: 91, or variants and derivatives thereof. In an aspect, the Rhizobiaceae virulence gene is a r-galls virulence gene having SEQ ID NO: 101, or variants and derivatives thereof. In an aspect, the vector further comprises one or more of Rhizobiaceae virulence genes virA, virD3, virD4, virD5, virE1, virE2, virE3, virH, virH1, virH2, virK, virL, virM, virP, virQ, r-virA , r-virD3, r-virD4, r-virD5, or r-virE3, r-virF or variants and derivatives thereof. In an aspect, the Rhizobiaceae virulence gene is a virA virulence gene having SEQ ID NO: 26 or a r-virA virulence gene having SEQ ID NO: 79, or variants and derivatives thereof. In an aspect, the Rhizobiaceae virulence genes are virD3-D5 virulence genes having SEQ ID NOS: 20-22, respectively, or r-virD3-D5 virulence genes having SEQ ID NO: 96-98, respectively, or variants and derivatives thereof. In an aspect, the Rhizobiaceae virulence genes are virE1-E3 virulence genes having SEQ ID NOS: 23-25, respectively, or a r-virE3 virulence gene having SEQ ID NO: 100, or variants and derivatives thereof. In an aspect, the Rhizobiaceae virulence genes are virH-H1 virulence genes having, SEQ ID NOS: 42-43, respectively, or variants and derivatives thereof. In an aspect, the Rhizobiaceae virulence gene is a virK virulence gene having SEQ ID NO: 45, or variants and derivatives thereof. In an aspect, the Rhizobiaceae virulence gene is a virL virulence gene having SEQ ID NO: 46, or variants and derivatives thereof. In an aspect, the Rhizobiaceae virulence gene is a virM virulence gene having SEQ ID NO: 47, or variants and derivatives thereof. In an aspect, the Rhizobiaceae virulence gene is a virP virulence gene having SEQ ID NO: 48, or variants and derivatives thereof. In an aspect, the Rhizobiaceae virulence gene is a virQ virulence gene having SEQ ID NO: 49, or variants and derivatives thereof. In an aspect, the vector further comprises the Rhizobiaceae virulence genes virD3-D5 and virE1-E3 or r-virD3-D5 and r-vir E3, or variants and derivatives thereof. In an aspect, the vector further comprises the Rhizobiaceae virulence genes virA, virD3-D5, and virE1-E3, or r-virA, r-virD3-D5, and r-virE3, or variants and derivatives thereof. In an aspect, the origin of replication for propagation and stable maintenance in Escherichia coli is derived from a Col E1, a pSC101, a p15A, or a R6K origin of replication, or functional variants and derivatives thereof. In an aspect, the origin of replication for propagation and stable maintenance in Escherichia coli is derived from a Col E1 origin of replication. In an aspect, the origin of replication derived from the ColE1 origin of replication has SEQ ID NO: 2, or variants and fragments thereof. In an aspect, the origin of replication for propagation and stable maintenance in Escherichia coli is derived from a pSC101 origin of replication. In an aspect, the origin of replication derived from the pSC101 origin of replication has SEQ ID NO: 50, or variants and fragments thereof. In an aspect, the origin of replication for propagation and stable maintenance in Escherichia coli is derived from a p15A origin of replication. In an aspect, the origin of replication derived from the p15A origin of replication has SEQ ID NO: 51, or variants and fragments thereof. In an aspect, the origin of replication for propagation and stable maintenance in Escherichia coli is derived from a R6K origin of replication. In an aspect, the origin of replication derived from the R6K origin of replication has SEQ ID NO: 52, or variants and fragments thereof. In an aspect, the origin of replication for propagation and stable maintenance in Agrobacterium spp. is a high copy number origin of replication. In an aspect, the origin of replication for propagation and stable maintenance in Agrobacterium spp. is an intermediate copy number origin of replication. In an aspect, the origin of replication for propagation and stable maintenance in Agrobacterium spp. is a low copy number origin of replication. In an aspect, the origin of replication for propagation and stable maintenance in Agrobacterium spp. is derived from a pRi, a pVS1, a pRSF1010, a pRK2, a pSa, or a pBBR1 origin of replication. In an aspect, the origin of replication for propagation and stable maintenance in Agrobacterium spp. is a variant of the pRK2 origin of replication. In an aspect, the origin of replication for propagation and stable maintenance in Agrobacterium spp. is derived from the pRSF1010 origin of replication. In an aspect, the origin of replication for propagation and stable maintenance in Agrobacterium spp. is derived from the pVS1 origin of replication. In an aspect, the origin of replication for propagation and stable maintenance in Agrobacterium spp. is derived from the pSa origin of replication. In an aspect, the origin of replication for propagation and stable maintenance in Agrobacterium spp. is an origin of replication having any one of SEQ ID NOS: 3, 37, 38, 53, 57, 58, 59, or 60, or variants and fragments thereof. In an aspect, the origin of replication for propagation and stable maintenance in Agrobacterium spp. is a repABC compatible origin of replication. In an aspect, the repABC compatible origin of replication has any one of SEQ ID NOS: 57, 58, 59, or 60, or variants and fragments thereof. In an aspect, the origin of replication for propagation and stable maintenance in Escherichia coli and the origin of replication for propagation and stable maintenance in Agrobacterium spp. are the same origin of replication. In an aspect, the origin of replication is derived from a pRK2 origin of replication, from a pSa origin of replication, or a pRSF1010 origin of replication. In an aspect, the origin of replication is derived from the pRK2 origin of replication. In an aspect, the pRK2 origin of replication has SEQ ID NO: 38, or variants and fragments thereof. In an aspect, the pRK2 origin of replication is a mini or micro pRK2 origin of replication. In an aspect, the pRK2 origin of replication is a micro pRK2 origin of replication. In an aspect, the micro pRK2 origin of replication has SEQ ID NO: 54, or variants and fragments thereof. In an aspect, the pRK2 origin of replication is a mini pRK2 origin of replication. In an aspect, the mini pRK2 has SEQ ID NO: 66, or variants and fragments thereof. In an aspect, the pRK2 origin of replication comprises the trfA and OriV sequences. In an aspect, the pRK2 origin of replication comprises SEQ ID NOS: 64 and 65, or variants and fragments thereof. In an aspect, the origin of replication is derived from the pSa origin of replication. In an aspect, the pSa origin of replication has SEQ ID NO: 53, or variants and fragments thereof. In an aspect, the origin of replication is derived from the pRSF1010 origin of replication. In an aspect, the pRSF1010 origin of replication has SEQ ID NO: 37, or variants and fragments thereof. In an aspect, the vector further comprises a sequence derived from the par DE operon. In an aspect, the par DE operon has SEQ ID NO: 55, or variants and fragments thereof. In an aspect, the selectable marker gene provides resistance to gentamicin, neomycin/kanamycin, hygromycin, or spectinomycin. In an aspect, the selectable marker gene is an aacC1 gene, a npt1gene, a npt2 gene, a hpt gene, an aadA gene, a SpcN gene, or an aph gene. In an aspect, the selectable marker gene is aacC1. In an aspect, the aacC1 selectable marker gene has SEQ ID NO: 1, or variants and fragments thereof. In an aspect, the selectable marker gene is aadA. In an aspect, the aadA selectable marker gene has SEQ ID NO: 39, or variants and fragments thereof. In an aspect, the selectable marker gene is npt1. In an aspect, the nptl selectable marker gene has SEQ ID NO: 40, or variants and fragments thereof. In an aspect, the selectable marker gene is npt2. In an aspect, the npt2 selectable marker gene has SEQ ID NO: 41, or variants and fragments thereof. In an aspect, the selectable marker gene is hpt. In an aspect, the hpt selectable marker gene has SEQ ID NO: 67, or variants and fragments thereof. In an aspect, the selectable marker gene is SpcN. In an aspect, the SpcN selectable marker gene has SEQ ID NO: 77, or variants and fragments thereof. In an aspect, the selectable marker gene is aph. In an aspect, the aph selectable marker gene has SEQ ID NO: 78, or variants and fragments thereof. In an aspect, the selectable marker gene does not provide resistance to tetracycline. In an aspect, the selectable marker gene is not a tetAR gene. In an aspect, the selectable marker gene is a counter-selectable marker gene. In an aspect, the counter-selectable marker gene is a sacB gene, a rpsL (strA) gene, a pheS gene, adhfr (folA) gene, a lacY gene, a Gata-1 gene, a ccdB gene, or a thyA− gene. In an aspect, the vector does not comprise SEQ ID NO: 61, or variants or fragments thereof. In an aspect, the vector does not comprise SEQ ID NO: 62, or variants or fragments thereof. In an aspect, the vector does not comprise a tra operon sequence or a trb operon sequence, or variants or fragments thereof. In an aspect, the vector does not comprise SEQ ID NO: 63, or variants or fragments thereof. In an aspect, the vector has SEQ ID NO: 34, or variants and fragments thereof. In an aspect, the vector has SEQ ID NO: 35, or variants and fragments thereof. In an aspect, the vector has SEQ ID NO: 36, or variants and fragments thereof.

In an aspect, the present disclosure further provides a method for transformation of a plant comprising the steps of: (a) contacting a tissue from the plant with an Agrobacterium strain or an Ochrobactrum strain comprising a first vector comprising: (i) an origin of replication for propagation in Escherichia coli having SEQ ID NO: 2, or variants and fragments thereof; (ii) an origin of replication for propagation in Agrobacterium spp. having SEQ ID NO: 3, or variants and fragments thereof; (iii) a selectable marker gene having SEQ ID NO: 1; and (iv) virulence genes comprising Agrobacterium spp. virulence genes a virA virulence gene having SEQ ID NO: 26 or a r-virA virulence gene having SEQ ID NO: 79, virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D5 virulence genes having SEQ ID NOS: 18-22, respectively or r-virD1-D5 virulence genes having SEQ ID NOS: 94-98, respectively, virE1-E3 virulence genes having SEQ ID NOS: 23-25, respectively or a r-virE3 virulence gene having SEQ ID NOS: 100, and a virG virulence gene having SEQ ID NO: 15 or a r-virG virulence gene having SEQ ID NO: 91, or variants and derivatives thereof, wherein the vector comprising the virulence genes r-virA, r-virB1-B11, r-virC1-C2, r-virD1-D5, r-virE3, and r-virG further comprises a r-galls virulence gene having SEQ ID NO: 101, or variants and derivatives thereof, and a second vector comprising T-DNA borders and a polynucleotide sequence of interest for transfer to the plant; (b) co-cultivating the tissue with the Agrobacterium strain or the Ochrobactrum strain; and (c) regenerating a transformed plant from the tissue that expresses the polynucleotide sequence of interest. In an aspect, the Rhizobiaceae virulence genes are Agrobacterium spp., Rhizobium spp., Sinorhizobium spp., Mesorhizobium spp., Phyllobacterium spp., Ochrobactrum spp., or Bradyrhizobium spp. virulence genes. In an aspect, the Rhizobiaceae virulence genes are Agrobacterium spp. virulence genes. In an aspect, the Agrobacterium spp. virulence genes are Agrobacterium albertimagni, Agrobacterium larrymoorei, Agrobacterium radiobacter, Agrobacterium rhizogenes, Agrobacterium rubi, Agrobacterium tumefaciens, or Agrobacterium vitis virulence genes. In an aspect, the Agrobacterium spp. virulence genes are Agrobacterium rhizogenes or Agrobacterium tumefaciens virulence genes. In an aspect, the Agrobacterium spp. virulence genes are Agrobacterium rhizogenes virulence genes. In an aspect, the Agrobacterium spp. virulence genes are Agrobacterium tumefaciens virulence genes. In an aspect, the Rhizobiaceae virulence genes are virB1-virB11 virulence genes having SEQ ID NOS: 4-14, respectively, or r-virB1-B11 virulence genes having SEQ ID NOS: 80-90, respectively, or variants and derivatives thereof. In an aspect, the Rhizobiaceae virulence genes are virC1-C2 virulence genes having SEQ ID NOS: 16-17, respectively, or r-virC1-C2 virulence genes having SEQ ID NOS: 92-93, respectively, or variants and derivatives thereof. In an aspect, the Rhizobiaceae virulence genes are virD1-D2 virulence genes having SEQ ID NOS: 18-19, respectively, or r-virD1-D2 virulence genes having SEQ ID NOS: 94-95, respectively, or variants and derivatives thereof. In an aspect, the Rhizobiaceae virulence gene is a virG virulence gene having SEQ ID NO: 15, or a r-virG virulence gene having SEQ ID NO: 91, or variants and derivatives thereof. In an aspect, the Rhizobiaceae virulence gene is a r-galls virulence gene having SEQ ID NO: 101, or variants and derivatives thereof. In an aspect, the vector further comprises one or more of Rhizobiaceae virulence genes virA, virD3, virD4, virD5, virE1, virE2, virE3, virH, virH1, virH2, virK, virL, virM, virP, virQ, r-virA, r-virD3, r-virD4, r-virD5, r-virE3, or r-virF or variants and derivatives thereof. In an aspect, the Rhizobiaceae virulence gene is a virA virulence gene having SEQ ID NO: 26 or a r-virA virulence gene having SEQ ID NO: 79, or variants and derivatives thereof. In an aspect, the Rhizobiaceae virulence genes are virD3-D5 virulence genes having SEQ ID NOS: 20-22, respectively, or r-virD3-D5 virulence genes having SEQ ID NO: 96-98, respectively, or variants and derivatives thereof. In an aspect, the Rhizobiaceae virulence genes are virE1-E3 virulence genes having SEQ ID NOS: 23-25, respectively, or a r-virE3 virulence gene having SEQ ID NO: 100, or variants and derivatives thereof. In an aspect, the Rhizobiaceae virulence genes are virH-H1 virulence genes having, SEQ ID NOS: 42-43, respectively, or variants and derivatives thereof. In an aspect, the Rhizobiaceae virulence gene is a virK virulence gene having SEQ ID NO: 45, or variants and derivatives thereof. In an aspect, the Rhizobiaceae virulence gene is a virL virulence gene having SEQ ID NO: 46, or variants and derivatives thereof. In an aspect, the Rhizobiaceae virulence gene is a virM virulence gene having SEQ ID NO: 47, or variants and derivatives thereof. In an aspect, the Rhizobiaceae virulence gene is a virP virulence gene having SEQ ID NO: 48, or variants and derivatives thereof. In an aspect, the Rhizobiaceae virulence gene is a virQ virulence gene having SEQ ID NO: 49, or variants and derivatives thereof. In an aspect, the vector further comprises the Rhizobiaceae virulence genes virD3-D5 and virE1-E3 or r-virD3-D5 and r-vir E3, or variants and derivatives thereof. In an aspect, the vector further comprises the Rhizobiaceae virulence genes virA, virD3-D5, and virE1-E3, or r-virA, r-virD3-D5, and r-virE3, or variants and derivatives thereof. In an aspect, the origin of replication for propagation and stable maintenance in Escherichia coli is derived from a Col E1, a pSC101, a p15A, or a R6K origin of replication, or functional variants and derivatives thereof. In an aspect, the origin of replication for propagation and stable maintenance in Escherichia coli is derived from a Col E1 origin of replication. In an aspect, the origin of replication derived from the ColE1 origin of replication has SEQ ID NO: 2, or variants and fragments thereof. In an aspect, the origin of replication for propagation and stable maintenance in Escherichia coli is derived from a pSC101 origin of replication. In an aspect, the origin of replication derived from the pSC101 origin of replication has SEQ ID NO: 50, or variants and fragments thereof. In an aspect, the origin of replication for propagation and stable maintenance in Escherichia coli is derived from a p15A origin of replication. In an aspect, the origin of replication derived from the p15A origin of replication has SEQ ID NO: 51, or variants and fragments thereof. In an aspect, the origin of replication for propagation and stable maintenance in Escherichia coli is derived from a R6K origin of replication. In an aspect, the origin of replication derived from the R6K origin of replication has SEQ ID NO: 52, or variants and fragments thereof. In an aspect, the origin of replication for propagation and stable maintenance in Agrobacterium spp. is a high copy number origin of replication. In an aspect, the origin of replication for propagation and stable maintenance in Agrobacterium spp. is an intermediate copy number origin of replication. In an aspect, the origin of replication for propagation and stable maintenance in Agrobacterium spp. is a low copy number origin of replication. In an aspect, the origin of replication for propagation and stable maintenance in Agrobacterium spp. is derived from a pRi, a pVS1, a pRSF1010, a pRK2, a pSa, or a pBBR1 origin of replication. In an aspect, the origin of replication for propagation and stable maintenance in Agrobacterium spp. is a variant of the pRK2 origin of replication. In an aspect, the origin of replication for propagation and stable maintenance in Agrobacterium spp. is derived from the pRSF1010 origin of replication. In an aspect, the origin of replication for propagation and stable maintenance in Agrobacterium spp. is derived from the pVS1 origin of replication. In an aspect, the origin of replication for propagation and stable maintenance in Agrobacterium spp. is derived from the pSa origin of replication. In an aspect, the origin of replication for propagation and stable maintenance in Agrobacterium spp. is an origin of replication having any one of SEQ ID NOS: 3, 37, 38, 53, 57, 58, 59, or 60, or variants and fragments thereof. In an aspect, the origin of replication for propagation and stable maintenance in Agrobacterium spp. is a repABC compatible origin of replication. In an aspect, the repABC compatible origin of replication has any one of SEQ ID NOS: 57, 58, 59, or 60, or variants and fragments thereof. In an aspect, the origin of replication for propagation and stable maintenance in Escherichia coli and the origin of replication for propagation and stable maintenance in Agrobacterium spp. are the same origin of replication. In an aspect, the origin of replication is derived from a pRK2 origin of replication, from a pSa origin of replication, or a pRSF1010 origin of replication. In an aspect, the origin of replication is derived from the pRK2 origin of replication. In an aspect, the pRK2 origin of replication has SEQ ID NO: 38, or variants and fragments thereof. In an aspect, the pRK2 origin of replication is a mini or micro pRK2 origin of replication. In an aspect, the pRK2 origin of replication is a micro pRK2 origin of replication. In an aspect, the micro pRK2 origin of replication has SEQ ID NO: 54, or variants and fragments thereof. In an aspect, the pRK2 origin of replication is a mini pRK2 origin of replication. In an aspect, the mini pRK2 has SEQ ID NO: 66, or variants and fragments thereof. In an aspect, the pRK2 origin of replication comprises the trfA and OriV sequences. In an aspect, the pRK2 origin of replication comprises SEQ ID NOS: 64 and 65, or variants and fragments thereof. In an aspect, the origin of replication is derived from the pSa origin of replication. In an aspect, the pSa origin of replication has SEQ ID NO: 53, or variants and fragments thereof. In an aspect, the origin of replication is derived from the pRSF1010 origin of replication. In an aspect, the pRSF1010 origin of replication has SEQ ID NO: 37, or variants and fragments thereof. In an aspect, the vector further comprises a sequence derived from the par DE operon. In an aspect, the par DE operon has SEQ ID NO: 55, or variants and fragments thereof. In an aspect, the selectable marker gene provides resistance to gentamicin, neomycin/kanamycin, hygromycin, or spectinomycin. In an aspect, the selectable marker gene is an aacC1 gene, a npt1gene, a npt2 gene, a hpt gene, an aadA gene, a SpcN gene, or an aph gene. In an aspect, the selectable marker gene is aacC1. In an aspect, the aacC1 selectable marker gene has SEQ ID NO: 1, or variants and fragments thereof. In an aspect, the selectable marker gene is aadA. In an aspect, the aadA selectable marker gene has SEQ ID NO: 39, or variants and fragments thereof. In an aspect, the selectable marker gene is npt1. In an aspect, the npt1 selectable marker gene has SEQ ID NO: 40, or variants and fragments thereof. In an aspect, the selectable marker gene is npt2. In an aspect, the npt2 selectable marker gene has SEQ ID NO: 41, or variants and fragments thereof. In an aspect, the selectable marker gene is hpt. In an aspect, the hpt selectable marker gene has SEQ ID NO: 67, or variants and fragments thereof. In an aspect, the selectable marker gene is SpcN. In an aspect, the SpcN selectable marker gene has SEQ ID NO: 77, or variants and fragments thereof. In an aspect, the selectable marker gene is aph. In an aspect, the aph selectable marker gene has SEQ ID NO: 78, or variants and fragments thereof. In an aspect, the selectable marker gene does not provide resistance to tetracycline. In an aspect, the selectable marker gene is not a tetAR gene. In an aspect, the selectable marker gene is a counter-selectable marker gene. In an aspect, the counter-selectable marker gene is a sacB gene, a rpsL (strA) gene, a pheS gene, adhfr (folA) gene, a lacY gene, a Gata-1 gene, a ccdB gene, or a thyA− gene. In an aspect, the vector does not comprise SEQ ID NO: 61, or variants or fragments thereof. In an aspect, the vector does not comprise SEQ ID NO: 62, or variants or fragments thereof. In an aspect, the vector does not comprise a tra operon sequence or a trb operon sequence, or variants or fragments thereof. In an aspect, the vector does not comprise SEQ ID NO: 63, or variants or fragments thereof. In an aspect, the vector has SEQ ID NO: 34, or variants and fragments thereof. In an aspect, the vector has SEQ ID NO: 35, or variants and fragments thereof. In an aspect, the vector has SEQ ID NO: 36, or variants and fragments thereof.

In an aspect, the present disclosure further provides a kit comprising: (a) a vector comprising: (i) an origin of replication for propagation and stable maintenance in Escherichia coli; (ii) an origin of replication for propagation and stable maintenance in Agrobacterium spp.; (iii) a selectable marker gene; and (iv) Rhizobiaceae virulence genes virB1-B11 or r-virB1-B11, virC1-C2 or r-virC1-C2, virD1-D2 or r-virD1-D2, and virG or r-virG, or variants and derivatives thereof, wherein the vector comprising the virulence genes r-virB1-B11, r-virC1-C2, r-virD1-D2, and r-virG further comprises a r-galls virulence gene, or variants and derivatives thereof; and (b) instructions for use in transformation of a plant using Agrobacterium or Ochrobactrum. In an aspect, the Rhizobiaceae virulence genes are Agrobacterium spp., Rhizobium spp., Sinorhizobium spp., Mesorhizobium spp., Phyllobacterium spp., Ochrobactrum spp., or Bradyrhizobium spp. virulence genes. In an aspect, the Rhizobiaceae virulence genes are Agrobacterium spp. virulence genes. In an aspect, the Agrobacterium spp. virulence genes are Agrobacterium albertimagni, Agrobacterium larrymoorei, Agrobacterium radiobacter, Agrobacterium rhizogenes, Agrobacterium rubi, Agrobacterium tumefaciens, or Agrobacterium vitis virulence genes. In an aspect, the Agrobacterium spp. virulence genes are Agrobacterium rhizogenes or Agrobacterium tumefaciens virulence genes. In an aspect, the Agrobacterium spp. virulence genes are Agrobacterium rhizogenes virulence genes. In an aspect, the Agrobacterium spp. virulence genes are Agrobacterium tumefaciens virulence genes. In an aspect, the Rhizobiaceae virulence genes are virB1-virB11 virulence genes having SEQ ID NOS: 4-14, respectively, or r-virB1-B11 virulence genes having SEQ ID NOS: 80-90, respectively, or variants and derivatives thereof. In an aspect, the Rhizobiaceae virulence genes are virC1-C2 virulence genes having SEQ ID NOS: 16-17, respectively, or r-virC1-C2 virulence genes having SEQ ID NOS: 92-93, respectively, or variants and derivatives thereof. In an aspect, the Rhizobiaceae virulence genes are virD1-D2 virulence genes having SEQ ID NOS: 18-19, respectively, or r-virD1-D2 virulence genes having SEQ ID NOS: 94-95, respectively, or variants and derivatives thereof. In an aspect, the Rhizobiaceae virulence gene is a virG virulence gene having SEQ ID NO: 15, or a r-virG virulence gene having SEQ ID NO: 91, or variants and derivatives thereof. In an aspect, the Rhizobiaceae virulence gene is a r-galls virulence gene having SEQ ID NO: 101, or variants and derivatives thereof. In an aspect, the vector further comprises one or more of Rhizobiaceae virulence genes virA, virD3, virD4, virD5, virE1, virE2, virE3, virH, virH1, virH2, virK, virL, virM, virP, virQ, r-virA , r-virD3, r-virD4, r-virD5, r-virE3, or r-virF or variants and derivatives thereof. In an aspect, the Rhizobiaceae virulence gene is a virA virulence gene having SEQ ID NO: 26 or a r-virA virulence gene having SEQ ID NO: 79, or variants and derivatives thereof. In an aspect, the Rhizobiaceae virulence genes are virD3-D5 virulence genes having SEQ ID NOS: 20-22, respectively, or r-virD3-D5 virulence genes having SEQ ID NO: 96-98, respectively, or variants and derivatives thereof. In an aspect, the Rhizobiaceae virulence genes are virE1-E3 virulence genes having SEQ ID NOS: 23-25, respectively, or a r-virE3 virulence gene having SEQ ID NO: 100, or variants and derivatives thereof. In an aspect, the Rhizobiaceae virulence genes are virH-H1 virulence genes having, SEQ ID NOS: 42-43, respectively, or variants and derivatives thereof. In an aspect, the Rhizobiaceae virulence gene is a virK virulence gene having SEQ ID NO: 45, or variants and derivatives thereof. In an aspect, the Rhizobiaceae virulence gene is a virL virulence gene having SEQ ID NO: 46, or variants and derivatives thereof. In an aspect, the Rhizobiaceae virulence gene is a virM virulence gene having SEQ ID NO: 47, or variants and derivatives thereof. In an aspect, the Rhizobiaceae virulence gene is a virP virulence gene having SEQ ID NO: 48, or variants and derivatives thereof. In an aspect, the Rhizobiaceae virulence gene is a virQ virulence gene having SEQ ID NO: 49, or variants and derivatives thereof. In an aspect, the vector further comprises the Rhizobiaceae virulence genes virD3-D5 and virE1-E3 or r-virD3-D5 and r-vir E3, or variants and derivatives thereof. In an aspect, the vector further comprises the Rhizobiaceae virulence genes virA, virD3-D5, and virE1-E3, or r-virA, r-virD3-D5, and r-virE3, or variants and derivatives thereof. In an aspect, the origin of replication for propagation and stable maintenance in Escherichia coli is derived from a Col E1, a pSC101, a p15A, or a R6K origin of replication, or functional variants and derivatives thereof. In an aspect, the origin of replication for propagation and stable maintenance in Escherichia coli is derived from a Col E1 origin of replication. In an aspect, the origin of replication derived from the ColE1 origin of replication has SEQ ID NO: 2, or variants and fragments thereof. In an aspect, the origin of replication for propagation and stable maintenance in Escherichia coli is derived from a pSC101 origin of replication. In an aspect, the origin of replication derived from the pSC101 origin of replication has SEQ ID NO: 50, or variants and fragments thereof. In an aspect, the origin of replication for propagation and stable maintenance in Escherichia coli is derived from a p 15A origin of replication. In an aspect, the origin of replication derived from the p15A origin of replication has SEQ ID NO: 51, or variants and fragments thereof. In an aspect, the origin of replication for propagation and stable maintenance in Escherichia coli is derived from a R6K origin of replication. In an aspect, the origin of replication derived from the R6K origin of replication has SEQ ID NO: 52, or variants and fragments thereof. In an aspect, the origin of replication for propagation and stable maintenance in Agrobacterium spp. is a high copy number origin of replication. In an aspect, the origin of replication for propagation and stable maintenance in Agrobacterium spp. is an intermediate copy number origin of replication. In an aspect, the origin of replication for propagation and stable maintenance in Agrobacterium spp. is a low copy number origin of replication. In an aspect, the origin of replication for propagation and stable maintenance in Agrobacterium spp. is derived from a pRi, a pVS1, a pRSF1010, a pRK2, a pSa, or a pBBR1 origin of replication. In an aspect, the origin of replication for propagation and stable maintenance in Agrobacterium spp. is a variant of the pRK2 origin of replication. In an aspect, the origin of replication for propagation and stable maintenance in Agrobacterium spp. is derived from the pRSF1010 origin of replication. In an aspect, the origin of replication for propagation and stable maintenance in Agrobacterium spp. is derived from the pVS1 origin of replication. In an aspect, the origin of replication for propagation and stable maintenance in Agrobacterium spp. is derived from the pSa origin of replication. In an aspect, the origin of replication for propagation and stable maintenance in Agrobacterium spp. is an origin of replication having any one of SEQ ID NOS: 3, 37, 38, 53, 57, 58, 59, or 60, or variants and fragments thereof. In an aspect, the origin of replication for propagation and stable maintenance in Agrobacterium spp. is a repABC compatible origin of replication. In an aspect, the repABC compatible origin of replication has any one of SEQ ID NOS: 57, 58, 59, or 60, or variants and fragments thereof. In an aspect, the origin of replication for propagation and stable maintenance in Escherichia coli and the origin of replication for propagation and stable maintenance in Agrobacterium spp. are the same origin of replication. In an aspect, the origin of replication is derived from a pRK2 origin of replication, from a pSa origin of replication, or a pRSF1010 origin of replication. In an aspect, the origin of replication is derived from the pRK2 origin of replication. In an aspect, the pRK2 origin of replication has SEQ ID NO: 38, or variants and fragments thereof. In an aspect, the pRK2 origin of replication is a mini or micro pRK2 origin of replication. In an aspect, the pRK2 origin of replication is a micro pRK2 origin of replication. In an aspect, the micro pRK2 origin of replication has SEQ ID NO: 54, or variants and fragments thereof. In an aspect, the pRK2 origin of replication is a mini pRK2 origin of replication. In an aspect, the mini pRK2 has SEQ ID NO: 66, or variants and fragments thereof. In an aspect, the pRK2 origin of replication comprises the trfA and OriV sequences. In an aspect, the pRK2 origin of replication comprises SEQ ID NOS: 64 and 65, or variants and fragments thereof. In an aspect, the origin of replication is derived from the pSa origin of replication. In an aspect, the pSa origin of replication has SEQ ID NO: 53, or variants and fragments thereof. In an aspect, the origin of replication is derived from the pRSF1010 origin of replication. In an aspect, the pRSF1010 origin of replication has SEQ ID NO: 37, or variants and fragments thereof. In an aspect, the vector further comprises a sequence derived from the par DE operon. In an aspect, the par DE operon has SEQ ID NO: 55, or variants and fragments thereof. In an aspect, the selectable marker gene provides resistance to gentamicin, neomycin/kanamycin, hygromycin, or spectinomycin. In an aspect, the selectable marker gene is an aacC1 gene, a npt1gene, a npt2 gene, a hpt gene, an aadA gene, a SpcN gene, or an aph gene. In an aspect, the selectable marker gene is aacC1. In an aspect, the aacC1 selectable marker gene has SEQ ID NO: 1, or variants and fragments thereof. In an aspect, the selectable marker gene is aadA. In an aspect, the aadA selectable marker gene has SEQ ID NO: 39, or variants and fragments thereof. In an aspect, the selectable marker gene is npt1. In an aspect, the npt1 selectable marker gene has SEQ ID NO: 40, or variants and fragments thereof. In an aspect, the selectable marker gene is npt2. In an aspect, the npt2 selectable marker gene has SEQ ID NO: 41, or variants and fragments thereof. In an aspect, the selectable marker gene is hpt. In an aspect, the hpt selectable marker gene has SEQ ID NO: 67, or variants and fragments thereof. In an aspect, the selectable marker gene is SpcN. In an aspect, the SpcN selectable marker gene has SEQ ID NO: 77, or variants and fragments thereof. In an aspect, the selectable marker gene is aph. In an aspect, the aph selectable marker gene has SEQ ID NO: 78, or variants and fragments thereof. In an aspect, the selectable marker gene does not provide resistance to tetracycline. In an aspect, the selectable marker gene is not a tetAR gene. In an aspect, the selectable marker gene is a counter-selectable marker gene. In an aspect, the counter-selectable marker gene is a sacB gene, a rpsL (strA) gene, a pheS gene, adhfr (folA) gene, a lacY gene, a Gata-1 gene, a ccdB gene, or a thyA− gene. In an aspect, the vector does not comprise SEQ ID NO: 61, or variants or fragments thereof. In an aspect, the vector does not comprise SEQ ID NO: 62, or variants or fragments thereof. In an aspect, the vector does not comprise a tra operon sequence or a trb operon sequence, or variants or fragments thereof. In an aspect, the vector does not comprise SEQ ID NO: 63, or variants or fragments thereof. In an aspect, the vector has SEQ ID NO: 34, or variants and fragments thereof. In an aspect, the vector has SEQ ID NO: 35, or variants and fragments thereof. In an aspect, the vector has SEQ ID NO: 36, or variants and fragments thereof.

In an aspect, the present disclosure further provides a kit comprising: (a) a vector comprising: (i) an origin of replication for propagation in Escherichia coli having SEQ ID NO: 2, or variants and fragments thereof; (ii) an origin of replication for propagation in Agrobacterium spp. having SEQ ID NO: 3, or variants and fragments thereof; (iii) a selectable marker gene having SEQ ID NO: 1, or variants and fragments thereof; and (iv) virulence genes comprising Agrobacterium spp. virulence genes virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D2 virulence genes having SEQ ID NOS: 18-19, respectively or r-virD1-D2 virulence genes having SEQ ID NOS: 94-95, respectively, and a virG virulence gene having SEQ ID NO: 15 or a r-virG virulence gene having SEQ ID NO: 91, or variants and derivatives thereof, wherein the vector comprising the virulence genes r-virB1-B11, r-virC1-C2, r-virD1-D2, and r-virG further comprises a r-galls virulence gene having SEQ ID NO: 101, or variants and derivatives thereof; and (b) instructions for use in transformation of a plant using Agrobacterium or Ochrobactrum. In an aspect, the Rhizobiaceae virulence genes are Agrobacterium spp., Rhizobium spp., Sinorhizobium spp., Mesorhizobium spp., Phyllobacterium spp., Ochrobactrum spp., or Bradyrhizobium spp. virulence genes. In an aspect, the Rhizobiaceae virulence genes are Agrobacterium spp. virulence genes. In an aspect, the Agrobacterium spp. virulence genes are Agrobacterium albertimagni, Agrobacterium larrymoorei, Agrobacterium radiobacter, Agrobacterium rhizogenes, Agrobacterium rubi, Agrobacterium tumefaciens, or Agrobacterium vitis virulence genes. In an aspect, the Agrobacterium spp. virulence genes are Agrobacterium rhizogenes or Agrobacterium tumefaciens virulence genes. In an aspect, the Agrobacterium spp. virulence genes are Agrobacterium rhizogenes virulence genes. In an aspect, the Agrobacterium spp. virulence genes are Agrobacterium tumefaciens virulence genes. In an aspect, the Rhizobiaceae virulence genes are virB1-virB11 virulence genes having SEQ ID NOS: 4-14, respectively, or r-virB1-B11 virulence genes having SEQ ID NOS: 80-90, respectively, or variants and derivatives thereof. In an aspect, the Rhizobiaceae virulence genes are virC1-C2 virulence genes having SEQ ID NOS: 16-17, respectively, or r-virC1-C2 virulence genes having SEQ ID NOS: 92-93, respectively, or variants and derivatives thereof. In an aspect, the Rhizobiaceae virulence genes are virD1-D2 virulence genes having SEQ ID NOS: 18-19, respectively, or r-virD1-D2 virulence genes having SEQ ID NOS: 94-95, respectively, or variants and derivatives thereof. In an aspect, the Rhizobiaceae virulence gene is a virG virulence gene having SEQ ID NO: 15, or a r-virG virulence gene having SEQ ID NO: 91, or variants and derivatives thereof. In an aspect, the Rhizobiaceae virulence gene is a r-galls virulence gene having SEQ ID NO: 101, or variants and derivatives thereof. In an aspect, the vector further comprises one or more of Rhizobiaceae virulence genes virA, virD3, virD4, virD5, virE1, virE2, virE3, virH, virH1, virH2, virK, virL, virM, virP, virQ, r-virA, r-virD3, r-virD4, r-virD5, r-virE3, or r-virF or variants and derivatives thereof. In an aspect, the Rhizobiaceae virulence gene is a virA virulence gene having SEQ ID NO: 26 or a r-virA virulence gene having SEQ ID NO: 79, or variants and derivatives thereof. In an aspect, the Rhizobiaceae virulence genes are virD3-D5 virulence genes having SEQ ID NOS: 20-22, respectively, or r-virD3-D5 virulence genes having SEQ ID NO: 96-98, respectively, or variants and derivatives thereof. In an aspect, the Rhizobiaceae virulence genes are virE1-E3 virulence genes having SEQ ID NOS: 23-25, respectively, or a r-virE3 virulence gene having SEQ ID NO: 100, or variants and derivatives thereof. In an aspect, the Rhizobiaceae virulence genes are virH-H1 virulence genes having, SEQ ID NOS: 42-43, respectively, or variants and derivatives thereof. In an aspect, the Rhizobiaceae virulence gene is a virK virulence gene having SEQ ID NO: 45, or variants and derivatives thereof. In an aspect, the Rhizobiaceae virulence gene is a virL virulence gene having SEQ ID NO: 46, or variants and derivatives thereof. In an aspect, the Rhizobiaceae virulence gene is a virM virulence gene having SEQ ID NO: 47, or variants and derivatives thereof. In an aspect, the Rhizobiaceae virulence gene is a virP virulence gene having SEQ ID NO: 48, or variants and derivatives thereof. In an aspect, the Rhizobiaceae virulence gene is a virQ virulence gene having SEQ ID NO: 49, or variants and derivatives thereof. In an aspect, the vector further comprises the Rhizobiaceae virulence genes virD3-D5 and virE1-E3 or r-virD3-D5 and r-vir E3, or variants and derivatives thereof. In an aspect, the vector further comprises the Rhizobiaceae virulence genes virA, virD3-D5, and virE1-E3, or r-virA, r-virD3-D5, and r-virE3, or variants and derivatives thereof. In an aspect, the origin of replication for propagation and stable maintenance in Escherichia coli is derived from a Col E1, a pSC101, a p15A, or a R6K origin of replication, or functional variants and derivatives thereof. In an aspect, the origin of replication for propagation and stable maintenance in Escherichia coli is derived from a Col E1 origin of replication. In an aspect, the origin of replication derived from the ColE1 origin of replication has SEQ ID NO: 2, or variants and fragments thereof. In an aspect, the origin of replication for propagation and stable maintenance in Escherichia coli is derived from a pSC101 origin of replication. In an aspect, the origin of replication derived from the pSC101 origin of replication has SEQ ID NO: 50, or variants and fragments thereof. In an aspect, the origin of replication for propagation and stable maintenance in Escherichia coli is derived from a p15A origin of replication. In an aspect, the origin of replication derived from the p15A origin of replication has SEQ ID NO: 51, or variants and fragments thereof. In an aspect, the origin of replication for propagation and stable maintenance in Escherichia coli is derived from a R6K origin of replication. In an aspect, the origin of replication derived from the R6K origin of replication has SEQ ID NO: 52, or variants and fragments thereof. In an aspect, the origin of replication for propagation and stable maintenance in Agrobacterium spp. is a high copy number origin of replication. In an aspect, the origin of replication for propagation and stable maintenance in Agrobacterium spp. is an intermediate copy number origin of replication. In an aspect, the origin of replication for propagation and stable maintenance in Agrobacterium spp. is a low copy number origin of replication. In an aspect, the origin of replication for propagation and stable maintenance in Agrobacterium spp. is derived from a pRi, a pVS1, a pRSF1010, a pRK2, a pSa, or a pBBR1 origin of replication. In an aspect, the origin of replication for propagation and stable maintenance in Agrobacterium spp. is a variant of the pRK2 origin of replication. In an aspect, the origin of replication for propagation and stable maintenance in Agrobacterium spp. is derived from the pRSF1010 origin of replication. In an aspect, the origin of replication for propagation and stable maintenance in Agrobacterium spp. is derived from the pVS1 origin of replication. In an aspect, the origin of replication for propagation and stable maintenance in Agrobacterium spp. is derived from the pSa origin of replication. In an aspect, the origin of replication for propagation and stable maintenance in Agrobacterium spp. is an origin of replication having any one of SEQ ID NOS: 3, 37, 38, 53, 57, 58, 59, or 60, or variants and fragments thereof. In an aspect, the origin of replication for propagation and stable maintenance in Agrobacterium spp. is a repABC compatible origin of replication. In an aspect, the repABC compatible origin of replication has any one of SEQ ID NOS: 57, 58, 59, or 60, or variants and fragments thereof. In an aspect, the origin of replication for propagation and stable maintenance in Escherichia coli and the origin of replication for propagation and stable maintenance in Agrobacterium spp. are the same origin of replication. In an aspect, the origin of replication is derived from a pRK2 origin of replication, from a pSa origin of replication, or a pRSF1010 origin of replication. In an aspect, the origin of replication is derived from the pRK2 origin of replication. In an aspect, the pRK2 origin of replication has SEQ ID NO: 38, or variants and fragments thereof. In an aspect, the pRK2 origin of replication is a mini or micro pRK2 origin of replication. In an aspect, the pRK2 origin of replication is a micro pRK2 origin of replication. In an aspect, the micro pRK2 origin of replication has SEQ ID NO: 54, or variants and fragments thereof. In an aspect, the pRK2 origin of replication is a mini pRK2 origin of replication. In an aspect, the mini pRK2 has SEQ ID NO: 66, or variants and fragments thereof. In an aspect, the pRK2 origin of replication comprises the trfA and OriV sequences. In an aspect, the pRK2 origin of replication comprises SEQ ID NOS: 64 and 65, or variants and fragments thereof. In an aspect, the origin of replication is derived from the pSa origin of replication. In an aspect, the pSa origin of replication has SEQ ID NO: 53, or variants and fragments thereof. In an aspect, the origin of replication is derived from the pRSF1010 origin of replication. In an aspect, the pRSF1010 origin of replication has SEQ ID NO: 37, or variants and fragments thereof. In an aspect, the vector further comprises a sequence derived from the par DE operon. In an aspect, the par DE operon has SEQ ID NO: 55, or variants and fragments thereof. In an aspect, the selectable marker gene provides resistance to gentamicin, neomycin/kanamycin, hygromycin, or spectinomycin. In an aspect, the selectable marker gene is an aacC1 gene, a npt1gene, a npt2 gene, a hpt gene, an aadA gene, a SpcN gene, or an aph gene. In an aspect, the selectable marker gene is aacC1. In an aspect, the aacC1 selectable marker gene has SEQ ID NO: 1, or variants and fragments thereof. In an aspect, the selectable marker gene is aadA. In an aspect, the aadA selectable marker gene has SEQ ID NO: 39, or variants and fragments thereof. In an aspect, the selectable marker gene is npt1. In an aspect, the nptl selectable marker gene has SEQ ID NO: 40, or variants and fragments thereof. In an aspect, the selectable marker gene is npt2. In an aspect, the npt2 selectable marker gene has SEQ ID NO: 41, or variants and fragments thereof. In an aspect, the selectable marker gene is hpt. In an aspect, the hpt selectable marker gene has SEQ ID NO: 67, or variants and fragments thereof. In an aspect, the selectable marker gene is SpcN. In an aspect, the SpcN selectable marker gene has SEQ ID NO: 77, or variants and fragments thereof. In an aspect, the selectable marker gene is aph. In an aspect, the aph selectable marker gene has SEQ ID NO: 78, or variants and fragments thereof. In an aspect, the selectable marker gene does not provide resistance to tetracycline. In an aspect, the selectable marker gene is not a tetAR gene. In an aspect, the selectable marker gene is a counter-selectable marker gene. In an aspect, the counter-selectable marker gene is a sacB gene, a rpsL (strA) gene, a pheS gene, adhfr (folA) gene, a lacY gene, a Gata-1 gene, a ccdB gene, or a thyA− gene. In an aspect, the vector does not comprise SEQ ID NO: 61, or variants or fragments thereof. In an aspect, the vector does not comprise SEQ ID NO: 62, or variants or fragments thereof. In an aspect, the vector does not comprise a tra operon sequence or a trb operon sequence, or variants or fragments thereof. In an aspect, the vector does not comprise SEQ ID NO: 63, or variants or fragments thereof. In an aspect, the vector has SEQ ID NO: 34, or variants and fragments thereof. In an aspect, the vector has SEQ ID NO: 35, or variants and fragments thereof. In an aspect, the vector has SEQ ID NO: 36, or variants and fragments thereof.

In an aspect, the present disclosure further provides a kit comprising: (a) a vector comprising: (i) an origin of replication for propagation in Escherichia coli having SEQ ID NO: 2, or variants and fragments thereof; (ii) an origin of replication for propagation in Agrobacterium spp. having SEQ ID NO: 3, or variants and fragments thereof; (iii) a selectable marker gene having SEQ ID NO: 1, or variants and fragments thereof; and (iv) virulence genes comprising Agrobacterium spp. virulence genes virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D5 virulence genes having SEQ ID NOS: 18-22, respectively or r-virD1-D5 virulence genes having SEQ ID NOS: 94-98, respectively, virE1-E3 virulence genes having SEQ ID NOS: 23-25, respectively or a r-virE3 virulence gene having SEQ ID NO: 100, and a virG virulence gene having SEQ ID NO: 15 or a r-virG virulence gene having SEQ ID NO: 91, or variants and derivatives thereof, wherein the vector comprising the virulence genes r-virB1-B11, r-virC1-C2, r-virD1-D5, r-virE3, and r-virG further comprises a r-galls virulence gene having SEQ ID NO: 101, or variants and derivatives thereof; and (b) instructions for use in transformation of a plant using Agrobacterium or Ochrobactrum. In an aspect, the Rhizobiaceae virulence genes are Agrobacterium spp., Rhizobium spp., Sinorhizobium spp., Mesorhizobium spp., Phyllobacterium spp., Ochrobactrum spp., or Bradyrhizobium spp. virulence genes. In an aspect, the Rhizobiaceae virulence genes are Agrobacterium spp. virulence genes. In an aspect, the Agrobacterium spp. virulence genes are Agrobacterium albertimagni, Agrobacterium larrymoorei, Agrobacterium radiobacter, Agrobacterium rhizogenes, Agrobacterium rubi, Agrobacterium tumefaciens, or Agrobacterium vitis virulence genes. In an aspect, the Agrobacterium spp. virulence genes are Agrobacterium rhizogenes or Agrobacterium tumefaciens virulence genes. In an aspect, the Agrobacterium spp. virulence genes are Agrobacterium rhizogenes virulence genes. In an aspect, the Agrobacterium spp. virulence genes are Agrobacterium tumefaciens virulence genes. In an aspect, the Rhizobiaceae virulence genes are virB1-virB11 virulence genes having SEQ ID NOS: 4-14, respectively, or r-virB1-B11 virulence genes having SEQ ID NOS: 80-90, respectively, or variants and derivatives thereof. In an aspect, the Rhizobiaceae virulence genes are virC1-C2 virulence genes having SEQ ID NOS: 16-17, respectively, or r-virC1-C2 virulence genes having SEQ ID NOS: 92-93, respectively, or variants and derivatives thereof. In an aspect, the Rhizobiaceae virulence genes are virD1-D2 virulence genes having SEQ ID NOS: 18-19, respectively, or r-virD1-D2 virulence genes having SEQ ID NOS: 94-95, respectively, or variants and derivatives thereof. In an aspect, the Rhizobiaceae virulence gene is a virG virulence gene having SEQ ID NO: 15, or a r-virG virulence gene having SEQ ID NO: 91, or variants and derivatives thereof. In an aspect, the Rhizobiaceae virulence gene is a r-galls virulence gene having SEQ ID NO: 101, or variants and derivatives thereof. In an aspect, the vector further comprises one or more of Rhizobiaceae virulence genes virA, virD3, virD4, virD5, virE1, virE2, virE3, virH, virH1, virH2, virK, virL, virM, virP, virQ, r-virA , r-virD3, r-virD4, r-virD5, r-virE3, or r-virF or variants and derivatives thereof. In an aspect, the Rhizobiaceae virulence gene is a virA virulence gene having SEQ ID NO: 26 or a r-virA virulence gene having SEQ ID NO: 79, or variants and derivatives thereof. In an aspect, the Rhizobiaceae virulence genes are virD3-D5 virulence genes having SEQ ID NOS: 20-22, respectively, or r-virD3-D5 virulence genes having SEQ ID NO: 96-98, respectively, or variants and derivatives thereof. In an aspect, the Rhizobiaceae virulence genes are virE1-E3 virulence genes having SEQ ID NOS: 23-25, respectively, or a r-virE3 virulence gene having SEQ ID NO: 100, or variants and derivatives thereof. In an aspect, the Rhizobiaceae virulence genes are virH-H1 virulence genes having, SEQ ID NOS: 42-43, respectively, or variants and derivatives thereof. In an aspect, the Rhizobiaceae virulence gene is a virK virulence gene having SEQ ID NO: 45, or variants and derivatives thereof. In an aspect, the Rhizobiaceae virulence gene is a virL virulence gene having SEQ ID NO: 46, or variants and derivatives thereof. In an aspect, the Rhizobiaceae virulence gene is a virM virulence gene having SEQ ID NO: 47, or variants and derivatives thereof. In an aspect, the Rhizobiaceae virulence gene is a virP virulence gene having SEQ ID NO: 48, or variants and derivatives thereof. In an aspect, the Rhizobiaceae virulence gene is a virQ virulence gene having SEQ ID NO: 49, or variants and derivatives thereof. In an aspect, the vector further comprises the Rhizobiaceae virulence genes virD3-D5 and virE1-E3 or r-virD3-D5 and r-vir E3, or variants and derivatives thereof. In an aspect, the vector further comprises the Rhizobiaceae virulence genes virA, virD3-D5, and virE1-E3, or r-virA, r-virD3-D5, or r-virE3, or variants and derivatives thereof. In an aspect, the origin of replication for propagation and stable maintenance in Escherichia coli is derived from a Col E1, a pSC101, a p15A, or a R6K origin of replication, or functional variants and derivatives thereof. In an aspect, the origin of replication for propagation and stable maintenance in Escherichia coli is derived from a Col E1 origin of replication. In an aspect, the origin of replication derived from the ColE1 origin of replication has SEQ ID NO: 2, or variants and fragments thereof. In an aspect, the origin of replication for propagation and stable maintenance in Escherichia coli is derived from a pSC101 origin of replication. In an aspect, the origin of replication derived from the pSC101 origin of replication has SEQ ID NO: 50, or variants and fragments thereof. In an aspect, the origin of replication for propagation and stable maintenance in Escherichia coli is derived from a p15A origin of replication. In an aspect, the origin of replication derived from the p15A origin of replication has SEQ ID NO: 51, or variants and fragments thereof. In an aspect, the origin of replication for propagation and stable maintenance in Escherichia coli is derived from a R6K origin of replication. In an aspect, the origin of replication derived from the R6K origin of replication has SEQ ID NO: 52, or variants and fragments thereof. In an aspect, the origin of replication for propagation and stable maintenance in Agrobacterium spp. is a high copy number origin of replication. In an aspect, the origin of replication for propagation and stable maintenance in Agrobacterium spp. is an intermediate copy number origin of replication. In an aspect, the origin of replication for propagation and stable maintenance in Agrobacterium spp. is a low copy number origin of replication. In an aspect, the origin of replication for propagation and stable maintenance in Agrobacterium spp. is derived from a pRi, a pVS1, a pRSF1010, a pRK2, a pSa, or a pBBR1 origin of replication. In an aspect, the origin of replication for propagation and stable maintenance in Agrobacterium spp. is a variant of the pRK2 origin of replication. In an aspect, the origin of replication for propagation and stable maintenance in Agrobacterium spp. is derived from the pRSF1010 origin of replication. In an aspect, the origin of replication for propagation and stable maintenance in Agrobacterium spp. is derived from the pVS1 origin of replication. In an aspect, the origin of replication for propagation and stable maintenance in Agrobacterium spp. is derived from the pSa origin of replication. In an aspect, the origin of replication for propagation and stable maintenance in Agrobacterium spp. is an origin of replication having any one of SEQ ID NOS: 3, 37, 38, 53, 57, 58, 59, or 60, or variants and fragments thereof. In an aspect, the origin of replication for propagation and stable maintenance in Agrobacterium spp. is a repABC compatible origin of replication. In an aspect, the repABC compatible origin of replication has any one of SEQ ID NOS: 57, 58, 59, or 60, or variants and fragments thereof. In an aspect, the origin of replication for propagation and stable maintenance in Escherichia coli and the origin of replication for propagation and stable maintenance in Agrobacterium spp. are the same origin of replication. In an aspect, the origin of replication is derived from a pRK2 origin of replication, from a pSa origin of replication, or a pRSF1010 origin of replication. In an aspect, the origin of replication is derived from the pRK2 origin of replication. In an aspect, the pRK2 origin of replication has SEQ ID NO: 38, or variants and fragments thereof. In an aspect, the pRK2 origin of replication is a mini or micro pRK2 origin of replication. In an aspect, the pRK2 origin of replication is a micro pRK2 origin of replication. In an aspect, the micro pRK2 origin of replication has SEQ ID NO: 54, or variants and fragments thereof. In an aspect, the pRK2 origin of replication is a mini pRK2 origin of replication. In an aspect, the mini pRK2 has SEQ ID NO: 66, or variants and fragments thereof. In an aspect, the pRK2 origin of replication comprises the trfA and OriV sequences. In an aspect, the pRK2 origin of replication comprises SEQ ID NOS: 64 and 65, or variants and fragments thereof. In an aspect, the origin of replication is derived from the pSa origin of replication. In an aspect, the pSa origin of replication has SEQ ID NO: 53, or variants and fragments thereof. In an aspect, the origin of replication is derived from the pRSF1010 origin of replication. In an aspect, the pRSF1010 origin of replication has SEQ ID NO: 37, or variants and fragments thereof. In an aspect, the vector further comprises a sequence derived from the par DE operon. In an aspect, the par DE operon has SEQ ID NO: 55, or variants and fragments thereof. In an aspect, the selectable marker gene provides resistance to gentamicin, neomycin/kanamycin, hygromycin, or spectinomycin. In an aspect, the selectable marker gene is an aacC1 gene, a npt1gene, a npt2 gene, a hpt gene, an aadA gene, a SpcN gene, or an aph gene. In an aspect, the selectable marker gene is aacC1. In an aspect, the aacC1 selectable marker gene has SEQ ID NO: 1, or variants and fragments thereof. In an aspect, the selectable marker gene is aadA. In an aspect, the aadA selectable marker gene has SEQ ID NO: 39, or variants and fragments thereof. In an aspect, the selectable marker gene is npt1. In an aspect, the npt1 selectable marker gene has SEQ ID NO: 40, or variants and fragments thereof. In an aspect, the selectable marker gene is npt2. In an aspect, the npt2 selectable marker gene has SEQ ID NO: 41, or variants and fragments thereof. In an aspect, the selectable marker gene is hpt. In an aspect, the hpt selectable marker gene has SEQ ID NO: 67, or variants and fragments thereof. In an aspect, the selectable marker gene is SpcN. In an aspect, the SpcN selectable marker gene has SEQ ID NO: 77, or variants and fragments thereof. In an aspect, the selectable marker gene is aph. In an aspect, the aph selectable marker gene has SEQ ID NO: 78, or variants and fragments thereof. In an aspect, the selectable marker gene does not provide resistance to tetracycline. In an aspect, the selectable marker gene is not a tetAR gene. In an aspect, the selectable marker gene is a counter-selectable marker gene. In an aspect, the counter-selectable marker gene is a sacB gene, a rpsL (strA) gene, a pheS gene, adhfr (folA) gene, a lacY gene, a Gata-1 gene, a ccdB gene, or a thyA− gene. In an aspect, the vector does not comprise SEQ ID NO: 61, or variants or fragments thereof. In an aspect, the vector does not comprise SEQ ID NO: 62, or variants or fragments thereof. In an aspect, the vector does not comprise a tra operon sequence or a trb operon sequence, or variants or fragments thereof. In an aspect, the vector does not comprise SEQ ID NO: 63, or variants or fragments thereof. In an aspect, the vector has SEQ ID NO: 34, or variants and fragments thereof. In an aspect, the vector has SEQ ID NO: 35, or variants and fragments thereof. In an aspect, the vector has SEQ ID NO: 36, or variants and fragments thereof.

In an aspect, the present disclosure further provides a kit comprising: (a) a vector comprising: (i) an origin of replication for propagation in Escherichia coli having SEQ ID NO: 2, or variants and fragments thereof; (ii) an origin of replication for propagation in Agrobacterium spp. having SEQ ID NO: 3, or variants and fragments thereof; (iii) a selectable marker gene having SEQ ID NO: 1; and (iv) virulence genes comprising Agrobacterium spp. virulence genes a virA virulence gene having SEQ ID NO: 26 or a r-virA virulence gene having SEQ ID NO: 79, virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D5 virulence genes having SEQ ID NOS: 18-22, respectively or r-virD1-D5 virulence genes having SEQ ID NOS: 94-98, respectively, virE1-E3 virulence genes having SEQ ID NOS: 23-25, respectively or a r-virE3 virulence gene having SEQ ID NOS: 100, and a virG virulence gene having SEQ ID NO: 15 or a r-virG virulence gene having SEQ ID NO: 91, or variants and derivatives thereof, wherein the vector comprising the virulence genes r-virA, r-virB1-B11, r-virC1-C2, r-virD1-D5, r-virE3, and r-virG further comprises a r-galls virulence gene having SEQ ID NO: 101, or variants and derivatives thereof; and (b) instructions for use in transformation of a plant using Agrobacterium or Ochrobactrum. In an aspect, the Rhizobiaceae virulence genes are Agrobacterium spp., Rhizobium spp., Sinorhizobium spp., Mesorhizobium spp., Phyllobacterium spp., Ochrobactrum spp., or Bradyrhizobium spp. virulence genes. In an aspect, the Rhizobiaceae virulence genes are Agrobacterium spp. virulence genes. In an aspect, the Agrobacterium spp. virulence genes are Agrobacterium albertimagni, Agrobacterium larrymoorei, Agrobacterium radiobacter, Agrobacterium rhizogenes, Agrobacterium rubi, Agrobacterium tumefaciens, or Agrobacterium vitis virulence genes. In an aspect, the Agrobacterium spp. virulence genes are Agrobacterium rhizogenes or Agrobacterium tumefaciens virulence genes. In an aspect, the Agrobacterium spp. virulence genes are Agrobacterium rhizogenes virulence genes. In an aspect, the Agrobacterium spp. virulence genes are Agrobacterium tumefaciens virulence genes. In an aspect, the Rhizobiaceae virulence genes are virB1-virB11 virulence genes having SEQ ID NOS: 4-14, respectively, or r-virB1-B11 virulence genes having SEQ ID NOS: 80-90, respectively, or variants and derivatives thereof. In an aspect, the Rhizobiaceae virulence genes are virC1-C2 virulence genes having SEQ ID NOS: 16-17, respectively, or r-virC1-C2 virulence genes having SEQ ID NOS: 92-93, respectively, or variants and derivatives thereof. In an aspect, the Rhizobiaceae virulence genes are virD1-D2 virulence genes having SEQ ID NOS: 18-19, respectively, or r-virD1-D2 virulence genes having SEQ ID NOS: 94-95, respectively, or variants and derivatives thereof. In an aspect, the Rhizobiaceae virulence gene is a virG virulence gene having SEQ ID NO: 15, or a r-virG virulence gene having SEQ ID NO: 91, or variants and derivatives thereof. In an aspect, the Rhizobiaceae virulence gene is a r-galls virulence gene having SEQ ID NO: 101, or variants and derivatives thereof. In an aspect, the vector further comprises one or more of Rhizobiaceae virulence genes virA, virD3, virD4, virD5, virE1, virE2, virE3, virH, virH1, virH2, virK, virL, virM, virP, virQ, r-virA , r-virD3, r-virD4, r-virD5, or r-virE3, r-virF or variants and derivatives thereof. In an aspect, the Rhizobiaceae virulence gene is a virA virulence gene having SEQ ID NO: 26 or a r-virA virulence gene having SEQ ID NO: 79, or variants and derivatives thereof. In an aspect, the Rhizobiaceae virulence genes are virD3-D5 virulence genes having SEQ ID NOS: 20-22, respectively, or r-virD3-D5 virulence genes having SEQ ID NO: 96-98, respectively, or variants and derivatives thereof. In an aspect, the Rhizobiaceae virulence genes are virE1-E3 virulence genes having SEQ ID NOS: 23-25, respectively, or a r-virE3 virulence gene having SEQ ID NO: 100, or variants and derivatives thereof. In an aspect, the Rhizobiaceae virulence genes are virH-H1 virulence genes having, SEQ ID NOS: 42-43, respectively, or variants and derivatives thereof. In an aspect, the Rhizobiaceae virulence gene is a virK virulence gene having SEQ ID NO: 45, or variants and derivatives thereof. In an aspect, the Rhizobiaceae virulence gene is a virL virulence gene having SEQ ID NO: 46, or variants and derivatives thereof. In an aspect, the Rhizobiaceae virulence gene is a virM virulence gene having SEQ ID NO: 47, or variants and derivatives thereof. In an aspect, the Rhizobiaceae virulence gene is a virP virulence gene having SEQ ID NO: 48, or variants and derivatives thereof. In an aspect, the Rhizobiaceae virulence gene is a virQ virulence gene having SEQ ID NO: 49, or variants and derivatives thereof. In an aspect, the vector further comprises the Rhizobiaceae virulence genes virD3-D5 and virE1-E3 or r-virD3-D5 and r-vir E3, or variants and derivatives thereof. In an aspect, the vector further comprises the Rhizobiaceae virulence genes virA, virD3-D5, and virE1-E3, or r-virA, r-virD3-D5, and r-virE3, or variants and derivatives thereof. In an aspect, the origin of replication for propagation and stable maintenance in Escherichia coli is derived from a Col E1, a pSC101, a p15A, or a R6K origin of replication, or functional variants and derivatives thereof. In an aspect, the origin of replication for propagation and stable maintenance in Escherichia coli is derived from a Col E1 origin of replication. In an aspect, the origin of replication derived from the ColE1 origin of replication has SEQ ID NO: 2, or variants and fragments thereof. In an aspect, the origin of replication for propagation and stable maintenance in Escherichia coli is derived from a pSC101 origin of replication. In an aspect, the origin of replication derived from the pSC101 origin of replication has SEQ ID NO: 50, or variants and fragments thereof. In an aspect, the origin of replication for propagation and stable maintenance in Escherichia coli is derived from a p15A origin of replication. In an aspect, the origin of replication derived from the p15A origin of replication has SEQ ID NO: 51, or variants and fragments thereof. In an aspect, the origin of replication for propagation and stable maintenance in Escherichia coli is derived from a R6K origin of replication. In an aspect, the origin of replication derived from the R6K origin of replication has SEQ ID NO: 52, or variants and fragments thereof. In an aspect, the origin of replication for propagation and stable maintenance in Agrobacterium spp. is a high copy number origin of replication. In an aspect, the origin of replication for propagation and stable maintenance in Agrobacterium spp. is an intermediate copy number origin of replication. In an aspect, the origin of replication for propagation and stable maintenance in Agrobacterium spp. is a low copy number origin of replication. In an aspect, the origin of replication for propagation and stable maintenance in Agrobacterium spp. is derived from a pRi, a pVS1, a pRSF1010, a pRK2, a pSa, or a pBBR1 origin of replication. In an aspect, the origin of replication for propagation and stable maintenance in Agrobacterium spp. is a variant of the pRK2 origin of replication. In an aspect, the origin of replication for propagation and stable maintenance in Agrobacterium spp. is derived from the pRSF1010 origin of replication. In an aspect, the origin of replication for propagation and stable maintenance in Agrobacterium spp. is derived from the pVS1 origin of replication. In an aspect, the origin of replication for propagation and stable maintenance in Agrobacterium spp. is derived from the pSa origin of replication. In an aspect, the origin of replication for propagation and stable maintenance in Agrobacterium spp. is an origin of replication having any one of SEQ ID NOS: 3, 37, 38, 53, 57, 58, 59, or 60, or variants and fragments thereof. In an aspect, the origin of replication for propagation and stable maintenance in Agrobacterium spp. is a repABC compatible origin of replication. In an aspect, the repABC compatible origin of replication has any one of SEQ ID NOS: 57, 58, 59, or 60, or variants and fragments thereof. In an aspect, the origin of replication for propagation and stable maintenance in Escherichia coli and the origin of replication for propagation and stable maintenance in Agrobacterium spp. are the same origin of replication. In an aspect, the origin of replication is derived from a pRK2 origin of replication, from a pSa origin of replication, or a pRSF1010 origin of replication. In an aspect, the origin of replication is derived from the pRK2 origin of replication. In an aspect, the pRK2 origin of replication has SEQ ID NO: 38, or variants and fragments thereof. In an aspect, the pRK2 origin of replication is a mini or micro pRK2 origin of replication. In an aspect, the pRK2 origin of replication is a micro pRK2 origin of replication. In an aspect, the micro pRK2 origin of replication has SEQ ID NO: 54, or variants and fragments thereof. In an aspect, the pRK2 origin of replication is a mini pRK2 origin of replication. In an aspect, the mini pRK2 has SEQ ID NO: 66, or variants and fragments thereof. In an aspect, the pRK2 origin of replication comprises the trfA and OriV sequences. In an aspect, the pRK2 origin of replication comprises SEQ ID NOS: 64 and 65, or variants and fragments thereof. In an aspect, the origin of replication is derived from the pSa origin of replication. In an aspect, the pSa origin of replication has SEQ ID NO: 53, or variants and fragments thereof. In an aspect, the origin of replication is derived from the pRSF1010 origin of replication. In an aspect, the pRSF1010 origin of replication has SEQ ID NO: 37, or variants and fragments thereof. In an aspect, the vector further comprises a sequence derived from the par DE operon. In an aspect, the par DE operon has SEQ ID NO: 55, or variants and fragments thereof. In an aspect, the selectable marker gene provides resistance to gentamicin, neomycin/kanamycin, hygromycin, or spectinomycin. In an aspect, the selectable marker gene is an aacC1 gene, a npt1 gene, a npt2 gene, a hpt gene, an aadA gene, a SpcN gene, or an aph gene. In an aspect, the selectable marker gene is aacC1. In an aspect, the aacC1 selectable marker gene has SEQ ID NO: 1, or variants and fragments thereof. In an aspect, the selectable marker gene is aadA. In an aspect, the aadA selectable marker gene has SEQ ID NO: 39, or variants and fragments thereof. In an aspect, the selectable marker gene is npt1. In an aspect, the nptl selectable marker gene has SEQ ID NO: 40, or variants and fragments thereof. In an aspect, the selectable marker gene is npt2. In an aspect, the npt2 selectable marker gene has SEQ ID NO: 41, or variants and fragments thereof. In an aspect, the selectable marker gene is hpt. In an aspect, the hpt selectable marker gene has SEQ ID NO: 67, or variants and fragments thereof. In an aspect, the selectable marker gene is SpcN. In an aspect, the SpcN selectable marker gene has SEQ ID NO: 77, or variants and fragments thereof. In an aspect, the selectable marker gene is aph. In an aspect, the aph selectable marker gene has SEQ ID NO: 78, or variant and fragments thereof. In an aspect, the selectable marker gene does not provide resistance to tetracycline. In an aspect, the selectable marker gene is not a tetAR gene. In an aspect, the selectable marker gene is a counter-selectable marker gene. In an aspect, the counter-selectable marker gene is a sacB gene, a rpsL (strA) gene, a pheS gene, adhfr (folA) gene, a lacY gene, a Gata-1 gene, a ccdB gene, or a thyA− gene. In an aspect, the vector does not comprise SEQ ID NO: 61, or variants or fragments thereof. In an aspect, the vector does not comprise SEQ ID NO: 62, or variants or fragments thereof. In an aspect, the vector does not comprise a tra operon sequence or a trb operon sequence, or variants or fragments thereof. In an aspect, the vector does not comprise SEQ ID NO: 63, or variants or fragments thereof. In an aspect, the vector has SEQ ID NO: 34, or variants and fragments thereof. In an aspect, the vector has SEQ ID NO: 35, or variants and fragments thereof. In an aspect, the vector has SEQ ID NO: 36, or variants and fragments thereof.

### BRIEF DESCRIPTION OF THE DRAWINGS

FIG. 1 shows a plasmid map of vector pSB1.

FIG. 2 shows a plasmid map of vector pPHP70298, which has the sequence of SEQ ID NO: 34.

FIG. 3 shows a plasmid map of vector of pPHP71539, which has the sequence of SEQ ID NO: 35.

FIG. 4 shows a plasmid map of vector of pPHP79761, which has the sequence of SEQ ID NO: 36, with location of unique restriction enzyme sites. These restriction sites were included between functional elements to facilitate their exchange with other functionally equivalent elements.

FIG. 5 shows a diagram showing the relationship of the indicated genes on a 37,171 bp fragment from the Agrobacterium tumefaciens Ti plasmid pTiBo542, NCBI Reference Sequence: NC_010929.1

FIG. 6 shows a diagram showing the relationship of the indicated genes 2,600 bp fragment comprising the pVS1 origin of replication.

FIG. 7A shows transient RFP (Ds-RED) expression in wheat cultivar HC0456D 15 days post infection; strain AGL1. FIG. 7B shows transient RFP expression after 15 days post infenction; strain LBA4404Thy-71539. Arrows indicate the areas with strong Ds-RED expression. Scale bar equals 2 mm.

### DETAILED DESCRIPTION

The disclosures herein will be described more fully hereinafter with reference to the accompanying drawings, in which some, but not all possible aspects are shown. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed compositions and methods belong. In this specification and in the claims which follow, reference will be made to a number of terms which shall be defined herein.

### I. Methods and Compositions for Improved Vectors Comprising Vir Genes

The present disclosure comprises methods and compositions for the vectors comprising vir genes.

As used herein, “pPHP” refers to plasmid PHP, which is than followed by numerical digits. For example, pPHP70298 refers to plasmid PHP70298. For example, pVIR7 refers to plasmid VIR7.

Table 23 provides a list of sequence identification numbers (SEQ ID NO:) provided in this disclosure.

In an aspect, the present disclosure provides a vector comprising: (a) an origin of replication for propagation and stable maintenance in Escherichia coli; (b) an origin of replication for propagation and stable maintenance in Agrobacterium spp.; (c) a selectable marker gene; and (d) Rhizobiaceae virulence genes virB1-B11, virC1-C2, virD1-D2, and virG or Rhizobiaceae virulence genes r-virB1-B11, r-virC1-C2, r-virD1-D2, r-virG, and r-galls, or variants and derivatives thereof. In an aspect, the Rhizobiaceae virulence genes virB1-virB11 have SEQ ID NOS: 4-14, respectively, or the Rhizobiaceae virulence genes r-virB1-B11 have SEQ ID NOS: 80-90, respectively, or variants and derivatives thereof; the Rhizobiaceae virulence genes virC1-C2 have SEQ ID NOS: 16-17, respectively, or the Rhizobiaceae virulence genes r-virC1-C2 have SEQ ID NOS: 92-93, respectively, or variants and derivatives thereof; the Rhizobiaceae virulence genes virD1-D2 have SEQ ID NOS: 18-19, or the Rhizobiaceae virulence genes r-virD1-D2 have SEQ ID NOS: 94-95, respectively, or variants and derivatives thereof; the Rhizobiaceae virulence gene virG has SEQ ID NO: 15, or the Rhizobiaceae virulence gene r-virG has SEQ ID NO: 91, respectively, or variants and derivatives thereof; and the Rhizobiaceae virulence gene r-galls has SEQ ID NO: 101, or variants and derivatives thereof.

In aspects, the Rhizobiaceae virulence genes are Agrobacterium spp., Rhizobium spp., Sinorhizobium spp., Mesorhizobium spp., Phyllobacterium spp., Ochrobactrum spp., or Bradyrhizobium spp. genes. In an aspect, the Rhizobiaceae virulence genes are Rhizobium spp. genes. In an aspect, the Rhizobiaceae virulence genes are Sinorhizobium spp. genes. In an aspect, the Rhizobiaceae virulence genes are Mesorhizobium spp. genes. In an aspect, the Rhizobiaceae virulence genes are Phyllobacterium spp. genes. In an aspect, the Rhizobiaceae virulence genes are Ochrobactrum spp. genes. In an aspect, the Rhizobiaceae virulence genes are Bradyrhizobium spp. genes.

In an aspect, the Rhizobiaceae virulence genes are Agrobacterium spp. genes. In an aspect, the Agrobacterium spp. genes are Agrobacterium albertimagni, Agrobacterium larrymoorei, Agrobacterium radiobacter, Agrobacterium rhizogenes, Agrobacterium rubi, Agrobacterium tumefaciens, or Agrobacterium vitis genes. In an aspect, the Agrobacterium spp. genes are Agrobacterium rhizogenes or Agrobacterium tumefaciens. In an aspect, the Agrobacterium spp. genes are Agrobacterium rhizogenes. In an aspect, the Agrobacterium spp. genes are Agrobacterium tumefaciens.

A number of wild-type and disarmed (non-pathogenic) strains of Agrobacterium tumefaciens and Agrobacterium rhizogenes harboring Ti or Ri plasmids can be used for gene transfer into plants. Phytohormone synthesis genes located in the T-DNA of wild type Agrobacteria harboring a Ti or Ri plasmid are expressed in plant cells following transformation, and cause tumor formation or a hairy root phenotype depending on the Agrobacterium strain or species. The T-DNA of Agrobacteria can be engineered to replace many of its virulence and pathogenicity determinants (by disarming) with one or more sequences of interest and retain the ability to transfer the modified T-DNA into a plant cell and be integrated into a genome. Strains containing such disarmed Ti plasmids are widely used for plant transformation.

In some aspects, a construct comprises a Ti plasmid (Agrobacterium tumefaciens) or a Ri plasmid (Agrobacterium rhizogenes). In some aspects, the construct comprises one or more virulence genes. The virulence genes can be from a Ti plasmid and are represented herein as SEQ ID NOS: 4-27 and SEQ ID NOS: 42-49. The virulence genes can be from a Ri plasmid and are represented herein as SEQ ID NOS: 79-101. The Ri plasmid virulence genes disclosed herein are represented using a “r” before the vir gene name. For example, r-virA (SEQ ID NO: 79), r-virB1 (SEQ ID NO: 80), r-virB2 (SEQ ID NO: 81), r-virB3 (SEQ ID NO: 82), r-virB4 (SEQ ID NO: 83), r-virB5 (SEQ ID NO: 84), r-virB6 (SEQ ID NO: 85), r-virB7 (SEQ ID NO: 86), r-virB8 (SEQ ID NO: 87), r-virB9 (SEQ ID NO: 88), r-virB10 (SEQ ID NO: 89), r-virB11 (SEQ ID NO: 90), r-virG (SEQ ID NO: 91), r-virC1 (SEQ ID NO: 92), r-virC2 (SEQ ID NO: 93), r-virD1 (SEQ ID NO: 94), r-virD2 (SEQ ID NO: 95), r-virD3 (SEQ ID NO: 96), r-virD4 (SEQ ID NO: 97), r-virD5 (SEQ ID NO: 98), r-virF (SEQ ID NO: 99), r-virE3 (SEQ ID NO: 100), and r-galls (SEQ ID NO: 101). See Table 23 herein. Different combinations of the virulence genes may be used herein. The r-galls gene (SEQ ID NO: 101) is necessary for virulence with the Ri plasmid vir genes described herein.

The Vir region on the Ti/Ri plasmid is a collection of genes whose aggregate function is to excise the T-DNA region of the plasmid and promote its transfer and integration into the plant genome. The vir system is induced by signals produced by plants in response to wounding. Phenolic compounds such as acetosyringone, syringealdehyde, or acetovanillone activate the virA gene, which encodes a receptor that is a constitutively expressed trans-membrane protein. The activated virA gene acts as a kinase, phosphorylating the virG gene. In its phosphorylated form, virG acts as a transcriptional activator for the remaining vir gene operons. The virB operon encodes proteins which produce a pore/pilus-like structure. VirC binds to the overdrive sequence. VirD1 and virD2 have endonuclease activity, and make single-stranded cuts within the left and right borders, and virD4 is a coupling protein. VirE binds to the single stranded T-DNA, protecting it during the transport phase of the process. Once in the plant cell, the complementary strand of the T-DNA is synthesized.

These and other vir genes, function in trans, so none of these genes need to be included in the cloning vectors. For example, modified Agrobacterium strains can provide all the necessary Vir functions on plasmids where the T-DNA region has been deleted, allowing the cell to provide the vir functions for T-DNA transfer. In one example, there are C58-derived strains in which a portion of pBR322 was used to replace the T-DNA region, and providing resistance to ampicillin.

Provided are constructs which include one or more sequence of interest for expression and/or insertion in a cell genome. The constructs may be contained within a vector such as binary, ternary or T-DNA vectors. A construct refers to a polynucleotide molecule comprised of various types of nucleotide sequences having different functions and/or activities. Various types of sequences include linkers, adapters, regulatory regions, introns, restriction sites, enhancers, insulators, screenable markers, selectable markers, promoters, expression cassettes, coding polynucleotides, silencing polynucleotides, termination sequences, origins of replication, recombination sites, excision cassettes, recombinases, cell proliferation factors, promoter traps, other sites that aid in vector construction or analysis, or any combination thereof. In some examples a construct comprises one or more expression cassettes, wherein a polynucleotide is operably linked to a regulatory sequence. Operably linked is a functional linkage between two or more elements. For example, an operable linkage between a coding polynucleotide and a regulatory sequence (e.g., a promoter) is a functional link that allows for expression of the coding polynucleotide. Operably linked elements may be contiguous or non-contiguous. When used to refer to the joining of two protein coding regions, by operably linked is intended that the coding regions are in the same reading frame. A coding polynucleotide includes any polynucleotide that either encodes a polypeptide, or that encodes a silencing polynucleotide that reduces the expression of target genes. Non-limiting examples of a silencing polynucleotide include a small interfering RNA, micro RNA, antisense RNA, a hairpin structure, and the like. The construct may also contain a number of genetic components to facilitate transformation of the plant cell or tissue and to regulate expression of any structural nucleic acid sequence. In some examples, the genetic components are oriented so as to express a mRNA, optionally the mRNA is translated into a protein. The expression of a plant structural coding sequence (a gene, cDNA, synthetic DNA, or other DNA) that exists in double-stranded form involves transcription of messenger RNA (mRNA) from one strand of the DNA by RNA polymerase enzyme and subsequent processing of the mRNA primary transcript inside the nucleus. This processing involves a 3′ non-translated region that polyadenylates the 3′ ends of the mRNA.

In an aspect, the present disclosure provides a vector comprising: (a) an origin of replication for propagation and stable maintenance in Escherichia coli; (b) an origin of replication for propagation and stable maintenance in Agrobacterium spp.; (c) a selectable marker gene; and (d) Agrobacterium virulence genes virB1-B11; virC1-C2; virD1-D2; and virG, or the Agrobacterium virulence genes r-virB1-B11, r-virC1-C2, r-virD1-D2, r-virG, and r-galls, or variants and derivatives thereof. In an aspect, the Agrobacterium virulence genes virB1-B11 have SEQ ID NOS: 4-14, respectively, or variants and derivatives thereof; the Agrobacterium virulence genes virC1-C2 have SEQ ID NOS: 16-17, respectively, or variants and derivatives thereof; the Agrobacterium spp. virulence genes virD1-D2 have SEQ ID NOS: 18-19, respectively, or variants and derivatives thereof; and the Agrobacterium virulence gene virG has SEQ ID NO: 15, or variants and derivatives thereof; or the Agrobacterium virulence genes r-virB1-B11 have SEQ ID NOS: 80-90, respectively, or variants and derivatives thereof, r-virC1-C2 have SEQ ID NOS: 92-93, respectively, or variants and derivatives thereof, r-virD1-D2 have SEQ ID NOS: 95-96, respectively, or variants and derivatives thereof, r-virG has SEQ ID NO: 91, or variants and derivatives thereof, and r-galls has SEQ ID NO: 101, or variants and derivatives thereof.

In an aspect, the vector further comprises one or more Rhizobiaceae virulence genes virA, virD3, virD4, virD5, virE1, virE2, virE3, virH, virH1, virH2, vir J, virK, virL, virM, virP, or virQ, or variants and derivatives thereof, or one or more Rhizobiaceae virulence genes r-virA , r-virD3, r-virD4, r-virD5, r-virE3, or r-virF, or variants and derivatives thereof, wherein the vector comprising the virulence genes r-virA, r-virD3, r-virD4, r-virD5, r-virE3, and r-virF further comprises a r-galls virulence gene, or variants and derivatives thereof. Thus, in an aspect, the present disclosure provides a vector comprising: (a) an origin of replication for propagation and stable maintenance in Escherichia coli; (b) an origin of replication for propagation and stable maintenance in Agrobacterium spp.; (c) a selectable marker gene; and (d) Rhizobiaceae virulence genes virB1-B11; virC1-C2; virD1-D2, and virG, or variants and derivatives thereof, or the Rhizobiaceae virulence genes r-virB1-B11, r-virC1-C2, r-virD1-D2, and r-virG, or variants and derivatives thereof and r-galls, or variants and derivatives thereof; and optionally one or more Rhizobiaceae virulence genes virA, virD3, virD4, virD5, virE1, virE2, virE3, virH, virH1, virH2, vir J, virK, virL, virM, virP, or virQ, or variants and derivatives thereof or one or more Rhizobiaceae virulence genes r-virA , r-virD3, r-virD4, r-virD5, r-virE3, or r-virF, or variants and derivatives thereof and r-galls, or variants and derivatives thereof. In an aspect, the Rhizobiaceae virulence gene virA has SEQ ID NO: 26 or the Rhizobiaceae virulence gene r-virA has SEQ ID NO: 79, or variants and derivatives thereof. In an aspect, the Rhizobiaceae virulence genes virD3-D5 have SEQ ID NOS: 20-22, respectively, or the Rhizobiaceae virulence genes r-virD3-D5 have SEQ ID NO: 94-96, respectively, or variants and derivatives thereof. In an aspect, the Rhizobiaceae virulence genes virE1-E3 have SEQ ID NOS: 23-25, respectively, or the Rhizobiaceae virulence gene r-virE3 has SEQ ID NO: 100, or variants and derivatives thereof. In an aspect, the Rhizobiaceae virulence genes virH-H1 have SEQ ID NOS: 42-43 respectively, or variants and derivatives thereof. In an aspect, the Rhizobiaceae virulence gene virJ has SEQ ID NO: 27, or variants and derivatives thereof. In an aspect, the Rhizobiaceae virulence gene virK has SEQ ID NO: 45, or variants and derivatives thereof. In an aspect, the Rhizobiaceae virulence gene virL has SEQ ID NO: 46, or variants and derivatives thereof. In an aspect, the Rhizobiaceae virulence gene virM has SEQ ID NO: 47, or variants and derivatives thereof. In an aspect, the Rhizobiaceae virulence gene virP has SEQ ID NO: 48, or variants and derivatives thereof. In an aspect, the Rhizobiaceae virulence gene virQ has SEQ ID NO: 49, or variants and derivatives thereof.

In an aspect, the vector further comprises one or more Agrobacterium virulence genes virA, virD3, virD4, virD5, virE1, virE2, virE3, virH, virH1, virH2, vir J, virK, virL, virM, virP, or virQ, or variants and derivatives thereof, or one or more Agrobacterium virulence genes r-virA , r-virD3, r-virD4, r-virD5, r-virE3, or r-virF, or variants and derivatives thereof, and r-galls, or variants and derivatives thereof. Thus, in an aspect, the present disclosure provides a vector comprising: (a) an origin of replication for propagation and stable maintenance in Escherichia coli; (b) an origin of replication for propagation and stable maintenance in Agrobacterium spp.; (c) a selectable marker gene; and (d) Agrobacterium virulence genes virB1-B11; virC1-C2; virD1-D2; and virG, or variants and derivatives thereof, or the Agrobacterium virulence genes r-virB1-B11, r-virC1-C2, r-virD1-D2, r-virG, and r-galls, or variants and derivatives thereof; and optionally one or more Agrobacterium virulence genes virA, virD3, virD4, virD5, virE1, virE2, virE3, virH, virH1, virH2, vir J, virK, virL, virM, virP, or virQ, or variants and derivatives thereof, or one or more Agrobacterium virulence genes r-virA, r-virD3, r-virD4, r-virD5, r-virE3, or r-virF or variants and derivatives thereof, and r-galls, or variants and derivatives thereof. In an aspect, the Rhizobiaceae virulence gene virA has SEQ ID NO: 26 or the Rhizobiaceae virulence gene r-virA has SEQ ID NO: 79, or variants and derivatives thereof. In an aspect, the Rhizobiaceae virulence genes virD3-D5 have SEQ ID NOS: 20-22, respectively, or the Rhizobiaceae virulence genes r-virD3-D5 have SEQ ID NO: 94-96, respectively, or variants and derivatives thereof. In an aspect, the Rhizobiaceae virulence genes virE1-E3 have SEQ ID NOS: 23-25, respectively, or the Rhizobiaceae virulence gene r-virE3 has SEQ ID NO: 100, or variants and derivatives thereof. In an aspect, the Rhizobiaceae virulence genes virH-H2 have SEQ ID NOS: 42-43, respectively, or variants and derivatives thereof. In an aspect, the Rhizobiaceae virulence gene virJ has SEQ ID NO: 27, or variants and derivatives thereof. In an aspect, the Rhizobiaceae virulence gene virK has SEQ ID NO: 45, or variants and derivatives thereof. In an aspect, the Rhizobiaceae virulence gene virL has SEQ ID NO: 46, or variants and derivatives thereof. In an aspect, the Rhizobiaceae virulence gene virM has SEQ ID NO: 47, or variants and derivatives thereof. In an aspect, the Rhizobiaceae virulence gene virP has SEQ ID NO: 48, or variants and derivatives thereof. In an aspect, the Rhizobiaceae virulence gene virQ has SEQ ID NO: 49, or variants and derivatives thereof. In an aspect, the Rhizobiaceae virulence gene r-virF has SEQ ID NO: 99, or variants and derivatives thereof. In an aspect, the Rhizobiaceae virulence gene r-galls has SEQ ID NO: 101, or variants and derivatives thereof.

In an aspect, the present disclosure provides a vector comprising: (a) an origin of replication for propagation and stable maintenance in Escherichia coli; (b) an origin of replication for propagation and stable maintenance in Agrobacterium spp.; (c) a selectable marker gene; and (d) Agrobacterium virulence genes virB1-B11; virC1-C2; virD1-D5; virE1-E3; and virG, or variants and derivatives thereof, or the Agrobacterium virulence genes r-virB1-B11 having SEQ ID NOS: 80-90, respectively, r-virC1-C2 having SEQ ID NOS: 92-93, respectively, r-virD1-D5 having SEQ ID NOS: 94-98, respectively, r-vir-E3 having SEQ ID NOS: 100, r-virG having SEQ ID NO: 91, and r-galls having SEQ ID NO: 101, or variants and derivatives thereof.

In an aspect, the present disclosure provides a vector comprising: (a) an origin of replication for propagation and stable maintenance in Escherichia coli; (b) an origin of replication for propagation and stable maintenance in Agrobacterium spp.; (c) a selectable marker gene; and (d) Agrobacterium virulence genes virA; virB1-B11; virC1-C2; virD1-D5; virE1-E3; and virG, or variants and derivatives thereof, or the Agrobacterium virulence genes r-virB1-B11; r-virC1-C2; r-virD1-D2; r-virG; and r-galls, or variants and derivatives thereof.

In an aspect, the present disclosure provides a vector comprising: (a) an origin of replication for propagation and stable maintenance in Escherichia coli; (b) an origin of replication for propagation and stable maintenance in Agrobacterium spp.; (c) a selectable marker gene; and (d) Agrobacterium virulence genes virA; virB1-B11; virC1-C2; virD1-D5; virE1-E3; virG; and virJ, or variants and derivatives thereof, or the Agrobacterium virulence genes r-virA having SEQ ID NO: 79, r-virB1-B11 having SEQ ID NOS: 80-90, respectively, r-virC1-C2 having SEQ ID NOS: 92-93, respectively, r-virD1-D5 having SEQ ID NOS: 94-98, respectively, r-virE3 having SEQ ID NO: 100, r-virG having SEQ ID NO: 91, and r-galls having SEQ ID NO: 101, or variants and derivatives thereof.

The present disclosure provides a vector comprising an origin of replication for propagation and stable maintenance in Escherichia coli derived from a Col E1, pSC101, p15A, or R6K origin of replication, or functional variants and derivatives thereof. In an aspect, any origin(s) of replication functional in Agrobacterium can be used in constructing the disclosed vectors. For example, different origins of replication can be selected in order to achieve different frequencies and qualities (single T-DNA copy, no backbone) of transformation events. In an aspect, the origin(s) of replication is an origin that is functional in Agrobacterium, E. coli, or both. In an aspect, the origin(s) of replication is selected from the group consisting of pVS1, pSa, RK2, pRi, incPa, incW, Co lE1, pRSF1010, pBBR1 or functional variants and derivatives thereof.

In an aspect, the vector comprises an origin of replication for propagation and stable maintenance in Escherichia coli derived from a Col E1 origin of replication. In a further aspect, the vector comprises an origin of replication for propagation and stable maintenance in Escherichia coli derived from a Col E1 origin of replication, having SEQ ID NO: 2, or variants and fragments thereof.

In an aspect, the vector comprises an origin of replication for propagation and stable maintenance in Escherichia coli derived from a pSC101 origin of replication. In a further aspect, the vector comprises an origin of replication for propagation and stable maintenance in Escherichia coli derived from a pSC101 origin of replication, having SEQ ID NO: 50, or variants and fragments thereof.

In an aspect, the vector comprises an origin of replication for propagation and stable maintenance in Escherichia coli derived from a p 15A origin of replication. In a further aspect, the vector comprises an origin of replication for propagation and stable maintenance in Escherichia coli derived from a p15A origin of replication, having SEQ ID NO: 51, or variants and fragments thereof.

In an aspect, the vector comprises an origin of replication for propagation and stable maintenance in Escherichia coli derived from a R6K origin of replication. In a further aspect, the vector comprises an origin of replication for propagation and stable maintenance in Escherichia coli derived from a R6K origin of replication, having SEQ ID NO: 52, or variants and fragments thereof.

In various aspects, the origin of replication for propagation and stable maintenance in Agrobacterium spp. can be a low copy number origin of replication, an intermediate copy number origin of replication, or a high copy number origin of replication. It is understood that a low copy number of origin of replication provides for about 1-2 copies of the vector per cell (e.g., see Li et al., Plant Cell Report (2015) 34:745-54; and Cho and Winans Proc. Natl. Acad. Sci. USA (2005) 102:14843-848). Further, it is understood that an intermediate copy number of origin of replication provides for about 7-12 copies of the vector per cell (e.g., see Oltmanns, et al., Plant Physiol. (2010) 152:1158-1166). Exemplary, but non-limiting examples, of intermediate copy number origins of replication include those derived from pRK2 and copy down variants of pVS1. It is to be appreciated that a high copy number of origin of replication provides for about 15-20 and greater copies of the vector per cell (e.g., see Oltmanns, et al., Plant Physiol. (2010) 152:1158-1166; and Li et al., Plant Cell Report (2015) 34:745-54). Exemplary, but non-limiting examples, of intermediate copy number origins of replication include those derived from repABC and copy up variants of pVS1.

In an aspect, origin of replication for propagation and stable maintenance in Agrobacterium spp. is derived from a pRi, pVS1, pRSF1010, pRK2, pSa, or pBBR1 origin of replication. In an aspect, the origin of replication for propagation and stable maintenance in Agrobacterium spp. is derived from the pRK2 origin of replication. In an aspect, the origin of replication for propagation and stable maintenance in Agrobacterium spp. is derived from the pRSF1010 origin of replication. In an aspect, the origin of replication for propagation and stable maintenance in Agrobacterium spp. is derived from the pVS1 origin of replication. In an aspect, the origin of replication for propagation and stable maintenance in Agrobacterium spp. is derived from the pSa origin of replication. In various aspects, the origin of replication for propagation and stable maintenance in Agrobacterium spp. has SEQ ID NO: 3, 37, 38, 57, 58, 59, or 60, or variants and fragments thereof.

In an aspect, the origin of replication for propagation and stable maintenance in Agrobacterium spp. is a repABC compatible origin of replication. The repABC compatible origin of replication can have SEQ ID NOS: 57, 58, 59, or 60, or variants and fragments thereof.

In some aspects, the origin of replication for propagation and stable maintenance in Escherichia coli and the origin of replication for propagation and stable maintenance in Agrobacterium spp. are the same origin of replication. For example, the origin of replication for propagation and stable maintenance in Escherichia coli and the origin of replication for propagation and stable maintenance in Agrobacterium spp. can be derived from a pRK2 origin of replication, from a pSa origin of replication, or a pRSF1010 origin of replication. In an aspect, the origin of replication for propagation and stable maintenance in Escherichia coli and the origin of replication for propagation and stable maintenance in Agrobacterium spp. can be derived from the pRK2 origin of replication. In a further aspect, the origin of replication for propagation and stable maintenance in Escherichia coli and the origin of replication for propagation and stable maintenance in Agrobacterium spp. can be derived from the pRK2 origin of replication has SEQ ID NO: 38, or variants and fragments thereof. In an aspect, the origin of replication is derived from the pSa origin of replication. In an aspect, the origin of replication for propagation and stable maintenance in Escherichia coli and the origin of replication for propagation and stable maintenance in Agrobacterium spp. can be derived from the pSa origin of replication (SEQ ID NO: 53), or variants and fragments thereof. In a further aspect, the origin of replication for propagation and stable maintenance in Escherichia coli and the origin of replication for propagation and stable maintenance in Agrobacterium spp. can be derived from the origin of replication pRSF1010 origin of replication. In a further aspect, the pRSF1010 origin of replication has SEQ ID NO: 37, or variants and fragments thereof.

Variants of the pRK2 origin of replication include a mini or micro pRK2 origin of replication. In an aspect, the origin of replication for propagation and stable maintenance in Escherichia coli and the origin of replication for propagation and stable maintenance in Agrobacterium spp. is a micro pRK2 origin of replication. In a further aspect, the origin of replication for propagation and stable maintenance in Escherichia coli and the origin of replication for propagation and stable maintenance in Agrobacterium spp. is a micro pRK2 origin of replication and has SEQ ID NO: 54, or variants and fragments thereof.

In aspects, the disclosed vector further comprises a sequence derived from the par DE operon. In a further aspect, the disclosed vector comprising a pRK2 origin of replication, in particular a micro or mini pRK2 origin of replication, can further comprise a sequence derived from the par DE operon. The par DE operon sequence can have SEQ ID NO: 55, or variants and fragments thereof.

In an aspect, the selectable marker provides resistance to gentamicin, neomycin/kanamycin, hygromycin, or spectinomycin. In a further aspect, the selectable marker is aacC1, npt1, npt2, hpt, aadA, SpcN, or aph. In an aspect, the selectable marker has SEQ ID NO: 1, 39, 40, 41, 67, 77 or 78, or variants and fragments thereof, corresponding, respectively, to aacC1, aadA, npt1, npt2, hpt, SpcN, and aph. In an aspect, the selectable marker is aacC1. In a further aspect, the selectable marker is aacC1, and has SEQ ID NO: 1, or variants and fragments thereof. In an aspect, the selectable marker is aadA. In a further aspect, the selectable marker is aadA, and has SEQ ID NO: 39, or variants and fragments thereof. In an aspect, the selectable marker is npt1. In a further aspect, the selectable marker is npt1, and has SEQ ID NO: 40, or variants and fragments thereof. In an aspect, the selectable marker is npt2. In a further aspect, the selectable marker is npt2, and has SEQ ID NO: 41. In an aspect, the selectable marker is hpt. In a further aspect, the selectable marker is hpt, and has SEQ ID NO: 67, or variants and fragments thereof. In a further aspect, the selectable marker is SpcN, and has SEQ ID NO: 77, or variants and fragments thereof. In a further aspect, the selectable marker is aph, and has SEQ ID NO: 78, or variants and fragments thereof. In various aspects, the selectable marker is not a tetracycline selectable marker. In an aspect, the selectable marker is not tetAR.

In an aspect, the selectable marker is a counter-selectable marker or negative selectable marker. As can be appreciated, it is understood that the disclosed vector can comprise particular combinations of virulence (vir) genes, origins of replication, and selectable markers. Table 1 below provides exemplary, but not limiting, combinations comprising virulence genes, selectable marker(s), and origins of replication. In an aspect, the counter-selectable marker is sacB, rpsL (strA), pheS, dhfr (folA), lacY, Gata-1, ccdB, or thyA−.

TABLE 1

| Combination | | Origin of | Selectable |
|---|---|---|---|
| No. | Virulence Genes | Replication | marker |
| 1 | virB1-B11; virC1-C2; | pVS1 | aacC1 |
| | virD1-D2; and virG | | |
| 2 | virB1-B11; virC1-C2; | pVS1 | aacC1 |
| | virD1-D5; virE1-E3; and | | |
| | virG | | |
| 3 | virA; virB1-B11; virC1- | pVS1 | aacC1 |
| | C2; virD1-D5; virE1-E3; | | |
| | virG; and virJ | | |
| 4 | virB1-B11; virC1-C2; | pRi repABC | aacC1 |
| | virD1-D5; virE1-E3; and | | |
| | virG | | |
| 5 | virA; virB1-B11; virC1- | pRi repABC | aacC1 |
| | C2; virD1-D5; virE1-E3; | | |
| | virG; and virJ | | |
| 6 | virA; virB1-B11; virC1- | pRK2 | aacC1 |
| | C2; virD1-D5; virE1-E3; | | |
| | virG; and virJ | | |
| 7 | virA; virB1-B11; virC1- | pSa | aacC1 |
| | C2; virD1-D5; virE1-E3; | | |
| | virG; and virJ | | |
| 8 | virA; virB1-B11; virC1- | pSa and par DE | aacC1 |
| | C2; virD1-D5; virE1-E3; | | |
| | virG; and virJ | | |
| 9 | virA; virB1-B11; virC1- | pBBR1 | aacC1 |
| | C2; virD1-D5; virE1-E3; | | |
| | virG; and virJ | | |
| 10 | virA; virB1 -B11; virC1- | RFS1010 | aacC1 |
| | C2; virD1-D5; virE1-E3; | | |
| | virG; and virJ | | |

In an aspect, the present disclosure provides for a vector that does not comprise SEQ ID NO: 61, or variants or fragments thereof.

In an aspect, the present disclosure provides for a vector that does not comprise SEQ ID NO: 62, or variants or fragments thereof.

In an aspect, the present disclosure provides for a vector that does not comprise a tra or trb operon sequence, or variants or fragments thereof. In a further aspect, the present disclosure provides for a vector that does not comprise a tra or trb operon sequence, wherein the tra or trb operon sequence has SEQ ID NO: 63, or variants or fragments thereof.

In an aspect, the present disclosure provides a vector having SEQ ID NOS: 34, 35, or 36, or variants and fragments thereof.

In an aspect, the present disclosure provides a vector comprising: (a) an origin of replication for propagation in Escherichia coli having SEQ ID NO: 2, or variants and fragments thereof; (b) an origin of replication for propagation in Agrobacterium spp. having SEQ ID NO: 3, or variants and fragments thereof; (c) a selectable marker gene having SEQ ID NO: 1, or variants and fragments thereof; and (d) virulence genes comprising Agrobacterium virulence genes virB1-B11 having SEQ ID NOS: 4-14, respectively, or variants and derivatives thereof; virC1-C2 having SEQ ID NOS: 16-17, respectively, or variants and derivatives thereof; virD1-D2 having SEQ ID NOS: 18-19, respectively, or variants and derivatives thereof; and virG having SEQ ID NO: 15, or variants and derivatives thereof, or the Agrobacterium virulence genes r-virB1-B11 having SEQ ID NOS: 80-90, respectively, or variants and derivatives thereof, r-virC1 -C2 having SEQ ID NOS: 92-93, respectively, or variants and derivatives thereof, r-virD1-D2 having SEQ ID NOS: 95-96, respectively, or variants and derivatives thereof, r-virG having SEQ ID NO: 91, or variants and derivatives thereof, and r-galls having SEQ ID NO: 101, or variants and derivatives thereof.

In an aspect, the present disclosure provides a vector comprising: (a) an origin of replication for propagation in Escherichia coli having SEQ ID NO: 2, or variants and fragments thereof; (b) an origin of replication for propagation in Agrobacterium spp. having SEQ ID NO: 3, or variants and fragments thereof; (c) a selectable marker gene having SEQ ID NO: 1, or variants and fragments thereof; and (d) virulence genes comprising Agrobacterium virulence genes virB1-B11 having SEQ ID NOS: 4-14, respectively, or variants and derivatives thereof; virC1-C2 having SEQ ID NOS: 16-17, respectively, or variants and derivatives thereof; virD1-D5 having SEQ ID NOS: 18-22, respectively, or variants and derivatives thereof; virE1-E3 having SEQ ID NOS: 23-25, or variants and derivatives thereof; and virG having SEQ ID NO: 15, or variants and derivatives thereof, or or the Agrobacterium virulence genes r-virB1-B11 having SEQ ID NOS: 80-90, respectively, or variants and derivatives thereof; r-virC1-C2 having SEQ ID NOS: 92-93, respectively, or variants and derivatives thereof; r-virD1-D5 having SEQ ID NOS: 94-98, respectively, or variants and derivatives thereof; r-vir-E3 having SEQ ID NO: 100, or variants and derivatives thereof; r-virG having SEQ ID NO: 91, or variants and derivatives thereof; and r-galls having SEQ ID NO: 101, or variants and derivatives thereof.

In an aspect, the present disclosure provides a vector comprising: (a) an origin of replication for propagation in Escherichia coli having SEQ ID NO: 2, or variants and fragments thereof; (b) an origin of replication for propagation in Agrobacterium spp. having SEQ ID NO: 3, or variants and fragments thereof; (c) a selectable marker gene having SEQ ID NO: 1, or variants and fragments thereof; and (d) virulence genes comprising Agrobacterium virA having SEQ ID NO: 26, or variants and derivatoves thereof; virB1-B11 having SEQ ID NOS: 4-14, respectively, or variants and derivatives thereof; virC1-C2 having SEQ ID NOS: 16-17, respectively, or variants and derivatives thereof; virD1-D5 having SEQ ID NOS: 18-22, respectively, or variants and derivatives thereof; virE1-E3 having SEQ ID NOS: 23-25, or variants and derivatives thereof; virG having SEQ ID NO: 15, or variants and derivatives thereof; and virJ having SEQ ID NO: 27, or variants and derivatives thereof, or the Agrobacterium virulence genes r-virA having SEQ ID NO: 79, or variants and derivatives thereof; r-virB1-B11 having SEQ ID NOS: 80-90, respectively, or variants and derivatives thereof; r-virC1-C2 having SEQ ID NOS: 92-93, respectively, or variants and derivatives thereof; r-virD1-D5 having SEQ ID NOS: 94-98, respectively, or variants and derivatives thereof; r-virE3 having SEQ ID NOS: 100, or variants and derivatives thereof; r-virG having SEQ ID NO: 91, or variants and derivatives thereof; and r-galls having SEQ ID NO: 101, or variants and derivatives thereof.

In an aspect, the present disclosure further provides methods for transformation of a plant comprising the steps of: (a) contacting a tissue from the plant with an Agrobacterium strain comprising a first vector comprising: (i) an origin of replication for propagation and stable maintenance in Escherichia coli; (ii) an origin of replication for propagation and stable maintenance in Agrobacterium spp.; (iii) a selectable marker gene; and (iv) Rhizobiaceae virulence genes virB1-B11, virC1-C2, virD1-D2, and virG genes, or Rhizobiaceae virulence genes r-virB1-B11, r-virC1-C, r-virD1-D2, r-virG, and r-galls, or variants and derivatives thereof, and a second vector comprising T-DNA borders and a polynucleotide sequence of interest for transfer to the plant; (b) co-cultivatiing the tissue with the Agrobacterium; and (c) regenerating a transformed plant from the tissue that expresses the polynucleotide sequence of interest.

In an aspect, the present disclosure further provides methods for transformation of a plant comprising the steps of: (a) contacting a tissue from the plant with an Agrobacterium strain comprising a first vector comprising: (i) an origin of replication for propagation and stable maintenance in Escherichia coli; (ii) an origin of replication for propagation and stable maintenance in Agrobacterium spp.; (iii) a selectable marker gene; and (iv) Agrobacterium virulence genes virB1-B11, virC1-C2, virD1-D2, and virG genes, or Agrobacterium virulence genes r-virB1-B11, r-virC1-C, r-virD1-D2, r-virG, and r-galls, or variants and derivatives thereof, and a second vector comprising T-DNA borders and a polynucleotide sequence of interest for transfer to the plant; (b) co-cultivatiing the tissue with the Agrobacterium; and (c) regenerating a transformed plant from the tissue that expresses the polynucleotide sequence of interest.

In an aspect, the present disclosure further provides kits comprising: (a) a vector comprising: (i) an origin of replication for propagation and stable maintenance in Escherichia coli; (ii) an origin of replication for propagation and stable maintenance in Agrobacterium spp.; (iii) a selectable marker gene; and (iv) Rhizobiaceae virulence genes virB1-B11; virC1-C2; virD1-D2; and virG genes, or variants and derivatives thereof; and (b) instructions for use in transformation of a plant using Agrobacterium.

In an aspect, the present disclosure further provides kits comprising: (a) a vector comprising: (i) an origin of replication for propagation and stable maintenance in Escherichia coli; (ii) an origin of replication for propagation and stable maintenance in Agrobacterium spp.; (iii) a selectable marker gene; and (iv) Agrobacterium virulence genes virB1-B11; virC1-C2; virD1-D2; and virG genes, or variants and derivatives thereof, or Agrobacterium virulence genes r-virB1-B11, r-virC1-C2, r-virD1-D2, r-virG, and r-galls; and (b) instructions for use in transformation of a plant using Agrobacterium.

“Plant” includes reference to whole plants, plant organs, plant tissues, seeds and plant cells and progeny of same. Progeny, variants, and mutants of the regenerated plants are also included within the scope of the present disclosure, provided that these parts comprise the introduced polynucleotides or were transformed using the vectors of the present disclosure. Plant cells include, without limitation, cells from seeds, suspension cultures, embryos, meristematic regions, callus tissue, leaves, roots, shoots, gametophytes, sporophytes, pollen and microspores.

As used herein, “regeneration” refers to the process of growing a plant from a plant cell or cells (e.g., plant protoplast, callus, or explant).

As used herein, the term “protoplast” refers to an isolated plant cell without cell walls which has the potency for regeneration into cell culture or a whole plant.

Plant parts include differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in a plant or in a plant organ, tissue or cell culture.

The present disclosure also includes plants obtained by any of the disclosed methods or compositions herein.

The present disclosure also includes seeds from a plant obtained by any of the disclosed methods or compositions herein.

### Variants and Fragments

By “fragment” is intended a portion of a polynucleotide or a portion of the amino acid sequence and hence protein encoded thereby. Fragments of a polynucleotide may encode protein fragments that retain the biological activity of the native protein. Thus, fragments of a nucleotide sequence may range from at least about 10 nucleotides, about 15 nucleotides, about 16 nucleotides, about 17 nucleotides, about 18 nucleotides, about 19 nucleotides, about 20 nucleotides, about 22 nucleotides, about 50 nucleotides, about 75 nucleotides, about 100 nucleotides, about 200 nucleotides, about 300 nucleotides, about 400 nucleotides, about 500 nucleotides, about 600 nucleotides, and up to the full-length polynucleotide employed.

By “derivative” is intended a polynucleotide or a portion of a polynucleotide that possesses activity that is substantially similar to the biological activity of the reference polynucleotide. A derivative of a virulence gene polynucleotide will be functional and will retain the virulence gene activity.

“Variant” is intended to mean a substantially similar sequence. For polynucleotides, a variant comprises a deletion and/or addition and/or substitution of one or more nucleotides at one or more internal sites within the native polynucleotide and/or a substitution of one or more nucleotides at one or more sites in the native polynucleotide. A variant of a virulence gene polynucleotide will retain the virulence gene activity. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of a polypeptide encoded by a virulence gene. Variant polynucleotides also include synthetically derived polynucleotide, such as those generated, for example, by using site-directed mutagenesis, but continue to retain the desired activity. Generally, variants of a particular disclosed polynucleotide (i.e., a virulence gene) will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular disclosed polynucleotide (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of disclosed polynucleotides employed is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

The following terms are used to describe the sequence relationships between two or more polynucleotides or polypeptides: (a) “reference sequence,” (b) “comparison window.” (c) “sequence identity,” and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

A method is further provided for identifying a virulence gene variant set forth in SEQ ID NOS.: 4-27 and 42-49, or variants and derivatives thereof. Such methods comprise obtaining a candidate derivative of any one of SEQ ID NOS.: 4-27 and 42-49, or variants and derivatives thereof, which is of sufficient length to retain the subject virulence gene activity; replacing the related virulence gene in a control vector to produce a candidate variant vector and determining if the candidate virulence polynucleotide derivative has the activity of the related virulence gene and thereby provide the desired transformation function of the vector as described herein. Methods of identifying such candidate variants based on the desired transformation efffect, in light of the teachings provided herein, are known. In various aspects, it is to be understood that the term “ . . . SEQ ID NOS.: 4-27 and 42-49, or variants or derivatives thereof . . . ” is intended to mean that the disclosed sequences comprise SEQ ID NOS.: 4-27 and 42-49, and/or derivatives of SEQ ID NOS.: 4-27 and 42-49, the variants of SEQ ID NOS.: 4-27 and 42-49, and/or the derivatives of SEQ ID NOS.: 4-27 and 42-49, individually (or) or inclusive of some or all listed sequences.

### II. Transformation of Plants

In aspects, the methods of the present disclosure involve introducing a polynucleotide into a plant cell. “Introducing” is intended to mean presenting to the plant cell the polynucleotide in such a manner that the sequence gains access to the interior of a plant cell. The methods of the present disclosure involve introducing a polynucleotide into a plant cell using methods such as Agrobacterium-mediated transformation (U.S. Pat. No. 5,563,055 and U.S. Pat. No. 5,981,840).

In aspects, the vectors of the present disclosure can be used to improve the efficiency and speed of introducing a polynucleotide into a plant cell.

In aspects, the vectors of the present disclosure are useful for transforming one or more cells of an explant. The explant, including mature and immature somatic plant tissue, can be used as a source or explant material in the present disclousre as long as it is capable of producing embryogenic material or somatic embryos. Suitable somatic plant tissue includes tissue from staminate (i.e., male flowers), pistolate (i.e., female flowers), perfect flowers, corm discs, flowering stems, bracts, and the like. Immature flowers and corm discs are the preferred somatic plant tissue sources. In an aspect, the plant-derived explant used for transformation includes immature embryos, 1-5 mm zygotic embryos, and 3.5-5 mm embryos.

The explant used in the disclosed methods can be derived from a monocot, including, but not limited to, barley, maize, millet, oats, rice, rye, Setaria spp., sorghum, sugarcane, switchgrass, triticale, turfgrass, or wheat. Alternatively, the explant used in the disclosed methods can be derived from a dicot, including, but not limited to, kale, cauliflower, broccoli, mustard plant, cabbage, pea, clover, alfalfa, broad bean, tomato, cassava, soybean, canola, alfalfa, sunflower, safflower, tobacco, Arabidopsis, or cotton.

In a further aspect, the explant used in the disclosed methods can be derived from a plant that is a member of the family Poaceae. Non-limiting examples of suitable plants from which an explant of the disclosed can be derived include grain crops, including, but not limited to, barley, maize (corn), oats, rice, rye, sorghum, wheat, millet, triticale; leaf and stem crops, including, but not limited to, bamboo, marram grass, meadow-grass, reeds, ryegrass, sugarcane; lawn grasses, ornamental grasses, and other grasses such as switchgrass and turfgrass.

In a further aspect, the explant used in the disclosed methods can be derived from any plant, including higher plants, e.g., classes of Angiospermae and Gymnospermae. Plants of the subclasses of the Dicotylodenae and the Monocotyledonae are suitable. Suitable species may come from the family Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae, Theaceae, and Vitaceae.

Suitable species from which the explant used in the disclosed methods can be derived include members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Daucus, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Juglans, Lactuca, Lavendula, Linum, Lolium, Lupinus, Lycopersicon, Lycopodium, Manihot, Medicago, Mentha, Miscanthus, Moringa, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Rosmarinus, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

In a further aspect, the explant used in the disclosed methods can be derived from a plant that is important or interesting for agriculture, horticulture, biomass for the production of liquid fuel molecules and other chemicals, and/or forestry. Non-limiting examples include, for instance, Panicum virgatum (switchgrass), Sorghum bicolor (sorghum, sudangrass), Miscanthus giganteus (miscanthus), Saccharum spp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Salix spp. (willow), Eucalyptus spp. (eucalyptus), Triticosecale spp. (triticum—wheat X rye), Bamboo, Carthamus tinctorius (safflower), Jatropha curcas (jatropha), Ricinus communis (castor), Elaeis guineensis (palm), Linum usitatissimum (flax), Brassica juncea, Manihot esculenta (cassava), Lycopersicon esculentum (tomato), Lactuca sativa (lettuce), Musa paradisiaca (banana), Solanum tuberosum (potato), Brassica oleracea (broccoli, cauliflower, brusselsprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis sativa, Camptotheca acuminate, Catharanthus roseus, Vinca rosea, Cinchona officinalis, Colchicum autumnale, Veratrum californica., Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (=Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass), Phleum pratense (timothy), and conifers. Of interest are plants grown for energy production, so called energy crops, such as cellulose-based energy crops like Panicum virgatum (switchgrass), Sorghum bicolor (sorghum, sudangrass), Miscanthus giganteus (miscanthus), Saccharum spp. (energycane), Populus balsamifera (poplar), Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale spp. (triticum—wheat X rye), and Bamboo; and starch-based energy crops like Zea mays (corn) and Manihot esculenta (cassava); and sucrose-based energy crops like Saccharum spp. (sugarcane) and Beta vulgaris (sugarbeet); and biodiesel-producing energy crops like Glycine max (soybean), Brassica napus (canola), Helianthus annuus (sunflower), Carthamus tinctorius (safflower), Jatropha curcas (jatropha), Ricinus communis (castor), Elaeis guineensis (palm), Linum usitatissimum (flax), and Brassica juncea.

As used herein, a “biomass renewable energy source plant” or “biomass for the production of liquid fuel molecules and other chemicals” means a plant having or producing material (either raw or processed) that comprises stored solar energy that can be converted to electrical energy, liquid fuels, and other useful chemicals. In general terms, such plants comprise dedicated energy crops as well as agricultural and woody plants. Examples of biomass renewable energy source plants include: Panicum virgatum (switchgrass), Sorghum bicolor (sorghum, sudangrass), Miscanthus giganteus (miscanthus), Saccharum spp. (energycane), Populus balsamifera (poplar), Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale spp. (triticum—wheat X rye), Bamboo, Zea mays (corn), Manihot esculenta (cassava), Saccharum spp. (sugarcane), Beta vulgaris (sugarbeet), Glycine max (soybean), Brassica napus (canola), Helianthus annuus (sunflower), Carthamus tinctorius (safflower), Jatropha curcas (j atropha), Ricinus communis (castor), Elaeis guineensis (palm), Linum usitatissimum (flax), and Brassica juncea.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the compositions and methods described herein provide transformed seeds (also referred to as “transgenic seed”) comprising a polynucleotide that has been introduced into a plant using a vector of the present disclosure, stably incorporated into their genome.

Thus, the methods and compositions of the present disclosure may be used for transformation of any plant species and development of transgenic plants of any species, including, but not limited to, monocots and dicots. Examples of plant species of interest include, but are not limited to, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present disclosure include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Eastern or Canadian hemlock (Tsuga canadensis); Western hemlock (Tsuga heterophylla); Mountain hemlock (Tsuga mertensiana); Tamarack or Larch (Larix occidentalis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Eucalyptus species may be employed in practicing the present disclosure, including E. grandis (and its hybrids, as “urograndis”), E. globulus, E. camaldulensis, E. tereticornis, E.viminalis, E. nitens, E. saligna and E. urophylla. Optimally, plants of the present disclosure are crop plants (for example, corn, alfalfa, sunflower, Brassica, soybean, cotton, safflower, peanut, sorghum, wheat, millet, tobacco, etc.), more optimally corn and soybean plants, yet more optimally corn plants.

Plants of particular interest include grain plants that provide seeds of interest, oil-seed plants, and leguminous plants. Seeds of interest include, but are not limited to, grain seeds, such as corn, wheat, barley, rice, sorghum, and rye. Oil-seed plants include, but are not limited to, cotton, soybean, safflower, sunflower, Brassica, maize, alfalfa, palm, and coconut, Leguminous plants include, but are not limited to, beans and peas. Beans include guar, locust bean, fenugreek, soybean, garden beans, cowpea, mungbean, lima bean, fava bean, lentils, chickpea.

### III. Methods to Improve Plant Traits and Characteristics

The present disclosure provides novel compositions and methods for producing transformed plants with increased efficiency. The disclosed methods and compositions can further comprise polynucleotides that provide for improved traits and characteristics. Thus, the present disclosure further provides methods for transformation of a plant, the method comprising the steps of: (a) contacting a tissue from the plant with an Agrobacterium strain comprising a first vector comprising: (i) an origin of replication for propagation and stable maintenance in Escherichia coli; (ii) an origin of replication for propagation and stable maintenance in Agrobacterium spp.; (iii) a selectable marker gene; and (iv) Rhizobiaceae virulence genes virB1-B11 or r-virB1-B11, virC1-C2 or r-virC1-C2, virD1-D2 or r-virD1-D2, and virG or r-virG, or variants and derivatives thereof, wherein the vector comprising the virulence genes r-virB1-B11, r-virC1-C2, r-virD1-D2, and r-virG further comprises a r-galls virulence gene, or variants and derivatives thereof, and a second vector comprising T-DNA borders and a polynucleotide sequence of interest for transfer to the plant; (b) co-cultivatiing the tissue with the Agrobacterium; and (c) regenerating a transformed plant from the tissue that expresses the polynucleotide sequence of interest; wherein the polynucleotide sequence provides for an improved trait or characteristic.

As used herein, “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern analysis, RT-PCR, microarray gene expression assays, or reporter gene expression systems, or by agricultural observations such as stress tolerance, yield, or pathogen tolerance. An “enhanced trait” as used in describing the aspects of the present disclosure includes, for example, improved or enhanced water use efficiency or drought tolerance, osmotic stress tolerance, high salinity stress tolerance, heat stress tolerance, enhanced cold tolerance, including cold germination tolerance, increased yield, enhanced nitrogen use efficiency, early plant growth and development, late plant growth and development, enhanced seed protein, and enhanced seed oil production.

Any polynucleotide of interest can be used in the methods of the present disclosure. Various changes in phenotype are of interest including, but not limited to, modifying the fatty acid composition in a plant, altering the amino acid content, starch content, or carbohydrate content of a plant, altering a plant's pathogen defense mechanism, affecting kernel size, sucrose loading, and the like. The gene of interest may also be involved in regulating the influx of nutrients, and in regulating expression of phytate genes particularly to lower phytate levels in the seed. These results can be achieved by providing expression of heterologous products or increased expression of endogenous products in plants. Alternatively, the results can be achieved by providing for a reduction of expression of one or more endogenous products, particularly enzymes or cofactors in the plant.

These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

Polynucleotides introduced into a target tissue by the disclosed methods and compositions can be operably linked to a suitable promoter. A target tissue may include, but is not limited to, a somatic embryo, mature seeds, meristems, leaf explant, seeds, suspension cultures, embryos, meristematic regions, callus tissue, leaves, roots, shoots, gametophytes, sporophytes, pollen, microspores and other plant explants. “Promoter” means a region of DNA that is upstream from the start of transcription and is involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such as Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissues are referred to as “tissue specific”. A “cell type” specific promoter primarily drives expression in certain cell types in one or more organs, for example, vascular cells in roots or leaves. An “inducible” or “repressible” promoter can be a promoter which is under either environmental or exogenous control. Examples of environmental conditions that may effect transcription by inducible promoters include anaerobic conditions, or certain chemicals, or the presence of light. Alternatively, exogenous control of an inducible or repressible promoter can be affected by providing a suitable chemical or other agent that via interaction with target polypeptides result in induction or repression of the promoter. Tissue specific, tissue preferred, cell type specific, and inducible promoters constitute the class of “non-constitutive” promoters. A “constitutive” promoter is a promoter which is active under most conditions. As used herein, “antisense orientation” includes reference to a polynucleotide sequence that is operably linked to a promoter in an orientation where the antisense strand is transcribed. The antisense strand is sufficiently complementary to an endogenous transcription product such that translation of the endogenous transcription product is often inhibited. “Operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference) could be used. Other agronomically important genes encode latex, Floury 2, growth factors, seed storage factors, and transcription factors.

Insect resistance genes may confer resistance to pests such as rootworm, cutworm, European Corn Borer, and the like which cause significant crop damage resulting int have great yield drag. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), glyphosate (e.g., the EPSPS gene and the GAT gene; see, for example, U.S. Publication No. 20040082770 and WO 03/092360) or other such genes known in the art. The bar gene encodes resistance to the herbicide basta, the nptll gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389.

Commercial traits can also be encoded on a gene or genes for improved trait composition for example, starch for ethanol production, or enhanced expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

In an aspect, further agronomic traits of interest that can be introduced into a target tissue with increased efficiency and speed are such traits as increased yield or other traits that provide increased plant value, including, for example, improved seed quality. Of particular interest are traits that provide improved or enhanced water use efficiency or drought tolerance, osmotic stress tolerance, high salinity stress tolerance, heat stress tolerance, enhanced cold tolerance, including cold germination tolerance, increased yield, enhanced nitrogen use efficiency, early plant growth and development, late plant growth and development, enhanced seed protein, and enhanced seed oil production.

Many agronomic traits can affect “yield”, including without limitation, plant height, pod number, pod position on the plant, number of internodes, incidence of pod shatter, grain size, efficiencyof nodulation and nitrogen fixation, efficiency of nutrient assimilation, resistance to biotic and abiotic stress, carbon assimilation, plant architecture, resistance to lodging, percent seed germination, seedling vigor, and juvenile traits. Other traits that can affect yield include, efficiency of germination (including germination in stressed conditions), growth rate (including growth rate in stressed conditions), ear number, seed number per ear, seed size, composition of seed (starch, oil, protein) and characteristics of seed fill. Also of interest is the generation of transgenic plants that demonstrate desirable phenotypic properties that may or may not confer an increase in overall plant yield. Such properties include enhanced plant morphology, plant physiology or improved components of the mature seed harvested from the transgenic plant.

“Increased yield” of a transgenic plant of the present disclosure may be evidenced and measured in a number of ways, including test weight, seed number per plant, seed weight, seed number per unit area (i.e. seeds, or weight of seeds, per acre), bushels per acre, tons per acre, and kilo per hectare. For example, maize yield may be measured as production of shelled corn kernels per unit of production area, e.g. in bushels per acre or metric tons per hectare, often reported on a moisture adjusted basis, e.g., at 15.5% moisture. Increased yield may result from improved utilization of key biochemical compounds, such as nitrogen, phosphorous and carbohydrate, or from improved tolerance to environmental stresses, such as cold, heat, drought, salt, and attack by pests or pathogens. Trait-enhancing recombinant DNA may also be used to provide transgenic plants having improved growth and development, and ultimately increased yield, as the result of modified expression of plant growth regulators or modification of cell cycle or photosynthesis pathways.

Many agronomic traits can affect “yield”, including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Other traits that can affect yield include, but are not limited to, efficiency of germination (including germination in stressed conditions), growth rate (including growth rate in stressed conditions), ear number, seed number per ear, seed size, composition of seed (starch, oil, protein) and characteristics of seed fill. Also of interest is the generation of transgenic plants that demonstrate desirable phenotypic properties that may or may not confer an increase in overall plant yield. Such properties include, but are not limited to, enhanced plant morphology, plant physiology and improved components of the mature seed harvested from the transgenic plant.

### IV. Methods to Suppress Genes

In an aspect, the disclosed methods and compositions can be used to introduce into a plant cell polynucleotides useful for gene suppression of a target gene in a plant derived from the plant cell. Reduction of the activity of specific genes (also known as gene silencing or gene suppression) is desirable for several aspects of genetic engineering in plants. Many techniques for gene silencing are well known to one of skill in the art, including but not limited to antisense technology (see, e.g., Sheehy et al. (1988) Proc. Natl. Acad. Sci. USA 85:8805-8809; and U.S. Pat. Nos. 5,107,065; 5,453,566; and 5,759,829); cosuppression (e.g., Taylor (1997) Plant Cell 9:1245; Jorgensen (1990) Trends Biotech. 8(12):340-344; Flavell (1994) Proc. Natl. Acad. Sci. USA 91:3490-3496; Finnegan et al. (1994) Bio/Technology 12: 883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; Javier (2003) Nature 425:257-263; and, Montgomery et al. (1998) Proc. Natl. Acad. Sci. USA 95:15502-15507), virus-induced gene silencing (Burton, et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; WO 02/00904; and WO 98/53083); ribozymes (Steinecke et al. (1992) EMBO J. 11:1525; U.S. Pat. No. 4,987,071; and, Perriman et al. (1993) Antisense Res. Dev. 3:253); oligonucleotide mediated targeted modification (e.g., WO 03/076574 and WO 99/25853); Zn-finger targeted molecules (e.g., WO 01/52620; WO 03/048345; and WO 00/42219); artificial micro RNAs (US8106180; Schwab et al. (2006) Plant Cell 18:1121-1133); and other methods or combinations of the above methods known to those of skill in the art.

V. Methods to Introduce Genome Editing Technologies into Plants

In an aspect, the disclosed methods and compositions can be used to introduce into a plant cell polynucleotides useful to target a specific site for modification in the genome of a plant derived from the plant cell. Site specific modifications that can be introduced with the disclosed methods and compositions include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. For example, the disclosed methods and compositions can be used to introduce a CRISPR-Cas system into plant cells, for the purpose of genome modification of a target sequence in the genome of a plant cell or plant derived from the plant cell, for selecting plants, for deleting a base or a sequence, for gene editing, and for inserting a polynucleotide of interest into the genome of a plant derived from a plant cell. Thus, the disclosed methods and compositions can be used together with a CRISPR-Cas system to provide for an effective system for modifying or altering target sites and nucleotides of interest within the genome of a plant, plant cell or seed.

In an aspect, the present disclosure comprises methods and compositions for transformation, wherein the method comprises the steps of: (a) contacting a plant cell with an Agrobacterium strain comprising a first vector comprising: (i) an origin of replication for propagation and stable maintenance in Escherichia coli; (ii) an origin of replication for propagation and stable maintenance in Agrobacterium spp.; (iii) a selectable marker gene; and (iv) Agrobacterium virulence genes virB1-B11; virC1-C2; virD1-D2; and virG genes; a second vector capable of expressing a guide nucleotide; and a third construct capable of expressing a Cas endonuclease, wherein the guide nucleotide and Cas endonuclease are capable of forming a complex that enables the Cas endonuclease to introduce a double strand break at the target site; (b) co-cultivatiing the tissue with the Agrobacterium; and (c) regenerating a transformed plant from the tissue that expresses the polynucleotide sequence of interest.

In an aspect, the Cas endonuclease gene is a plant optimized Cas9 endonuclease, wherein the plant optimized Cas9 endonuclease is capable of binding to and creating a double strand break in a genomic target sequence of the plant genome.

The Cas endonuclease is guided by the guide nucleotide to recognize and optionally introduce a double strand break at a specific target site into the genome of a plant cell. The CRISPR-Cas system provides for an effective system for modifying target sites within the genome of a plant, plant cell or seed. Further provided are methods and compositions employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest. The disclosed compositions and methods can be used to introduce a CRISPR-Cas system for editing a nucleotide sequence in the genome of a cell. The nucleotide sequence to be edited (the nucleotide sequence of interest) can be located within or outside a target site that is recognized by a Cas endonuclease.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times-also referred to as CRISPR-repeats) which are partially palindromic. The repeated sequences (usually specific to a species) are interspaced by variable sequences of constant length (typically 20 to 58 by depending on the CRISPR locus (WO2007/025097 published March 1, 2007).

CRISPR loci were first recognized in E. coli (Ishino et al. (1987) J. Bacterial. 169:5429-5433; Nakata et al. (1989) J. Bacterial. 171 :3553-3556). Similar interspersed short sequence repeats have been identified in Haloferax mediterranei, Streptococcus pyogenes, Anabaena, and Mycobacterium tuberculosis (Groenen et al. (1993) Mol. Microbiol. 10:1057-1065; Hoe et al. (1999) Emerg. Infect. Dis. 5:254-263; Masepohl et al. (1996) Biochim. Biophys. Acta 1307:26-30; Mojica et al. (1995) Mol. Microbiol. 17:85-93). The CRISPR loci differ from other SSRs by the structure of the repeats, which have been termed short regularly spaced repeats (SRSRs) (Janssen et al. (2002) OMICS J. Integ. Biol. 6:23-33; Mojica et al. (2000) Mol. Microbiol. 36:244-246). The repeats are short elements that occur in clusters, that are always regularly spaced by variable sequences of constant length (Mojica et al. (2000) Mol. Microbiol. 36:244-246).

Cas gene includes a gene that is generally coupled, associated or close to or in the vicinity of flanking CRISPR loci. The terms “Cas gene” and “CRISPR-associated (Cas) gene” are used interchangeably herein. A comprehensive review of the Cas protein family is presented in Haft et al. (2005) Computational Biology, PLoS Comput Biol 1 (6): e60. doi:10.1371/journal.pcbi.0010060.

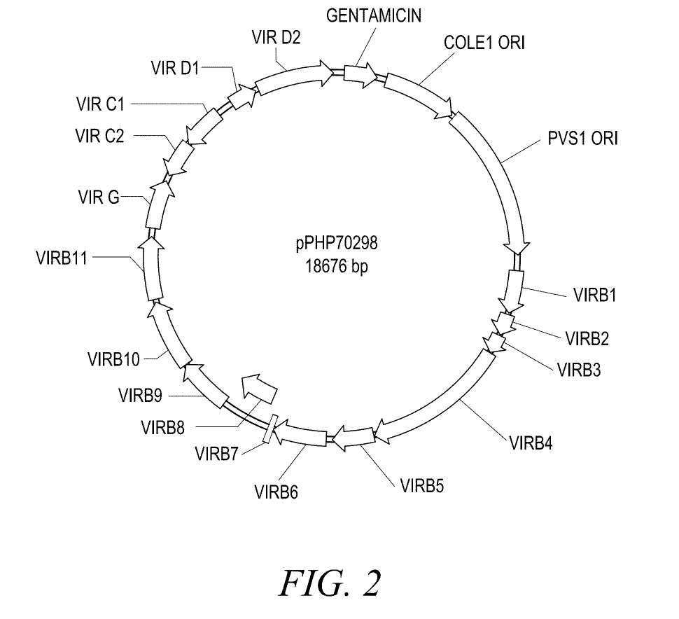

In addition to the four initially described gene families, an additional 41 CRISPR-associated (Cas) gene families have been described in WO/2015/026883, which is incorporated herein by reference. This reference shows that CRISPR systems belong to different classes, with different repeat patterns, sets of genes, and species ranges. The number of Cas genes at a given CRISPR locus can vary between species. Cas endonuclease relates to a Cas protein encoded by a Cas gene, wherein the Cas protein is capable of introducing a double strand break into a DNA target sequence. The Cas endonuclease is guided by the guide polynucleotide to recognize and optionally introduce a double strand break at a specific target site into the genome of a cell. As used herein, the term “guide polynucleotide/Cas endonuclease system” includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide nucleotide, but only if the correct protospacer-adjacent motif (PAM) is approximately oriented at the 3′ end of the target sequence (see FIG. 2A and FIG. 2B of WO/2015/026883, published Feb. 26, 2015).

In an aspect, the Cas endonuclease gene is a Cas9 endonuclease , such as, but not limited to, Cas9 genes listed and disclosed in WO2007/025097, published Mar. 1, 2007, and incorporated herein by reference. In another aspect, the Cas endonuclease gene is plant, maize or soybean optimized Cas9 endonuclease, such as, but not limited to those disclosed WO/2015/026883. In another aspect, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region.

In an aspect, the Cas endonuclease gene is a Cas9 endonuclease gene, or any functional fragment or variant thereof, disclosed in WO/2015/026883.

The terms “functional fragment,” “fragment that is functionally equivalent,” and “functionally equivalent fragment” are used interchangeably herein. These terms refer to a portion or subsequence of the Cas endonuclease sequence of the present disclosure in which the ability to create a double-strand break is retained.

The terms “functional variant,” “variant that is functionally equivalent” and “functionally equivalent variant” are used interchangeably herein. These terms refer to a variant of the Cas endonuclease of the present disclosure in which the ability to create a double-strand break is retained. Fragments and variants and derivatives can be obtained via methods such as site-directed mutagenesis and synthetic construction.

In an aspect, the Cas endonuclease gene is a plant codon optimized Streptococcus pyogenes Cas9 gene that can recognize any genomic sequence of the form N(12-30)NGG, which can be targeted.

Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more. (patent application PCT/US 12/30061 filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. Meganucleases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr Op Biotechnol 5:521 -7; and Sadowski (1993) FASEB 7:760-7. In some examples the recombinase is from the Integrase or Resolvase families. TAL effector nucleases are a new class of sequence-specific nucleases that can be used to make double-strand breaks at specific target sequences in the genome of a plant or other organism. (Miller, et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprises two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ms endonuclease such as Fok1. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognizes a sequence of 9 contiguous nucleotides for binding upon dimerization, while two sets of zinc finger triplets are used to bind an 18 nucleotide recognition sequence.

Bacteria and Archaea have evolved adaptive immune defenses termed clustered regularly interspaced short palindromic repeats (CRISPR)/CRISPR-associated (Cas) systems that use short RNA to direct degradation of foreign nucleic acids (WO2007/025097published Mar. 1, 2007). The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target.

As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that can interact with a Cas endonuclease.

As used herein, the term “guide polynucleotide” relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide nucleotide”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In an aspect, the first molecule of the duplex guide polynucleotide comprising a VT domain linked to a CER domain is referred to as “crDNA” (when comprised of a contiguous stretch of DNA nucleotides) or “crRNA” (when comprised of a contiguous stretch of RNA nucleotides), or “crDNA-RNA” (when comprised of a combination of DNA and RNA nucleotides). The crNucleotide can comprise a fragment of the cRNA naturally occurring in Bacteria and Archaea. In an aspect, the size of the fragment of the cRNA naturally occurring in Bacteria and Archaea that is present in a crNucleotide disclosed herein can range from, but is not limited to, 2, 3, 4, 5, 6, 7, 8, 9,10, 11, 12, 13, 14, 15, 16, 17, 18, 19, 20 or more nucleotides.

In an aspect, the second molecule of the duplex guide polynucleotide comprising a CER domain is referred to as “tracrRNA” (when comprised of a contiguous stretch of RNA nucleotides) or “tracrDNA” (when comprised of a contiguous stretch of DNA nucleotides) or “tracrDNA-RNA” (when comprised of a combination of DNA and RNA nucleotides). In an aspect, the RNA that guides the RNA Cas9 endonuclease complex is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as Cas endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In an aspect the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide nucleotide” (when comprised of a contiguous stretch of RNA nucleotides) or “single guide DNA” (when comprised of a contiguous stretch of DNA nucleotides) or “single guide nucleotide-DNA” (when comprised of a combination of RNA and DNA nucleotides). In an aspect of the disclosure, the single guide nucleotide comprises a cRNA or cRNA fragment and a tracrRNA or tracrRNA fragment of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. One aspect of using a single guide polynucleotide versus a duplex guide polynucleotide is that only one expression cassette needs to be made to express the single guide polynucleotide.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In an aspect, the variable targeting domain comprises a contiguous stretch of 12 to 30 nucleotides. The variable targeting domain can be comprised of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be comprised of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In an aspect, the nucleotide sequence linking the crNucleotide and the tracrNucleotide of a single guide polynucleotide can be at least 3, 4, 5, 6, 7, 8, 9, 10, 11 , 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another aspect, the nucleotide sequence linking the crNucleotide and the tracrNucleotide of a single guide polynucleotide can comprise a tetraloop sequence, such as, but not limiting to a GAAA tetraloop sequence.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide polynucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In aspects, the guide nucleotide and Cas endonuclease are capable of forming a complex that enables the Cas endonuclease to introduce a double strand break at a DNA target site.

In aspects, the variable target domain is 12, 13, 14, 15, 16, 17, 18, 19, 20, 21, 22, 23, 24, 25, 26, 27, 28, 29 or 30 nucleotides in length.

In aspects, the guide nucleotide comprises a cRNA (or cRNA fragment) and a tracrRNA (or tracrRNA fragment) of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. In an aspect the guide nucleotide can be introduced into a plant or plant cell directly using any method known in the art such as, but not limited to, particle bombardment or topical applications.

In aspects, the guide nucleotide can be introduced indirectly by introducing a recombinant DNA molecule comprising the corresponding guide DNA sequence operably linked to a plant specific promoter that is capable of transcribing the guide nucleotide in the plant cell. The term “corresponding guide DNA” includes a DNA molecule that is identical to the RNA molecule but has a “T” substituted for each “U” of the RNA molecule.

In aspects, the guide nucleotide is introduced via particle bombardment or using the disclosed methods and compositions for Agrobacterium transformation of a recombinant DNA construct comprising the corresponding guide DNA operably linked to a plant U6 polymerase III promoter.

In aspects, the RNA that guides the RNA Cas9 endonuclease complex is a duplexed RNA comprising a duplex crRNA-tracrRNA. One advantage of using a guide nucleotide versus a duplexed crRNA-tracrRNA is that only one expression cassette needs to be made to express the fused guide nucleotide.

The terms “target site,” “target sequence,” “target DNA,” “target locus,” “genomic target site,” “genomic target sequence,” and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature.

As used herein, terms “endogenous target sequence” and “native target sequence” are used interchangeably herein to refer to a target sequence that is endogenous or native to the genome of a plant and is at the endogenous or native position of that target sequence in the genome of the plant. In an aspect, the target site can be similar to a DNA recognition site or target site that is specifically recognized and/or bound by a double-strand break inducing agent such as a LIG3-4 endonuclease (US patent publication 2009-0133152 A1 (published May 21, 2009) or a MS26++ meganuclease (U.S. patent application Ser. No. 13/526,912 filed Jun. 19, 2012).

An “artificial target site” or “artificial target sequence” are used interchangeably herein and refer to a target sequence that has been introduced into the genome of a plant. Such an artificial target sequence can be identical in sequence to an endogenous or native target sequence in the genome of a plant but be located in a different position (i.e., a non-endogenous or non-native position) in the genome of a plant.

An “altered target site,” “altered target sequence,” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

### VI. Methods to Introduce Nucleotides for Site-Specific Integration

In an aspect, the disclosed methods and compositions can be used to introduce into a plant cell with increased efficiency and speed polynucleotides useful for the targeted integration of nucleotide sequences into a plant derived from the plant cell. For example, the disclosed methods and compositions can be used to introduce transfer cassettes comprising nucleotide sequences of interest flanked by non-identical recombination sites to transform a plant comprising a target site. In an aspect, the target site contains at least a set of non-identical recombination sites corresponding to those on the transfer cassette. The exchange of the nucleotide sequences flanked by the recombination sites is effected by a recombinase. Thus, the disclosed methods and compositions can be used for the introduction of transfer cassettes for targeted integration of nucleotide sequences, wherein the transfer cassettes which are flanked by non-identical recombination sites are recognized by a recombinase that recognizes and implements recombination at the nonidentical recombination sites. Accordingly, the disclosed methods and compositions can be used to improve efficiency and speed of development of plants, derived from plant cells, containing non-identical recombination sites.

In an aspect, the present disclosure further provides methods for transformation of a plant, wherein the method comprises introducing a polynucleotide of interest into a target site in the genome of a plant cell, the method comprising the steps of: (a) contacting an explant from the plant with an Agrobacterium strain comprising a first vector comprising: (i) an origin of replication for propagation and stable maintenance in Escherichia coli; (ii) an origin of replication for propagation and stable maintenance in Agrobacterium spp.; (iii) a selectable marker gene; and (iv) Agrobacterium virulence genes virB1-B11; virC1-C2; virD1-D2; and virG genes, and a second vector comprising a transfer cassette comprising the polynucleotide of interest flanked by nonidentical recombination sites; (b) co-cultivatiing the tissue with the Agrobacterium; and (c) regenerating a transformed plant from the tissue that expresses the polynucleotide sequence of interest; wherein the explant is derived from a plant with a genome comprising a target site flanked by non identical recombination sites which correspond to the flanking sites of the transfer cassette. The method can further comprise providing a recombinase that recognizes and implements recombination at the nonidentical recombination sites, the recombinase being provided to the plant explant, a plantlet derived from the somatic embryo, or a plant derived from a plantlet derived from a plant cell.

Thus, the disclosed methods and compositions can further comprise compositions and methods for the directional, targeted integration of exogenous nucleotides into a transformed plant. In an aspect, the disclosed methods use novel recombination sites in a gene targeting system which facilitates directional targeting of desired genes and nucleotide sequences into corresponding recombination sites previously introduced into the target plant genome.

In an aspect, a nucleotide sequence flanked by two non-identical recombination sites is introduced into one or more cells of an explant derived from the target organism's genome establishing a target site for insertion of nucleotide sequences of interest. Once a stable plant or cultured tissue is established, a second construct, or nucleotide sequence of interest, flanked by corresponding recombination sites as those flanking the target site, is introduced into the stably transformed plant or tissues in the presence of a recombinase protein. This process results in exchange of the nucleotide sequences between the non-identical recombination sites of the target site and the transfer cassette.

It is recognized that the transformed plant prepared in this manner may comprise multiple target sites; i.e., sets of non-identical recombination sites. In this manner, multiple manipulations of the target site in the transformed plant are available. By target site in the transformed plant is intended a DNA sequence that has been inserted into the transformed plant's genome and comprises non-identical recombination sites.

Examples of recombination sites for use in the disclosed method are known in the art and include FRT sites (See, for example, Schlake and Bode (1994) Biochemistry 33: 12746-12751; Huang et al. (1991) Nucleic Acids Research 19: 443-448; Paul D. Sadowski (1995) In Progress in Nucleic Acid Research and Molecular Biology vol. 51, pp. 53-91; Michael M. Cox (1989) In Mobile DNA, Berg and Howe (eds) American Society of Microbiology, Washington D.C., pp. 116-670; Dixon et al. (1995) 18: 449-458; Umlauf and Cox (1988) The EMBO Journal 7: 1845-1852; Buchholz et al. (1996) Nucleic Acids Research 24: 3118-3119; Kilby et al. (1993) Trends Genet. 9: 413-421: Rossant and Geagy (1995) Nat. Med. 1: 592-594; Albert et al. (1995) The Plant J. 7: 649-659: Bayley et al. (1992) Plant Mol. Biol. 18: 353-361; Odell et al. (1990) Mol. Gen. Genet. 223: 369-378; and Dale and Ow (1991) Proc. Natl. Acad. Sci. USA 88: 10558-105620; all of which are herein incorporated by reference.); Lox (Albert et al. (1995) Plant J. 7: 649-659; Qui et al. (1994) Proc. Natl. Acad. Sci. USA 91: 1706-1710; Stuurman et al. (1996) Plant Mol. Biol. 32: 901-913; Odell et al. (1990) Mol. Gen. Gevet. 223: 369-378; Dale et al. (1990) Gene 91: 79-85; and Bayley et al. (1992) Plant Mol. Biol. 18: 353-361.) The two-micron plasmid, found in most naturally occurring strains of Saccharomyces cerevisiae, encodes a site-specific recombinase that promotes an inversion of the DNA between two inverted repeats. This inversion plays a central role in plasmid copy-number amplification.

The protein, designated FLP protein, catalyzes site-specific recombination events. The minimal recombination site (FRT) has been defined and contains two inverted 13-base pair (bp) repeats surrounding an asymmetric 8-bp spacer. The FLP protein cleaves the site at the junctions of the repeats and the spacer and is covalently linked to the DNA via a 3′phosphate. Site specific recombinases like FLP cleave and religate DNA at specific target sequences, resulting in a precisely defined recombination between two identical sites. To function, the system needs the recombination sites and the recombinase. No auxiliary factors are needed. Thus, the entire system can be inserted into and function in plant cells. The yeast FLP\FRT site specific recombination system has been shown to function in plants. To date, the system has been utilized for excision of unwanted DNA. See, Lyznik et at. (1993) Nucleic Acid Res. 21: 969-975. In contrast, the present disclosure utilizes non-identical FRTs for the exchange, targeting, arrangement, insertion and control of expression of nucleotide sequences in the plant genome.

In an aspect, a transformed organism of interest, such as an explant from a plant, containing a target site integrated into its genome is needed. The target site is characterized by being flanked by non-identical recombination sites. A targeting cassette is additionally required containing a nucleotide sequence flanked by corresponding non-identical recombination sites as those sites contained in the target site of the transformed organism. A recombinase which recognizes the non-identical recombination sites and catalyzes site-specific recombination is required.

It is recognized that the recombinase can be provided by any means known in the art. That is, it can be provided in the organism or plant cell by transforming the organism with an expression cassette capable of expressing the recombinase in the organism, by transient expression, or by providing messenger RNA (mRNA) for the recombinase or the recombinase protein.

By “non-identical recombination sites” it is intended that the flanking recombination sites are not identical in sequence and will not recombine or recombination between the sites will be minimal. That is, one flanking recombination site may be a FRT site where the second recombination site may be a mutated FRT site. The non-identical recombination sites used in the methods of the present disclosure prevent or greatly suppress recombination between the two flanking recombination sites and excision of the nucleotide sequence contained therein. Accordingly, it is recognized that any suitable non-identical recombination sites may be utilized in the present disclosure, including FRT and mutant FRT sites, FRT and lox sites, lox and mutant lox sites, as well as other recombination sites known in the art.

By suitable non-identical recombination site implies that in the presence of active recombinase, excision of sequences between two non-identical recombination sites occurs, if at all, with an efficiency considerably lower than the recombinationally-mediated exchange targeting arrangement of nucleotide sequences into the plant genome. Thus, suitable non-identical sites for use in the present disclosure include those sites where the efficiency of recombination between the sites is low; for example, where the efficiency is less than about 30 to about 50%, preferably less than about 10 to about 30%, more preferably less than about 5 to about 10%.

As noted above, the recombination sites in the targeting cassette correspond to those in the target site of the transformed plant. That is, if the target site of the transformed plant contains flanking non-identical recombination sites of FRT and a mutant FRT, the targeting cassette will contain the same FRT and mutant FRT non-identical recombination sites.

It is furthermore recognized that the recombinase, which is used in the disclosed methods, will depend upon the recombination sites in the target site of the transformed plant and the targeting cassette. That is, if FRT sites are utilized, the FLP recombinase will be needed. In the same manner, where lox sites are utilized, the Cre recombinase is required. If the non-identical recombination sites comprise both a FRT and a lox site, both the FLP and Cre recombinase will be required in the plant cell.

The FLP recombinase is a protein which catalyzes a site-specific rection that is involved in amplifying the copy number of the two micron plasmid of S. cerevisiae during DNA replication. FLP protein has been cloned and expressed. See, for example, Cox (1993) Proc. Natl. Acad. Sci. U. S. A. 80: 4223-4227. The FLP recombinase for use in the disclosed methods for targeted integration can be derived from the genus Saccharomyces. It may be preferable to synthesize the recombinase using plant preferred codons for optimum expression in a plant of interest. See, for example, U.S. application Ser. No. 08/972,258 filed Nov. 18, 1997, entitled “Novel Nucleic Acid Sequence Encoding FLP Recombinase,” herein incorporated by reference.

The bacteriophage recombinase Cre catalyzes site-specific recombination between two lox sites. The Cre recombinase is known in the art. See, for example, Guo et al. (1997) Nature 389: 40-46; Abremski et al. (1984) J. Biol. Chem. 259: 1509-1514; Chen et al. (1996) Somat. Cell Mol. Genet. 22: 477-488; and Shaikh et al. (1977) J. Biol. Chem. 272: 5695-5702; all of which are herein incorporated by reference. Such Cre sequence(s) may also be synthesized using plant preferred codons.

Where appropriate, the nucleotide sequences to be inserted in the plant genome may be optimized for increased expression in the transformed plant. Where mammalian, yeast, or bacterial genes are used in the present disclosure, they can be synthesized using plant preferred codons for improved expression. It is recognized that for expression in monocots, dicot genes can also be synthesized using monocot preferred codons. Methods are available in the art for synthesizing plant preferred genes. See, for example, U.S. Pat. Nos. 5,380,831, 5,436,391, and Murray et al. (1989) Nucleic Acids Res. 17 : 477-498, herein incorporated by reference. The plant preferred codons may be determined from the codons utilized more frequently in the proteins expressed in the plant of interest. It is recognized that monocot or dicot preferred sequences may be constructed as well as plant preferred sequences for particular plant species. See, for example, EPA 0359472; EPA 0385962; WO 91/16432; Perlak et al. (1991) Proc. Natl. Acad. Sci. USA, 88: 3324-3328; and Murray et al. (1989) Nucleic Acids Research, 17: 477-498. U.S. Pat. No. 5,380,831; U.S. Pat. No. 5,436,391; and the like, herein incorporated by reference. It is further recognized that all or any part of the gene sequence may be optimized or synthetic. That is, fully optimized or partially optimized sequences may also be used.

Additional sequence modifications are known to enhance gene expression in a cellular host and can be used in the present disclosure. These include elimination of sequences encoding spurious polyadenylation signals, exon-intron splice site signals, transposon-like repeats, and other such well-characterized sequences, which may be deleterious to gene expression. The G-C content of the sequence may be adjusted to levels average for a given cellular host, as calculated by reference to known genes expressed in the host cell. When possible, the sequence is modified to avoid predicted hairpin secondary MARNA structures.

The present disclosure also encompasses novel FLP recombination target sites (FRT). The FRT has been identified as a minimal sequence comprising two 13 base pair repeats, separated by an 8 base spacer, as follows: 5′-GAAGTTCCTATTC [TCTAGAAA] GTATAGGAACTTC3′ wherein the nucleotides within the brackets indicate the spacer region. The nucleotides in the spacer region can be replaced with a combination of nucleotides, so long as the two 13-base repeats are separated by eight nucleotides. Some substitutions of nucleotides in the spacer region may work better than others and determining which substitutions is within the skill of the art. The eight base pair spacer is involved in DNA-DNA pairing during strand exchange. The asymmetry of the region determines the direction of site alignment in the recombination event, which will subsequently lead to either inversion or excision. As indicated above, most of the spacer can be mutated without a loss of function. See, for example, Schlake and Bode (1994) Biochemistry 33: 12746-12751, herein incorporated by reference.

Novel FRT mutant sites can be used in the practice of the disclosed methods. Such mutant sites may be constructed by PCR-based mutagenesis. Although mutant FRT sites are known (e.g., see WO/1999/025821, published May 27, 1999), it is recognized that other mutant FRT sites may be used in the practice of the present disclosure. In aspects, the methods and compositions of the present disclosure can use non-identical recombination sites or FRT sites for targeted insertion and expression of nucleotide sequences in a plant genome.

As discussed above, bringing genomic DNA containing a target site with non-identical recombination sites together with a vector containing a transfer cassette with corresponding non-identical recombination sites, in the presence of the recombinase, results in recombination. The nucleotide sequence of the transfer cassette located between the flanking recombination sites is exchanged with the nucleotide sequence of the target site located between the flanking recombination sites. In this manner, nucleotide sequences of interest may be precisely incorporated into the genome of the host.

It is recognized that many variations of the present disclosure can be practiced. For example, target sites can be constructed having multiple non-identical recombination sites. Thus, multiple genes or nucleotide sequences can be stacked or ordered at precise locations in the plant genome. Likewise, once a target site has been established within the genome, additional recombination sites may be introduced by incorporating such sites within the nucleotide sequence of the transfer cassette and the transfer of the sites to the target sequence. Thus, once a target site has been established, it is possible to subsequently add sites, or alter sites through recombination.

Another variation includes providing a promoter or transcription initiation region operably linked with the target site in an organism. Preferably, the promoter will be 5′ to the first recombination site. By transforming the organism with a transfer cassette comprising a coding region, expression of the coding region will occur upon integration of the transfer cassette into the target site. This aspect provides for a method to select transformed cells, particularly plant cells, by providing a selectable marker sequence as the coding sequence.

Other advantages of the present system include the ability to reduce the complexity of integration of transgenes or transferred DNA in an organism by utilizing transfer cassettes as discussed above and selecting organisms with simple integration patterns. In the same manner, preferred sites within the genome can be identified by comparing several transformation events. A preferred site within the genome includes one that does not disrupt expression of essential sequences and provides for adequate expression of the transgene sequence.

The disclosed methods also provide for means to combine multiple cassettes at one location within the genome. Recombination sites may be added or deleted at target sites within the genome.

Any means known in the art for bringing the three components of the system together may be used in the present disclosure. For example, a plant can be stably transformed to harbor the target site in its genome. The recombinase may be transiently expressed or provided. Alternatively, a nucleotide sequence capable of expressing the recombinase may be stably integrated into the genome of the plant. In the presence of the corresponding target site and the recombinase, the transfer cassette, flanked by corresponding non-identical recombination sites, is inserted into the transformed plant's genome.

Alternatively, the components of the system may be brought together by sexually crossing transformed plants. In this aspect, a transformed plant, parent one, containing a target site integrated in its genome can be sexually crossed with a second plant, parent two, that has been genetically transformed with a transfer cassette containing flanking non-identical recombination sites, which correspond to those in plant one. Either plant one or plant two contains within its genome a nucleotide sequence expressing recombinase. The recombinase may be under the control of a constitutive or inducible promoter.

Inducible promoters include those described herein above, as well as, heat-inducible promoters, estradiol-responsive promoters, chemical inducible promoters, and the like. Pathogen inducible promoters include those from pathogenesis-related proteins (PR proteins), which are induced following infection by a pathogen; e. g., PR proteins, SAR proteins, beta-1,3-glucanase, chitinase, etc. See, for example, Redolfi et al. (1983) Neth. J. Plant Pathol. 89: 245-254; Uknes et al. (1992) The Plant Cell 4: 645-656; and Van Loon (1985) Plant Mol. Virol. 4: 111-116. In this manner, expression of recombinase and subsequent activity at the recombination sites can be controlled.

Constitutive promoters for use in expression of genes in plants are known in the art. Such promoters include, but are not limited to 35S promoter of cauliflower mosaic virus (Depicker et al. (1982) Mol. Appl. Genet. 1: 561-573; Odell et al. (1985) Nature 313: 810-812), ubiquitin promoter (Christensen et al. (1992) Plant Mol. Biol. 18: 675-689), promoters from genes such as ribulose bisphosphate carboxylase (De Almeida et al. (1989) Mol. Gen. Genet. 218: 78-98), actin (McElroy et al. (1990) Plant J. 2: 163-171), histone, DnaJ (Baszczynski et al. (1997) Maydica 42: 189-201), and the like.

The disclosed compositions and methods are useful in targeting the integration of transferred nucleotide sequences to a specific chromosomal site. The nucleotide sequence may encode any nucleotide sequence of interest. Particular genes of interest include those which provide a readily analyzable functional feature to the host cell and/or organism, such as marker genes, as well as other genes that alter the phenotype of the recipient cells, and the like. Thus, genes effecting plant growth, height, susceptibility to disease, insects, nutritional value, and the like may be utilized in the present disclosure. The nucleotide sequence also may encode an ‘antisense’ sequence to turn off or modify gene expression.

It is recognized that the nucleotide sequences may be utilized in a functional expression unit or cassette. By functional expression unit or cassette is intended, the nucleotide sequence of interest with a functional promoter, and in most instances a termination region. There are various ways to achieve the functional expression unit within the practice of the present disclosure. In one aspect of the present disclosure, the nucleic acid of interest is transferred or inserted into the genome as a functional expression unit.

Alternatively, the nucleotide sequence may be inserted into a site within the genome which is 3′ to a promoter region. In this latter instance, the insertion of the coding sequence 3′ to the promoter region is such that a functional expression unit is achieved upon integration. For convenience, for expression in plants, the nucleic acid encoding target sites and the transfer cassettes, including the nucleotide sequences of interest, can be contained within expression cassettes. The expression cassette will comprise a transcriptional initiation region, or promoter, operably linked to the nucleic acid encoding the peptide of interest. Such an expression cassette is provided with a plurality of restriction sites for insertion of the gene or genes of interest to be under the transcriptional regulation of the regulatory regions.

The transcriptional initiation region, the promoter, may be native or homologous or foreign or heterologous to the host, or could be the natural sequence or a synthetic sequence. By foreign is intended that the transcriptional initiation region is not found in the wild-type host into which the transcriptional initiation region is introduced. Either a native or heterologous promoter may be used with respect to the coding sequence of interest.

The transcriptional cassette may include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the potato proteinase inhibitor (PinII) gene or sequences from Ti-plasmid of A. tumefaciens, such as the nopaline synthase, octopine synthase and opaline synthase termination regions. See also, Guerineau et al., (1991) Mol. Gen. Genet. 262: 141-144; Proudfoot (1991) Cell 64: 671-674; Sanfacon et al. (1991) Genes Dev. 5: 141-149; Mogen et al. (1990) Plant Cell 2: 1261-1272; Munroe et al. (1990) Gene 91: 151-158; Ballas et al. 1989) Nucleic Acids Res. 17 : 7891-7903; Joshi et al. (1987) Nucleic Acid Res. 15: 9627-9639.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′noncoding region) (Elroy-Stein, O., Fuerst, T. R., and Moss, B. (1989) PNAS USA, 86: 6126-6130); potyvirus leaders, for example, TEV leader (Tobacco Etch Virus) (Allison et al. (1986); MDMV leader (Maize Dwarf Mosaic Virus); Virology, 154: 9-20), and human immunoglobulin heavy-chain binding protein (BiP), (Macejak, D. G., and P. Sarnow (1991) Nature, 353: 90-94; untranslated leader from the coat protein MARNA of alfalfa mosaic virus (AMV RNA 4), (Jobling, S. A., and Gehrke, L., (1987) Nature, 325: 622-625; tobacco mosaic virus leader (TMV), (Gallie et al. (1989) Molecular Biology of RNA, pages 237-256, Gallie et al. (1987) Nucl. Acids Res. 15: 3257-3273; maize chlorotic mottle virus leader (MCMV) (Lornmel, S. A. et al. (1991) Virology, 81: 382-385). See also, Della-Cioppa et al. (1987) Plant Physiology, 84: 965-968; and endogenous maize 5′ untranslated sequences. Other methods known to enhance translation can also be utilized, for example, introns, and the like.

The expression cassettes may contain one or more than one gene or nucleic acid sequence to be transferred and expressed in the transformed plant. Thus, each nucleic acid sequence will be operably linked to 5′ and 3′ regulatory sequences. Alternatively, multiple expression cassettes may be provided.

### EXPERIMENTAL

### Example 1

### Vir Plasmids with Improved Features

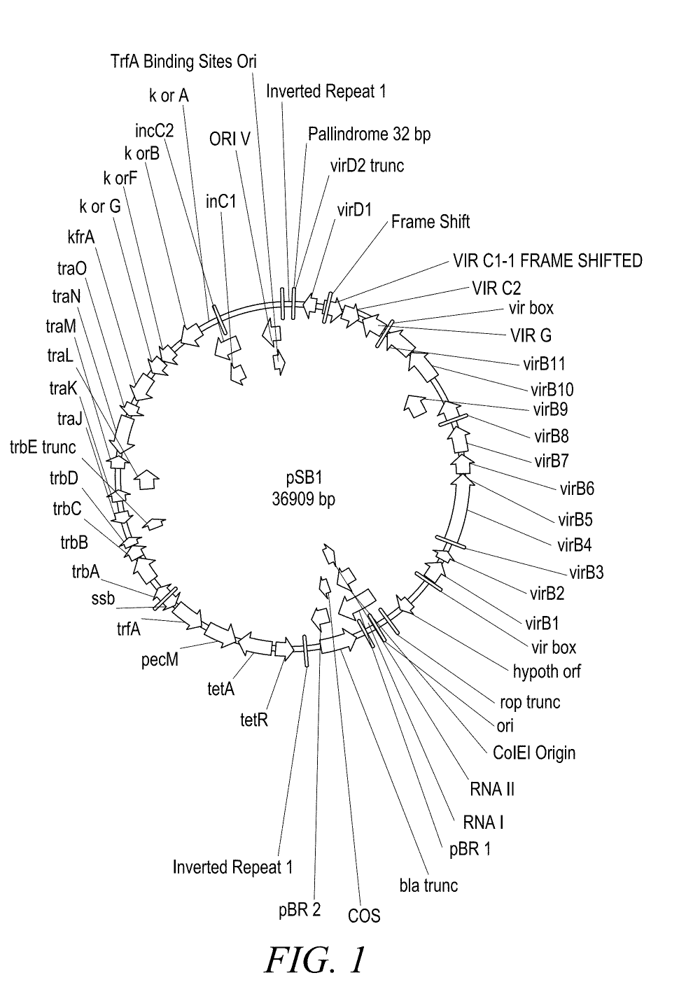

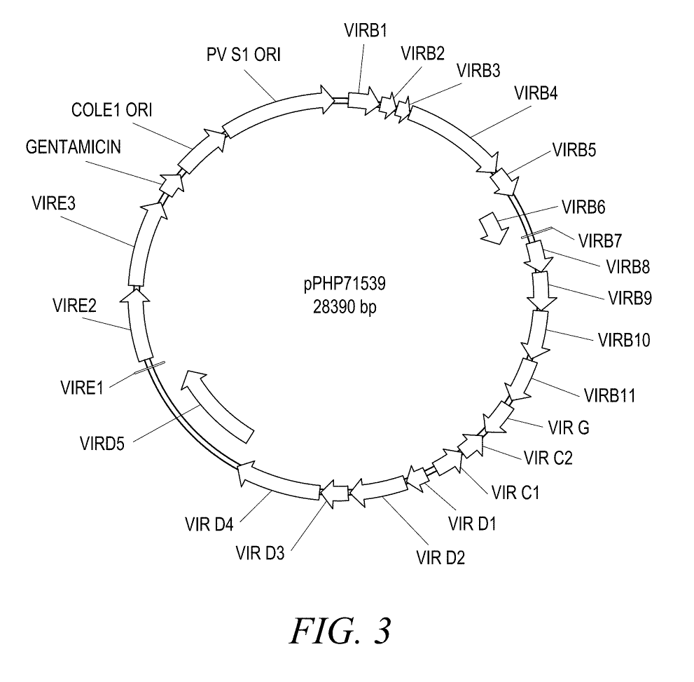

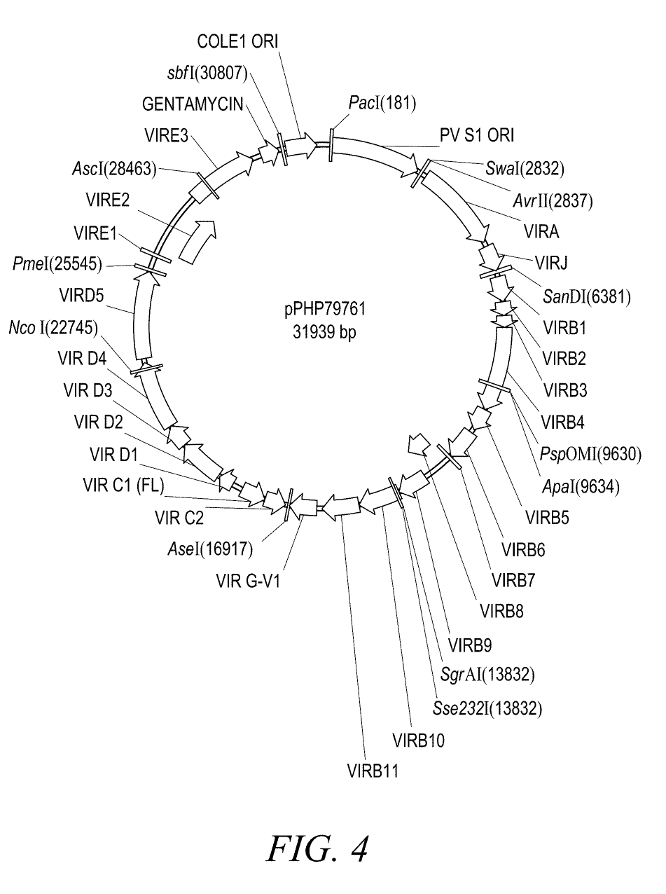

A series of improved superbinary vectors were prepared. Sequence identification numbers (SEQ ID NO:) described herein are listed in Table 23. The vectors, pPHP70298, pPHP71539, and pPHP79761, are shown as plasmid maps in FIGS. 2, 3, and 4, respectively, and have the sequences set forth in SEQ ID NOS: 34, 35, and 36, respectively (see Table 23). For comparison, a plasmid map of the vir gene plasmid pSB1 (Komari, T., et al., Plant Cell Rep. (1990), 9:303-306) is shown in FIG. 1. Among the improvements to pPHP70298 (SEQ ID NO. 34), pPHP71539 (SEQ ID NO. 35), and pPHP79761 SEQ ID NO. 36) is the presence of a smaller, more stable origin of replication, pVS1, instead of a large, unstable origin of replication (RK2). In addition, the frameshift in virC1-2 operon is repaired, which encodes the overdrive-binding protein for enhanced T-DNA transfer. It is possible that the frame-shift decreases translation of virC1-2 as the second gene in a dicistronic mRNA. Improved virulence gene functionality was also achieved by introducing either virD1 and virD2 (pPHP70298); the complete virD operon (i.e., virD1-D5) along with the virE1-3 and virG genes (pPHP71539); or the complete virD operon (i.e., virD1-D5) along with the virE1-3, virA, virG, and virJ genes (pPHP71539). The arrangement of the vir genes in the pPHP70298 and pPHP71539 vectors (see FIGS. 2 and 3) are a contiguous fragment as present in the parent pTiBo542 plasmid (see FIG. 4). However, in pPHP79761, the arrangement of the virA and virJ genes are arranged in a continguous fashion (see FIG. 4), whereas these genes are not contiguous in the parent pTiBo542 plasmid (see FIG. 4). In particular, the arrangement of the the virA and virJ genes in pPHP79761 removes a probable transposable DNA element (IS292) flanked by repeated sequences and four potential coding sequences (see CDS 51-54 in GenBank entry NC 010929). These constructs are smaller than pSB1, despite having an increased number of virulence genes.

Compared to pSB1, nonessential DNA and repetitive elements have been eliminated. For example, the vectors, pPHP70298, pPHP71539, and pPHP79761, do not contain the 7.0 kbp truncated tra and trb operons or flanking genes included in the 16.2 kb RK2 origin of replication found in pSB1 (see FIG. 1). There were two additional sequences found in pSB1 (see FIG. 1) that were removed in the vectors pPHP70298, pPHP71539, and pPHP79761: (a) a 32 bp palindromic sequence (SEQ ID NO: 61); and (b) a 146 bp inverted repeat sequence (SEQ ID NO: 62). It is believed that these elements in vector backbone decrease overall plasmid stability, and accordingly, their removal from the present vectors should increase overall plasmid stability.

The vectors, pPHP70298, pPHP71539, and pPHP79761, also lack the 2.7 kbp pBR322 fragment found in pSB1 comprising the origin of replication, a beta lactamase coding sequence, and unstable 18 bp poly-G flanked lambda COS sites. The vectors, pPHP70298, pPHP71539, and pPHP79761, instead use a 1.2 kbp ColE1 origin of replication which provides for stable maintenance in Escherichia coli.

The selectable marker is a gentamycin cassette, which provides for enhanced plasmid stability and faster growth of Agrobacterium. The tetracycline resistance gene tetAR in pSB1 often leads to slow growth or plasmid-free colonies in Agrobacterium strain C58 or mutant Agrobacterium colonies resistant to tetracycline (C58 and its derivatives such as AGL0, AGL1, have been described as giving rise to spontaneous tet resistant mutants at high frequency; see Luo, Z. K. and Farrand, S. K. (1999) J Bacteriol. 181:618-626). The gentamycin cassette used was the GmR synthetic aacC1 (based on GenBank Accession No. DQ530421; SEQ ID NO:1), conferring resistance to gentamycin, which does not lead to slow growth or reduced virulence in the recombinant Agrobacterium strain harboring the plasmid.

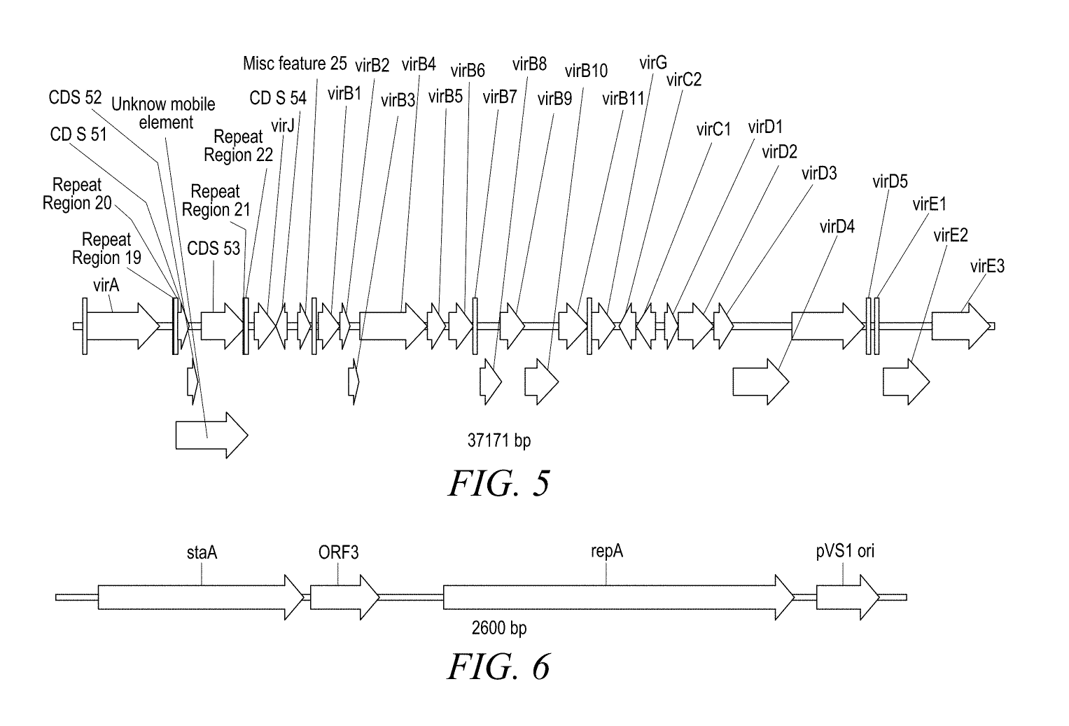

Briefly, the vectors were constructed in a similar manner. The construction of pPHP79761 is described here as an example of the methods used in the construction of pPHP70298 and pPHP71539. The virulence genes were obtained from the Agrobacterium tumefaciens Ti plasmid, pTiBo542 (NCBI Reference Sequence: NC_010929.1; see FIG. 5). One skilled in the art would appreciate that vir genes from Agrobacterium Rᵢ plasmid would also be useful in the vectors disclosed herein.

PCR primers were designed to amplify the virA, virJ, and virB1-virE3 coding and regulatory regions from a genomic DNA prep of Agrobacterium tumefaciens strain AGL1. The 24 kb virB1-E3 sequence was amplified in 5 pieces to facilitate PCR. Fragments/derivatives were designed with 40 bp overlapping ends to facilitate seamless cloning methods. Unique restriction enzyme sites were included between functional elements to facilitate their exchange with other functionally equivalent elements as shown in FIG. 4. A mobile genetic element labeled as IS292 in the pTiBo542 NCBI Reference Sequence that is present between the virA and virJ genes was not included in pPHP79761 to reduce size and the possibility of its transfer into plants. A gentamicin acetyltransferase gene (based on GenBank Accession No. DQ530421) was synthesized with flanking restriction sites to confer gentamicin resistance to its bacterial hosts. A ColE1 origin of replication corresponding to base pairs 2348-3247 of pBR322 (GenBank Accession No. J01749) was included for stable replication in E. coli. A 2.6 kb fragment encoding the pVS1 origin of replication (see Stanisich, V. J. Bacteriol. (1984) 129:1227-1233) was included for stable maintenance in Agrobacterium (see FIG. 6).

### Example 2

### Transgenic Maize Events via Agrobacterium Using the Helper pPHP70298

The vector, pPHP70298, was tested as a helper vector for corn transformation in two different maize cultivars, PHR03 and PH184C, using Agrobacterium mediated immature embryo transformation as described herein below. Agrobacterium tumefaciens strain LBA4404THY-harboring the ternary vector containing an expression cassette ZmUbi:PMI:PINII and ZmUbi:ZsYellow:PINII (pPHP45981; T-DNA; SEQ ID NO: 28) was tested. Side-by-side experiments were performed with pSB1 plus pPHP45981 and pPHP70298 plus pPHP45981 to compare the effect of these two vectors, pSB1 and pPHP70298, on maize transformation. The transformation was evaluated in terms of callus transformation frequency (Callus Tx %), T0 plant transformation frequency (T0 Tx %), quality event frequency (QE) (defined as the percentage of events with all genes of interest being single copy and not having any vector backbone DNA −QE %) and usable event quality (UE)) (defined as the number of QE events recovered for every 100 embryos infected UE %) for the two vectors. The transformation experiments were performed with a minimum of 600 embryos and the transformation data is summarized in Tables 2 and 3 for genotypes PHR03 and PH184C, respectively. The data in the tables demonstrate that the vector, pPHP70298, yielded a significant improvement in the overall callus and T0 plant transformation frequency in both of the maize cultivars tested.

TABLE 2
Transformation (Tx) data obtained in PHR03.

| Helper | Callus Tx | T0 Tx | QE | UE |
|---|---|---|---|---|
| Plasmid | (%) | (% ) | (%) | (%) |
| pSB1 | 54 | 14 | 40.7 | 5.7 |
| pPHP70298 | 67 | 29 | 29.5 | 8.5 |

TABLE 3
Transformation (Tx) data obtained in PH184C.

| Helper | Callus Tx | T0 Tx | QE | |
|---|---|---|---|---|
| Plasmid | (%) | (%) | (%) | UE |
| pSB1 | 63 | 45 | 41.5 | 18.6 |
| pPHP70298 | 84 | 61 | 20.2 | 12.3 |

Growing Agrobacterium on solid medium: Five mL Agrobacterium infection medium (700 medium; see Table 21) and 5 μL of 100 mM 3′-5′-Dimethoxy-4′-hydroxyacetophenone (acetosyringone) were added to a 14 mL Falcon tube in a hood. About 3 full loops of Agrobacterium were suspended in the tube and the tube was then vortexed to make an even suspension. One mL of the suspension was transferred to a spectrophotometer tube and the OD of the suspension was adjusted to 0.35 at 550 nm. The Agrobacterium concentration was approximately 0.5×10⁹ cfu/mL. The final Agrobacterium suspension was aliquoted into 2 mL microcentrifuge tubes, each containing 1 mL of the suspension. The suspensions were then used as soon as possible.

Growing Agrobacterium in liquid medium: One day before infection, a 125 ml flask was set up with 30 mL of 557A (see Table 21) with 30 μL spectinomycin (50 mg/mL) and 30 μL acetosyringone (20 mg/mL). A half loopful of Agrobacterium was suspended into the flasks and placed on a 200 rpm shaker at 28° C. overnight. The Agrobacterium culture was centrifuged at 5000 rpm for 10 min. The supernatant was removed and the Agrobacterium infection medium (700 medium; see Table 21) with acetosyringone solution was added. The bacteria were resuspended by vortex and the OD of Agrobacterium suspension was adjusted to 0.35 at 550 nm.

Maize Transformation: Ears of a maize (Zea mays L.) cultivar, PHRO3 or PH184C, were surface-sterilized for 15-20 min in 20% (v/v) bleach (5.25% sodium hypochlorite) plus 1 drop of Tween 20 followed by 3 washes in sterile water. Immature embryos (IEs) were isolated from ears and were placed in 2 ml of the Agrobacterium infection medium (700 medium; see Table 21) with acetosyringone solution. The optimal size of the embryos was 1.5-1.8 mm for PHR03, respectively. The solution was drawn off and 1 ml of Agrobacterium suspension was added to the embryos and the tube vortexed for 5-10 sec. The microfuge tube was allowed to stand for 5 min in the hood. The suspension of Agrobacterium and embryos were poured onto co-cultivation medium (710I; see Table 21). Any embryos left in the tube were transferred to the plate using a sterile spatula. The Agrobacterium suspension was drawn off and the embryos placed axis side down on the media. The plate was sealed with Parafilm™ film (moisture resistant flexible plastic, available at Bemis Company, Inc., 1 Neenah Center 4^{th} floor, PO Box 669, Neenah, Wis. 54957) and incubated in the dark at 21° C. for 1-3 days of co-cultivation.

Embryos were transferred to resting medium (605 W; see Table 21) without selection. Three to 7 days later, they were transferred to selection media 13152T or 13152Z (see Table 21) supplemented with mannose (12.5 g/L) or another selective agent (G418, 150 mg/L). Three weeks after the first round of selection, cultures were transferred to fresh 13152T or 13152Z (see Table 21) containing a selective agent at 3- to 4-week intervals. Once transformed, the tissues were transferred to maturatiom medium (289Q or 289M; see Table 21) supplemented with appropriate selective agent.

### Example 3

### Comparison of pPHP70298 and pPHP71539 to Plasmid pSB1 for Maize Transformation

The impact of pPHP70298 and pPHP71539 for maize transformation in the maize genotype PH184C using Agrobacterium mediated immature embryo transformation was determined. In this study, ternary production vectors were utilized that harbor at least one or more genes of interest (GOI) operably linked to marker genes, phosphomannose-isomerase (PMI) and phosphinothricin acetyl transferase (moPAT), for plant selection within the T-DNA. The transformation data are presented in Table 4, and these data show that the transformation frequency was at least 1.5- 2-fold higher in the experiments at the callus and T0 plant level for PH184C using pPHP70298 and pPHP71539 compared pSB1. The quality event (QE) frequency (defined as the percentage of events with all genes of interest being single copy and not having any vector backbone DNA) was not significantly different between the three plasmids pSB1, pPHP70298, and pPHP71539, but the usable event frequency (UE) (defined as the number of QE events recovered for every 100 embryos infected) was higher for both pPHP70298 and pPHP71539 for three independent production vectors. The average transformation frequency, QE % and UE for the three production vectors are summarized in Table 4.

TABLE 4
Summary of transformation data in the genotype PH184C.

| | | Callus Tx | T0 Tx | QE | |
|---|---|---|---|---|---|
| Construct | Helper | (%) | (%) | (%) | UE |
| A | pSB1 | 21.9 | 12.9 | 40.8 | 5.3 |
| | pPHP70298 | 36.9 | 24.1 | 26.3 | 6.3 |
| | pPHP71539 | 48.5 | 33.2 | 28.4 | 9.4 |
| B | pSB1 | 23.7 | 14.5 | 39.1 | 5.7 |
| | pPHP70298 | 32.3 | 18.9 | 32.4 | 6.1 |
| | pPHP71539 | 51 | 29 | 34 | 9.9 |
| C | pSB1 | 23.1 | 13.7 | 30.8 | 4.2 |
| | pPHP70298 | 30.8 | 17 | 29.3 | 5 |
| | pPHP71539 | 56.7 | 31.1 | 27.4 | 8.5 |
| Average | pSB1 | 22.9 | 13.7 | 36.9 | 5 |
| | pPHP70298 | 33.3 | 19.9 | 29.1 | 5.8 |
| | pPHP71539 | 52.3 | 31.1 | 29.9 | 9.3 |

### Example 4

### Improved Transformation with pPHP71539

The impact of pPHP71539 on maize transformation was further tested in another maize cultivar, PHR03, via Agrobacterium mediated immature embryo transformation. In this study, production vectors were utilized that harbor at least one or more genes of interest (GOI) operably linked to marker genes, phosphomannose-isomerase (PMI) and phosphinothricin acetyl transferase (moPAT), for plant selection within the T-DNA. As shown in Table 5, pPHP71539 markedly improved transformation frequency without negatively impacting the quality event frequency resulting in significant gains in the usable event recovery in the genotype PHR03 when compared to pSB1. These data further document the improvement achieved with the disclosed plasmids for maize transformation in multiple genotypes.

TABLE 5
Transformation (Tx) data using pPHP71539 in
the genotype PHR03 with indicated construct.

| | | Callus Tx | T0 Tx | QE | |
|---|---|---|---|---|---|
| Construct | Helper | (%) | (%) | (%) | UE |
| D | pSB1 | 23 | 4 | 27 | 1.2 |
| | pPHP71539 | 47 | 22 | 23 | 5.1 |
| E | pSB1 | 8 | 3 | 20 | 0.7 |
| | pPHP71539 | 43 | 16 | 38 | 6 |
| F | pSB1 | 28 | 11 | 33 | 3.5 |
| | pPHP71539 | 45 | 20 | 27 | 5.4 |

One skilled in the art would appreciate that Agrobacteria with helper plasmid pVIR10 (SEQ ID NO: 36; see Table 23) can significantly improve transformation frequency, recovery of quality events and usable quality events in multiple corn inbreds.

### Example 5

### Improved Transient DNA Delivery and Gene Expression in Plants

The plasmid, pPHP71539, was tested for transient T-DNA delivery in leaf explants using an Agroinfection method. For Agroinfection, young leaf explants were vacuum infiltrated with an Agrobacterium strain harboring a production cassette (i.e., a plant protection GOI) and leaves were harvested 2-3 days post infiltration for protein measurements. The protein measurements were made with standard ELISA using replicate samples and repeated twice. The data on transient DNA delivery in maize leaves is presented in Table 6. The protein amount measured in leaves infiltrated with pPHP71539 was significantly higher than the protein amounts seen in the leaves infiltrated with the same gene expression cassette with pSB1. This demonstrates that pPHP71539 facilitated higher T-DNA delivery and improved transient protein expression in maize.

TABLE 6
Transient gene expression in maize leaves.

| | | Protein expression | Average expression |
|---|---|---|---|
| Construct | Helper Vector | (ppm) | (ppm) |
| G | pSB1 | 418 | 429 |
| | | 396 | |
| | | 377 | |
| | | 525 | |
| | pPHP71539 | 537 | 660 |
| | | 604 | |
| | | 810 | |
| | | 691 | |

One skilled in the art would appreciate that Agrobacteria with helper plasmid pVIR7, pVIR9 or pVIR10 can significantly improve transient protein expression in different plant explants in multiple corn inbreds.

### Example 6

### Improved Sorghum Transformation using Agrobacterium Carrying pPHP70298

To demonstrate the improved functionality of the disclosed vectors for improving plant transformation in other monocots, the transformation frequency was tested for Agrobacterium-mediated sorghum transformation using immature embryos from sorghum variety TX430. In the first experiment, Agrobacterium tumefaciens strain LBA4404THY-harboring the ternary vector, pPHP45981, containing an expression cassette ZmUbi PMI:PINII and ZmUbi:ZsYellow:PINII (Seq ID NO. 28) was used. Side-by-side experiments were performed with pPHP45981 plus pSB1 and pPHP45981 plus pPHP70298 to determine the callus transformation frequency (Callus Tx %), T0 plant transformation frequency (T0 Tx %), quality event frequency (QE %) and usable event quality (UE). The transformation data from two independent experiments with a minimum of 300 immature embryos is summarized in Table 7. The strain LBA4404THY-harboring, pPHP45981 plus pPHP70298 plasmid showed improved performance at all stages in sorghum transformation including callus transformation, T0 plant transformation, quality event and overall usable quality event. Additional testing was carried out with constructs containing at least one trait stack and PMI expression cassette plus pPHP71539. The average transformation frequency with pPHP71539 (Table 8) was higher than the normal transformation frequency observed with the ternary vectors containing pSB1 (Table 7).

TABLE 7
Transformation data obtained using pPHP70298
in sorghum with construct pPHP45981.

| | Callus Tx | T0 Tx | QE | |
|---|---|---|---|---|
| Helper | (%) | (%) | (%) | UE |
| pSB1 | 4.2 | 2.1 | 64.7 | 1.4 |
| pPHP70298 | 10.5 | 5.7 | 71.8 | 4.1 |

TABLE 8
The T0 transformation frequency observed
using pPHP71539 in sorghum.

| | Callus Tx | T0 Tx |
|---|---|---|
| Constructs | (%) | (%) |
| H | 11 | 5 |
| I | 37 | 19 |
| J | 7 | 4 |

One skilled in the art would appreciate that Agrobacteria with helper plasmid pVIR7 (SEQ ID NO: 34), pVIR9 (SEQ ID NO: 35) or pVIR10 (SEQ ID NO: 36) can significantly improve transformation frequency, recovery of quality events and usable quality events in different sorghum lines.

### Example 7

### Improved Site-Specific Integration Using pPHP71539

pPHP71539 for Agrobacterium-mediated site-specific integration (“SSI”) was assessed on a target line harboring the target locus created in maize cultivar, PHR03, as described in U.S. Pat. No. 6,187,994 and U.S. Provisional Appl. No. 62/296639, both herein incorporated in entirety by reference. A target site operably linked to a promoter trap was used to aid in target event identification, and SSI event identification. Lines comprising a promoter trap target site were generated by transformation with a construct comprising pPHP64484 ZmProUbi-FRT1-NptII::PinII+ZmUbiPro::AmCyan::PinII-FRT87 (SEQ ID NO: 29). The binary vectors were generated with a promoter trap selectable marker plus a constitutive promoter (ZmUbiPro; SEQ ID NO: 30) driving the reporter gene (DsRed (SEQ ID NO: 31) flanked by non-identical FRT recombination site pairs (e.g., FRT1/FRT87 (SEQ ID NOS: 32 and 33) within the T-DNA, referred to as the donor. The binary vectors were mobilized into the Agrobacterium strain AGL1 with and without pPHP71539 for evaluating the effect of pPHP71539 on the recovery of SSI events and usable SSI frequency (precise SSI events). The molecular characterization of the putative events was carried out using qPCR/PCR assays to determine the excision of target gene (NptII), integration of the donor genes (PMI and DsRED), absence of FLP gene (random T-DNA integration), and presence of the FRT pair junction (1/87). A multiplex PCR was performed for vector backbone analysis. A precise SSI event (usable SSI event) was characterized as one which meets the following criteria: (1) single intact copy of the donor genes (PMI and DsRed); (2) absence of the target gene (NptII), FLP, ODP/MoCre; (3) presence of both FRT1 and FRT87 junctions; and, (4) free of any vector backbone insertion. Table 9 summarizes transformation frequency and precise SSI frequency obtained using the construct pPHP71518 in the Agrobacterium strain AGL1 with or without the helper plasmid, pPHP71539, in maize cultivar PHR03. Significant improvement in the recovery of putative SSI events and recovery of precise SSI T0 events was observed with the Agro strain containing the pPHP71518 plus pPHP71539 as compared to Agrobacterium strain with pPHP71518 alone.

TABLE 9
Data representing the transformation frequency and SSI frequency in PHR03 with
and without pVIR9 helper plasmid containing the donor construct pPHP71518

| | Total | Callus | | T0 | | SSI | |
|---|---|---|---|---|---|---|---|
| Construct | Embryo | # Events | Frequency | #Events | Frequency | #Events | Frequency |
| 71518 | 817 | 151 | 18.8% | 78 | 9.5% | 12 | 1.5% |
| 71518 + | 821 | 190 | 23.3% | 96 | 11.7% | 22 | 2.7% |
| pPHP71539 | | | | | | | |

One skilled in the art would appreciate that Agrobacteria with helper plasmid pVIR7 or pVIR10 can significantly improve TO transformation frequency and precise SSI frequency in multiple corn inbreds.

### Example 8

### Improved Wheat Transformation Using Pvir Plasmid (High Efficiency Transformation and Rapid to Platn Production in Wheat

### A. Wheat Transformation Methods

An aliquot of Agrobacterium strain LBA4404 THY-containing the vector of interest is removed from storage at −80° and streaked onto solid LB medium (12V) containing a selective agent spectinomycin. The Agrobacterium was cultured on the 12V at 21° C. in the dark for 2-3 days, at which time a single colony is selected from the plate, streaked onto 810D medium plate containing the selective agent and then incubated at 28° C. in the dark overnight. Two to three Agrobacterium colonies were picked using a sterile spatula and suspended in ˜5 mL wheat infection medium (WI4) (WI4, see Table 10) with 400 uM acetosyringone (AS). The optical density (600 nm) of the suspension was adjusted to about 0.1 to 0.7 using the same medium.

Four to five spikes containing immature seeds (with 1.4-2.3 mm embryos) were collected, and the immature embryos were isolated from the immature seeds. The wheat grains were surface sterilized for 15 min in 20% (v/v) bleach (5.25% sodium hypochlorite) plus 1 drop of Tween 20, followed with 2-3 washes in sterile water. After sterilization, the immature embryos (IEs) were isolated from the wheat grains and placed in 1.5 ml of WI4 medium. The immature embryos were transferred to a 2 mL microcentrifuge tube containing 0.25 mL sterile sand plus 1 mL WI4 medium, and then centrifuged at 10,000 RPM for 30 seconds. Following first centrifugation, the micricentrifuge tube was vortexed at a medium setting for 10 seconds, and again centrifuged a second time at 10,000 RPM for 30 seconds. The embryos were allowed to sit in this suspension for 20 minutes.

In the next step, WI4 medium was decanted and infection of the immature embryos was initiated by adding 1.0 ml of Agrobacterium suspension. The infected embryos were allowed to sit for 20 minutes. The suspension of Agrobacterium and IEs was poured onto wheat co-cultivation medium #10 (see Table 11). Embryos were poured to the plate using a sterile spatula, with axis side placed down on the media, making sure the embryos are immersed in the solution. The plate was sealed with Parafilm® tape film (moisture resistant flexible plastic, available at Bemis Company, Inc., 1 Neenah Center 4^{th} floor, PO Box 669, Neenah, Wis. 54957) and incubated in the dark at 21° C. for 3 days of co-cultivation. Tables 10-15 describe liquid wheat infection medium (WI4), wheat co-cultivation medium (WC#10), first round DBC4 medium, second round DBC6 medium, regeneration MSA medium, and regeneration MSB medium.

TABLE 10
Composition of wheat liquid infection medium WI4.
WI 4

| DI water | 1000 | mL |
|---|---|---|
| MS salt + Vitamins(M519) | 4.43 | g |
| Maltose | 30 | g |
| Glucose | 10 | g |
| MES | 1.95 | g |
| 2,4-D (.5 mg/L) | 1 | ml |
| Picloram (10 mg/ml) | 200 | μl |
| BAP (1 mg/L) | .5 | ml |
| Adjust PH to 5.8 with KOH | | |
| Post sterilization add: | | |
| Acetosyringone (400 μM) | 400 | μl |

TABLE 11
Composition of wheat co-cultivation medium WC#10.
WC # 10

| DI water | 1000 | mL |
|---|---|---|
| MS salt + Vitamins(M519) | 4.43 | g |
| Maltose | 30 | g |
| Glucose | 1 | g |
| MES | 1.95 | g |
| 2,4-D (.5 mg/L) | 1 | ml |
| Picloram (10 mg/ml) | 200 | μl |
| BAP (1 mg/L) | .5 | ml |
| 50X CuSO4 (.1M) | 49 | μl |
| Adjust PH to 5.8 with KOH and add 2.5 g/L of Phytagel. | | |
| Post sterilization add: | | |
| Acetosyringone (400 μM) | 400 | μl |

TABLE 12
Composition of wheat green tissue culture medium DBC4.
DBC4

| dd H20 | 1000 | mL |
|---|---|---|
| MS salt | 4.3 | g |
| Maltose | 30 | g |
| Myo-inositol | 0.25 | g |
| N-Z-Amine-A | 1 | g |
| Proline | 0.69 | g |
| Thiamine-HCl (0.1 mg/mL) | 10 | mL |
| 50X CuSO4 (0.1M) | 49 | μL |
| 2,4-D (0.5 mg/mL) | 2 | mL |
| BAP | 1 | mL |
| Adjust PH to 5.8 with KOH and then add 3.5 g/L of Phytagel. | | |
| Post sterilization add: | | |
| Cef(100 mg/ml) | 1 | ml |

TABLE 13
Composition of wheat green tissue induction medium DBC6.
DBC6

| dd H20 | 1000 | mL |
|---|---|---|
| MS salt | 4.3 | g |
| Maltose | 30 | g |
| Myo-inositol | 0.25 | g |
| N-Z-Amine-A | 1 | g |
| Proline | 0.69 | g |
| Thiamine-HCl (0.1 mg/mL) | 10 | mL |
| 50X CuSO4 (0.1M) | 49 | μL |
| 2,4-D (0.5 mg/mL) | 1 | mL |
| BAP | 2 | mL |
| Adjust PH to 5.8 with KOH and then add 3.5 g/L of Phytagel. | | |
| Post sterilization add: | | |
| Cef(100 mg/ml) | 1 | ml |

TABLE 14
Composition of wheat regeneration medium MSA.
MSA

| dd H20 | 1000 | mL |
|---|---|---|
| MS salt + Vitamins(M519) | 4.43 | g |
| Sucrose | 20 | g |
| Myo- Inositol | 1 | g |
| Adjust PH to 5.8 with KOH and then add 3.5 g/L of Phytagel. | | |
| Post steriliaztion add: | | |
| Cef(100 mg/ml) | 1 | ml |

TABLE 15
Composition of wheat regeneration medium MSB.
MSB

| dd H20 | 1000 | mL |
|---|---|---|
| MS salt + Vitamins(M519) | 4.43 | g |
| Sucrose | 20 | g |
| Myo- Inositol | 1 | g |
| Adjust PH to 5.8 with KOH and then add 3.5 g/L of Phytagel. | | |
| Post steriliaztion add: | | |
| Cef (100 mg/ml) | 1 | ml |
| IBA | .5 | ml |

### B. Wheat Embryo Selection

The immature embryos are transferred to DBC4 green tissue (see Table 12) (GT medium with 100 mg/L cefotaxime (PhytoTechnology Lab., Shawnee Mission, Kans.) induction medium without selection, in the orientation of the embryo axis being in contact with the medium. The embryos are incubated on this medium at 26-28° C. in dim light for two weeks then transfered to DBC6 medium (see Table 13) containing 100 mg/L cefotaxime for another two weeks. At this time, all tissue expressing the fluorescent protein is separated from the non-transformed tissues under a fluorescence microscope and placed on DBC6 GT induction medium (see Table 13) containing 100 mg/L cefotaxime for tissue proliferation.

### C. Regeneration of Plantlets and Transfer to the Greenhouse

The fluorescing tissue is transferred to MSA regeneration medium (see Table 14), and incubated at 26-28° C. in bright light for 2-4 weeks. At this point, the tissue is checked for uniform expression of the fluorescent marker genes in transgenic plantlets and for healthy roots. The plantlets are transferred into soil in pots in the greenhouse.

D. The Introduction of the Super Binary Plasmids pPHP71539 and pPHP70298 in Agrobacteria Containing Plant Expression Cassettes Resulted in Improved Transient T-DNA Delivery and Improved Recovery of Transgenic T0 Events in Wheat

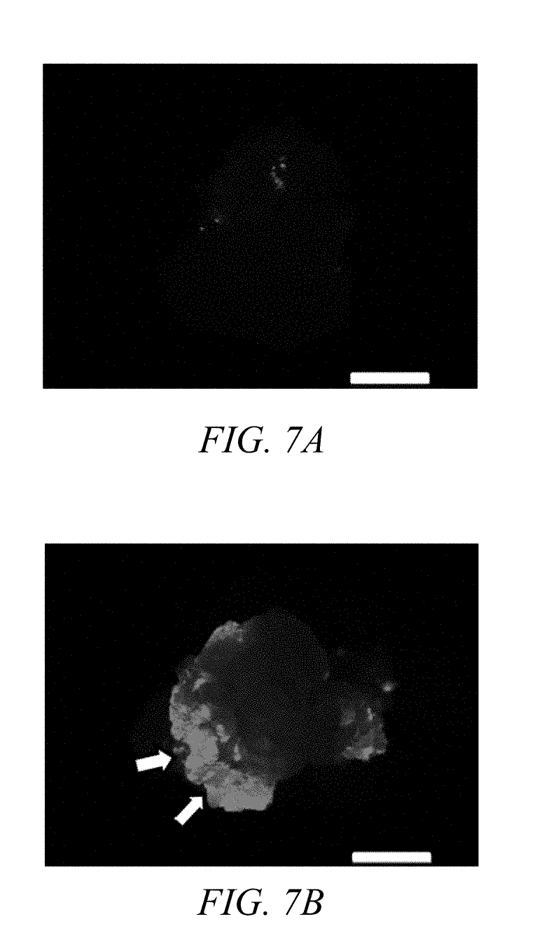

Immature embryos were harvested from wheat cultivar HC0456D and were infected with Agrobacterium strains LBA4404 THY-pPHP71539 and AGL1 containing a T-DNA binary plasmid with the following composition—RB-UBI-ZMPRO:MO-PAT:PROTEIN LINKER:DS-RED:PINII-LB (SEQ ID NO.68. Agrobacterium strain LBA4404 THY- is a weaker strain which previously was shown to result in weak transient T-DNA delivery and has been described elsewhere as a poor strain for wheat transformation, therefore excluded from the study. For transient T-DNA expression 20-30 embryos of the cultivar were infected and DS-RED expression was monitored at 7 days and 15 days post infection. In the experiment, two different Agrobacterium strains AGL1 and LBA4404 Thy-pPHP71539 were tested to characterize the effect of pVIR9 on stable transformation (15 DPI) in wheat (FIG. 7). The native AGL1 strain showed several fold lower transient DsRed expression at 7DPI and continued to show fewer DsRed expressing sectors at 15DPI (FIG.7), representing stable T-DNA expression in the infected embryos as compared to strain LBA4404THY-pPHP71539.

In a separate experiment, Agrobacteria containing the pVIR helper plasmid improved stable transformation and increased recovery of T0 events in multiple wheat cultivars. To this end, side-by-side wheat transformation experiments were performed in two different wheat cultivars (Fielder and HC0456D) with Agrobacterium strain AGL1, with and without pVIR helper pPHP70298. Immature embryos were harvested from two wheat cultivars (Fielder and HC0456D) and were infected with Agrobacteria AGL1 and AGL1 plus pPHP70298 containing two different binary plasmids (A and B) containing the following expression cassette; RB-UBI-ZMPRO: MO-PAT: PROTEIN LINKER: DS-RED:PINII-LB (SEQ ID NO.68) to capture T0 plant transformation frequency. The transformation data is summarized in Table 16.

TABLE 16
Transformation data using pPHP70298 in multiple wheat cultivars.

| | | AGL1 | | AGL1 + pPHP70298 | |
|---|---|---|---|---|---|
| Cultivar | Construct | (T0 TX %) | | (T0 TX %) | |
| Fielder | A | 0.5% | (26/5748) | 1.4% | (4/278) |
| HC0456D | A | 0% | (0/1918) | 1.1% | (4/375) |
| Fielder | B | 0.4% | (9/2294) | 5.4% | (47/863) |

| | | | | |
|---|---|---|---|---|
| HC0456D | B | 0% | 5.3% | (36/678) |

Wheat immature embryos transformed with AGL1 plus pPHP70298 showed remarkably improved performance at all stages in wheat transformation including callus transformation and T0 plant transformation. The T0 plant transformation frequency with Agrobacteria containing the pVIR7 plasmid was determined to be much higher than the wild-type strain of AGL1 without the pVIR7 helper plasmid. Ochrobactrum containing the pVIR7, the pVIR9 or the pVIR10 plasmids has been used to successfully transform plants (data not shown) as described in U.S. Provisional Appl. No. 62/211267, herein incorporated by reference in its entirety.

One skilled in the art would appreciate that Agrobacteria with helper plasmid pVIR9 (SEQ ID NO: 35, see Table 23) or pVIR10 can significantly improve transformation frequency, recovery of quality events and usable quality events in multiple wheat lines.

### Example 9

### PVIR9 Plasmid Improved Maize Transformation

The pVIR9 plasmid was tested for maize transformation using the ternary vector system to transform corn genotype PH184C as described in U.S. Provisional Appl. No. 62/248578, herein incorporated in entirity by reference. The introduction of the super binary plasmid pPHP71539 in Agrobacteria containing plant expression cassettes resulted in improved transient T-DNA delivery, somatic embryos phenotype and improved recovery of transgenic T0 events in corn.

Briefly, immature embryos (2-2.5 mm in length) were harvested from Pioneer maize inbred PH184C) approximately 11 days after pollination, and were infected with Agrobacterium strain LBA4404 THY-containing T-DNA plasmids 1) pPHP80561 or 2) pPHP80559 with the following composition; pPHP80561-RB+LOX P-ZM-AXIG1 1XOP-WUS2::IN2-1 TERM+ZM-PLTP PRO::ZM-ODP2::OS-T28 TERM+PINII+GZ-W64A TERM+OLE PRO:MO-CRE EXON1:ST-LS1 INTRON2:MO-CRE EXON2-PINII TERM+SB-UBI PRO: SB-UBI INTRON1:UBI PRO:ZS-GREEN:OS-UBI TERM+LOXP+SB-ALS PRO:: HRA::PINII TERM+LB (SEQ ID NO.69) and, pPHP80559-RB+LOX P-ZM-AXIG1 1XOP-WUS2::IN2-1 TERM+ZM-PLTP PRO::ZM-ODP2::OS-T28 TERM+PINII+GZ-W64A TERM+LTP2 PRO:MO-CRE EXON1:ST-LS1 INTRON2:MO-CRE EXON2-PINII TERM+SB-UBI PRO:SB-UBI INTRON1:UBI PRO:ZS-GREEN:OS-UBI TERM+LOXP+SB-ALS PRO::HRA::PINII TERM+LB (SEQ ID NO. 70). These plasmids were mobilized into two different Agrobacterium strains LBA4404THY-pSB1 and LBA4404THY-pVIR9 (pPHP71539). The Agrobacteria with the plasmid was grown in liquid medium to an optical density of 0.5 (at 520 nm) and the immature embryos (˜600 plus embryos, split ear, three replicates) were incubated in the Agrobacterium suspension for 5 minutes before removal from the liquid to be placed on solid 7101 medium. After 24 hours, the embryos were moved to 605T medium (see Table 21) to begin selection against the Agrobacterium. After 6 days, numerous small somatic embryos were observed on the surface of the treated immature embryos. Seven days after Agro-infection, the embryos were transferred to maturation medium (289Q medium with 0.1 mg/l imazapyr), using the imidazolinone herbicide to select for transgenic embryos. After 14 days on the maturation medium, the mature embryos were moved onto rooting medium (13158H medium; 13158 medium plus 25 mg/l cefotaxime) and leaf pieces were sampled for PCR analysis. The data on TX frequency, excised QE frequency and UE frequency is represented in Table 17.

TABLE 17
Transformation data in inbred PH184C comparing pVIR9 and
pSB1plasmid with two different expression vectors.

| | | | | | | | Excised | QE | |
|---|---|---|---|---|---|---|---|---|---|
| | | | | | # T0 | | QE | excision | |
| Treatment | pPHP | Helper | Pro:CRE | # Emb | plant | TX % | events | % | UQE % |
| A | pPHP80561 | pVIR9 | Ole | 610 | 120 | 20% | 21 | 18% | 3.4% |
| | pPHP80561 | pSB1 | Ole | 607 | 19 | 3% | 4 | 21% | 0.7% |
| B | pPHP80559 | pVIR9 | LTP2 | 610 | 113 | 19% | 23 | 20% | 3.8% |
| | pPHP80559 | pSB1 | LTP2 | 610 | 42 | 7% | 6 | 14% | 1.0% |

The immature embryos transformed with Agrobacteria containing the pPHP71539 (pVIR9) plasmid produced very high number of T0 plants and resulted in higher T0 transformation frequency as compared to Agrobacteria transformed with the pSB1 plasmid. The Agrobacteria with the helper plasmid pVIR9 also resulted in significantly higher frequency of useable quality events when compared to immature embryos infected with plasmid pSB1.

One skilled in the art would appreciate that Agrobacteria with helper plasmid pVIR7 or pVIR10 instead of pVIR9 can significantly improve maize transformation, quality events and usable quality events.

### Example 10

### PVIR Plasmid with Different Origins of Replication for Plant Transformation

For characterizing the effect of different origins of replication on the helper plasmid for corn transformation, the ternary vector system was used to transform corn genotype HC69. Using the maize transformation approach as described above in Example 9 (U.S. Provisional Appl. No. 62/248578, herein incorporated in entirity by reference), four pVIR plasmids containing different bacterial origins of replication namely pVS1, pSaparDE and RK2parDE (Table 18) were tested. Immature embryos were harvested from maize inbred (HC69) and were infected with Agrobacterium (strain LBA4404 THY-) containing pPHP79066 (SEQ ID NO.71) with the following T-DNA composition and the helper plasmids with different bacterial origins of replication (“ori”) namely the following: pVS1 (pVIR10; pPHP79761), pSaparDE (pVIR10; pPHP80399), RK2parDE (pVIR10; pPHP80566) and control (pVIR9; pPHP71539) as detailed in Table 18. For each construct, 150 embryos of the inbred HC69 were transformed using the split ear method to determine the transformation frequency. All the constructs transformed corn at very high frequency demonstrating the differnt ori combinations worked in the pVIR plasmid. The transformation data for inbred HC69 is presented in Table 19.

TABLE 18
Testing of different Ori's on pVIR plasmid.

| Vector# | Binary | pVIR | Ori |
|---|---|---|---|
| 1 | pPHP79066 | pPHP79761 | pVS1 |
| 2 | pPHP79066 | pPHP80399 | pSA parDE |
| 3 | pPHP79066 | pPHP80566 | RK2micro parDE |
| 4 | pPHP79066 | pPHP71539 | pVS1 |

TABLE 19
Transformation data in inbred HC69 with
pVIR plasmid containing different ori.

| Binary/pVIR | Total | Total | Transformation |
|---|---|---|---|
| combinations | Embryos | T0 plants | frequency |
| pPHP79066/pPHP79761 | 183 | 154 | 84% |
| pPHP79066/pPHP80399 | 160 | 116 | 73% |
| pPHP79066/pPHP80566 | 176 | 173 | 98% |
| pPHP79066/pPHP71539 | 170 | 175 | 103% |

The pVIR plasmids with different ori transformed corn and regenerated T0 events. The T0 transformation frequency was comparable across all the different ori tested.

### Example 11

### Enhanced Delivery System for Crispr/Cas9 Reagents and Gene Mutations with PVIR Plasmid in Corn

To characterize the effect of the helper plasmid on CRIPSR/Cas9 delivery and the rate of mutants recovered from the different helper plasmids, a ternary vector system was used to transform corn genotype PH2HT. Using the random transformation protocol, two different constructs were transformed, pPHP78147 (SEQ ID NO.72) and pPHP78148 (SEQ ID NO. 73), each comprising of two guide RNA (gRNA) sequences, specific for generating dropout deletions in target genes-ZM-ARGOS8 and ZM-GCN2. The sequences of the guide RNA sequences and the expression cassettes are described in SEQ ID NO: 72 and SEQ ID NO: 73, respectively. Immature embryos were harvested from maize inbred PH184C and were infected with Agrobacterium (strain LBA4404 THY-) containing helper plasmids pPSB1 or pVIR9 containing the T-DNA expression cassettes pPHP78147 or pPHP78148. For each construct approximately 280 embryos of the inbred PH2RT was transformed using the split ear method to determine the transformation frequency, mutation rates, single dropout deletions and biallelic dropout deletion rates. For determining the mutation rates, the T0 plants were subjected to deep sequencing analysis and the molecular event quality was determined by running PCR and qPCR assays on target genes as described previously. The transformation data and the gene edit types resulted from each construct in PH2RT are presented in Table 20.

TABLE 20
Types of gene edits resulted from each construct.

| | | | | | | | Single | | |
|---|---|---|---|---|---|---|---|---|---|
| | | # | | | | # 1 | allele | # | Biallelic |
| | | Embryo* | # T0 | T0 | Mutation | allele | dropout | Biallelic | dropout |
| pPHP | Helper | (no.) | Events | Txn % | rate | dropout | rate | dropout | rate |
| pPHP78147 | pSB1 | 285 | 130 | 61.8% | 42.3% | 3 | 2.3% | 7 | 5.4% |
| (ARGOS8) | | | | | | | | | |
| pPHP78147 | pVIR9 | 285 | 129 | 80.3% | 46.5% | 10 | 7.7% | 6 | 4.7% |
| (ARGOS8) | | | | | | | | | |
| pPHP78148 | pSB1 | 280 | 112 | 47.1% | 86.6% | 18 | 16.1% | 5 | 4.5% |
| (GCN2) | | | | | | | | | |
| pPHP78148 | pVIR9 | 280 | 127 | 59.6% | 92.1% | 22 | 17.3% | 8 | 6.3% |
| (GCN2) | | | | | | | | | |

The transformation frequency and mutation rates recovered from the new pVIR superbinary vectors were much higher than that observed with pSB1. The transformation frequency varied depending on the gRNA sequences and the deletion types. For ARGOS8, wherein a larger dropout deletion was targeted (619 bp), observed transformation frequency with pVIR9 was about 80.3% compared to 61.8% with pSB1. The frequency at which single allele dropouts were recovered with the ARGOS8 construct was higher with pVIR9 (7.7%) when compared to pSB1 (2.3%). With the second construct targeting a smaller dropout deletion in ZM-GCN2 (52 bp), embryos transformed with Agro containing the helper plasmid pVIR9 resulted in higher T0 event generation and recovery of mutant events (mutations, single allele and biallelic) as compared to the events recovered from the Agro with the pSB1 helper (Table 20).

One skilled in the art would appreciate that Agrobacteria with helper plasmid pVIR7 or pVIR10 can enhance delivery of CRISPR/Cas9 nucleases or related nucleases to improve genome editing and genome modifications in multiple corn inbreds.

### Example 12

### Improved Recovery of Site-Specific Integrations (ssi) Events on Agrobacterium Strain LBA4404THY-Harboring Superbinary Plasmid

To determine the effect of the super binary vectors (pVIR9; pPHP71539), on the recovery of precise SSI events, the pVIR9 helper plasmid in Agrobacterium strain LBA4404THY- was mobilized. Strain LBA4404THY- has been shown to result in low recovery of precise SSI events (U.S. Provisional Appl. No. 62/296639, herein incorporated in entirity by reference). To test whether pVIR9 in Agro strain LBA4404THY-improved recovery of SSI events, a binary vector containing the donor cassette with the following expression cassette RB-OS-ACTIN PRO:OS-ACTIN INTRON::ZM-WUS2::PINII+UBI PRO::UBI1ZM INTRON::ZM-ODP2::PINII TERM::UBI PRO:UBI1ZM INTRON::MO-FLP::PINII TERM::CaMV35S TERM+FRT1:PMI::PINII TERM+TRAIT GENE+FRT87-LB was mobilized into two different Agrobacterium strains AGL1 and LBA4404THY-pPHP71539 (pPHP79366; SEQ ID NO. 74). Donor cassettes were delivered via Agro-mediated transformation into multiple target sites with FRT1-87 landing sites in the genotype PH184C as described below.

Briefly, Agro-mediated transformation into target lines with FRT1-FRT87 landing sites was carried out as follows. Ears of a maize (Zea mays L.) cultivar, PH184C, were surface-sterilized for 15-20 min in 20% (v/v) bleach (5.25% sodium hypochlorite) plus 1 drop of Tween 20 followed by 3 washes in sterile water. Immature embryos (IEs), typically 1.5-1.8 mm, were isolated from ears and were placed in 2 ml of the Agrobacterium infection medium with acetosyringone solution. The solution was drawn off and 1 ml of Agrobacterium suspension was added to the embryos, vortexed for 5-10 seconds, and then incubated 5 min at room temperature. The suspension of Agrobacterium and embryos were poured onto 710I co-cultivation medium (see Table 21). Any embryos left in the tube were transferred to the plate using a sterile spatula. The Agrobacterium suspension was drawn off and the embryos placed axis side down on the media. The plate was sealed with Parafilm™ tape (moisture resistant flexible plastic, available at Bemis Company, Inc., 1 Neenah Center 4^{th} floor, PO Box 669, Neenah, Wis. 54957) and incubated in the dark at 21° C. for 1-3 days of co-cultivation.

Embryos were transferred to resting medium 13265A (see Table 21) without selection. Three to 7 days later, they were transferred to selection media 13152Z (see Table 21) supplemented with mannose or other appropriate selective agent. Three weeks after the first round of selection, cultures were transferred to second round of selection media 13152Z (see Table 21) containing a selective agent at 3- to 4-week intervals. Once transformed, transgenic green tissues are selected and cultured essentially as described in U.S. Pat. No. 7,1020,56, U.S. Pat. No. 8,404,930, and publication US20130055472.

TABLE 21
Media formations for maize transformation, selection and regeneration.

| | Units | | | | | | | | | |
|---|---|---|---|---|---|---|---|---|---|---|
| Medium components | per liter | 12V | 810I | 700 | 710I | 605J | 605T | 289Q | 605W | 289M |
| MS BASAL SALT MIXTURE | G | | | 4.3 | 4.3 | 4.3 | 4.3 | 4.3 | 4.3 | 4.3 |
| N6 MACRONUTRIENTS 10X | ml | | | | | 60.0 | 60.0 | | 60.0 | |
| POTASSIUM NITRATE | G | | | | | 1.7 | 1.7 | | 1.7 | |
| B5H MINOR SALTS 1000X | ml | | | | | 0.6 | 0.6 | | 0.6 | |
| NaFe EDTA FOR B5H 100X | ml | | | | | 6.0 | 6.0 | | 6.0 | |
| ERIKSSON'S VITAMINS | ml | | | | | 0.4 | 0.4 | | 0.4 | |
| 1000X | | | | | | | | | | |
| S&H VITAMIN STOCK 100X | ml | | | | | 6.0 | 6.0 | | 6.0 | |
| THIAMINE•HCL | mg | | | 1.0 | 1.0 | 0.2 | 0.2 | | 0.2 | |
| L-PROLINE | g | | | | 0.7 | 2.0 | 2.0 | 0.7 | 2.0 | 0.7 |
| CASEIN HYDROLYSATE | g | | | | | 0.3 | 0.3 | | 0.3 | |
| (ACID) | | | | | | | | | | |
| SUCROSE | g | | | 68.5 | 20.0 | 20.0 | 20.0 | 60.0 | 20.0 | 60.0 |
| GLUCOSE | g | 5.0 | | 36.0 | 10.0 | 0.6 | 0.6 | | 10.0 | |
| MALTOSE | g | | | | | | | | | |
| 2,4-D | mg | | | 1.5 | 2.0 | 0.8 | 0.8 | | 0.8 | |
| AGAR | g | 15.0 | 15.0 | | 8.0 | 6.0 | 6.0 | 8.0 | | |
| PHYTAGEL | g | | | | | | | | 3.5 | |
| DICAMBA | g | | | | | 1.2 | 1.2 | | 1.2 | |
| SILVER NITRATE | mg | | | | | 3.4 | 3.4 | | | |
| AGRIBIO Carbenicillin | mg | | | | | 100.0 | | | 100.0 | |
| Timentin | mg | | | | | | 150.0 | 150.0 | | |
| Cefotaxime | mg | | | | | | 100.0 | 100.0 | | |
| MYO-INOSITOL | g | | | 0.1 | 0.1 | | | 0.1 | | 0.1 |
| NICOTINIC ACID | mg | | | 0.5 | 0.5 | | | | | |
| PYRIDOXINE•HCL | mg | | | 0.5 | 0.5 | | | | | |
| VITAMIN ASSAY CASAMINO | g | | | 1.0 | | | | | | |
| ACIDS | | | | | | | | | | |
| MES BUFFER | g | | | | 0.5 | | | | | |
| ACETOSYRINGONE | uM | | | | 100.0 | | | | | |
| ASCORBIC ACID 10 MG/ML | mg | | | | 10.0 | | | | | |
| (7S) | | | | | | | | | | |
| MS VITAMIN STOCK SOL. | ml | | | | | | | 5.0 | | 5.0 |
| ZEATIN | mg | | | | | | | 0.5 | | 0.5 |
| CUPRIC SULFATE | mg | | | | | | | 1.3 | 0.05 | 1.3 |
| IAA 0.5 MG/ML (28A) | ml | | | | | | | 2.0 | | 2.0 |
| ABA 0.1 mm | ml | | | | | | | 1.0 | | 1.0 |
| THIDIAZURON | mg | | | | | | | 0.1 | | 0.1 |
| AGRIBIO Carbenicillin | mg | | | | | | | 100.0 | | 100.0 |
| PPT(GLUFOSINATE-NH4) | mg | | | | | | | | | |
| BAP | mg | | | | | | | 1.0 | 0.1 | |
| YEAST EXTRACT (BD Difco) | g | | 5.0 | | | | | | | |
| PEPTONE | g | | 10.0 | | | | | | | |
| SODIUM CHLORIDE | g | | 5.0 | | | | | | | |
| SPECTINOMYCIN | mg | 50.0 | 100.0 | | | | | | | |
| FERROUS SULFATE•7H20 | ml | 2.0 | | | | | | | | |
| AB BUFFER 20X (12D) | ml | 50.0 | | | | | | | | |
| AB SALTS 20X (12E) | ml | 50.0 | | | | | | | | |
| Benomyl | mg | | | | | | | | | |
| pH | | 5.6 | 5.6 | 5.6 | 5.6 | 5.6 | 5.6 | 5.6 | 5.6 | 5.6 |

| | Units | | | | | | | | | | |
|---|---|---|---|---|---|---|---|---|---|---|---|
| Medium components | per liter | 289R | 13158H | 13224B | 13266K | 272X | 272V | 13158 | 13265A | 13152T | 13152Z |
| MS BASAL SALT | G | 4.3 | 4.3 | | 4.3 | 4.3 | 4.3 | 4.3 | 4.3 | 4.3 | 4.3 |
| MIXTURE | | | | | | | | | | | |
| N6 MACRONUTRIENTS | ml | | | 4.0 | 60.0 | | | | 60.0 | | |
| 10X | | | | | | | | | | | |
| POTASSIUM NITRATE | G | | | | 1.7 | | | | 1.7 | | |
| B5H MINOR SALTS | ml | | | | 0.6 | | | | 0.6 | | |
| 1000X | | | | | | | | | | | |
| NaFe EDTA FOR B5H | ml | | | | 6.0 | | | | 6.0 | | |
| 100X | | | | | | | | | | | |
| ERIKSSON'S | ml | | | 1.0 | 0.4 | | | | 0.4 | | |
| VITAMINS 1000X | | | | | | | | | | | |
| S&H VITAMIN STOCK | ml | | | | 6.0 | | | | 6.0 | | |
| 100X | | | | | | | | | | | |
| THIAMINE•HCL | mg | | | 0.5 | 0.5 | | | | 0.5 | 1.0 | 1.0 |
| L-PROLINE | G | 0.7 | 0.7 | 2.9 | 2.0 | | | | 2.0 | 0.7 | 0.7 |
| CASEIN | G | | | | 0.3 | | | | 0.3 | 1.0 | 1.0 |
| HYDROLYSATE | | | | | | | | | | | |
| (ACID) | | | | | | | | | | | |
| SUCROSE | G | 60.0 | 60.0 | 190.0 | 20.0 | 40.0 | 40.0 | 40.0 | 20.0 | | |
| GLUCOSE | G | | | | 0.6 | | | | 10.0 | | |
| MANNOSE | G | | | | | | | | | 12.5 | 12.5 |
| MALTOSE | G | | | | | | | | | 5.0 | 5.0 |
| 2,4-D | mg | | | | 1.6 | | | | 1.6 | 1.0 | 1.0 |
| AGAR | G | | 8.0 | 6.4 | 6.0 | 6.0 | 6.0 | 6.0 | | | |
| PHYTAGEL | G | | | | | | | | 3.5 | | |
| DICAMBA | G | | | | 1.2 | | | | 1.2 | | |
| SILVER NITRATE | mg | | | 8.5 | 1.7 | | | | | | |
| AGRIBIO Carbenicillin | mg | | | | 2.0 | | | | | 100 | |
| Timentin | mg | 150.0 | 150.0 | | | | | | 150.0 | | 150.0 |
| Cefotaxime | mg | 100.0 | 100.0 | 25 | 25 | | | | 100.0 | | 100.0 |
| MYO-INOSITOL | G | 0.1 | 0.1 | | | 0.1 | 0.1 | 0.1 | | 0.25 | 0.25 |
| NICOTINIC ACID | mg | | | | | | | | | | |
| PYRIDOXINE•HCL | mg | | | | | | | | | | |
| VITAMIN ASSAY | G | | | | | | | | | | |
| CASAMINO ACIDS | | | | | | | | | | | |
| MES BUFFER | G | | | | | | | | | | |
| ACETOSYRINGONE | uM | | | | | | | | | | |
| ASCORBIC ACID | mg | | | | | | | | | | |
| 10 MG/ML (7S) | | | | | | | | | | | |
| MS VITAMIN STOCK | ml | 5.0 | 5.0 | | | 5.0 | 5.0 | 5.0 | | | |
| SOL. | | | | | | | | | | | |
| ZEATIN | mg | 0.5 | 0.5 | | | | | | | | |
| CUPRIC SULFATE | mg | 1.3 | 1.3 | | | | | | 0.05 | 1.2 | 1.2 |
| IAA 0.5 MG/ML (28A) | ml | 2.0 | 2.0 | | | | | | | | |
| ABA 0.1 mm | ml | 1.0 | 1.0 | | | | | | | | |
| THIDIAZURON | mg | 0.1 | 0.1 | | | | | | | | |
| AGRIBIO Carbenicillin | mg | | | | | | | | | | |
| PPT(GLUFOSINATE- | mg | | | | | | | | | | |
| NH4) | | | | | | | | | | | |
| BAP | mg | | | | | | | | 0.1 | 0.5 | 0.5 |
| YEAST EXTRACT (BD | G | | | | | | | | | | |
| Difco) | | | | | | | | | | | |
| PEPTONE | G | | | | | | | | | | |
| SODIUM CHLORIDE | G | | | | | | | | | | |
| SPECTINOMYCIN | mg | | | | | | | | | | |
| FERROUS | ml | | | | | | | | | | |
| SULFATE•7H20 | | | | | | | | | | | |
| AB BUFFER 20X (12D) | ml | | | | | | | | | | |
| AB SALTS 20X (12E) | ml | | | | | | | | | | |
| Benomyl | mg | | | | | | | 100.0 | | | |
| pH | | 5.6 | 5.6 | 5.6 | 5.6 | 5.6 | 5.6 | 5.6 | 5.6 | 5.6 | 5.6 |

| | Units | | |
|---|---|---|---|
| Medium components | per liter | 557A | 810D |
| MS BASAL SALT MIXTURE | G | | |
| N6 MACRONUTRIENTS 10X | ml | | |
| POTASSIUM NITRATE | G | | |
| B5H MINOR SALTS 1000X | ml | | |
| NaFe EDTA FOR B5H 100X | ml | | |
| ERIKSSON'S VITAMINS 1000X | ml | | |
| S&H VITAMIN STOCK 100X | ml | | |
| THIAMINE•HCL | mg | | |
| L-PROLINE | g | | |
| CASEIN HYDROLYSATE (ACID) | g | | |
| SUCROSE | g | 20.0 | |
| GLUCOSE | g | | |
| MALTOSE | g | | |
| 2,4-D | mg | | |
| AGAR | g | | |
| PHYTAGEL | g | | |
| DICAMBA | g | | |
| SILVER NITRATE | mg | | |
| AGRIBIO Carbenicillin | mg | | |
| Timentin | mg | | |
| Cefotaxime | mg | | |
| MYO-INOSITOL | g | | |
| NICOTINIC ACID | mg | | |
| PYRIDOXINE•HCL | mg | | |
| VITAMIN ASSAY CASAMINO ACIDS | g | | |
| MES BUFFER | g | | |
| ACETOSYRINGONE | uM | | |
| ASCORBIC ACID 10 MG/ML (7S) | mg | | |
| MS VITAMIN STOCK SOL. | ml | | |
| ZEATIN | mg | | |
| CUPRIC SULFATE | mg | | |
| IAA 0.5 MG/ML (28A) | ml | | |
| ABA 0.1 mm | ml | | |
| THIDIAZURON | mg | | |
| AGRIBIO Carbenicillin | mg | | |
| PPT(GLUFOSINATE-NH4) | mg | | |
| BAP | mg | | |
| YEAST EXTRACT (BD Difco) | g | | 5.0 |
| PEPTONE | g | | 10.0 |
| SODIUM CHLORIDE | g | | 5.0 |
| SPECTINOMYCIN | mg | | 50.0 |
| FERROUS SULFATE•7H20 | ml | | |
| AB BUFFER 20X (12D) | ml | | |
| AB SALTS 20X (12E) | ml | | |
| Benomyl | mg | | |
| POTASSIUM PHOSPHATE DIBASIC | g | 10.5 | |
| POTASSIUM PHOSPHATE MONOBASIC ANHYDROUS | g | 4.5 | |
| AMMONIUM SULFATE | g | 1.0 | |
| SODIUM CITRATE DIHYDRATE | g | 0.5 | |
| MAGNESIUM SULFATE 1.0M | ml | 1.0 | |
| pH | | 5.6 | 6.8 |

SSI events were identified using a multiplex PCR assay to detect the presence/absence of random Agrobacterium T-DNA and vector backbone analysis, and qPCR to determine the excision of the target gene, insertion of FLP (random T-DNA integration), and the FRT pair junction (1/87). The data are summarized in Table 22. A preciseSSl event was identified to have a single copy of the donor gene with intact FRT junctions (1/87), minus the target and FLP having no backbone insertion. The use of the helper plasmid (pVIR9) in the strain LBA4404THY-improved the recovery of SSI events from this strain, compared to the normal SSI recovery using the strain LBA4404THY- without the helper plasmid. In absence of the helper plasmid (pVIR9), LBA4404THY-strain harboring ternary vectors comprising two T-DNA expression cassettes; first T-DNA consisting of ZmUbiPro::FLP::PINII+FRT1-PMI::PINII+ZmUbiPro::DsRED::PINII-FRT87 (pPHP60577; SEQ ID NO.75) and, second T-DNA comprising of; loxP-Rab17Pro::Cre+NosPro::WUS::PINII+ZmUbiPro::BBM::PINII-loxP (pPHP44542; SEQ ID NO.76) resulted in very low SSI frequency (0.1%) in the genotype PHR03 (U.S. Provisional Appl. No. 62/296639, herein incorporated in entirity by reference) compared to the SSI frequency (0.4-0.5%) observed at multiple RTLs in PH184C, a inbred recalcitrant to Agro SSI, with LBA4404THY-71539 strain.

TABLE 22
Transformation frequency and SSI frequency in
PH184C with strain AGL1 and LBA4404 with pVIR9

| | | T0 plants | SSI events | SSI |
|---|---|---|---|---|
| Construct/Target | Agro | (#) | (#) | (%) |
| 79366/ZMRTLA | AGL1 | 11 | 5 | 0.6% |
| 79366/ZMRTLB | AGL1 | 26 | 10 | 1.1% |
| 79366/ZMRTL0C | AGL1 | 39 | 17 | 2.3% |
| 79366/ZMRTLA | LBA4404 + pVIR9 | 6 | 3 | 0.4% |
| 79366/ZMRTLB | LBA4404 + pVIR9 | 19 | 4 | 0.5% |
| 79366/ZMRTLC | LBA4404 + pVIR9 | 32 | 4 | 0.5% |

One skilled in the art would appreciate that incorporation of the helper plasmid pVIR7 (SEQ ID NO: 34) or pVIR10 (SEQ ID NO: 36) in different Agrobacterium strain can enhance the virulence of the Agro strain to improve SSI frequency in multiple corn inbreds.

### Example 13

### Improving Agro SSI using Donor Cassettes and/or Helper Plasmids with Different Origins of Replication

Another way to improve Agro SSI is to build donor cassettes with different bacterial Origins of Replication (Ori) that vary in plasmid copy number (High, medium, low; for eg. RepABC, pRi, pVS1, RK2 etc) allowing to titrate (high to low) the amount of donor DNA molecules delivered into plant cell. Alternatively, the donor cassettes harboring different Ori can be combined with helper plasmids that carry additional virulence genes. These helper plasmids may have different bacterial origins of replication (RepABC, pRi, pVS1, RK2), with varied plasmid copy number. This method may improve the efficiency of SSI event and recovery.

### Example 14

### Sequence Identification Numbers (SEQ ID NO:)

TABLE 23

| SEQID | DESCRIPTION |
|---|---|
| 1 | aacC1 gene; Pseudomonas aeruginosa |
| 2 | ColE1 ori; Escherichia coli |
| 3 | pVS1 ori; Pseudomonas aeruginosa |
| 4 | VirB1; Agrobacterium tumefaciens |
| 5 | VirB2; Agrobacterium tumefaciens |
| 6 | VirB3; Agrobacterium tumefaciens |
| 7 | VirB4; Agrobacterium tumefaciens |
| 8 | VirB5; Agrobacterium tumefaciens |
| 9 | VirB6; Agrobacterium tumefaciens |
| 10 | VirB7; Agrobacterium tumefaciens |
| 11 | VirB8; Agrobacterium tumefaciens |
| 12 | VirB9; Agrobacterium tumefaciens |
| 13 | VirB10; Agrobacterium tumefaciens |
| 14 | VirB11; Agrobacterium tumefaciens |
| 15 | VirG; Agrobacterium tumefaciens |
| 16 | VirC1; Agrobacterium tumefaciens |
| 17 | VirC2; Agrobacterium tumefaciens |
| 18 | VirD1; Agrobacterium tumefaciens |
| 19 | VirD2; Agrobacterium tumefaciens |
| 20 | VirD3; Agrobacterium tumefaciens |
| 21 | VirD4; Agrobacterium tumefaciens |
| 22 | VirD5; Agrobacterium tumefaciens |
| 23 | VirE1 Agrobacterium tumefaciens |
| 24 | VirE2; Agrobacterium tumefaciens |
| 25 | VirE3; Agrobacterium tumefaciens |
| 26 | VirA; Agrobacterium tumefaciens |
| 27 | VirJ; Agrobacterium tumefaciens |
| 28 | PHP45981; Artificial sequence |
| 29 | PHP64484; Artificial sequence |
| 30 | ZmUbiPro; Artificial sequence |
| 31 | DsRED; Discosoma sp. |
| 32 | FRT1; Saccharomyces cerevisiae |
| 33 | FRT87; Saccharomyces cerevisiae |
| 34 | pVIR7; PHP70298; Artificial sequence |
| 35 | pVIR9; PHP71539; Artificial sequence |
| 36 | pVIR10; PHP79761; Artificial sequence |
| 37 | pRSF1010 ori; Escherichia coli |
| 38 | pRK2 ori; Escherichia coli |
| 39 | aadA selection cassette; Escherichia coli |
| 40 | npt1 selection cassette; Escherichia coli |
| 41 | npt2 selection cassette; Escherichia coli |
| 42 | VirH; Agrobacterium tumefaciens |
| 43 | VirH1; Agrobacterium tumefaciens |
| 44 | VirH2; Agrobacterium tumefaciens |
| 45 | VirK; Agrobacterium tumefaciens |
| 46 | VirL; Agrobacterium tumefaciens |
| 47 | VirM; Agrobacterium tumefaciens |
| 48 | VirP; Agrobacterium tumefaciens |
| 49 | VirQ; Agrobacterium tumefaciens |
| 50 | pSC101 ori; Salmonella tymphimurium |
| 51 | p15A ori, Escherichia coli |
| 52 | R6K ori gamma pir; Escherichia coli |
| 53 | pSa repA ori; Escherichia coli |
| 54 | pRK2 micro; Escherichia coli |
| 55 | PARDE; Escherichia coli |
| 56 | pSaPARDE; Artificial sequence |
| 57 | repABC-pRi1724; Agrobacterium rhizogenes |
| 58 | repABC-pTi-SAKURA; Agrobacterium tumefaciens |
| 59 | repABC; PR1b plasmid, Ruegeria sp. |
| 60 | repABC; pNGR234, Sinorhizobium fredii |
| 61 | pSB1 32 bp palindrome; Artificial sequence |
| 62 | pSB1 142 bp inverted repeat; Artificial sequence |
| 63 | pSB1 tra-trb region; Artificial sequence |
| 64 | trfA; Escherichia coli |
| 65 | oriV; Escherichia coli |
| 66 | pRK2 mini (oriV-nptIII-trfA); Escherichia coli |
| 67 | hpt selection cassette; Escherichia coli |
| 68 | UBI-ZMPRO: MO-PAT: PROTEIN LINKER: DS- |
| | RED: PINII |
| 69 | pPHP80561 |
| 70 | pPHP80559 |
| 71 | pPHP79066 |
| 72 | pPHP78147 |
| 73 | pPHP78148 |
| 74 | pPHP79366 |
| 75 | pPHP60577 |
| 76 | pPHP44542 |
| 77 | SpcN; Streptomyces spectabilis |
| 78 | aph; Legionella pneumophila |
| 79 | AR-VIRA; Agrobacterium rhizogenes |
| 80 | AR-VIRB1; Agrobacterium rhizogenes |
| 81 | AR-VIRB2; Agrobacterium rhizogenes |
| 82 | AR-VIRB3; Agrobacterium rhizogenes |
| 83 | AR-VIRB4; Agrobacterium rhizogenes |
| 84 | AR-VIRB5; Agrobacterium rhizogenes |
| 85 | AR-VIRB6; Agrobacterium rhizogenes |
| 86 | AR-VIRB7; Agrobacterium rhizogenes |
| 87 | AR-VIRB8; Agrobacterium rhizogenes |
| 88 | AR-VIRB9; Agrobacterium rhizogenes |
| 89 | AR-VIRB10; Agrobacterium rhizogenes |
| 90 | AR-VIRB11; Agrobacterium rhizogenes |
| 91 | AR-VIRG; Agrobacterium rhizogenes |
| 92 | AR-VIRC1; Agrobacterium rhizogenes |
| 93 | AR-VIRC2; Agrobacterium rhizogenes |
| 94 | AR-VIRD1; Agrobacterium rhizogenes |
| 95 | AR-VIRD2; Agrobacterium rhizogenes |
| 96 | AR-VIRD3; Agrobacterium rhizogenes |
| 97 | AR-VIRBD4; Agrobacterium rhizogenes |
| 98 | AR-VIRD5; Agrobacterium rhizogenes |
| 99 | AR-VIRF; Agrobacterium rhizogenes |
| 100 | AR-VIRE3; Agrobacterium rhizogenes |
| 101 | AR-GALLS; Agrobacterium rhizogenes |
| 102 | BBR1 ori; Bordetella bronchiseptica |

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

All patents, publications and patent applications mentioned in the specification are indicative of the level of those skilled in the art to which this disclosure pertains. All patents, publications and patent applications are herein incorporated by reference in the entirety to the same extent as if each individual patent, publication or patent application was specifically and individually indicated to be incorporated by reference in its entirety.

Although the foregoing disclosure has been described in some detail by way of illustration and example for purposes of clarity of understanding, certain changes and modifications may be practiced within the scope of the appended claims.

1. A vector comprising:
(a) an origin of replication for propagation and stable maintenance in Escherichia coli;
(b) an origin of replication for propagation and stable maintenance in Agrobacterium spp.;
(c) a selectable marker gene; and
(d) Rhizobiaceae virulence genes virB1-811 or r-virB1-B11, virC1-C2 or r-virC1-C2, virD1-D2 or r-virD1-D2, and virG or r-virG, or variants and derivatives thereof, wherein the vector comprising the virulence genes r-virB1-B11, r-virC1-C2, r-virD1-D2, and r-virG further comprises a r-galls virulence gene, or variants and derivatives thereof.

2. The vector of claim 1, wherein the Rhizobiaceae virulence genes are Agrobacterium spp., Rhizobium spp., Sinorhizobium spp., Mesorhizobium spp., Phyllobacterium spp., Ochrobactrum spp., or Bradyrhizobium spp. virulence genes.

3. The vector of claim 2, wherein the Rhizobiaceae virulence genes are Agrobacterium spp. virulence genes.

4. The vector of claim 3, wherein the Agrobacterium spp. virulence genes are Agrobacterium albertimagni, Agrobacterium larrymoorei, Agrobacterium radiobacter, Agrobacterium rhizogenes, Agrobacterium rubi, Agrobacterium tumefaciens, or Agrobacterium vitis virulence genes.

5. The vector of claim 4, wherein the Agrobacterium spp. virulence genes are Agrobacterium rhizogenes or Agrobacterium tumefaciens virulence genes.

6. The vector of claim 4, wherein the Agrobacterium spp. virulence genes are Agrobacterium rhizogenes virulence genes.

7. The vector of claim 4, wherein the Agrobacterium spp. virulence genes are Agrobacterium tumefaciens virulence genes.

8. The vector of claim 1, wherein the Rhizobiaceae virulence genes are virB1-virB11 virulence genes having SEQ ID NOS: 4-14, respectively, or r-virB1-B11 virulence genes having SEQ ID NOS: 80-90, respectively, or variants and derivatives thereof.

9. The vector of claim 1, wherein the Rhizobiaceae virulence genes are virC1-C2 virulence genes having SEQ ID NOS: 16-17, respectively, or r-virC1-C2 virulence genes having SEQ ID NOS: 92-93, respectively, or variants and derivatives thereof.

10. The vector of claim 1, wherein the Rhizobiaceae virulence genes are virD1-D2 virulence genes having SEQ ID NOS: 18-19, respectively, or r-virD1-D2 virulence genes having SEQ ID NOS: 94-95, respectively, or variants and derivatives thereof.

11. The vector of claim 1, wherein the Rhizobiaceae virulence gene is a virG virulence gene having SEQ ID NO: 15, or a r-virG virulence gene having SEQ ID NO: 91, or variants and derivatives thereof.

12. The vector of claim 1, wherein the Rhizobiaceae virulence gene is a r-galls virulence gene having SEQ ID NO: 101, or variants and derivatives thereof.

13. The vector of claim 1, further comprising one or more of Rhizobiaceae virulence genes virA, virD3, virD4, virD5, virE1, virE2, virE3, virH, virH1, virH2, virK, virL, virM, virP, virQ, r-virA , r-virD3, r-virD4, r-virD5, r-virE3, or r-virF or variants and derivatives thereof.

14. The vector of claim 13, wherein the Rhizobiaceae virulence gene is a virA virulence gene having SEQ ID NO: 26 or a r-virA virulence gene having SEQ ID NO: 79, or variants and derivatives thereof.

15. The vector of claim 13, wherein the Rhizobiaceae virulence genes are virD3-D5 virulence genes having SEQ ID NOS: 20-22, respectively, or r-virD3-D5 virulence genes having SEQ ID NO: 96-98, respectively, or variants and derivatives thereof.

16. The vector of claim 13, wherein the Rhizobiaceae virulence genes are virE1-E3 virulence genes having SEQ ID NOS: 23-25, respectively, or a r-virE3 virulence gene having SEQ ID NO: 100, or variants and derivatives thereof.

17. The vector of claim 13, wherein the Rhizobiaceae virulence genes are virH-H1 virulence genes having SEQ ID NOS: 42-43, respectively, or variants and derivatives thereof.

18. The vector of claim 13, wherein the Rhizobiaceae virulence gene is a virK virulence gene having SEQ ID NO: 45, or variants and derivatives thereof.

19. The vector of claim 13, wherein the Rhizobiaceae virulence gene is a virL virulence gene having SEQ ID NO: 46, or variants and derivatives thereof.

20. The vector of claim 13, wherein the Rhizobiaceae virulence gene is a virM virulence gene having SEQ ID NO: 47, or variants and derivatives thereof.

21. The vector of claim 13, wherein the Rhizobiaceae virulence gene is a virP virulence gene having SEQ ID NO: 48, or variants and derivatives thereof.

22. The vector of claim 13, wherein the Rhizobiaceae virulence gene is a virQ virulence gene having SEQ ID NO: 49, or variants and derivatives thereof.

23. The vector of claim 13, comprising the Rhizobiaceae virulence genes virD3-D5 and virE1-E3 or r-virD3-D5 and r-vir E3, or variants and derivatives thereof.

24. The vector of claim 13, comprising the Rhizobiaceae virulence genes virA, virD3-D5, and virE1-E3, or r-virA, r-virD3-D5, and r-virE3, or variants and derivatives thereof.

25. The vector of claim 1, wherein the origin of replication for propagation and stable maintenance in Escherichia coli is derived from a Col E1, a pSC101, a p15A, or a R6K origin of replication, or functional variants and derivatives thereof.

26. The vector of claim 25, wherein the origin of replication for propagation and stable maintenance in Escherichia coli is derived from a Col E1 origin of replication.

27. The vector of claim 26, wherein the origin of replication derived from the ColE1 origin of replication has SEQ ID NO: 2, or variants and fragments thereof.

28. The vector of claim 25, wherein the origin of replication for propagation and stable maintenance in Escherichia coli is derived from a pSC101 origin of replication.

29. The vector of claim 28, wherein the origin of replication derived from the pSC101 origin of replication has SEQ ID NO: 50, or variants and fragments thereof.

30. The vector of claim 25, wherein the origin of replication for propagation and stable maintenance in Escherichia coli is derived from a p15A origin of replication.

31. The vector of claim 30, wherein the origin of replication derived from the p15A origin of replication has SEQ ID NO: 51, or variants and fragments thereof.

32. The vector of claim 25, wherein the origin of replication for propagation and stable maintenance in Escherichia coli is derived from a R6K origin of replication.

33. The vector of claim 32, wherein the origin of replication derived from the R6K origin of replication has SEQ ID NO: 52, or variants and fragments thereof.

34. The vector of claim 1, wherein the origin of replication for propagation and stable maintenance in Agrobacterium spp. is a high copy number origin of replication.

35. The vector of claim 1, wherein the origin of replication for propagation and stable maintenance in Agrobacterium spp. is an intermediate copy number origin of replication.

36. The vector of claim 1, wherein the origin of replication for propagation and stable maintenance in Agrobacterium spp. is a low copy number origin of replication.

37. The vector of claim 1, wherein the origin of replication for propagation and stable maintenance in Agrobacterium spp. is derived from a pRi, a pVS1, a pRSF1010, a pRK2, a pSa, or a pBBR1 origin of replication.

38. The vector of claim 37, wherein the origin of replication for propagation and stable maintenance in Agrobacterium spp. is a variant of the pRK2 origin of replication.

39. The vector of claim 37, wherein the origin of replication for propagation and stable maintenance in Agrobacterium spp. is derived from the pRSF1010 origin of replication.

40. The vector of claim 37, wherein the origin of replication for propagation and stable maintenance in Agrobacterium spp. is derived from the pVS1 origin of replication.

41. The vector of claim 37, wherein the origin of replication for propagation and stable maintenance in Agrobacterium spp. is derived from the pSa origin of replication.

42. The vector of claim 37, wherein the origin of replication for propagation and stable maintenance in Agrobacterium spp. is an origin of replication having any one of SEQ ID NOS: 3, 37, 38, 53, 57, 58, 59, or 60, or variants and fragments thereof.

43. The vector of claim 1, wherein the origin of replication for propagation and stable maintenance in Agrobacterium spp. is a repABC compatible origin of replication.

44. The vector of claim 43, wherein the repABC compatible origin of replication has any one of SEQ ID NOS: 57, 58, 59, or 60, or variants and fragments thereof.

45. The vector of claim 1, wherein the origin of replication for propagation and stable maintenance in Escherichia coli and the origin of replication for propagation and stable maintenance in Agrobacterium spp. are the same origin of replication.

46. The vector of claim 45, wherein the origin of replication is derived from a pRK2 origin of replication, from a pSa origin of replication, or a pRSF1010 origin of replication.

47. The vector of claim 46, wherein the origin of replication is derived from the pRK2 origin of replication.

48. The vector of claim 47, wherein the pRK2 origin of replication has SEQ ID NO: 38, or variants and fragments thereof.

49. The vector of claim 47, wherein the pRK2 origin of replication is a mini or micro pRK2 origin of replication.

50. The vector of claim 47, wherein the pRK2 origin of replication is a micro pRK2 origin of replication.

51. The vector of claim 50, wherein the micro pRK2 origin of replication has SEQ ID NO: 54, or variants and fragments thereof.

52. The vector of claim 47, wherein the pRK2 origin of replication is a mini pRK2 origin of replication.

53. The vector of claim 52, wherein the mini pRK2 has SEQ ID NO: 66, or variants and fragments thereof.

54. The vector of claim 47, wherein the pRK2 origin of replication comprises the trfA and OriV sequences.

55. The vector of claim 54, wherein the pRK2 origin of replication comprises SEQ ID NOS: 64 and 65, or variants and fragments thereof.

56. The vector of claim 46, wherein the origin of replication is derived from the pSa origin of replication.

57. The vector of claim 56, wherein the pSa origin of replication has SEQ ID NO: 53, or variants and fragments thereof.

58. The vector of claim 46, wherein the origin of replication is derived from the pRSF1010 origin of replication.

59. The vector of claim 58, wherein the pRSF1010 origin of replication has SEQ ID NO: 37, or variants and fragments thereof.

60. The vector of claim 47, further comprising a sequence derived from the par DE operon.

61. The vector of claim 60, wherein the par DE operon has SEQ ID NO: 55, or variants and fragments thereof.

62. The vector of claim 1, wherein the selectable marker gene provides resistance to gentamicin, neomycin/kanamycin, hygromycin, or spectinomycin.

63. The vector of claim 62, wherein the selectable marker gene is an aacC1 gene, a npt1gene, a npt2 gene, a hpt gene, an aadA gene, a SpcN gene, or an aph gene.

64. The vector of claim 63, wherein the selectable marker gene is aacC1.

65. The vector of claim 64, wherein the aacC1 selectable marker gene has SEQ ID NO: 1, or variants and fragments thereof.

66. The vector of claim 63, wherein the selectable marker gene is aadA.

67. The vector of claim 66, wherein the aadA selectable marker gene has SEQ ID NO: 39, or variants and fragments thereof.

68. The vector of claim 63, wherein the selectable marker gene is npt1.

69. The vector of claim 68, wherein the nptl selectable marker gene has SEQ ID NO: 40, or variants and fragments thereof.

70. The vector of claim 63, wherein the selectable marker gene is npt2.

71. The vector of claim 70, wherein the npt2 selectable marker gene has SEQ ID NO: 41, or variants and fragments thereof.

72. The vector of claim 63, wherein the selectable marker gene is hpt.

73. The vector of claim 72, wherein the hpt selectable marker gene has SEQ ID NO: 67, or variants and fragments thereof.

74. The vector of claim 63, wherein the selectable marker gene is SpcN.

75. The vector of claim 74, wherein the SpcN selectable marker gene has SEQ ID NO: 77, or variants and fragments thereof.

76. The vector of claim 63, wherein the selectable marker gene is aph.

77. The vector of claim 76, wherein the aph selectable marker gene has SEQ ID NO: 78, or variants and fragments thereof.

78. The vector of claim 1, wherein the selectable marker gene does not provide resistance to tetracycline.

79. The vector of claim 1, wherein the selectable marker gene is not a tetAR gene.

80. The vector of claim 1, wherein the selectable marker gene is a counter-selectable marker gene.

81. The vector of claim 80, wherein the counter-selectable marker gene is a sacB gene, a rpsL (strA) gene, a pheS gene, a dhfr (folA) gene, a lacY gene, a Gata-1 gene, a ccdB gene, or a thyA− gene.

82. The vector of any one of claims 80, wherein the vector does not comprise SEQ ID NO: 61, or variants or fragments thereof.

83. The vector of claim 1, wherein the vector does not comprise SEQ ID NO: 62, or variants or fragments thereof.

84. The vector of claim 1, wherein the vector does not comprise a tra operon sequence or a trb operon sequence, or variants or fragments thereof.

85. The vector of claim 84, wherein the vector does not comprise SEQ ID NO: 63, or variants or fragments thereof.

86. The vector of claim 1, wherein the vector has SEQ ID NO: 34, or variants and fragments thereof.

87. The vector of claim 1, wherein the vector has SEQ ID NO: 35, or variants and fragments thereof.

88. The vector of claim 1, wherein the vector has SEQ ID NO: 36, or variants and fragments thereof.

89. A vector comprising:
(a) an origin of replication for propagation in Escherichia coli having SEQ ID NO: 2, or variants and fragments thereof;
(b) an origin of replication for propagation in Agrobacterium spp. having SEQ ID NO: 3, or variants and fragments thereof;
(c) a selectable marker gene having SEQ ID NO: 1, or variants and fragments thereof; and
(d) virulence genes comprising Agrobacterium spp. virulence genes virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D2 virulence genes having SEQ ID NOS: 18-19, respectively or r-virD1-D2 virulence genes having SEQ ID NOS: 94-95, respectively, and a virG virulence gene having SEQ ID NO: 15 or a r-virG virulence gene having SEQ ID NO: 91, or variants and derivatives thereof, wherein the vector comprising the virulence genes r-virB1-B11, r-virC1-C2, r-virD1-D2, and r-virG further comprises a r-galls virulence gene having SEQ ID NO: 101, or variants and derivatives thereof.

90. A vector comprising:
(a) an origin of replication for propagation in Escherichia coli having SEQ ID NO: 2, or variants and fragments thereof;
(b) an origin of replication for propagation in Agrobacterium spp. having SEQ ID NO: 3, or variants and fragments thereof;
(c) a selectable marker gene having SEQ ID NO: 1, or variants and fragments thereof; and
(d) virulence genes comprising Agrobacterium spp. virulence genes virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D5 virulence genes having SEQ ID NOS: 18-22, respectively or r-virD1-D5 virulence genes having SEQ ID NOS: 94-98, respectively, virE1-E3 virulence genes having SEQ ID NOS: 23-25, respectively or a r-virE3 virulence gene having SEQ ID NO: 100, and a virG virulence gene having SEQ ID NO: 15 or a r-virG virulence gene having SEQ ID NO: 91, or variants and derivatives thereof, wherein the vector comprising the virulence genes r-virB1-B11, r-virC1-C2, r-virD1-D5, r-virE3, and r-virG further comprises a r-galls virulence gene having SEQ ID NO: 101, or variants and derivatives thereof.

91. A vector comprising:
(a) an origin of replication for propagation in Escherichia coli having SEQ ID NO: 2, or variants and fragments thereof;
(b) an origin of replication for propagation in Agrobacterium spp. having SEQ ID NO: 3, or variants and fragments thereof;
(c) a selectable marker gene having SEQ ID NO: 1; and
(d) virulence genes comprising Agrobacterium spp. virulence genes a virA virulence gene having SEQ ID NO: 26 or a r-virA virulence gene having SEQ ID NO: 79, virB1-B11 virulence genes having SEQ ID NOS: 4-14, respectively or r-virB1-B11 virulence genes having SEQ ID NOS: 80-90, respectively, virC1-C2 virulence genes having SEQ ID NOS: 16-17, respectively or r-virC1-C2 virulence genes having SEQ ID NOS: 92-93, respectively, virD1-D5 virulence genes having SEQ ID NOS: 18-22, respectively or r-virD1-D5 virulence genes having SEQ ID NOS: 94-98, respectively, virE1-E3 virulence genes having SEQ ID NOS: 23-25, respectively or a r-virE3 virulence gene having SEQ ID NOS: 100, and a virG virulence gene having SEQ ID NO: 15 or a r-virG virulence gene having SEQ ID NO: 91, or variants and derivatives thereof, wherein the vector comprising the virulence genes r-virA, r-virB1-B11, r-virC1-C2, r-virD1-D5, r-virE3, and r-virG further comprises a r-galls virulence gene having SEQ ID NO: 101, or variants and derivatives thereof.

92. A method for transformation of a plant comprising the steps of:
(a) contacting a tissue from the plant with an Agrobacterium strain or an Ochrobactrum strain comprising a first vector of claim 1, and a second vector comprising T-DNA borders and a polynucleotide sequence of interest for transfer to the plant;
(b) co-cultivating the tissue with the Agrobacterium strain or the Ochrobactrum strain; and
(c) regenerating a transformed plant from the tissue that expresses the polynucleotide sequence of interest.

93. A kit comprising:
(a) a vector of claim 1; and
(b) instructions for use in transformation of a plant using Agrobacterium or Ochrobactrum.
